SEQUENCE LISTING (167 sequences)

SEQ ID NO: 1 - protein, 301 aa, unidentified
  source 1..301: note = Aspergillus Flavus
SAVKAARYGKDNVRVYKVHKDEKTGVQTVYEMTVCVLLEGEIETSYTKADNSVIVATDSIKNTIYITAKQNPVTPPELFGSILGTHFIEKYNHIHAAHVNIVCHRWTRMDIDGKPHPHSFIRDSEEKRNVQVDVVEGKGIDIKSSLSGLTVLKSTNSQFWGFLRDEYTTLKETWDRILSTDVDATWQWKNFSGLQEVRSHVPKFDATWATAREVTLKTFAEDNSASVQATMYKMAEQILARQQLIETVEYSLPNKHYFEIDLSWHKGLQNTGKNAEVFAPQSDPNGLIKCTVGRSSLKSKL

SEQ ID NO: 2 - protein, 301 aa, synthetic construct
  source 1..301: note = Asp.Uox Variant Subunit (G137)
  VARIANT 137: note = Xaa is unnatural amino acid containing tetrazine functional group, and/or triazine functional group
SAVKAARYGKDNVRVYKVHKDEKTGVQTVYEMTVCVLLEGEIETSYTKADNSVIVATDSIKNTIYITAKQNPVTPPELFGSILGTHFIEKYNHIHAAHVNIVCHRWTRMDIDGKPHPHSFIRDSEEKRNVQVDVVEXKGIDIKSSLSGLTVLKSTNSQFWGFLRDEYTTLKETWDRILSTDVDATWQWKNFSGLQEVRSHVPKFDATWATAREVTLKTFAEDNSASVQATMYKMAEQILARQQLIETVEYSLPNKHYFEIDLSWHKGLQNTGKNAEVFAPQSDPNGLIKCTVGRSSLKSKL

SEQ ID NO: 3 - protein, 301 aa, synthetic construct
  source 1..301: note = Asp. Uox Variant Subunit (E22)
  VARIANT 22: note = Xaa is unnatural amino acid containing tetrazine functionalgroup, and/or triazine functional group.
SAVKAARYGKDNVRVYKVHKDXKTGVQTVYEMTVCVLLEGEIETSYTKADNSVIVATDSIKNTIYITAKQNPVTPPELFGSILGTHFIEKYNHIHAAHVNIVCHRWTRMDIDGKPHPHSFIRDSEEKRNVQVDVVEGKGIDIKSSLSGLTVLKSTNSQFWGFLRDEYTTLKETWDRILSTDVDATWQWKNFSGLQEVRSHVPKFDATWATAREVTLKTFAEDNSASVQATMYKMAEQILARQQLIETVEYSLPNKHYFEIDLSWHKGLQNTGKNAEVFAPQSDPNGLIKCTVGRSSLKSKL

SEQ ID NO: 4 - protein, 301 aa, synthetic construct
  source 1..301: note = Asp. Uox Variant Subunit (N92)
  VARIANT 92: note = Xaa is unnatural amino acid containing tetrazine functionalgroup, and/or triazine functional group.
SAVKAARYGKDNVRVYKVHKDEKTGVQTVYEMTVCVLLEGEIETSYTKADNSVIVATDSIKNTIYITAKQNPVTPPELFGSILGTHFIEKYXHIHAAHVNIVCHRWTRMDIDGKPHPHSFIRDSEEKRNVQVDVVEGKGIDIKSSLSGLTVLKSTNSQFWGFLRDEYTTLKETWDRILSTDVDATWQWKNFSGLQEVRSHVPKFDATWATAREVTLKTFAEDNSASVQATMYKMAEQILARQQLIETVEYSLPNKHYFEIDLSWHKGLQNTGKNAEVFAPQSDPNGLIKCTVGRSSLKSKL

SEQ ID NO: 5 - protein, 301 aa, synthetic construct
  source 1..301: note = Asp. Uox Variant Subunit (K23)
  VARIANT 23: note = Xaa is unnatural amino acid containing tetrazine functionalgroup, and/or triazine functional group.
SAVKAARYGKDNVRVYKVHKDEXTGVQTVYEMTVCVLLEGEIETSYTKADNSVIVATDSIKNTIYITAKQNPVTPPELFGSILGTHFIEKYNHIHAAHVNIVCHRWTRMDIDGKPHPHSFIRDSEEKRNVQVDVVEGKGIDIKSSLSGLTVLKSTNSQFWGFLRDEYTTLKETWDRILSTDVDATWQWKNFSGLQEVRSHVPKFDATWATAREVTLKTFAEDNSASVQATMYKMAEQILARQQLIETVEYSLPNKHYFEIDLSWHKGLQNTGKNAEVFAPQSDPNGLIKCTVGRSSLKSKL

SEQ ID NO: 6 - protein, 301 aa, synthetic construct
  source 1..301: note = Asp. Uox Variant Subunit (S295)
  VARIANT 295: note = Xaa is unnatural amino acid containing tetrazine functionalgroup, and/or triazine functional group.
SAVKAARYGKDNVRVYKVHKDEKTGVQTVYEMTVCVLLEGEIETSYTKADNSVIVATDSIKNTIYITAKQNPVTPPELFGSILGTHFIEKYNHIHAAHVNIVCHRWTRMDIDGKPHPHSFIRDSEEKRNVQVDVVEGKGIDIKSSLSGLTVLKSTNSQFWGFLRDEYTTLKETWDRILSTDVDATWQWKNFSGLQEVRSHVPKFDATWATAREVTLKTFAEDNSASVQATMYKMAEQILARQQLIETVEYSLPNKHYFEIDLSWHKGLQNTGKNAEVFAPQSDPNGLIKCTVGRXSLKSKL

SEQ ID NO: 7 - protein, 301 aa, synthetic construct
  source 1..301: note = Asp.Uox Variant Subunit (G113)
  VARIANT 113: note = Xaa is unnatural amino acid containing tetrazine functionalgroup, and/or triazine functional group.
SAVKAARYGKDNVRVYKVHKDEKTGVQTVYEMTVCVLLEGEIETSYTKADNSVIVATDSIKNTIYITAKQNPVTPPELFGSILGTHFIEKYNHIHAAHVNIVCHRWTRMDIDXKPHPHSFIRDSEEKRNVQVDVVEGKGIDIKSSLSGLTVLKSTNSQFWGFLRDEYTTLKETWDRILSTDVDATWQWKNFSGLQEVRSHVPKFDATWATAREVTLKTFAEDNSASVQATMYKMAEQILARQQLIETVEYSLPNKHYFEIDLSWHKGLQNTGKNAEVFAPQSDPNGLIKCTVGRSSLKSKL

SEQ ID NO: 8 - protein, 301 aa, synthetic construct
  source 1..301: note = Asp.Uox Variant Subunit (K273)
  VARIANT 273: note = Xaa is unnatural amino acid containing tetrazine functionalgroup, and/or triazine functional group.
SAVKAARYGKDNVRVYKVHKDEKTGVQTVYEMTVCVLLEGEIETSYTKADNSVIVATDSIKNTIYITAKQNPVTPPELFGSILGTHFIEKYNHIHAAHVNIVCHRWTRMDIDGKPHPHSFIRDSEEKRNVQVDVVEGKGIDIKSSLSGLTVLKSTNSQFWGFLRDEYTTLKETWDRILSTDVDATWQWKNFSGLQEVRSHVPKFDATWATAREVTLKTFAEDNSASVQATMYKMAEQILARQQLIETVEYSLPNKHYFEIDLSWHKGLQNTGXNAEVFAPQSDPNGLIKCTVGRSSLKSKL

SEQ ID NO: 9 - protein, 301 aa, synthetic construct
  source 1..301: note = Asp.Uox Variant Subunit (K171)
  VARIANT 171: note = Xaa is unnatural amino acid containing tetrazine functionalgroup, and/or triazine functional group.
SAVKAARYGKDNVRVYKVHKDEKTGVQTVYEMTVCVLLEGEIETSYTKADNSVIVATDSIKNTIYITAKQNPVTPPELFGSILGTHFIEKYNHIHAAHVNIVCHRWTRMDIDGKPHPHSFIRDSEEKRNVQVDVVEGKGIDIKSSLSGLTVLKSTNSQFWGFLRDEYTTLXETWDRILSTDVDATWQWKNFSGLQEVRSHVPKFDATWATAREVTLKTFAEDNSASVQATMYKMAEQILARQQLIETVEYSLPNKHYFEIDLSWHKGLQNTGKNAEVFAPQSDPNGLIKCTVGRSSLKSKL

SEQ ID NO: 10 - protein, 301 aa, synthetic construct
  source 1..301: note = Asp.Uox Variant Subunit (A240)
  VARIANT 240: note = Xaa is unnatural amino acid containing tetrazine functionalgroup, and/or triazine functional group.
SAVKAARYGKDNVRVYKVHKDEKTGVQTVYEMTVCVLLEGEIETSYTKADNSVIVATDSIKNTIYITAKQNPVTPPELFGSILGTHFIEKYNHIHAAHVNIVCHRWTRMDIDGKPHPHSFIRDSEEKRNVQVDVVEGKGIDIKSSLSGLTVLKSTNSQFWGFLRDEYTTLKETWDRILSTDVDATWQWKNFSGLQEVRSHVPKFDATWATAREVTLKTFAEDNSASVQATMYKMAEQILXRQQLIETVEYSLPNKHYFEIDLSWHKGLQNTGKNAEVFAPQSDPNGLIKCTVGRSSLKSKL

SEQ ID NO: 11 - protein, 301 aa, synthetic construct
  source 1..301: note = Asp.Uox Variant Subunit (E89)
  VARIANT 89: note = Xaa is unnatural amino acid containing tetrazine functionalgroup, and/or triazine functional group.
SAVKAARYGKDNVRVYKVHKDEKTGVQTVYEMTVCVLLEGEIETSYTKADNSVIVATDSIKNTIYITAKQNPVTPPELFGSILGTHFIXKYNHIHAAHVNIVCHRWTRMDIDGKPHPHSFIRDSEEKRNVQVDVVEGKGIDIKSSLSGLTVLKSTNSQFWGFLRDEYTTLKETWDRILSTDVDATWQWKNFSGLQEVRSHVPKFDATWATAREVTLKTFAEDNSASVQATMYKMAEQILARQQLIETVEYSLPNKHYFEIDLSWHKGLQNTGKNAEVFAPQSDPNGLIKCTVGRSSLKSKL

SEQ ID NO: 12 - protein, 301 aa, synthetic construct
  source 1..301: note = Asp.Uox Variant Subunit (K266)
  VARIANT 266: note = Xaa is unnatural amino acid containing tetrazine functionalgroup, and/or triazine functional group.
SAVKAARYGKDNVRVYKVHKDEKTGVQTVYEMTVCVLLEGEIETSYTKADNSVIVATDSIKNTIYITAKQNPVTPPELFGSILGTHFIEKYNHIHAAHVNIVCHRWTRMDIDGKPHPHSFIRDSEEKRNVQVDVVEGKGIDIKSSLSGLTVLKSTNSQFWGFLRDEYTTLKETWDRILSTDVDATWQWKNFSGLQEVRSHVPKFDATWATAREVTLKTFAEDNSASVQATMYKMAEQILARQQLIETVEYSLPNKHYFEIDLSWHXGLQNTGKNAEVFAPQSDPNGLIKCTVGRSSLKSKL

SEQ ID NO: 13 - protein, 301 aa, synthetic construct
  source 1..301: note = Asp.Uox Variant Subunit (T24)
  VARIANT 24: note = Xaa is unnatural amino acid containing tetrazine functionalgroup, and/or triazine functional group.
SAVKAARYGKDNVRVYKVHKDEKXGVQTVYEMTVCVLLEGEIETSYTKADNSVIVATDSIKNTIYITAKQNPVTPPELFGSILGTHFIEKYNHIHAAHVNIVCHRWTRMDIDGKPHPHSFIRDSEEKRNVQVDVVEGKGIDIKSSLSGLTVLKSTNSQFWGFLRDEYTTLKETWDRILSTDVDATWQWKNFSGLQEVRSHVPKFDATWATAREVTLKTFAEDNSASVQATMYKMAEQILARQQLIETVEYSLPNKHYFEIDLSWHKGLQNTGKNAEVFAPQSDPNGLIKCTVGRSSLKSKL

SEQ ID NO: 14 - protein, 301 aa, synthetic construct
  source 1..301: note = Asp.Uox Variant Subunit (K48)
  VARIANT 48: note = Xaa is unnatural amino acid containing tetrazine functionalgroup, and/or triazine functional group.
SAVKAARYGKDNVRVYKVHKDEKTGVQTVYEMTVCVLLEGEIETSYTXADNSVIVATDSIKNTIYITAKQNPVTPPELFGSILGTHFIEKYNHIHAAHVNIVCHRWTRMDIDGKPHPHSFIRDSEEKRNVQVDVVEGKGIDIKSSLSGLTVLKSTNSQFWGFLRDEYTTLKETWDRILSTDVDATWQWKNFSGLQEVRSHVPKFDATWATAREVTLKTFAEDNSASVQATMYKMAEQILARQQLIETVEYSLPNKHYFEIDLSWHKGLQNTGKNAEVFAPQSDPNGLIKCTVGRSSLKSKL

SEQ ID NO: 15 - protein, 301 aa, synthetic construct
  source 1..301: note = Asp.Uox Variant Subunit (S192)
  VARIANT 192: note = Xaa is unnatural amino acid containing tetrazine functionalgroup, and/or triazine functional group.
SAVKAARYGKDNVRVYKVHKDEKTGVQTVYEMTVCVLLEGEIETSYTKADNSVIVATDSIKNTIYITAKQNPVTPPELFGSILGTHFIEKYNHIHAAHVNIVCHRWTRMDIDGKPHPHSFIRDSEEKRNVQVDVVEGKGIDIKSSLSGLTVLKSTNSQFWGFLRDEYTTLKETWDRILSTDVDATWQWKNFXGLQEVRSHVPKFDATWATAREVTLKTFAEDNSASVQATMYKMAEQILARQQLIETVEYSLPNKHYFEIDLSWHKGLQNTGKNAEVFAPQSDPNGLIKCTVGRSSLKSKL

SEQ ID NO: 16 - protein, 301 aa, synthetic construct
  source 1..301: note = Asp.Uox Variant Subunit (P202)
  VARIANT 202: note = Xaa is unnatural amino acid containing tetrazine functionalgroup, and/or triazine functional group.
SAVKAARYGKDNVRVYKVHKDEKTGVQTVYEMTVCVLLEGEIETSYTKADNSVIVATDSIKNTIYITAKQNPVTPPELFGSILGTHFIEKYNHIHAAHVNIVCHRWTRMDIDGKPHPHSFIRDSEEKRNVQVDVVEGKGIDIKSSLSGLTVLKSTNSQFWGFLRDEYTTLKETWDRILSTDVDATWQWKNFSGLQEVRSHVXKFDATWATAREVTLKTFAEDNSASVQATMYKMAEQILARQQLIETVEYSLPNKHYFEIDLSWHKGLQNTGKNAEVFAPQSDPNGLIKCTVGRSSLKSKL

SEQ ID NO: 17 - protein, 301 aa, synthetic construct
  source 1..301: note = Asp.Uox Variant Subunit (D110)
  VARIANT 110: note = Xaa is unnatural amino acid containing tetrazine functionalgroup, and/or triazine functional group.
SAVKAARYGKDNVRVYKVHKDEKTGVQTVYEMTVCVLLEGEIETSYTKADNSVIVATDSIKNTIYITAKQNPVTPPELFGSILGTHFIEKYNHIHAAHVNIVCHRWTRMXIDGKPHPHSFIRDSEEKRNVQVDVVEGKGIDIKSSLSGLTVLKSTNSQFWGFLRDEYTTLKETWDRILSTDVDATWQWKNFSGLQEVRSHVPKFDATWATAREVTLKTFAEDNSASVQATMYKMAEQILARQQLIETVEYSLPNKHYFEIDLSWHKGLQNTGKNAEVFAPQSDPNGLIKCTVGRSSLKSKL

SEQ ID NO: 18 - protein, 301 aa, synthetic construct
  source 1..301: note = Asp.Uox Variant Subunit (Q243)
  VARIANT 243: note = Xaa is unnatural amino acid containing tetrazine functionalgroup, and/or triazine functional group.
SAVKAARYGKDNVRVYKVHKDEKTGVQTVYEMTVCVLLEGEIETSYTKADNSVIVATDSIKNTIYITAKQNPVTPPELFGSILGTHFIEKYNHIHAAHVNIVCHRWTRMDIDGKPHPHSFIRDSEEKRNVQVDVVEGKGIDIKSSLSGLTVLKSTNSQFWGFLRDEYTTLKETWDRILSTDVDATWQWKNFSGLQEVRSHVPKFDATWATAREVTLKTFAEDNSASVQATMYKMAEQILARQXLIETVEYSLPNKHYFEIDLSWHKGLQNTGKNAEVFAPQSDPNGLIKCTVGRSSLKSKL

SEQ ID NO: 19 - protein, 301 aa, synthetic construct
  source 1..301: note = Asp.Uox Variant Subunit (Q195)
  VARIANT 195: note = Xaa is unnatural amino acid containing tetrazine functionalgroup, and/or triazine functional group.
SAVKAARYGKDNVRVYKVHKDEKTGVQTVYEMTVCVLLEGEIETSYTKADNSVIVATDSIKNTIYITAKQNPVTPPELFGSILGTHFIEKYNHIHAAHVNIVCHRWTRMDIDGKPHPHSFIRDSEEKRNVQVDVVEGKGIDIKSSLSGLTVLKSTNSQFWGFLRDEYTTLKETWDRILSTDVDATWQWKNFSGLXEVRSHVPKFDATWATAREVTLKTFAEDNSASVQATMYKMAEQILARQQLIETVEYSLPNKHYFEIDLSWHKGLQNTGKNAEVFAPQSDPNGLIKCTVGRSSLKSKL

SEQ ID NO: 20 - protein, 301 aa, synthetic construct
  source 1..301: note = Asp.Uox Variant Subunit (K138)
  VARIANT 138: note = Xaa is unnatural amino acid containing tetrazine functionalgroup, and/or triazine functional group.
SAVKAARYGKDNVRVYKVHKDEKTGVQTVYEMTVCVLLEGEIETSYTKADNSVIVATDSIKNTIYITAKQNPVTPPELFGSILGTHFIEKYNHIHAAHVNIVCHRWTRMDIDGKPHPHSFIRDSEEKRNVQVDVVEGXGIDIKSSLSGLTVLKSTNSQFWGFLRDEYTTLKETWDRILSTDVDATWQWKNFSGLQEVRSHVPKFDATWATAREVTLKTFAEDNSASVQATMYKMAEQILARQQLIETVEYSLPNKHYFEIDLSWHKGLQNTGKNAEVFAPQSDPNGLIKCTVGRSSLKSKL

SEQ ID NO: 21 - protein, 301 aa, synthetic construct
  source 1..301: note = Asp.Uox Variant Subunit (P115)
  VARIANT 115: note = Xaa is unnatural amino acid containing tetrazine functionalgroup, and/or triazine functional group.
SAVKAARYGKDNVRVYKVHKDEKTGVQTVYEMTVCVLLEGEIETSYTKADNSVIVATDSIKNTIYITAKQNPVTPPELFGSILGTHFIEKYNHIHAAHVNIVCHRWTRMDIDGKXHPHSFIRDSEEKRNVQVDVVEGKGIDIKSSLSGLTVLKSTNSQFWGFLRDEYTTLKETWDRILSTDVDATWQWKNFSGLQEVRSHVPKFDATWATAREVTLKTFAEDNSASVQATMYKMAEQILARQQLIETVEYSLPNKHYFEIDLSWHKGLQNTGKNAEVFAPQSDPNGLIKCTVGRSSLKSKL

SEQ ID NO: 22 - protein, 301 aa, synthetic construct
  source 1..301: note = Asp.Uox Variant Subunit (S199)
  VARIANT 199: note = Xaa is unnatural amino acid containing tetrazine functionalgroup, and/or triazine functional group.
SAVKAARYGKDNVRVYKVHKDEKTGVQTVYEMTVCVLLEGEIETSYTKADNSVIVATDSIKNTIYITAKQNPVTPPELFGSILGTHFIEKYNHIHAAHVNIVCHRWTRMDIDGKPHPHSFIRDSEEKRNVQVDVVEGKGIDIKSSLSGLTVLKSTNSQFWGFLRDEYTTLKETWDRILSTDVDATWQWKNFSGLQEVRXHVPKFDATWATAREVTLKTFAEDNSASVQATMYKMAEQILARQQLIETVEYSLPNKHYFEIDLSWHKGLQNTGKNAEVFAPQSDPNGLIKCTVGRSSLKSKL

SEQ ID NO: 23 - protein, 301 aa, synthetic construct
  source 1..301: note = Asp.Uox Variant Subunit (G272)
  VARIANT 272: note = Xaa is unnatural amino acid containing tetrazine functionalgroup, and/or triazine functional group.
SAVKAARYGKDNVRVYKVHKDEKTGVQTVYEMTVCVLLEGEIETSYTKADNSVIVATDSIKNTIYITAKQNPVTPPELFGSILGTHFIEKYNHIHAAHVNIVCHRWTRMDIDGKPHPHSFIRDSEEKRNVQVDVVEGKGIDIKSSLSGLTVLKSTNSQFWGFLRDEYTTLKETWDRILSTDVDATWQWKNFSGLQEVRSHVPKFDATWATAREVTLKTFAEDNSASVQATMYKMAEQILARQQLIETVEYSLPNKHYFEIDLSWHKGLQNTXKNAEVFAPQSDPNGLIKCTVGRSSLKSKL

SEQ ID NO: 24 - protein, 301 aa, synthetic construct
  source 1..301: note = Asp.Uox Variant Subunit (K4)
  VARIANT 4: note = Xaa is unnatural amino acid containing tetrazine functionalgroup, and/or triazine functional group.
SAVXAARYGKDNVRVYKVHKDEKTGVQTVYEMTVCVLLEGEIETSYTKADNSVIVATDSIKNTIYITAKQNPVTPPELFGSILGTHFIEKYNHIHAAHVNIVCHRWTRMDIDGKPHPHSFIRDSEEKRNVQVDVVEGKGIDIKSSLSGLTVLKSTNSQFWGFLRDEYTTLKETWDRILSTDVDATWQWKNFSGLQEVRSHVPKFDATWATAREVTLKTFAEDNSASVQATMYKMAEQILARQQLIETVEYSLPNKHYFEIDLSWHKGLQNTGKNAEVFAPQSDPNGLIKCTVGRSSLKSKL

SEQ ID NO: 25 - protein, 301 aa, synthetic construct
  source 1..301: note = Asp.Uox Variant Subunit (D112)
  VARIANT 112: note = Xaa is unnatural amino acid containing tetrazine functionalgroup, and/or triazine functional group.
SAVKAARYGKDNVRVYKVHKDEKTGVQTVYEMTVCVLLEGEIETSYTKADNSVIVATDSIKNTIYITAKQNPVTPPELFGSILGTHFIEKYNHIHAAHVNIVCHRWTRMDIXGKPHPHSFIRDSEEKRNVQVDVVEGKGIDIKSSLSGLTVLKSTNSQFWGFLRDEYTTLKETWDRILSTDVDATWQWKNFSGLQEVRSHVPKFDATWATAREVTLKTFAEDNSASVQATMYKMAEQILARQQLIETVEYSLPNKHYFEIDLSWHKGLQNTGKNAEVFAPQSDPNGLIKCTVGRSSLKSKL

SEQ ID NO: 26 - protein, 301 aa, synthetic construct
  source 1..301: note = Asp.Uox Variant Subunit (G267)
  VARIANT 267: note = Xaa is unnatural amino acid containing tetrazine functionalgroup, and/or triazine functional group.
SAVKAARYGKDNVRVYKVHKDEKTGVQTVYEMTVCVLLEGEIETSYTKADNSVIVATDSIKNTIYITAKQNPVTPPELFGSILGTHFIEKYNHIHAAHVNIVCHRWTRMDIDGKPHPHSFIRDSEEKRNVQVDVVEGKGIDIKSSLSGLTVLKSTNSQFWGFLRDEYTTLKETWDRILSTDVDATWQWKNFSGLQEVRSHVPKFDATWATAREVTLKTFAEDNSASVQATMYKMAEQILARQQLIETVEYSLPNKHYFEIDLSWHKXLQNTGKNAEVFAPQSDPNGLIKCTVGRSSLKSKL

SEQ ID NO: 27 - protein, 301 aa, synthetic construct
  source 1..301: note = Asp.Uox Variant Subunit (K114)
  VARIANT 114: note = Xaa is unnatural amino acid containing tetrazine functionalgroup, and/or triazine functional group.
SAVKAARYGKDNVRVYKVHKDEKTGVQTVYEMTVCVLLEGEIETSYTKADNSVIVATDSIKNTIYITAKQNPVTPPELFGSILGTHFIEKYNHIHAAHVNIVCHRWTRMDIDGXPHPHSFIRDSEEKRNVQVDVVEGKGIDIKSSLSGLTVLKSTNSQFWGFLRDEYTTLKETWDRILSTDVDATWQWKNFSGLQEVRSHVPKFDATWATAREVTLKTFAEDNSASVQATMYKMAEQILARQQLIETVEYSLPNKHYFEIDLSWHKGLQNTGKNAEVFAPQSDPNGLIKCTVGRSSLKSKL

SEQ ID NO: 28 - protein, 301 aa, synthetic construct
  source 1..301: note = Asp.Uox Variant Subunit (Q70)
  VARIANT 70: note = Xaa is unnatural amino acid containing tetrazine functionalgroup, and/or triazine functional group.
SAVKAARYGKDNVRVYKVHKDEKTGVQTVYEMTVCVLLEGEIETSYTKADNSVIVATDSIKNTIYITAKXNPVTPPELFGSILGTHFIEKYNHIHAAHVNIVCHRWTRMDIDGKPHPHSFIRDSEEKRNVQVDVVEGKGIDIKSSLSGLTVLKSTNSQFWGFLRDEYTTLKETWDRILSTDVDATWQWKNFSGLQEVRSHVPKFDATWATAREVTLKTFAEDNSASVQATMYKMAEQILARQQLIETVEYSLPNKHYFEIDLSWHKGLQNTGKNAEVFAPQSDPNGLIKCTVGRSSLKSKL

SEQ ID NO: 29 - protein, 301 aa, synthetic construct
  source 1..301: note = Asp.Uox Variant Subunit (W174)
  VARIANT 174: note = Xaa is unnatural amino acid containing tetrazine functionalgroup, and/or triazine functional group.
SAVKAARYGKDNVRVYKVHKDEKTGVQTVYEMTVCVLLEGEIETSYTKADNSVIVATDSIKNTIYITAKQNPVTPPELFGSILGTHFIEKYNHIHAAHVNIVCHRWTRMDIDGKPHPHSFIRDSEEKRNVQVDVVEGKGIDIKSSLSGLTVLKSTNSQFWGFLRDEYTTLKETXDRILSTDVDATWQWKNFSGLQEVRSHVPKFDATWATAREVTLKTFAEDNSASVQATMYKMAEQILARQQLIETVEYSLPNKHYFEIDLSWHKGLQNTGKNAEVFAPQSDPNGLIKCTVGRSSLKSKL

SEQ ID NO: 30 - protein, 301 aa, synthetic construct
  source 1..301: note = Asp.Uox Variant Subunit (N223)
  VARIANT 223: note = Xaa is unnatural amino acid containing tetrazine functionalgroup, and/or triazine functional group.
SAVKAARYGKDNVRVYKVHKDEKTGVQTVYEMTVCVLLEGEIETSYTKADNSVIVATDSIKNTIYITAKQNPVTPPELFGSILGTHFIEKYNHIHAAHVNIVCHRWTRMDIDGKPHPHSFIRDSEEKRNVQVDVVEGKGIDIKSSLSGLTVLKSTNSQFWGFLRDEYTTLKETWDRILSTDVDATWQWKNFSGLQEVRSHVPKFDATWATAREVTLKTFAEDXSASVQATMYKMAEQILARQQLIETVEYSLPNKHYFEIDLSWHKGLQNTGKNAEVFAPQSDPNGLIKCTVGRSSLKSKL

SEQ ID NO: 31 - protein, 301 aa, synthetic construct
  source 1..301: note = Asp.Uox Variant Subunit (E41)
  VARIANT 41: note = Xaa is unnatural amino acid containing tetrazine functionalgroup, and/or triazine functional group.
SAVKAARYGKDNVRVYKVHKDEKTGVQTVYEMTVCVLLEGXIETSYTKADNSVIVATDSIKNTIYITAKQNPVTPPELFGSILGTHFIEKYNHIHAAHVNIVCHRWTRMDIDGKPHPHSFIRDSEEKRNVQVDVVEGKGIDIKSSLSGLTVLKSTNSQFWGFLRDEYTTLKETWDRILSTDVDATWQWKNFSGLQEVRSHVPKFDATWATAREVTLKTFAEDNSASVQATMYKMAEQILARQQLIETVEYSLPNKHYFEIDLSWHKGLQNTGKNAEVFAPQSDPNGLIKCTVGRSSLKSKL

SEQ ID NO: 32 - protein, 301 aa, synthetic construct
  source 1..301: note = Asp.Uox Variant Subunit (D261)
  VARIANT 261: note = Xaa is unnatural amino acid containing tetrazine functionalgroup, and/or triazine functional group.
SAVKAARYGKDNVRVYKVHKDEKTGVQTVYEMTVCVLLEGEIETSYTKADNSVIVATDSIKNTIYITAKQNPVTPPELFGSILGTHFIEKYNHIHAAHVNIVCHRWTRMDIDGKPHPHSFIRDSEEKRNVQVDVVEGKGIDIKSSLSGLTVLKSTNSQFWGFLRDEYTTLKETWDRILSTDVDATWQWKNFSGLQEVRSHVPKFDATWATAREVTLKTFAEDNSASVQATMYKMAEQILARQQLIETVEYSLPNKHYFEIXLSWHKGLQNTGKNAEVFAPQSDPNGLIKCTVGRSSLKSKL

SEQ ID NO: 33 - protein, 301 aa, synthetic construct
  source 1..301: note = Asp.Uox Variant Subunit (G25)
  VARIANT 25: note = Xaa is unnatural amino acid containing tetrazine functionalgroup, and/or triazine functional group.
SAVKAARYGKDNVRVYKVHKDEKTXVQTVYEMTVCVLLEGEIETSYTKADNSVIVATDSIKNTIYITAKQNPVTPPELFGSILGTHFIEKYNHIHAAHVNIVCHRWTRMDIDGKPHPHSFIRDSEEKRNVQVDVVEGKGIDIKSSLSGLTVLKSTNSQFWGFLRDEYTTLKETWDRILSTDVDATWQWKNFSGLQEVRSHVPKFDATWATAREVTLKTFAEDNSASVQATMYKMAEQILARQQLIETVEYSLPNKHYFEIDLSWHKGLQNTGKNAEVFAPQSDPNGLIKCTVGRSSLKSKL

SEQ ID NO: 34 - protein, 301 aa, synthetic construct
  source 1..301: note = Asp.Uox Variant Subunit (S52)
  VARIANT 52: note = Xaa is unnatural amino acid containing tetrazine functionalgroup, and/or triazine functional group.
SAVKAARYGKDNVRVYKVHKDEKTGVQTVYEMTVCVLLEGEIETSYTKADNXVIVATDSIKNTIYITAKQNPVTPPELFGSILGTHFIEKYNHIHAAHVNIVCHRWTRMDIDGKPHPHSFIRDSEEKRNVQVDVVEGKGIDIKSSLSGLTVLKSTNSQFWGFLRDEYTTLKETWDRILSTDVDATWQWKNFSGLQEVRSHVPKFDATWATAREVTLKTFAEDNSASVQATMYKMAEQILARQQLIETVEYSLPNKHYFEIDLSWHKGLQNTGKNAEVFAPQSDPNGLIKCTVGRSSLKSKL

SEQ ID NO: 35 - protein, 301 aa, synthetic construct
  source 1..301: note = Asp.Uox Variant Subunit (R241)
  VARIANT 241: note = Xaa is unnatural amino acid containing tetrazine functionalgroup, and/or triazine functional group.
SAVKAARYGKDNVRVYKVHKDEKTGVQTVYEMTVCVLLEGEIETSYTKADNSVIVATDSIKNTIYITAKQNPVTPPELFGSILGTHFIEKYNHIHAAHVNIVCHRWTRMDIDGKPHPHSFIRDSEEKRNVQVDVVEGKGIDIKSSLSGLTVLKSTNSQFWGFLRDEYTTLKETWDRILSTDVDATWQWKNFSGLQEVRSHVPKFDATWATAREVTLKTFAEDNSASVQATMYKMAEQILAXQQLIETVEYSLPNKHYFEIDLSWHKGLQNTGKNAEVFAPQSDPNGLIKCTVGRSSLKSKL

SEQ ID NO: 36 - protein, 301 aa, synthetic construct
  source 1..301: note = Asp.Uox Variant Subunit (E213)
  VARIANT 213: note = Xaa is unnatural amino acid containing tetrazine functionalgroup, and/or triazine functional group.
SAVKAARYGKDNVRVYKVHKDEKTGVQTVYEMTVCVLLEGEIETSYTKADNSVIVATDSIKNTIYITAKQNPVTPPELFGSILGTHFIEKYNHIHAAHVNIVCHRWTRMDIDGKPHPHSFIRDSEEKRNVQVDVVEGKGIDIKSSLSGLTVLKSTNSQFWGFLRDEYTTLKETWDRILSTDVDATWQWKNFSGLQEVRSHVPKFDATWATARXVTLKTFAEDNSASVQATMYKMAEQILARQQLIETVEYSLPNKHYFEIDLSWHKGLQNTGKNAEVFAPQSDPNGLIKCTVGRSSLKSKL

SEQ ID NO: 37 - protein, 301 aa, synthetic construct
  source 1..301: note = Asp.Uox Variant Subunit (N274)
  VARIANT 274: note = Xaa is unnatural amino acid containing tetrazine functionalgroup, and/or triazine functional group.
SAVKAARYGKDNVRVYKVHKDEKTGVQTVYEMTVCVLLEGEIETSYTKADNSVIVATDSIKNTIYITAKQNPVTPPELFGSILGTHFIEKYNHIHAAHVNIVCHRWTRMDIDGKPHPHSFIRDSEEKRNVQVDVVEGKGIDIKSSLSGLTVLKSTNSQFWGFLRDEYTTLKETWDRILSTDVDATWQWKNFSGLQEVRSHVPKFDATWATAREVTLKTFAEDNSASVQATMYKMAEQILARQQLIETVEYSLPNKHYFEIDLSWHKGLQNTGKXAEVFAPQSDPNGLIKCTVGRSSLKSKL

SEQ ID NO: 38 - protein, 301 aa, synthetic construct
  source 1..301: note = Asp.Uox Variant Subunit (E221)
  VARIANT 221: note = Xaa is unnatural amino acid containing tetrazine functionalgroup, and/or triazine functional group.
SAVKAARYGKDNVRVYKVHKDEKTGVQTVYEMTVCVLLEGEIETSYTKADNSVIVATDSIKNTIYITAKQNPVTPPELFGSILGTHFIEKYNHIHAAHVNIVCHRWTRMDIDGKPHPHSFIRDSEEKRNVQVDVVEGKGIDIKSSLSGLTVLKSTNSQFWGFLRDEYTTLKETWDRILSTDVDATWQWKNFSGLQEVRSHVPKFDATWATAREVTLKTFAXDNSASVQATMYKMAEQILARQQLIETVEYSLPNKHYFEIDLSWHKGLQNTGKNAEVFAPQSDPNGLIKCTVGRSSLKSKL

SEQ ID NO: 39 - protein, 301 aa, synthetic construct
  source 1..301: note = Asp.Uox Variant Subunit (A206)
  VARIANT 206: note = Xaa is unnatural amino acid containing tetrazine functionalgroup, and/or triazine functional group.
SAVKAARYGKDNVRVYKVHKDEKTGVQTVYEMTVCVLLEGEIETSYTKADNSVIVATDSIKNTIYITAKQNPVTPPELFGSILGTHFIEKYNHIHAAHVNIVCHRWTRMDIDGKPHPHSFIRDSEEKRNVQVDVVEGKGIDIKSSLSGLTVLKSTNSQFWGFLRDEYTTLKETWDRILSTDVDATWQWKNFSGLQEVRSHVPKFDXTWATAREVTLKTFAEDNSASVQATMYKMAEQILARQQLIETVEYSLPNKHYFEIDLSWHKGLQNTGKNAEVFAPQSDPNGLIKCTVGRSSLKSKL

SEQ ID NO: 40 - protein, 301 aa, synthetic construct
  source 1..301: note = Asp.Uox Variant Subunit (E236)
  VARIANT 236: note = Xaa is unnatural amino acid containing tetrazine functionalgroup, and/or triazine functional group.
SAVKAARYGKDNVRVYKVHKDEKTGVQTVYEMTVCVLLEGEIETSYTKADNSVIVATDSIKNTIYITAKQNPVTPPELFGSILGTHFIEKYNHIHAAHVNIVCHRWTRMDIDGKPHPHSFIRDSEEKRNVQVDVVEGKGIDIKSSLSGLTVLKSTNSQFWGFLRDEYTTLKETWDRILSTDVDATWQWKNFSGLQEVRSHVPKFDATWATAREVTLKTFAEDNSASVQATMYKMAXQILARQQLIETVEYSLPNKHYFEIDLSWHKGLQNTGKNAEVFAPQSDPNGLIKCTVGRSSLKSKL

SEQ ID NO: 41 - protein, 301 aa, synthetic construct
  source 1..301: note = Asp.Uox Variant Subunit (R164)
  VARIANT 164: note = Xaa is unnatural amino acid containing tetrazine functionalgroup, and/or triazine functional group.
SAVKAARYGKDNVRVYKVHKDEKTGVQTVYEMTVCVLLEGEIETSYTKADNSVIVATDSIKNTIYITAKQNPVTPPELFGSILGTHFIEKYNHIHAAHVNIVCHRWTRMDIDGKPHPHSFIRDSEEKRNVQVDVVEGKGIDIKSSLSGLTVLKSTNSQFWGFLXDEYTTLKETWDRILSTDVDATWQWKNFSGLQEVRSHVPKFDATWATAREVTLKTFAEDNSASVQATMYKMAEQILARQQLIETVEYSLPNKHYFEIDLSWHKGLQNTGKNAEVFAPQSDPNGLIKCTVGRSSLKSKL

SEQ ID NO: 42 - protein, 301 aa, synthetic construct
  source 1..301: note = Asp.Uox Variant Subunit (Q269)
  VARIANT 269: note = Xaa is unnatural amino acid containing tetrazine functionalgroup, and/or triazine functional group.
SAVKAARYGKDNVRVYKVHKDEKTGVQTVYEMTVCVLLEGEIETSYTKADNSVIVATDSIKNTIYITAKQNPVTPPELFGSILGTHFIEKYNHIHAAHVNIVCHRWTRMDIDGKPHPHSFIRDSEEKRNVQVDVVEGKGIDIKSSLSGLTVLKSTNSQFWGFLRDEYTTLKETWDRILSTDVDATWQWKNFSGLQEVRSHVPKFDATWATAREVTLKTFAEDNSASVQATMYKMAEQILARQQLIETVEYSLPNKHYFEIDLSWHKGLXNTGKNAEVFAPQSDPNGLIKCTVGRSSLKSKL

SEQ ID NO: 43 - protein, 301 aa, synthetic construct
  source 1..301: note = Asp.Uox Variant Subunit (E136)
  VARIANT 136: note = Xaa is unnatural amino acid containing tetrazine functionalgroup, and/or triazine functional group.
SAVKAARYGKDNVRVYKVHKDEKTGVQTVYEMTVCVLLEGEIETSYTKADNSVIVATDSIKNTIYITAKQNPVTPPELFGSILGTHFIEKYNHIHAAHVNIVCHRWTRMDIDGKPHPHSFIRDSEEKRNVQVDVVXGKGIDIKSSLSGLTVLKSTNSQFWGFLRDEYTTLKETWDRILSTDVDATWQWKNFSGLQEVRSHVPKFDATWATAREVTLKTFAEDNSASVQATMYKMAEQILARQQLIETVEYSLPNKHYFEIDLSWHKGLQNTGKNAEVFAPQSDPNGLIKCTVGRSSLKSKL

SEQ ID NO: 44 - protein, 301 aa, synthetic construct
  source 1..301: note = Asp.Uox Variant Subunit (E259)
  VARIANT 259: note = Xaa is unnatural amino acid containing tetrazine functionalgroup, and/or triazine functional group.
SAVKAARYGKDNVRVYKVHKDEKTGVQTVYEMTVCVLLEGEIETSYTKADNSVIVATDSIKNTIYITAKQNPVTPPELFGSILGTHFIEKYNHIHAAHVNIVCHRWTRMDIDGKPHPHSFIRDSEEKRNVQVDVVEGKGIDIKSSLSGLTVLKSTNSQFWGFLRDEYTTLKETWDRILSTDVDATWQWKNFSGLQEVRSHVPKFDATWATAREVTLKTFAEDNSASVQATMYKMAEQILARQQLIETVEYSLPNKHYFXIDLSWHKGLQNTGKNAEVFAPQSDPNGLIKCTVGRSSLKSKL

SEQ ID NO: 45 - protein, 301 aa, synthetic construct
  source 1..301: note = Asp.Uox Variant Subunit (E246)
  VARIANT 246: note = Xaa is unnatural amino acid containing tetrazine functionalgroup, and/or triazine functional group.
SAVKAARYGKDNVRVYKVHKDEKTGVQTVYEMTVCVLLEGEIETSYTKADNSVIVATDSIKNTIYITAKQNPVTPPELFGSILGTHFIEKYNHIHAAHVNIVCHRWTRMDIDGKPHPHSFIRDSEEKRNVQVDVVEGKGIDIKSSLSGLTVLKSTNSQFWGFLRDEYTTLKETWDRILSTDVDATWQWKNFSGLQEVRSHVPKFDATWATAREVTLKTFAEDNSASVQATMYKMAEQILARQQLIXTVEYSLPNKHYFEIDLSWHKGLQNTGKNAEVFAPQSDPNGLIKCTVGRSSLKSKL

SEQ ID NO: 46 - protein, 301 aa, synthetic construct
  source 1..301: note = Asp.Uox Variant Subunit (A49)
  VARIANT 49: note = Xaa is unnatural amino acid containing tetrazine functionalgroup, and/or triazine functional group.
SAVKAARYGKDNVRVYKVHKDEKTGVQTVYEMTVCVLLEGEIETSYTKXDNSVIVATDSIKNTIYITAKQNPVTPPELFGSILGTHFIEKYNHIHAAHVNIVCHRWTRMDIDGKPHPHSFIRDSEEKRNVQVDVVEGKGIDIKSSLSGLTVLKSTNSQFWGFLRDEYTTLKETWDRILSTDVDATWQWKNFSGLQEVRSHVPKFDATWATAREVTLKTFAEDNSASVQATMYKMAEQILARQQLIETVEYSLPNKHYFEIDLSWHKGLQNTGKNAEVFAPQSDPNGLIKCTVGRSSLKSKL

SEQ ID NO: 47 - protein, 301 aa, synthetic construct
  source 1..301: note = Asp.Uox Variant Subunit (G148)
  VARIANT 148: note = Xaa is unnatural amino acid containing tetrazine functionalgroup, and/or triazine functional group.
SAVKAARYGKDNVRVYKVHKDEKTGVQTVYEMTVCVLLEGEIETSYTKADNSVIVATDSIKNTIYITAKQNPVTPPELFGSILGTHFIEKYNHIHAAHVNIVCHRWTRMDIDGKPHPHSFIRDSEEKRNVQVDVVEGKGIDIKSSLSXLTVLKSTNSQFWGFLRDEYTTLKETWDRILSTDVDATWQWKNFSGLQEVRSHVPKFDATWATAREVTLKTFAEDNSASVQATMYKMAEQILARQQLIETVEYSLPNKHYFEIDLSWHKGLQNTGKNAEVFAPQSDPNGLIKCTVGRSSLKSKL

SEQ ID NO: 48 - protein, 301 aa, synthetic construct
  source 1..301: note = Asp.Uox Variant Subunit (H19)
  VARIANT 19: note = Xaa is unnatural amino acid containing tetrazine functionalgroup, and/or triazine functional group.
SAVKAARYGKDNVRVYKVXKDEKTGVQTVYEMTVCVLLEGEIETSYTKADNSVIVATDSIKNTIYITAKQNPVTPPELFGSILGTHFIEKYNHIHAAHVNIVCHRWTRMDIDGKPHPHSFIRDSEEKRNVQVDVVEGKGIDIKSSLSGLTVLKSTNSQFWGFLRDEYTTLKETWDRILSTDVDATWQWKNFSGLQEVRSHVPKFDATWATAREVTLKTFAEDNSASVQATMYKMAEQILARQQLIETVEYSLPNKHYFEIDLSWHKGLQNTGKNAEVFAPQSDPNGLIKCTVGRSSLKSKL

SEQ ID NO: 49 - protein, 301 aa, synthetic construct
  source 1..301: note = Asp.Uox Variant Subunit (S296)
  VARIANT 296: note = Xaa is unnatural amino acid containing tetrazine functionalgroup, and/or triazine functional group.
SAVKAARYGKDNVRVYKVHKDEKTGVQTVYEMTVCVLLEGEIETSYTKADNSVIVATDSIKNTIYITAKQNPVTPPELFGSILGTHFIEKYNHIHAAHVNIVCHRWTRMDIDGKPHPHSFIRDSEEKRNVQVDVVEGKGIDIKSSLSGLTVLKSTNSQFWGFLRDEYTTLKETWDRILSTDVDATWQWKNFSGLQEVRSHVPKFDATWATAREVTLKTFAEDNSASVQATMYKMAEQILARQQLIETVEYSLPNKHYFEIDLSWHKGLQNTGKNAEVFAPQSDPNGLIKCTVGRSXLKSKL

SEQ ID NO: 50 - protein, 301 aa, synthetic construct
  source 1..301: note = Asp.Uox Variant Subunit (T47)
  VARIANT 47: note = Xaa is unnatural amino acid containing tetrazine functionalgroup, and/or triazine functional group.
SAVKAARYGKDNVRVYKVHKDEKTGVQTVYEMTVCVLLEGEIETSYXKADNSVIVATDSIKNTIYITAKQNPVTPPELFGSILGTHFIEKYNHIHAAHVNIVCHRWTRMDIDGKPHPHSFIRDSEEKRNVQVDVVEGKGIDIKSSLSGLTVLKSTNSQFWGFLRDEYTTLKETWDRILSTDVDATWQWKNFSGLQEVRSHVPKFDATWATAREVTLKTFAEDNSASVQATMYKMAEQILARQQLIETVEYSLPNKHYFEIDLSWHKGLQNTGKNAEVFAPQSDPNGLIKCTVGRSSLKSKL

SEQ ID NO: 51 - protein, 303 aa, Candida utilis
MSTTLSSSTYGKDNVKFLKVKKDPQNPKKQEVMEATVTCLLEGGFDTSYTEADNSSIVPTDTVKNTILVLAKTTEIWPIERFAAKLATHFVEKYSHVSGVSVKIVQDRWVKYAVDGKPHDHSFIHEGGEKRITDLYYKRSGDYKLSSAIKDLTVLKSTGSMFYGYNKCDFTTLQPTTDRILSTDVDATWVWDNKKIGSVYDIAKAADKGIFDNVYNQAREITLTTFALENSPSVQATMFNMATQILEKACSVYSVSYALPNKHYFLIDLKWKGLENDNELFYPSPHPNGLIKCTVVRKEKTKL

SEQ ID NO: 52 - protein, 303 aa, synthetic construct
  source 1..303: note = Candida.Uox Variant Subunit (T301)
  VARIANT 301: note = Xaa is unnatural amino acid containing tetrazine functionalgroup, and/or triazine functional group.
MSTTLSSSTYGKDNVKFLKVKKDPQNPKKQEVMEATVTCLLEGGFDTSYTEADNSSIVPTDTVKNTILVLAKTTEIWPIERFAAKLATHFVEKYSHVSGVSVKIVQDRWVKYAVDGKPHDHSFIHEGGEKRITDLYYKRSGDYKLSSAIKDLTVLKSTGSMFYGYNKCDFTTLQPTTDRILSTDVDATWVWDNKKIGSVYDIAKAADKGIFDNVYNQAREITLTTFALENSPSVQATMFNMATQILEKACSVYSVSYALPNKHYFLIDLKWKGLENDNELFYPSPHPNGLIKCTVVRKEKXKL

SEQ ID NO: 53 - protein, 303 aa, synthetic construct
  source 1..303: note = Candida.Uox Variant Subunit (N26)
  VARIANT 26: note = Xaa is unnatural amino acid containing tetrazine functionalgroup, and/or triazine functional group.
MSTTLSSSTYGKDNVKFLKVKKDPQXPKKQEVMEATVTCLLEGGFDTSYTEADNSSIVPTDTVKNTILVLAKTTEIWPIERFAAKLATHFVEKYSHVSGVSVKIVQDRWVKYAVDGKPHDHSFIHEGGEKRITDLYYKRSGDYKLSSAIKDLTVLKSTGSMFYGYNKCDFTTLQPTTDRILSTDVDATWVWDNKKIGSVYDIAKAADKGIFDNVYNQAREITLTTFALENSPSVQATMFNMATQILEKACSVYSVSYALPNKHYFLIDLKWKGLENDNELFYPSPHPNGLIKCTVVRKEKTKL

SEQ ID NO: 54 - protein, 303 aa, synthetic construct
  source 1..303: note = Candida.Uox Variant Subunit (L303)
  VARIANT 303: note = Xaa is unnatural amino acid containing tetrazine functionalgroup, and/or triazine functional group.
MSTTLSSSTYGKDNVKFLKVKKDPQNPKKQEVMEATVTCLLEGGFDTSYTEADNSSIVPTDTVKNTILVLAKTTEIWPIERFAAKLATHFVEKYSHVSGVSVKIVQDRWVKYAVDGKPHDHSFIHEGGEKRITDLYYKRSGDYKLSSAIKDLTVLKSTGSMFYGYNKCDFTTLQPTTDRILSTDVDATWVWDNKKIGSVYDIAKAADKGIFDNVYNQAREITLTTFALENSPSVQATMFNMATQILEKACSVYSVSYALPNKHYFLIDLKWKGLENDNELFYPSPHPNGLIKCTVVRKEKTKX

SEQ ID NO: 55 - protein, 303 aa, synthetic construct
  source 1..303: note = Candida.Uox Variant Subunit (K194)
  VARIANT 194: note = Xaa is unnatural amino acid containing tetrazine functionalgroup, and/or triazine functional group.
MSTTLSSSTYGKDNVKFLKVKKDPQNPKKQEVMEATVTCLLEGGFDTSYTEADNSSIVPTDTVKNTILVLAKTTEIWPIERFAAKLATHFVEKYSHVSGVSVKIVQDRWVKYAVDGKPHDHSFIHEGGEKRITDLYYKRSGDYKLSSAIKDLTVLKSTGSMFYGYNKCDFTTLQPTTDRILSTDVDATWVWDNXKIGSVYDIAKAADKGIFDNVYNQAREITLTTFALENSPSVQATMFNMATQILEKACSVYSVSYALPNKHYFLIDLKWKGLENDNELFYPSPHPNGLIKCTVVRKEKTKL

SEQ ID NO: 56 - protein, 303 aa, synthetic construct
  source 1..303: note = Candida.Uox Variant Subunit (S95)
  VARIANT 95: note = Xaa is unnatural amino acid containing tetrazine functionalgroup, and/or triazine functional group.
MSTTLSSSTYGKDNVKFLKVKKDPQNPKKQEVMEATVTCLLEGGFDTSYTEADNSSIVPTDTVKNTILVLAKTTEIWPIERFAAKLATHFVEKYXHVSGVSVKIVQDRWVKYAVDGKPHDHSFIHEGGEKRITDLYYKRSGDYKLSSAIKDLTVLKSTGSMFYGYNKCDFTTLQPTTDRILSTDVDATWVWDNKKIGSVYDIAKAADKGIFDNVYNQAREITLTTFALENSPSVQATMFNMATQILEKACSVYSVSYALPNKHYFLIDLKWKGLENDNELFYPSPHPNGLIKCTVVRKEKTKL

SEQ ID NO: 57 - protein, 303 aa, synthetic construct
  source 1..303: note = Candida.Uox Variant Subunit (S140)
  VARIANT 140: note = Xaa is unnatural amino acid containing tetrazine functionalgroup, and/or triazine functional group.
MSTTLSSSTYGKDNVKFLKVKKDPQNPKKQEVMEATVTCLLEGGFDTSYTEADNSSIVPTDTVKNTILVLAKTTEIWPIERFAAKLATHFVEKYSHVSGVSVKIVQDRWVKYAVDGKPHDHSFIHEGGEKRITDLYYKRXGDYKLSSAIKDLTVLKSTGSMFYGYNKCDFTTLQPTTDRILSTDVDATWVWDNKKIGSVYDIAKAADKGIFDNVYNQAREITLTTFALENSPSVQATMFNMATQILEKACSVYSVSYALPNKHYFLIDLKWKGLENDNELFYPSPHPNGLIKCTVVRKEKTKL

SEQ ID NO: 58 - protein, 303 aa, synthetic construct
  source 1..303: note = Candida.Uox Variant Subunit (G116)
  VARIANT 116: note = Xaa is unnatural amino acid containing tetrazine functionalgroup, and/or triazine functional group.
MSTTLSSSTYGKDNVKFLKVKKDPQNPKKQEVMEATVTCLLEGGFDTSYTEADNSSIVPTDTVKNTILVLAKTTEIWPIERFAAKLATHFVEKYSHVSGVSVKIVQDRWVKYAVDXKPHDHSFIHEGGEKRITDLYYKRSGDYKLSSAIKDLTVLKSTGSMFYGYNKCDFTTLQPTTDRILSTDVDATWVWDNKKIGSVYDIAKAADKGIFDNVYNQAREITLTTFALENSPSVQATMFNMATQILEKACSVYSVSYALPNKHYFLIDLKWKGLENDNELFYPSPHPNGLIKCTVVRKEKTKL

SEQ ID NO: 59 - protein, 303 aa, synthetic construct
  source 1..303: note = Candida.Uox Variant Subunit (K302)
  VARIANT 302: note = Xaa is unnatural amino acid containing tetrazine functionalgroup, and/or triazine functional group.
MSTTLSSSTYGKDNVKFLKVKKDPQNPKKQEVMEATVTCLLEGGFDTSYTEADNSSIVPTDTVKNTILVLAKTTEIWPIERFAAKLATHFVEKYSHVSGVSVKIVQDRWVKYAVDGKPHDHSFIHEGGEKRITDLYYKRSGDYKLSSAIKDLTVLKSTGSMFYGYNKCDFTTLQPTTDRILSTDVDATWVWDNKKIGSVYDIAKAADKGIFDNVYNQAREITLTTFALENSPSVQATMFNMATQILEKACSVYSVSYALPNKHYFLIDLKWKGLENDNELFYPSPHPNGLIKCTVVRKEKTXL

SEQ ID NO: 60 - protein, 303 aa, synthetic construct
  source 1..303: note = Candida.Uox Variant Subunit (K167)
  VARIANT 167: note = Xaa is unnatural amino acid containing tetrazine functionalgroup, and/or triazine functional group.
MSTTLSSSTYGKDNVKFLKVKKDPQNPKKQEVMEATVTCLLEGGFDTSYTEADNSSIVPTDTVKNTILVLAKTTEIWPIERFAAKLATHFVEKYSHVSGVSVKIVQDRWVKYAVDGKPHDHSFIHEGGEKRITDLYYKRSGDYKLSSAIKDLTVLKSTGSMFYGYNXCDFTTLQPTTDRILSTDVDATWVWDNKKIGSVYDIAKAADKGIFDNVYNQAREITLTTFALENSPSVQATMFNMATQILEKACSVYSVSYALPNKHYFLIDLKWKGLENDNELFYPSPHPNGLIKCTVVRKEKTKL

SEQ ID NO: 61 - protein, 303 aa, synthetic construct
  source 1..303: note = Candida.Uox Variant Subunit (D115)
  VARIANT 115: note = Xaa is unnatural amino acid containing tetrazine functionalgroup, and/or triazine functional group.
MSTTLSSSTYGKDNVKFLKVKKDPQNPKKQEVMEATVTCLLEGGFDTSYTEADNSSIVPTDTVKNTILVLAKTTEIWPIERFAAKLATHFVEKYSHVSGVSVKIVQDRWVKYAVXGKPHDHSFIHEGGEKRITDLYYKRSGDYKLSSAIKDLTVLKSTGSMFYGYNKCDFTTLQPTTDRILSTDVDATWVWDNKKIGSVYDIAKAADKGIFDNVYNQAREITLTTFALENSPSVQATMFNMATQILEKACSVYSVSYALPNKHYFLIDLKWKGLENDNELFYPSPHPNGLIKCTVVRKEKTKL

SEQ ID NO: 62 - protein, 303 aa, synthetic construct
  source 1..303: note = Candida.Uox Variant Subunit (E299)
  VARIANT 299: note = Xaa is unnatural amino acid containing tetrazine functionalgroup, and/or triazine functional group.
MSTTLSSSTYGKDNVKFLKVKKDPQNPKKQEVMEATVTCLLEGGFDTSYTEADNSSIVPTDTVKNTILVLAKTTEIWPIERFAAKLATHFVEKYSHVSGVSVKIVQDRWVKYAVDGKPHDHSFIHEGGEKRITDLYYKRSGDYKLSSAIKDLTVLKSTGSMFYGYNKCDFTTLQPTTDRILSTDVDATWVWDNKKIGSVYDIAKAADKGIFDNVYNQAREITLTTFALENSPSVQATMFNMATQILEKACSVYSVSYALPNKHYFLIDLKWKGLENDNELFYPSPHPNGLIKCTVVRKXKTKL

SEQ ID NO: 63 - protein, 303 aa, synthetic construct
  source 1..303: note = Candida.Uox Variant Subunit (P24)
  VARIANT 24: note = Xaa is unnatural amino acid containing tetrazine functionalgroup, and/or triazine functional group.
MSTTLSSSTYGKDNVKFLKVKKDXQNPKKQEVMEATVTCLLEGGFDTSYTEADNSSIVPTDTVKNTILVLAKTTEIWPIERFAAKLATHFVEKYSHVSGVSVKIVQDRWVKYAVDGKPHDHSFIHEGGEKRITDLYYKRSGDYKLSSAIKDLTVLKSTGSMFYGYNKCDFTTLQPTTDRILSTDVDATWVWDNKKIGSVYDIAKAADKGIFDNVYNQAREITLTTFALENSPSVQATMFNMATQILEKACSVYSVSYALPNKHYFLIDLKWKGLENDNELFYPSPHPNGLIKCTVVRKEKTKL

SEQ ID NO: 64 - protein, 303 aa, synthetic construct
  source 1..303: note = Candida.Uox Variant Subunit (W271)
  VARIANT 271: note = Xaa is unnatural amino acid containing tetrazine functionalgroup, and/or triazine functional group.
MSTTLSSSTYGKDNVKFLKVKKDPQNPKKQEVMEATVTCLLEGGFDTSYTEADNSSIVPTDTVKNTILVLAKTTEIWPIERFAAKLATHFVEKYSHVSGVSVKIVQDRWVKYAVDGKPHDHSFIHEGGEKRITDLYYKRSGDYKLSSAIKDLTVLKSTGSMFYGYNKCDFTTLQPTTDRILSTDVDATWVWDNKKIGSVYDIAKAADKGIFDNVYNQAREITLTTFALENSPSVQATMFNMATQILEKACSVYSVSYALPNKHYFLIDLKXKGLENDNELFYPSPHPNGLIKCTVVRKEKTKL

SEQ ID NO: 65 - protein, 303 aa, synthetic construct
  source 1..303: note = Candida.Uox Variant Subunit (D277)
  VARIANT 277: note = Xaa is unnatural amino acid containing tetrazine functionalgroup, and/or triazine functional group.
MSTTLSSSTYGKDNVKFLKVKKDPQNPKKQEVMEATVTCLLEGGFDTSYTEADNSSIVPTDTVKNTILVLAKTTEIWPIERFAAKLATHFVEKYSHVSGVSVKIVQDRWVKYAVDGKPHDHSFIHEGGEKRITDLYYKRSGDYKLSSAIKDLTVLKSTGSMFYGYNKCDFTTLQPTTDRILSTDVDATWVWDNKKIGSVYDIAKAADKGIFDNVYNQAREITLTTFALENSPSVQATMFNMATQILEKACSVYSVSYALPNKHYFLIDLKWKGLENXNELFYPSPHPNGLIKCTVVRKEKTKL

SEQ ID NO: 66 - protein, 303 aa, synthetic construct
  source 1..303: note = Candida.Uox Variant Subunit (D169)
  VARIANT 169: note = Xaa is unnatural amino acid containing tetrazine functionalgroup, and/or triazine functional group.
MSTTLSSSTYGKDNVKFLKVKKDPQNPKKQEVMEATVTCLLEGGFDTSYTEADNSSIVPTDTVKNTILVLAKTTEIWPIERFAAKLATHFVEKYSHVSGVSVKIVQDRWVKYAVDGKPHDHSFIHEGGEKRITDLYYKRSGDYKLSSAIKDLTVLKSTGSMFYGYNKCXFTTLQPTTDRILSTDVDATWVWDNKKIGSVYDIAKAADKGIFDNVYNQAREITLTTFALENSPSVQATMFNMATQILEKACSVYSVSYALPNKHYFLIDLKWKGLENDNELFYPSPHPNGLIKCTVVRKEKTKL

SEQ ID NO: 67 - protein, 303 aa, synthetic construct
  source 1..303: note = Candida.Uox Variant Subunit (P118)
  VARIANT 118: note = Xaa is unnatural amino acid containing tetrazine functionalgroup, and/or triazine functional group.
MSTTLSSSTYGKDNVKFLKVKKDPQNPKKQEVMEATVTCLLEGGFDTSYTEADNSSIVPTDTVKNTILVLAKTTEIWPIERFAAKLATHFVEKYSHVSGVSVKIVQDRWVKYAVDGKXHDHSFIHEGGEKRITDLYYKRSGDYKLSSAIKDLTVLKSTGSMFYGYNKCDFTTLQPTTDRILSTDVDATWVWDNKKIGSVYDIAKAADKGIFDNVYNQAREITLTTFALENSPSVQATMFNMATQILEKACSVYSVSYALPNKHYFLIDLKWKGLENDNELFYPSPHPNGLIKCTVVRKEKTKL

SEQ ID NO: 68 - protein, 303 aa, synthetic construct
  source 1..303: note = Candida.Uox Variant Subunit (T177)
  VARIANT 177: note = Xaa is unnatural amino acid containing tetrazine functionalgroup, and/or triazine functional group.
MSTTLSSSTYGKDNVKFLKVKKDPQNPKKQEVMEATVTCLLEGGFDTSYTEADNSSIVPTDTVKNTILVLAKTTEIWPIERFAAKLATHFVEKYSHVSGVSVKIVQDRWVKYAVDGKPHDHSFIHEGGEKRITDLYYKRSGDYKLSSAIKDLTVLKSTGSMFYGYNKCDFTTLQPTXDRILSTDVDATWVWDNKKIGSVYDIAKAADKGIFDNVYNQAREITLTTFALENSPSVQATMFNMATQILEKACSVYSVSYALPNKHYFLIDLKWKGLENDNELFYPSPHPNGLIKCTVVRKEKTKL

SEQ ID NO: 69 - protein, 303 aa, synthetic construct
  source 1..303: note = Candida.Uox Variant Subunit (Q174)
  VARIANT 174: note = Xaa is unnatural amino acid containing tetrazine functionalgroup, and/or triazine functional group.
MSTTLSSSTYGKDNVKFLKVKKDPQNPKKQEVMEATVTCLLEGGFDTSYTEADNSSIVPTDTVKNTILVLAKTTEIWPIERFAAKLATHFVEKYSHVSGVSVKIVQDRWVKYAVDGKPHDHSFIHEGGEKRITDLYYKRSGDYKLSSAIKDLTVLKSTGSMFYGYNKCDFTTLXPTTDRILSTDVDATWVWDNKKIGSVYDIAKAADKGIFDNVYNQAREITLTTFALENSPSVQATMFNMATQILEKACSVYSVSYALPNKHYFLIDLKWKGLENDNELFYPSPHPNGLIKCTVVRKEKTKL

SEQ ID NO: 70 - protein, 303 aa, synthetic construct
  source 1..303: note = Candida.Uox Variant Subunit (K208)
  VARIANT 208: note = Xaa is unnatural amino acid containing tetrazine functionalgroup, and/or triazine functional group.
MSTTLSSSTYGKDNVKFLKVKKDPQNPKKQEVMEATVTCLLEGGFDTSYTEADNSSIVPTDTVKNTILVLAKTTEIWPIERFAAKLATHFVEKYSHVSGVSVKIVQDRWVKYAVDGKPHDHSFIHEGGEKRITDLYYKRSGDYKLSSAIKDLTVLKSTGSMFYGYNKCDFTTLQPTTDRILSTDVDATWVWDNKKIGSVYDIAKAADXGIFDNVYNQAREITLTTFALENSPSVQATMFNMATQILEKACSVYSVSYALPNKHYFLIDLKWKGLENDNELFYPSPHPNGLIKCTVVRKEKTKL

SEQ ID NO: 71 - protein, 303 aa, synthetic construct
  source 1..303: note = Candida.Uox Variant Subunit (E275)
  VARIANT 275: note = Xaa is unnatural amino acid containing tetrazine functionalgroup, and/or triazine functional group.
MSTTLSSSTYGKDNVKFLKVKKDPQNPKKQEVMEATVTCLLEGGFDTSYTEADNSSIVPTDTVKNTILVLAKTTEIWPIERFAAKLATHFVEKYSHVSGVSVKIVQDRWVKYAVDGKPHDHSFIHEGGEKRITDLYYKRSGDYKLSSAIKDLTVLKSTGSMFYGYNKCDFTTLQPTTDRILSTDVDATWVWDNKKIGSVYDIAKAADKGIFDNVYNQAREITLTTFALENSPSVQATMFNMATQILEKACSVYSVSYALPNKHYFLIDLKWKGLXNDNELFYPSPHPNGLIKCTVVRKEKTKL

SEQ ID NO: 72 - protein, 303 aa, synthetic construct
  source 1..303: note = Candida.Uox Variant Subunit (L266)
  VARIANT 266: note = Xaa is unnatural amino acid containing tetrazine functionalgroup, and/or triazine functional group.
MSTTLSSSTYGKDNVKFLKVKKDPQNPKKQEVMEATVTCLLEGGFDTSYTEADNSSIVPTDTVKNTILVLAKTTEIWPIERFAAKLATHFVEKYSHVSGVSVKIVQDRWVKYAVDGKPHDHSFIHEGGEKRITDLYYKRSGDYKLSSAIKDLTVLKSTGSMFYGYNKCDFTTLQPTTDRILSTDVDATWVWDNKKIGSVYDIAKAADKGIFDNVYNQAREITLTTFALENSPSVQATMFNMATQILEKACSVYSVSYALPNKHYFXIDLKWKGLENDNELFYPSPHPNGLIKCTVVRKEKTKL

SEQ ID NO: 73 - protein, 303 aa, synthetic construct
  source 1..303: note = Candida.Uox Variant Subunit (G273)
  VARIANT 273: note = Xaa is unnatural amino acid containing tetrazine functionalgroup, and/or triazine functional group.
MSTTLSSSTYGKDNVKFLKVKKDPQNPKKQEVMEATVTCLLEGGFDTSYTEADNSSIVPTDTVKNTILVLAKTTEIWPIERFAAKLATHFVEKYSHVSGVSVKIVQDRWVKYAVDGKPHDHSFIHEGGEKRITDLYYKRSGDYKLSSAIKDLTVLKSTGSMFYGYNKCDFTTLQPTTDRILSTDVDATWVWDNKKIGSVYDIAKAADKGIFDNVYNQAREITLTTFALENSPSVQATMFNMATQILEKACSVYSVSYALPNKHYFLIDLKWKXLENDNELFYPSPHPNGLIKCTVVRKEKTKL

SEQ ID NO: 74 - protein, 303 aa, synthetic construct
  source 1..303: note = Candida.Uox Variant Subunit (Y200)
  VARIANT 200: note = Xaa is unnatural amino acid containing tetrazine functionalgroup, and/or triazine functional group.
MSTTLSSSTYGKDNVKFLKVKKDPQNPKKQEVMEATVTCLLEGGFDTSYTEADNSSIVPTDTVKNTILVLAKTTEIWPIERFAAKLATHFVEKYSHVSGVSVKIVQDRWVKYAVDGKPHDHSFIHEGGEKRITDLYYKRSGDYKLSSAIKDLTVLKSTGSMFYGYNKCDFTTLQPTTDRILSTDVDATWVWDNKKIGSVXDIAKAADKGIFDNVYNQAREITLTTFALENSPSVQATMFNMATQILEKACSVYSVSYALPNKHYFLIDLKWKGLENDNELFYPSPHPNGLIKCTVVRKEKTKL

SEQ ID NO: 75 - protein, 303 aa, synthetic construct
  source 1..303: note = Candida.Uox Variant Subunit (E92)
  VARIANT 92: note = Xaa is unnatural amino acid containing tetrazine functionalgroup, and/or triazine functional group.
MSTTLSSSTYGKDNVKFLKVKKDPQNPKKQEVMEATVTCLLEGGFDTSYTEADNSSIVPTDTVKNTILVLAKTTEIWPIERFAAKLATHFVXKYSHVSGVSVKIVQDRWVKYAVDGKPHDHSFIHEGGEKRITDLYYKRSGDYKLSSAIKDLTVLKSTGSMFYGYNKCDFTTLQPTTDRILSTDVDATWVWDNKKIGSVYDIAKAADKGIFDNVYNQAREITLTTFALENSPSVQATMFNMATQILEKACSVYSVSYALPNKHYFLIDLKWKGLENDNELFYPSPHPNGLIKCTVVRKEKTKL

SEQ ID NO: 76 - protein, 303 aa, synthetic construct
  source 1..303: note = Candida.Uox Variant Subunit (E247)
  VARIANT 247: note = Xaa is unnatural amino acid containing tetrazine functionalgroup, and/or triazine functional group.
MSTTLSSSTYGKDNVKFLKVKKDPQNPKKQEVMEATVTCLLEGGFDTSYTEADNSSIVPTDTVKNTILVLAKTTEIWPIERFAAKLATHFVEKYSHVSGVSVKIVQDRWVKYAVDGKPHDHSFIHEGGEKRITDLYYKRSGDYKLSSAIKDLTVLKSTGSMFYGYNKCDFTTLQPTTDRILSTDVDATWVWDNKKIGSVYDIAKAADKGIFDNVYNQAREITLTTFALENSPSVQATMFNMATQILXKACSVYSVSYALPNKHYFLIDLKWKGLENDNELFYPSPHPNGLIKCTVVRKEKTKL

SEQ ID NO: 77 - protein, 303 aa, synthetic construct
  source 1..303: note = Candida.Uox Variant Subunit (L228)
  VARIANT 228: note = Xaa is unnatural amino acid containing tetrazine functionalgroup, and/or triazine functional group.
MSTTLSSSTYGKDNVKFLKVKKDPQNPKKQEVMEATVTCLLEGGFDTSYTEADNSSIVPTDTVKNTILVLAKTTEIWPIERFAAKLATHFVEKYSHVSGVSVKIVQDRWVKYAVDGKPHDHSFIHEGGEKRITDLYYKRSGDYKLSSAIKDLTVLKSTGSMFYGYNKCDFTTLQPTTDRILSTDVDATWVWDNKKIGSVYDIAKAADKGIFDNVYNQAREITLTTFAXENSPSVQATMFNMATQILEKACSVYSVSYALPNKHYFLIDLKWKGLENDNELFYPSPHPNGLIKCTVVRKEKTKL

SEQ ID NO: 78 - protein, 303 aa, synthetic construct
  source 1..303: note = Candida.Uox Variant Subunit (K300)
  VARIANT 300: note = Xaa is unnatural amino acid containing tetrazine functionalgroup, and/or triazine functional group.
MSTTLSSSTYGKDNVKFLKVKKDPQNPKKQEVMEATVTCLLEGGFDTSYTEADNSSIVPTDTVKNTILVLAKTTEIWPIERFAAKLATHFVEKYSHVSGVSVKIVQDRWVKYAVDGKPHDHSFIHEGGEKRITDLYYKRSGDYKLSSAIKDLTVLKSTGSMFYGYNKCDFTTLQPTTDRILSTDVDATWVWDNKKIGSVYDIAKAADKGIFDNVYNQAREITLTTFALENSPSVQATMFNMATQILEKACSVYSVSYALPNKHYFLIDLKWKGLENDNELFYPSPHPNGLIKCTVVRKEXTKL

SEQ ID NO: 79 - protein, 303 aa, synthetic construct
  source 1..303: note = Candida.Uox Variant Subunit (K204)
  VARIANT 204: note = Xaa is unnatural amino acid containing tetrazine functionalgroup, and/or triazine functional group.
MSTTLSSSTYGKDNVKFLKVKKDPQNPKKQEVMEATVTCLLEGGFDTSYTEADNSSIVPTDTVKNTILVLAKTTEIWPIERFAAKLATHFVEKYSHVSGVSVKIVQDRWVKYAVDGKPHDHSFIHEGGEKRITDLYYKRSGDYKLSSAIKDLTVLKSTGSMFYGYNKCDFTTLQPTTDRILSTDVDATWVWDNKKIGSVYDIAXAADKGIFDNVYNQAREITLTTFALENSPSVQATMFNMATQILEKACSVYSVSYALPNKHYFLIDLKWKGLENDNELFYPSPHPNGLIKCTVVRKEKTKL

SEQ ID NO: 80 - protein, 303 aa, synthetic construct
  source 1..303: note = Candida.Uox Variant Subunit (E51)
  VARIANT 51: note = Xaa is unnatural amino acid containing tetrazine functionalgroup, and/or triazine functional group.
MSTTLSSSTYGKDNVKFLKVKKDPQNPKKQEVMEATVTCLLEGGFDTSYTXADNSSIVPTDTVKNTILVLAKTTEIWPIERFAAKLATHFVEKYSHVSGVSVKIVQDRWVKYAVDGKPHDHSFIHEGGEKRITDLYYKRSGDYKLSSAIKDLTVLKSTGSMFYGYNKCDFTTLQPTTDRILSTDVDATWVWDNKKIGSVYDIAKAADKGIFDNVYNQAREITLTTFALENSPSVQATMFNMATQILEKACSVYSVSYALPNKHYFLIDLKWKGLENDNELFYPSPHPNGLIKCTVVRKEKTKL

SEQ ID NO: 81 - protein, 303 aa, synthetic construct
  source 1..303: note = Candida.Uox Variant Subunit (D207)
  VARIANT 207: note = Xaa is unnatural amino acid containing tetrazine functionalgroup, and/or triazine functional group.
MSTTLSSSTYGKDNVKFLKVKKDPQNPKKQEVMEATVTCLLEGGFDTSYTEADNSSIVPTDTVKNTILVLAKTTEIWPIERFAAKLATHFVEKYSHVSGVSVKIVQDRWVKYAVDGKPHDHSFIHEGGEKRITDLYYKRSGDYKLSSAIKDLTVLKSTGSMFYGYNKCDFTTLQPTTDRILSTDVDATWVWDNKKIGSVYDIAKAAXKGIFDNVYNQAREITLTTFALENSPSVQATMFNMATQILEKACSVYSVSYALPNKHYFLIDLKWKGLENDNELFYPSPHPNGLIKCTVVRKEKTKL

SEQ ID NO: 82 - protein, 303 aa, synthetic construct
  source 1..303: note = Candida.Uox Variant Subunit (K117)
  VARIANT 117: note = Xaa is unnatural amino acid containing tetrazine functionalgroup, and/or triazine functional group.
MSTTLSSSTYGKDNVKFLKVKKDPQNPKKQEVMEATVTCLLEGGFDTSYTEADNSSIVPTDTVKNTILVLAKTTEIWPIERFAAKLATHFVEKYSHVSGVSVKIVQDRWVKYAVDGXPHDHSFIHEGGEKRITDLYYKRSGDYKLSSAIKDLTVLKSTGSMFYGYNKCDFTTLQPTTDRILSTDVDATWVWDNKKIGSVYDIAKAADKGIFDNVYNQAREITLTTFALENSPSVQATMFNMATQILEKACSVYSVSYALPNKHYFLIDLKWKGLENDNELFYPSPHPNGLIKCTVVRKEKTKL

SEQ ID NO: 83 - protein, 303 aa, synthetic construct
  source 1..303: note = Candida.Uox Variant Subunit (C250)
  VARIANT 250: note = Xaa is unnatural amino acid containing tetrazine functionalgroup, and/or triazine functional group.
MSTTLSSSTYGKDNVKFLKVKKDPQNPKKQEVMEATVTCLLEGGFDTSYTEADNSSIVPTDTVKNTILVLAKTTEIWPIERFAAKLATHFVEKYSHVSGVSVKIVQDRWVKYAVDGKPHDHSFIHEGGEKRITDLYYKRSGDYKLSSAIKDLTVLKSTGSMFYGYNKCDFTTLQPTTDRILSTDVDATWVWDNKKIGSVYDIAKAADKGIFDNVYNQAREITLTTFALENSPSVQATMFNMATQILEKAXSVYSVSYALPNKHYFLIDLKWKGLENDNELFYPSPHPNGLIKCTVVRKEKTKL

SEQ ID NO: 84 - protein, 303 aa, synthetic construct
  source 1..303: note = Candida.Uox Variant Subunit (P175)
  VARIANT 175: note = Xaa is unnatural amino acid containing tetrazine functionalgroup, and/or triazine functional group.
MSTTLSSSTYGKDNVKFLKVKKDPQNPKKQEVMEATVTCLLEGGFDTSYTEADNSSIVPTDTVKNTILVLAKTTEIWPIERFAAKLATHFVEKYSHVSGVSVKIVQDRWVKYAVDGKPHDHSFIHEGGEKRITDLYYKRSGDYKLSSAIKDLTVLKSTGSMFYGYNKCDFTTLQXTTDRILSTDVDATWVWDNKKIGSVYDIAKAADKGIFDNVYNQAREITLTTFALENSPSVQATMFNMATQILEKACSVYSVSYALPNKHYFLIDLKWKGLENDNELFYPSPHPNGLIKCTVVRKEKTKL

SEQ ID NO: 85 - protein, 303 aa, synthetic construct
  source 1..303: note = Candida.Uox Variant Subunit (K270)
  VARIANT 270: note = Xaa is unnatural amino acid containing tetrazine functionalgroup, and/or triazine functional group.
MSTTLSSSTYGKDNVKFLKVKKDPQNPKKQEVMEATVTCLLEGGFDTSYTEADNSSIVPTDTVKNTILVLAKTTEIWPIERFAAKLATHFVEKYSHVSGVSVKIVQDRWVKYAVDGKPHDHSFIHEGGEKRITDLYYKRSGDYKLSSAIKDLTVLKSTGSMFYGYNKCDFTTLQPTTDRILSTDVDATWVWDNKKIGSVYDIAKAADKGIFDNVYNQAREITLTTFALENSPSVQATMFNMATQILEKACSVYSVSYALPNKHYFLIDLXWKGLENDNELFYPSPHPNGLIKCTVVRKEKTKL

SEQ ID NO: 86 - protein, 303 aa, synthetic construct
  source 1..303: note = Candida.Uox Variant Subunit (D268)
  VARIANT 268: note = Xaa is unnatural amino acid containing tetrazine functionalgroup, and/or triazine functional group.
MSTTLSSSTYGKDNVKFLKVKKDPQNPKKQEVMEATVTCLLEGGFDTSYTEADNSSIVPTDTVKNTILVLAKTTEIWPIERFAAKLATHFVEKYSHVSGVSVKIVQDRWVKYAVDGKPHDHSFIHEGGEKRITDLYYKRSGDYKLSSAIKDLTVLKSTGSMFYGYNKCDFTTLQPTTDRILSTDVDATWVWDNKKIGSVYDIAKAADKGIFDNVYNQAREITLTTFALENSPSVQATMFNMATQILEKACSVYSVSYALPNKHYFLIXLKWKGLENDNELFYPSPHPNGLIKCTVVRKEKTKL

SEQ ID NO: 87 - protein, 303 aa, synthetic construct
  source 1..303: note = Candida.Uox Variant Subunit (G44)
  VARIANT 44: note = Xaa is unnatural amino acid containing tetrazine functionalgroup, and/or triazine functional group.
MSTTLSSSTYGKDNVKFLKVKKDPQNPKKQEVMEATVTCLLEGXFDTSYTEADNSSIVPTDTVKNTILVLAKTTEIWPIERFAAKLATHFVEKYSHVSGVSVKIVQDRWVKYAVDGKPHDHSFIHEGGEKRITDLYYKRSGDYKLSSAIKDLTVLKSTGSMFYGYNKCDFTTLQPTTDRILSTDVDATWVWDNKKIGSVYDIAKAADKGIFDNVYNQAREITLTTFALENSPSVQATMFNMATQILEKACSVYSVSYALPNKHYFLIDLKWKGLENDNELFYPSPHPNGLIKCTVVRKEKTKL

SEQ ID NO: 88 - protein, 303 aa, synthetic construct
  source 1..303: note = Candida.Uox Variant Subunit (N193)
  VARIANT 193: note = Xaa is unnatural amino acid containing tetrazine functionalgroup, and/or triazine functional group.
MSTTLSSSTYGKDNVKFLKVKKDPQNPKKQEVMEATVTCLLEGGFDTSYTEADNSSIVPTDTVKNTILVLAKTTEIWPIERFAAKLATHFVEKYSHVSGVSVKIVQDRWVKYAVDGKPHDHSFIHEGGEKRITDLYYKRSGDYKLSSAIKDLTVLKSTGSMFYGYNKCDFTTLQPTTDRILSTDVDATWVWDXKKIGSVYDIAKAADKGIFDNVYNQAREITLTTFALENSPSVQATMFNMATQILEKACSVYSVSYALPNKHYFLIDLKWKGLENDNELFYPSPHPNGLIKCTVVRKEKTKL

SEQ ID NO: 89 - protein, 303 aa, synthetic construct
  source 1..303: note = Candida.Uox Variant Subunit (G164)
  VARIANT 164: note = Xaa is unnatural amino acid containing tetrazine functionalgroup, and/or triazine functional group.
MSTTLSSSTYGKDNVKFLKVKKDPQNPKKQEVMEATVTCLLEGGFDTSYTEADNSSIVPTDTVKNTILVLAKTTEIWPIERFAAKLATHFVEKYSHVSGVSVKIVQDRWVKYAVDGKPHDHSFIHEGGEKRITDLYYKRSGDYKLSSAIKDLTVLKSTGSMFYXYNKCDFTTLQPTTDRILSTDVDATWVWDNKKIGSVYDIAKAADKGIFDNVYNQAREITLTTFALENSPSVQATMFNMATQILEKACSVYSVSYALPNKHYFLIDLKWKGLENDNELFYPSPHPNGLIKCTVVRKEKTKL

SEQ ID NO: 90 - protein, 303 aa, synthetic construct
  source 1..303: note = Candida.Uox Variant Subunit (T73)
  VARIANT 73: note = Xaa is unnatural amino acid containing tetrazine functionalgroup, and/or triazine functional group.
MSTTLSSSTYGKDNVKFLKVKKDPQNPKKQEVMEATVTCLLEGGFDTSYTEADNSSIVPTDTVKNTILVLAKXTEIWPIERFAAKLATHFVEKYSHVSGVSVKIVQDRWVKYAVDGKPHDHSFIHEGGEKRITDLYYKRSGDYKLSSAIKDLTVLKSTGSMFYGYNKCDFTTLQPTTDRILSTDVDATWVWDNKKIGSVYDIAKAADKGIFDNVYNQAREITLTTFALENSPSVQATMFNMATQILEKACSVYSVSYALPNKHYFLIDLKWKGLENDNELFYPSPHPNGLIKCTVVRKEKTKL

SEQ ID NO: 91 - protein, 303 aa, synthetic construct
  source 1..303: note = Candida.Uox Variant Subunit (K29)
  VARIANT 29: note = Xaa is unnatural amino acid containing tetrazine functionalgroup, and/or triazine functional group.
MSTTLSSSTYGKDNVKFLKVKKDPQNPKXQEVMEATVTCLLEGGFDTSYTEADNSSIVPTDTVKNTILVLAKTTEIWPIERFAAKLATHFVEKYSHVSGVSVKIVQDRWVKYAVDGKPHDHSFIHEGGEKRITDLYYKRSGDYKLSSAIKDLTVLKSTGSMFYGYNKCDFTTLQPTTDRILSTDVDATWVWDNKKIGSVYDIAKAADKGIFDNVYNQAREITLTTFALENSPSVQATMFNMATQILEKACSVYSVSYALPNKHYFLIDLKWKGLENDNELFYPSPHPNGLIKCTVVRKEKTKL

SEQ ID NO: 92 - protein, 303 aa, synthetic construct
  source 1..303: note = Candida.Uox Variant Subunit (N230)
  VARIANT 230: note = Xaa is unnatural amino acid containing tetrazine functionalgroup, and/or triazine functional group.
MSTTLSSSTYGKDNVKFLKVKKDPQNPKKQEVMEATVTCLLEGGFDTSYTEADNSSIVPTDTVKNTILVLAKTTEIWPIERFAAKLATHFVEKYSHVSGVSVKIVQDRWVKYAVDGKPHDHSFIHEGGEKRITDLYYKRSGDYKLSSAIKDLTVLKSTGSMFYGYNKCDFTTLQPTTDRILSTDVDATWVWDNKKIGSVYDIAKAADKGIFDNVYNQAREITLTTFALEXSPSVQATMFNMATQILEKACSVYSVSYALPNKHYFLIDLKWKGLENDNELFYPSPHPNGLIKCTVVRKEKTKL

SEQ ID NO: 93 - protein, 303 aa, synthetic construct
  source 1..303: note = Candida.Uox Variant Subunit (Q25)
  VARIANT 25: note = Xaa is unnatural amino acid containing tetrazine functionalgroup, and/or triazine functional group.
MSTTLSSSTYGKDNVKFLKVKKDPXNPKKQEVMEATVTCLLEGGFDTSYTEADNSSIVPTDTVKNTILVLAKTTEIWPIERFAAKLATHFVEKYSHVSGVSVKIVQDRWVKYAVDGKPHDHSFIHEGGEKRITDLYYKRSGDYKLSSAIKDLTVLKSTGSMFYGYNKCDFTTLQPTTDRILSTDVDATWVWDNKKIGSVYDIAKAADKGIFDNVYNQAREITLTTFALENSPSVQATMFNMATQILEKACSVYSVSYALPNKHYFLIDLKWKGLENDNELFYPSPHPNGLIKCTVVRKEKTKL

SEQ ID NO: 94 - protein, 303 aa, synthetic construct
  source 1..303: note = Candida.Uox Variant Subunit (N216)
  VARIANT 216: note = Xaa is unnatural amino acid containing tetrazine functionalgroup, and/or triazine functional group.
MSTTLSSSTYGKDNVKFLKVKKDPQNPKKQEVMEATVTCLLEGGFDTSYTEADNSSIVPTDTVKNTILVLAKTTEIWPIERFAAKLATHFVEKYSHVSGVSVKIVQDRWVKYAVDGKPHDHSFIHEGGEKRITDLYYKRSGDYKLSSAIKDLTVLKSTGSMFYGYNKCDFTTLQPTTDRILSTDVDATWVWDNKKIGSVYDIAKAADKGIFDNVYXQAREITLTTFALENSPSVQATMFNMATQILEKACSVYSVSYALPNKHYFLIDLKWKGLENDNELFYPSPHPNGLIKCTVVRKEKTKL

SEQ ID NO: 95 - protein, 303 aa, synthetic construct
  source 1..303: note = Candida.Uox Variant Subunit (S55)
  VARIANT 55: note = Xaa is unnatural amino acid containing tetrazine functionalgroup, and/or triazine functional group.
MSTTLSSSTYGKDNVKFLKVKKDPQNPKKQEVMEATVTCLLEGGFDTSYTEADNXSIVPTDTVKNTILVLAKTTEIWPIERFAAKLATHFVEKYSHVSGVSVKIVQDRWVKYAVDGKPHDHSFIHEGGEKRITDLYYKRSGDYKLSSAIKDLTVLKSTGSMFYGYNKCDFTTLQPTTDRILSTDVDATWVWDNKKIGSVYDIAKAADKGIFDNVYNQAREITLTTFALENSPSVQATMFNMATQILEKACSVYSVSYALPNKHYFLIDLKWKGLENDNELFYPSPHPNGLIKCTVVRKEKTKL

SEQ ID NO: 96 - protein, 303 aa, synthetic construct
  source 1..303: note = Candida.Uox Variant Subunit (K28)
  VARIANT 28: note = Xaa is unnatural amino acid containing tetrazine functionalgroup, and/or triazine functional group.
MSTTLSSSTYGKDNVKFLKVKKDPQNPXKQEVMEATVTCLLEGGFDTSYTEADNSSIVPTDTVKNTILVLAKTTEIWPIERFAAKLATHFVEKYSHVSGVSVKIVQDRWVKYAVDGKPHDHSFIHEGGEKRITDLYYKRSGDYKLSSAIKDLTVLKSTGSMFYGYNKCDFTTLQPTTDRILSTDVDATWVWDNKKIGSVYDIAKAADKGIFDNVYNQAREITLTTFALENSPSVQATMFNMATQILEKACSVYSVSYALPNKHYFLIDLKWKGLENDNELFYPSPHPNGLIKCTVVRKEKTKL

SEQ ID NO: 97 - protein, 303 aa, synthetic construct
  source 1..303: note = Candida.Uox Variant Subunit (S6)
  VARIANT 6: note = Xaa is unnatural amino acid containing tetrazine functionalgroup, and/or triazine functional group.
MSTTLXSSTYGKDNVKFLKVKKDPQNPKKQEVMEATVTCLLEGGFDTSYTEADNSSIVPTDTVKNTILVLAKTTEIWPIERFAAKLATHFVEKYSHVSGVSVKIVQDRWVKYAVDGKPHDHSFIHEGGEKRITDLYYKRSGDYKLSSAIKDLTVLKSTGSMFYGYNKCDFTTLQPTTDRILSTDVDATWVWDNKKIGSVYDIAKAADKGIFDNVYNQAREITLTTFALENSPSVQATMFNMATQILEKACSVYSVSYALPNKHYFLIDLKWKGLENDNELFYPSPHPNGLIKCTVVRKEKTKL

SEQ ID NO: 98 - protein, 303 aa, synthetic construct
  source 1..303: note = Candida.Uox Variant Subunit (P27)
  VARIANT 27: note = Xaa is unnatural amino acid containing tetrazine functionalgroup, and/or triazine functional group.
MSTTLSSSTYGKDNVKFLKVKKDPQNXKKQEVMEATVTCLLEGGFDTSYTEADNSSIVPTDTVKNTILVLAKTTEIWPIERFAAKLATHFVEKYSHVSGVSVKIVQDRWVKYAVDGKPHDHSFIHEGGEKRITDLYYKRSGDYKLSSAIKDLTVLKSTGSMFYGYNKCDFTTLQPTTDRILSTDVDATWVWDNKKIGSVYDIAKAADKGIFDNVYNQAREITLTTFALENSPSVQATMFNMATQILEKACSVYSVSYALPNKHYFLIDLKWKGLENDNELFYPSPHPNGLIKCTVVRKEKTKL

SEQ ID NO: 99 - protein, 303 aa, synthetic construct
  source 1..303: note = Candida.Uox Variant Subunit (K298)
  VARIANT 298: note = Xaa is unnatural amino acid containing tetrazine functionalgroup, and/or triazine functional group.
MSTTLSSSTYGKDNVKFLKVKKDPQNPKKQEVMEATVTCLLEGGFDTSYTEADNSSIVPTDTVKNTILVLAKTTEIWPIERFAAKLATHFVEKYSHVSGVSVKIVQDRWVKYAVDGKPHDHSFIHEGGEKRITDLYYKRSGDYKLSSAIKDLTVLKSTGSMFYGYNKCDFTTLQPTTDRILSTDVDATWVWDNKKIGSVYDIAKAADKGIFDNVYNQAREITLTTFALENSPSVQATMFNMATQILEKACSVYSVSYALPNKHYFLIDLKWKGLENDNELFYPSPHPNGLIKCTVVRXEKTKL

SEQ ID NO: 100 - protein, 303 aa, synthetic construct
  source 1..303: note = Candida.Uox Variant Subunit (A113)
  VARIANT 113: note = Xaa is unnatural amino acid containing tetrazine functionalgroup, and/or triazine functional group.
MSTTLSSSTYGKDNVKFLKVKKDPQNPKKQEVMEATVTCLLEGGFDTSYTEADNSSIVPTDTVKNTILVLAKTTEIWPIERFAAKLATHFVEKYSHVSGVSVKIVQDRWVKYXVDGKPHDHSFIHEGGEKRITDLYYKRSGDYKLSSAIKDLTVLKSTGSMFYGYNKCDFTTLQPTTDRILSTDVDATWVWDNKKIGSVYDIAKAADKGIFDNVYNQAREITLTTFALENSPSVQATMFNMATQILEKACSVYSVSYALPNKHYFLIDLKWKGLENDNELFYPSPHPNGLIKCTVVRKEKTKL

SEQ ID NO: 101 - protein, 303 aa, synthetic construct
  source 1..303: note = Candida.Uox Variant Subunit (N213)
  VARIANT 213: note = Xaa is unnatural amino acid containing tetrazine functionalgroup, and/or triazine functional group.
MSTTLSSSTYGKDNVKFLKVKKDPQNPKKQEVMEATVTCLLEGGFDTSYTEADNSSIVPTDTVKNTILVLAKTTEIWPIERFAAKLATHFVEKYSHVSGVSVKIVQDRWVKYAVDGKPHDHSFIHEGGEKRITDLYYKRSGDYKLSSAIKDLTVLKSTGSMFYGYNKCDFTTLQPTTDRILSTDVDATWVWDNKKIGSVYDIAKAADKGIFDXVYNQAREITLTTFALENSPSVQATMFNMATQILEKACSVYSVSYALPNKHYFLIDLKWKGLENDNELFYPSPHPNGLIKCTVVRKEKTKL

SEQ ID NO: 102 - protein, 303 aa, synthetic construct
  source 1..303: note = Candida.Uox Variant Subunit (E220)
  VARIANT 220: note = Xaa is unnatural amino acid containing tetrazine functionalgroup, and/or triazine functional group.
MSTTLSSSTYGKDNVKFLKVKKDPQNPKKQEVMEATVTCLLEGGFDTSYTEADNSSIVPTDTVKNTILVLAKTTEIWPIERFAAKLATHFVEKYSHVSGVSVKIVQDRWVKYAVDGKPHDHSFIHEGGEKRITDLYYKRSGDYKLSSAIKDLTVLKSTGSMFYGYNKCDFTTLQPTTDRILSTDVDATWVWDNKKIGSVYDIAKAADKGIFDNVYNQARXITLTTFALENSPSVQATMFNMATQILEKACSVYSVSYALPNKHYFLIDLKWKGLENDNELFYPSPHPNGLIKCTVVRKEKTKL

SEQ ID NO: 103 - protein, 303 aa, synthetic construct
  source 1..303: note = Candida.Uox Variant Subunit (G141)
  VARIANT 141: note = Xaa is unnatural amino acid containing tetrazine functionalgroup, and/or triazine functional group.
MSTTLSSSTYGKDNVKFLKVKKDPQNPKKQEVMEATVTCLLEGGFDTSYTEADNSSIVPTDTVKNTILVLAKTTEIWPIERFAAKLATHFVEKYSHVSGVSVKIVQDRWVKYAVDGKPHDHSFIHEGGEKRITDLYYKRSXDYKLSSAIKDLTVLKSTGSMFYGYNKCDFTTLQPTTDRILSTDVDATWVWDNKKIGSVYDIAKAADKGIFDNVYNQAREITLTTFALENSPSVQATMFNMATQILEKACSVYSVSYALPNKHYFLIDLKWKGLENDNELFYPSPHPNGLIKCTVVRKEKTKL

SEQ ID NO: 104 - protein, 303 aa, synthetic construct
  source 1..303: note = Candida.Uox Variant Subunit (Y163)
  VARIANT 163: note = Xaa is unnatural amino acid containing tetrazine functionalgroup, and/or triazine functional group.
MSTTLSSSTYGKDNVKFLKVKKDPQNPKKQEVMEATVTCLLEGGFDTSYTEADNSSIVPTDTVKNTILVLAKTTEIWPIERFAAKLATHFVEKYSHVSGVSVKIVQDRWVKYAVDGKPHDHSFIHEGGEKRITDLYYKRSGDYKLSSAIKDLTVLKSTGSMFXGYNKCDFTTLQPTTDRILSTDVDATWVWDNKKIGSVYDIAKAADKGIFDNVYNQAREITLTTFALENSPSVQATMFNMATQILEKACSVYSVSYALPNKHYFLIDLKWKGLENDNELFYPSPHPNGLIKCTVVRKEKTKL

SEQ ID NO: 105 - protein, 303 aa, synthetic construct
  source 1..303: note = Candida.Uox Variant Subunit (Y253)
  VARIANT 253: note = Xaa is unnatural amino acid containing tetrazine functionalgroup, and/or triazine functional group.
MSTTLSSSTYGKDNVKFLKVKKDPQNPKKQEVMEATVTCLLEGGFDTSYTEADNSSIVPTDTVKNTILVLAKTTEIWPIERFAAKLATHFVEKYSHVSGVSVKIVQDRWVKYAVDGKPHDHSFIHEGGEKRITDLYYKRSGDYKLSSAIKDLTVLKSTGSMFYGYNKCDFTTLQPTTDRILSTDVDATWVWDNKKIGSVYDIAKAADKGIFDNVYNQAREITLTTFALENSPSVQATMFNMATQILEKACSVXSVSYALPNKHYFLIDLKWKGLENDNELFYPSPHPNGLIKCTVVRKEKTKL

SEQ ID NO: 106 - protein, 303 aa, synthetic construct
  source 1..303: note = Candida.Uox Variant Subunit (D178)
  VARIANT 178: note = Xaa is unnatural amino acid containing tetrazine functionalgroup, and/or triazine functional group.
MSTTLSSSTYGKDNVKFLKVKKDPQNPKKQEVMEATVTCLLEGGFDTSYTEADNSSIVPTDTVKNTILVLAKTTEIWPIERFAAKLATHFVEKYSHVSGVSVKIVQDRWVKYAVDGKPHDHSFIHEGGEKRITDLYYKRSGDYKLSSAIKDLTVLKSTGSMFYGYNKCDFTTLQPTTXRILSTDVDATWVWDNKKIGSVYDIAKAADKGIFDNVYNQAREITLTTFALENSPSVQATMFNMATQILEKACSVYSVSYALPNKHYFLIDLKWKGLENDNELFYPSPHPNGLIKCTVVRKEKTKL

SEQ ID NO: 107 - protein, 303 aa, synthetic construct
  source 1..303: note = Candida.Uox Variant Subunit (K93)
  VARIANT 93: note = Xaa is unnatural amino acid containing tetrazine functionalgroup, and/or triazine functional group.
MSTTLSSSTYGKDNVKFLKVKKDPQNPKKQEVMEATVTCLLEGGFDTSYTEADNSSIVPTDTVKNTILVLAKTTEIWPIERFAAKLATHFVEXYSHVSGVSVKIVQDRWVKYAVDGKPHDHSFIHEGGEKRITDLYYKRSGDYKLSSAIKDLTVLKSTGSMFYGYNKCDFTTLQPTTDRILSTDVDATWVWDNKKIGSVYDIAKAADKGIFDNVYNQAREITLTTFALENSPSVQATMFNMATQILEKACSVYSVSYALPNKHYFLIDLKWKGLENDNELFYPSPHPNGLIKCTVVRKEKTKL

SEQ ID NO: 108 - protein, 303 aa, synthetic construct
  source 1..303: note = Candida.Uox Variant Subunit (K103)
  VARIANT 103: note = Xaa is unnatural amino acid containing tetrazine functionalgroup, and/or triazine functional group.
MSTTLSSSTYGKDNVKFLKVKKDPQNPKKQEVMEATVTCLLEGGFDTSYTEADNSSIVPTDTVKNTILVLAKTTEIWPIERFAAKLATHFVEKYSHVSGVSVXIVQDRWVKYAVDGKPHDHSFIHEGGEKRITDLYYKRSGDYKLSSAIKDLTVLKSTGSMFYGYNKCDFTTLQPTTDRILSTDVDATWVWDNKKIGSVYDIAKAADKGIFDNVYNQAREITLTTFALENSPSVQATMFNMATQILEKACSVYSVSYALPNKHYFLIDLKWKGLENDNELFYPSPHPNGLIKCTVVRKEKTKL

SEQ ID NO: 109 - protein, 303 aa, synthetic construct
  source 1..303: note = Candida.Uox Variant Subunit (K144)
  VARIANT 144: note = Xaa is unnatural amino acid containing tetrazine functionalgroup, and/or triazine functional group.
MSTTLSSSTYGKDNVKFLKVKKDPQNPKKQEVMEATVTCLLEGGFDTSYTEADNSSIVPTDTVKNTILVLAKTTEIWPIERFAAKLATHFVEKYSHVSGVSVKIVQDRWVKYAVDGKPHDHSFIHEGGEKRITDLYYKRSGDYXLSSAIKDLTVLKSTGSMFYGYNKCDFTTLQPTTDRILSTDVDATWVWDNKKIGSVYDIAKAADKGIFDNVYNQAREITLTTFALENSPSVQATMFNMATQILEKACSVYSVSYALPNKHYFLIDLKWKGLENDNELFYPSPHPNGLIKCTVVRKEKTKL

SEQ ID NO: 110 - protein, 303 aa, synthetic construct
  source 1..303: note = Candida.Uox Variant Subunit (R139)
  VARIANT 139: note = Xaa is unnatural amino acid containing tetrazine functionalgroup, and/or triazine functional group.
MSTTLSSSTYGKDNVKFLKVKKDPQNPKKQEVMEATVTCLLEGGFDTSYTEADNSSIVPTDTVKNTILVLAKTTEIWPIERFAAKLATHFVEKYSHVSGVSVKIVQDRWVKYAVDGKPHDHSFIHEGGEKRITDLYYKXSGDYKLSSAIKDLTVLKSTGSMFYGYNKCDFTTLQPTTDRILSTDVDATWVWDNKKIGSVYDIAKAADKGIFDNVYNQAREITLTTFALENSPSVQATMFNMATQILEKACSVYSVSYALPNKHYFLIDLKWKGLENDNELFYPSPHPNGLIKCTVVRKEKTKL

SEQ ID NO: 111 - protein, 303 aa, synthetic construct
  source 1..303: note = Candida.Uox Variant Subunit (K138)
  VARIANT 138: note = Xaa is unnatural amino acid containing tetrazine functionalgroup, and/or triazine functional group.
MSTTLSSSTYGKDNVKFLKVKKDPQNPKKQEVMEATVTCLLEGGFDTSYTEADNSSIVPTDTVKNTILVLAKTTEIWPIERFAAKLATHFVEKYSHVSGVSVKIVQDRWVKYAVDGKPHDHSFIHEGGEKRITDLYYXRSGDYKLSSAIKDLTVLKSTGSMFYGYNKCDFTTLQPTTDRILSTDVDATWVWDNKKIGSVYDIAKAADKGIFDNVYNQAREITLTTFALENSPSVQATMFNMATQILEKACSVYSVSYALPNKHYFLIDLKWKGLENDNELFYPSPHPNGLIKCTVVRKEKTKL

SEQ ID NO: 112 - protein, 303 aa, synthetic construct
  source 1..303: note = Candida.Uox Variant Subunit (S7)
  VARIANT 7: note = Xaa is unnatural amino acid containing tetrazine functionalgroup, and/or triazine functional group.
MSTTLSXSTYGKDNVKFLKVKKDPQNPKKQEVMEATVTCLLEGGFDTSYTEADNSSIVPTDTVKNTILVLAKTTEIWPIERFAAKLATHFVEKYSHVSGVSVKIVQDRWVKYAVDGKPHDHSFIHEGGEKRITDLYYKRSGDYKLSSAIKDLTVLKSTGSMFYGYNKCDFTTLQPTTDRILSTDVDATWVWDNKKIGSVYDIAKAADKGIFDNVYNQAREITLTTFALENSPSVQATMFNMATQILEKACSVYSVSYALPNKHYFLIDLKWKGLENDNELFYPSPHPNGLIKCTVVRKEKTKL

SEQ ID NO: 113 - protein, 303 aa, synthetic construct
  source 1..303: note = Candida.Uox Variant Subunit (D151)
  VARIANT 151: note = Xaa is unnatural amino acid containing tetrazine functionalgroup, and/or triazine functional group.
MSTTLSSSTYGKDNVKFLKVKKDPQNPKKQEVMEATVTCLLEGGFDTSYTEADNSSIVPTDTVKNTILVLAKTTEIWPIERFAAKLATHFVEKYSHVSGVSVKIVQDRWVKYAVDGKPHDHSFIHEGGEKRITDLYYKRSGDYKLSSAIKXLTVLKSTGSMFYGYNKCDFTTLQPTTDRILSTDVDATWVWDNKKIGSVYDIAKAADKGIFDNVYNQAREITLTTFALENSPSVQATMFNMATQILEKACSVYSVSYALPNKHYFLIDLKWKGLENDNELFYPSPHPNGLIKCTVVRKEKTKL

SEQ ID NO: 114 - protein, 303 aa, synthetic construct
  source 1..303: note = Candida.Uox Variant Subunit (R297)
  VARIANT 297: note = Xaa is unnatural amino acid containing tetrazine functionalgroup, and/or triazine functional group.
MSTTLSSSTYGKDNVKFLKVKKDPQNPKKQEVMEATVTCLLEGGFDTSYTEADNSSIVPTDTVKNTILVLAKTTEIWPIERFAAKLATHFVEKYSHVSGVSVKIVQDRWVKYAVDGKPHDHSFIHEGGEKRITDLYYKRSGDYKLSSAIKDLTVLKSTGSMFYGYNKCDFTTLQPTTDRILSTDVDATWVWDNKKIGSVYDIAKAADKGIFDNVYNQAREITLTTFALENSPSVQATMFNMATQILEKACSVYSVSYALPNKHYFLIDLKWKGLENDNELFYPSPHPNGLIKCTVVXKEKTKL

SEQ ID NO: 115 - protein, 303 aa, synthetic construct
  source 1..303: note = Candida.Uox Variant Subunit (K272)
  VARIANT 272: note = Xaa is unnatural amino acid containing tetrazine functionalgroup, and/or triazine functional group.
MSTTLSSSTYGKDNVKFLKVKKDPQNPKKQEVMEATVTCLLEGGFDTSYTEADNSSIVPTDTVKNTILVLAKTTEIWPIERFAAKLATHFVEKYSHVSGVSVKIVQDRWVKYAVDGKPHDHSFIHEGGEKRITDLYYKRSGDYKLSSAIKDLTVLKSTGSMFYGYNKCDFTTLQPTTDRILSTDVDATWVWDNKKIGSVYDIAKAADKGIFDNVYNQAREITLTTFALENSPSVQATMFNMATQILEKACSVYSVSYALPNKHYFLIDLKWXGLENDNELFYPSPHPNGLIKCTVVRKEKTKL

SEQ ID NO: 116 - protein, 303 aa, synthetic construct
  source 1..303: note = Candida.Uox Variant Subunit (N278)
  VARIANT 278: note = Xaa is unnatural amino acid containing tetrazine functionalgroup, and/or triazine functional group.
MSTTLSSSTYGKDNVKFLKVKKDPQNPKKQEVMEATVTCLLEGGFDTSYTEADNSSIVPTDTVKNTILVLAKTTEIWPIERFAAKLATHFVEKYSHVSGVSVKIVQDRWVKYAVDGKPHDHSFIHEGGEKRITDLYYKRSGDYKLSSAIKDLTVLKSTGSMFYGYNKCDFTTLQPTTDRILSTDVDATWVWDNKKIGSVYDIAKAADKGIFDNVYNQAREITLTTFALENSPSVQATMFNMATQILEKACSVYSVSYALPNKHYFLIDLKWKGLENDXELFYPSPHPNGLIKCTVVRKEKTKL

SEQ ID NO: 117 - protein, 303 aa, synthetic construct
  source 1..303: note = Candida.Uox Variant Subunit (F265)
  VARIANT 265: note = Xaa is unnatural amino acid containing tetrazine functionalgroup, and/or triazine functional group.
MSTTLSSSTYGKDNVKFLKVKKDPQNPKKQEVMEATVTCLLEGGFDTSYTEADNSSIVPTDTVKNTILVLAKTTEIWPIERFAAKLATHFVEKYSHVSGVSVKIVQDRWVKYAVDGKPHDHSFIHEGGEKRITDLYYKRSGDYKLSSAIKDLTVLKSTGSMFYGYNKCDFTTLQPTTDRILSTDVDATWVWDNKKIGSVYDIAKAADKGIFDNVYNQAREITLTTFALENSPSVQATMFNMATQILEKACSVYSVSYALPNKHYXLIDLKWKGLENDNELFYPSPHPNGLIKCTVVRKEKTKL

SEQ ID NO: 118 - protein, 302 aa, Arthrobacter globiformis
MTATAETSTGTKVVLGQNQYGKAEVRLVKVTRNTARHEIQDLNVTSQLRGDFEAAHTAGDNAHVVATDTQKNTVYAFARDGFATTEEFLLRLGKHFTEGFDWVTGGRWAAQQFFWDRINDHDHAFSRNKSEVRTAVLEISGSEQAIVAGIEGLTVLKSTGSEFHGFPRDKYTTLQETTDRILATDVSARWRYNTVEVDFDAVYASVRGLLLKAFAETHSLALQQTMYEMGRAVIETHPEIDEIKMSLPNKHHFLVDLQPFGQDNPNEVFYAADRPYGLIEATIQREGSRADHPIWSNIAGFC

SEQ ID NO: 119 - protein, 302 aa, synthetic construct
  source 1..302: note = Arth.Uox Variant Subunit (D80)
  VARIANT 80: note = Xaa is unnatural amino acid containing tetrazine functionalgroup, and/or triazine functional group.
MTATAETSTGTKVVLGQNQYGKAEVRLVKVTRNTARHEIQDLNVTSQLRGDFEAAHTAGDNAHVVATDTQKNTVYAFARXGFATTEEFLLRLGKHFTEGFDWVTGGRWAAQQFFWDRINDHDHAFSRNKSEVRTAVLEISGSEQAIVAGIEGLTVLKSTGSEFHGFPRDKYTTLQETTDRILATDVSARWRYNTVEVDFDAVYASVRGLLLKAFAETHSLALQQTMYEMGRAVIETHPEIDEIKMSLPNKHHFLVDLQPFGQDNPNEVFYAADRPYGLIEATIQREGSRADHPIWSNIAGFC

SEQ ID NO: 120 - protein, 302 aa, synthetic construct
  source 1..302: note = Arth.Uox Variant Subunit (F82)
  VARIANT 82: note = Xaa is unnatural amino acid containing tetrazine functionalgroup, and/or triazine functional group.
MTATAETSTGTKVVLGQNQYGKAEVRLVKVTRNTARHEIQDLNVTSQLRGDFEAAHTAGDNAHVVATDTQKNTVYAFARDGXATTEEFLLRLGKHFTEGFDWVTGGRWAAQQFFWDRINDHDHAFSRNKSEVRTAVLEISGSEQAIVAGIEGLTVLKSTGSEFHGFPRDKYTTLQETTDRILATDVSARWRYNTVEVDFDAVYASVRGLLLKAFAETHSLALQQTMYEMGRAVIETHPEIDEIKMSLPNKHHFLVDLQPFGQDNPNEVFYAADRPYGLIEATIQREGSRADHPIWSNIAGFC

SEQ ID NO: 121 - protein, 302 aa, synthetic construct
  source 1..302: note = Arth.Uox Variant Subunit (F100)
  VARIANT 100: note = Xaa is unnatural amino acid containing tetrazine functionalgroup, and/or triazine functional group.
MTATAETSTGTKVVLGQNQYGKAEVRLVKVTRNTARHEIQDLNVTSQLRGDFEAAHTAGDNAHVVATDTQKNTVYAFARDGFATTEEFLLRLGKHFTEGXDWVTGGRWAAQQFFWDRINDHDHAFSRNKSEVRTAVLEISGSEQAIVAGIEGLTVLKSTGSEFHGFPRDKYTTLQETTDRILATDVSARWRYNTVEVDFDAVYASVRGLLLKAFAETHSLALQQTMYEMGRAVIETHPEIDEIKMSLPNKHHFLVDLQPFGQDNPNEVFYAADRPYGLIEATIQREGSRADHPIWSNIAGFC

SEQ ID NO: 122 - protein, 302 aa, synthetic construct
  source 1..302: note = Arth.Uox Variant Subunit (D101)
  VARIANT 101: note = Xaa is unnatural amino acid containing tetrazine functionalgroup, and/or triazine functional group.
MTATAETSTGTKVVLGQNQYGKAEVRLVKVTRNTARHEIQDLNVTSQLRGDFEAAHTAGDNAHVVATDTQKNTVYAFARDGFATTEEFLLRLGKHFTEGFXWVTGGRWAAQQFFWDRINDHDHAFSRNKSEVRTAVLEISGSEQAIVAGIEGLTVLKSTGSEFHGFPRDKYTTLQETTDRILATDVSARWRYNTVEVDFDAVYASVRGLLLKAFAETHSLALQQTMYEMGRAVIETHPEIDEIKMSLPNKHHFLVDLQPFGQDNPNEVFYAADRPYGLIEATIQREGSRADHPIWSNIAGFC

SEQ ID NO: 123 - protein, 302 aa, synthetic construct
  source 1..302: note = Arth.Uox Variant Subunit (F114)
  VARIANT 114: note = Xaa is unnatural amino acid containing tetrazine functionalgroup, and/or triazine functional group.
MTATAETSTGTKVVLGQNQYGKAEVRLVKVTRNTARHEIQDLNVTSQLRGDFEAAHTAGDNAHVVATDTQKNTVYAFARDGFATTEEFLLRLGKHFTEGFDWVTGGRWAAQQFXWDRINDHDHAFSRNKSEVRTAVLEISGSEQAIVAGIEGLTVLKSTGSEFHGFPRDKYTTLQETTDRILATDVSARWRYNTVEVDFDAVYASVRGLLLKAFAETHSLALQQTMYEMGRAVIETHPEIDEIKMSLPNKHHFLVDLQPFGQDNPNEVFYAADRPYGLIEATIQREGSRADHPIWSNIAGFC

SEQ ID NO: 124 - protein, 302 aa, synthetic construct
  source 1..302: note = Arth.Uox Variant Subunit (N119)
  VARIANT 119: note = Xaa is unnatural amino acid containing tetrazine functionalgroup, and/or triazine functional group.
MTATAETSTGTKVVLGQNQYGKAEVRLVKVTRNTARHEIQDLNVTSQLRGDFEAAHTAGDNAHVVATDTQKNTVYAFARDGFATTEEFLLRLGKHFTEGFDWVTGGRWAAQQFFWDRIXDHDHAFSRNKSEVRTAVLEISGSEQAIVAGIEGLTVLKSTGSEFHGFPRDKYTTLQETTDRILATDVSARWRYNTVEVDFDAVYASVRGLLLKAFAETHSLALQQTMYEMGRAVIETHPEIDEIKMSLPNKHHFLVDLQPFGQDNPNEVFYAADRPYGLIEATIQREGSRADHPIWSNIAGFC

SEQ ID NO: 125 - protein, 302 aa, synthetic construct
  source 1..302: note = Arth.Uox Variant Subunit (D120)
  VARIANT 120: note = Xaa is unnatural amino acid containing tetrazine functionalgroup, and/or triazine functional group.
MTATAETSTGTKVVLGQNQYGKAEVRLVKVTRNTARHEIQDLNVTSQLRGDFEAAHTAGDNAHVVATDTQKNTVYAFARDGFATTEEFLLRLGKHFTEGFDWVTGGRWAAQQFFWDRINXHDHAFSRNKSEVRTAVLEISGSEQAIVAGIEGLTVLKSTGSEFHGFPRDKYTTLQETTDRILATDVSARWRYNTVEVDFDAVYASVRGLLLKAFAETHSLALQQTMYEMGRAVIETHPEIDEIKMSLPNKHHFLVDLQPFGQDNPNEVFYAADRPYGLIEATIQREGSRADHPIWSNIAGFC

SEQ ID NO: 126 - protein, 302 aa, synthetic construct
  source 1..302: note = Arth.Uox Variant Subunit (S142)
  VARIANT 142: note = Xaa is unnatural amino acid containing tetrazine functionalgroup, and/or triazine functional group.
MTATAETSTGTKVVLGQNQYGKAEVRLVKVTRNTARHEIQDLNVTSQLRGDFEAAHTAGDNAHVVATDTQKNTVYAFARDGFATTEEFLLRLGKHFTEGFDWVTGGRWAAQQFFWDRINDHDHAFSRNKSEVRTAVLEISGXEQAIVAGIEGLTVLKSTGSEFHGFPRDKYTTLQETTDRILATDVSARWRYNTVEVDFDAVYASVRGLLLKAFAETHSLALQQTMYEMGRAVIETHPEIDEIKMSLPNKHHFLVDLQPFGQDNPNEVFYAADRPYGLIEATIQREGSRADHPIWSNIAGFC

SEQ ID NO: 127 - protein, 302 aa, synthetic construct
  source 1..302: note = Arth.Uox Variant Subunit (E143)
  VARIANT 143: note = Xaa is unnatural amino acid containing tetrazine functionalgroup, and/or triazine functional group.
MTATAETSTGTKVVLGQNQYGKAEVRLVKVTRNTARHEIQDLNVTSQLRGDFEAAHTAGDNAHVVATDTQKNTVYAFARDGFATTEEFLLRLGKHFTEGFDWVTGGRWAAQQFFWDRINDHDHAFSRNKSEVRTAVLEISGSXQAIVAGIEGLTVLKSTGSEFHGFPRDKYTTLQETTDRILATDVSARWRYNTVEVDFDAVYASVRGLLLKAFAETHSLALQQTMYEMGRAVIETHPEIDEIKMSLPNKHHFLVDLQPFGQDNPNEVFYAADRPYGLIEATIQREGSRADHPIWSNIAGFC

SEQ ID NO: 128 - protein, 302 aa, synthetic construct
  source 1..302: note = Arth.Uox Variant Subunit (G175)
  VARIANT 175: note = Xaa is unnatural amino acid containing tetrazine functionalgroup, and/or triazine functional group.
MTATAETSTGTKVVLGQNQYGKAEVRLVKVTRNTARHEIQDLNVTSQLRGDFEAAHTAGDNAHVVATDTQKNTVYAFARDGFATTEEFLLRLGKHFTEGFDWVTGGRWAAQQFFWDRINDHDHAFSRNKSEVRTAVLEISGSEQAIVAGIEGLTVLKSTGSEFHGFPRDKYTTLXETTDRILATDVSARWRYNTVEVDFDAVYASVRGLLLKAFAETHSLALQQTMYEMGRAVIETHPEIDEIKMSLPNKHHFLVDLQPFGQDNPNEVFYAADRPYGLIEATIQREGSRADHPIWSNIAGFC

SEQ ID NO: 129 - protein, 302 aa, synthetic construct
  source 1..302: note = Arth.Uox Variant Subunit (V195)
  VARIANT 195: note = Xaa is unnatural amino acid containing tetrazine functionalgroup, and/or triazine functional group.
MTATAETSTGTKVVLGQNQYGKAEVRLVKVTRNTARHEIQDLNVTSQLRGDFEAAHTAGDNAHVVATDTQKNTVYAFARDGFATTEEFLLRLGKHFTEGFDWVTGGRWAAQQFFWDRINDHDHAFSRNKSEVRTAVLEISGSEQAIVAGIEGLTVLKSTGSEFHGFPRDKYTTLQETTDRILATDVSARWRYNTXEVDFDAVYASVRGLLLKAFAETHSLALQQTMYEMGRAVIETHPEIDEIKMSLPNKHHFLVDLQPFGQDNPNEVFYAADRPYGLIEATIQREGSRADHPIWSNIAGFC

SEQ ID NO: 130 - protein, 302 aa, synthetic construct
  source 1..302: note = Arth.Uox Variant Subunit (E196)
  VARIANT 196: note = Xaa is unnatural amino acid containing tetrazine functionalgroup, and/or triazine functional group.
MTATAETSTGTKVVLGQNQYGKAEVRLVKVTRNTARHEIQDLNVTSQLRGDFEAAHTAGDNAHVVATDTQKNTVYAFARDGFATTEEFLLRLGKHFTEGFDWVTGGRWAAQQFFWDRINDHDHAFSRNKSEVRTAVLEISGSEQAIVAGIEGLTVLKSTGSEFHGFPRDKYTTLQETTDRILATDVSARWRYNTVXVDFDAVYASVRGLLLKAFAETHSLALQQTMYEMGRAVIETHPEIDEIKMSLPNKHHFLVDLQPFGQDNPNEVFYAADRPYGLIEATIQREGSRADHPIWSNIAGFC

SEQ ID NO: 131 - protein, 302 aa, synthetic construct
  source 1..302: note = Arth.Uox Variant Subunit (H218)
  VARIANT 218: note = Xaa is unnatural amino acid containing tetrazine functionalgroup, and/or triazine functional group.
MTATAETSTGTKVVLGQNQYGKAEVRLVKVTRNTARHEIQDLNVTSQLRGDFEAAHTAGDNAHVVATDTQKNTVYAFARDGFATTEEFLLRLGKHFTEGFDWVTGGRWAAQQFFWDRINDHDHAFSRNKSEVRTAVLEISGSEQAIVAGIEGLTVLKSTGSEFHGFPRDKYTTLQETTDRILATDVSARWRYNTVEVDFDAVYASVRGLLLKAFAETXSLALQQTMYEMGRAVIETHPEIDEIKMSLPNKHHFLVDLQPFGQDNPNEVFYAADRPYGLIEATIQREGSRADHPIWSNIAGFC

SEQ ID NO: 132 - protein, 302 aa, synthetic construct
  source 1..302: note = Arth.Uox Variant Subunit (P238)
  VARIANT 238: note = Xaa is unnatural amino acid containing tetrazine functionalgroup, and/or triazine functional group.
MTATAETSTGTKVVLGQNQYGKAEVRLVKVTRNTARHEIQDLNVTSQLRGDFEAAHTAGDNAHVVATDTQKNTVYAFARDGFATTEEFLLRLGKHFTEGFDWVTGGRWAAQQFFWDRINDHDHAFSRNKSEVRTAVLEISGSEQAIVAGIEGLTVLKSTGSEFHGFPRDKYTTLQETTDRILATDVSARWRYNTVEVDFDAVYASVRGLLLKAFAETHSLALQQTMYEMGRAVIETHXEIDEIKMSLPNKHHFLVDLQPFGQDNPNEVFYAADRPYGLIEATIQREGSRADHPIWSNIAGFC

SEQ ID NO: 133 - protein, 585 aa, Homo sapiens [sequence omitted]

SEQ ID NO: 134 - protein, 585 aa, synthetic construct
  source 1..585: note = HSA Variant (V148M)
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQMSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 135 - protein, 585 aa, synthetic construct
  source 1..585: note = HSA variant (T420A)
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSAPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 136 - protein, 585 aa, synthetic construct
  source 1..585: note = HSA variant (E505R)
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNARTFTFHADICTLSEKERQIKKQTALVELVKHKPKATKEQLKAVMDDFAAFVEKCCKADDKETCFAEEGKKLVAASQAALGL

SEQ ID NO: 137 - protein, 585 aa, synthetic construct
  source 1..585: note = HSA variant (E505G)
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAGTFTFHADICTLSEKERQIKKQTALVELVKHKPKATKEQLKAVMDDFAAFVEKCCKADDKETCFAEEGKKLVAASQAALGL

SEQ ID NO: 138 - protein, 585 aa, synthetic construct
  source 1..585: note = HSA variant (V547A)
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAAMDDFAAFVEKCCKADDKETCFAEEGKKLVAASQAALGL

SEQ ID NO: 139 - protein, 585 aa, synthetic construct
  source 1..585: note = HSA variant (K573Y)
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGYKLVAASQAALGL

SEQ ID NO: 140 - protein, 585 aa, synthetic construct
  source 1..585: note = HSA variant (V424I)
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLIEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 141 - protein, 585 aa, synthetic construct
  source 1..585: note = HSA variant (N429D)
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRDLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 142 - protein, 585 aa, synthetic construct
  source 1..585: note = HSA variant (A449V)
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCVEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 143 - protein, 585 aa, synthetic construct
  source 1..585: note = HSA variant (T467M)
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKMPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 144 - protein, 585 aa, synthetic construct
  source 1..585: note = HSA variant (A552T)
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFTAFVEKCCKADDKETCFAEEGKKLVAASQAALGL

SEQ ID NO: 145 - DNA, 20 bp, synthetic construct
  source 1..20: mol_type = other DNA; note = pTAC linearize-F
caagcttggctgttttggcg

SEQ ID NO: 146 - DNA, 25 bp, synthetic construct
  source 1..25: mol_type = other DNA; note = pTAC linearize-R
ctatatctccttcttaaagttaaac

SEQ ID NO: 147 - DNA, 34 bp, synthetic construct
  source 1..34: mol_type = other DNA; note = tacP-RBS-MCS-rrnBt1t2-F
aagaaggagatatagatgtctgctgtgaaggccg

SEQ ID NO: 148 - DNA, 36 bp, synthetic construct
  source 1..36: mol_type = other DNA; note = tacP-RBS-MCS-rrnBt1t2-R
aaacagccaagcttgttacagcttgctcttcagaga

SEQ ID NO: 149 - DNA, 20 bp, synthetic construct
  source 1..20: mol_type = other DNA; note = pTAC-sequencing-F
gcctagagcaagacgtttcc

SEQ ID NO: 150 - DNA, 19 bp, synthetic construct
  source 1..19: mol_type = other DNA; note = pTAC-sequencing-R
ttaatgcagctggcacgac

SEQ ID NO: 151 - DNA, 23 bp, synthetic construct
  source 1..23: mol_type = other DNA; note = Ribosome binding site sequence
tttgtttaactttaagaaggaga

SEQ ID NO: 152 - DNA, 909 bp, synthetic construct
  source 1..909: mol_type = other DNA; note = Asp.Uox Variant Coding DNA (D112UAG)
atgtctgctgtgaaggccgcaagatatggcaaggataatgtgagggtgtacaaggtgcataaggacgaaaagactggcgtgcagacagtgtacgagatgaccgtgtgcgtcctgctggagggcgaaatcgagacttcttataccaaagccgacaactccgtgattgtggccacagattctatcaagaacactatctatatcaccgccaaacagaacccagtgacaccacctgaactgttcggcagcattctcggcacacactttattgagaagtacaaccacatccatgctgcacacgtgaatatcgtgtgtcatcgctggacccgcatggacatctagggaaagccacacccccactcttttatcagagactctgaagaaaagagaaacgtgcaggtcgacgtggtggagggaaaaggtatcgacatcaagagctcactctccggcctgaccgtgctgaagagtaccaattcacagttttgggggtttctgagagacgaatacactacactgaaggagacttgggatagaatcctgagtaccgacgtggatgcaacctggcagtggaagaatttttccgggctgcaggaagtgcggtcccacgtgcccaagtttgatgcaacctgggcaaccgcaagggaggtgacactgaaaacctttgccgaggacaactccgctagcgtgcaggccacaatgtacaagatggccgaacagatcctggccagacagcagctgattgagactgtggagtactctctgcctaacaagcactatttcgaaatcgacctgtcctggcacaagggactgcagaatactggtaaaaacgcagaggtgttcgcccctcagagtgatcccaatggtctgatcaaatgcacagtggggagatcctctctgaagagcaagctgtaa

SEQ ID NO: 153 - DNA, 909 bp, synthetic construct
  source 1..909: mol_type = other DNA; note = Asp.Uox Variant Coding DNA (W160UAG)
atgtctgctgtgaaggccgcaagatatggcaaggataatgtgagggtgtacaaggtgcataaggacgaaaagactggcgtgcagacagtgtacgagatgaccgtgtgcgtcctgctggagggcgaaatcgagacttcttataccaaagccgacaactccgtgattgtggccacagattctatcaagaacactatctatatcaccgccaaacagaacccagtgacaccacctgaactgttcggcagcattctcggcacacactttattgagaagtacaaccacatccatgctgcacacgtgaatatcgtgtgtcatcgctggacccgcatggacatcgacggaaagccacacccccactcttttatcagagactctgaagaaaagagaaacgtgcaggtcgacgtggtggagggaaaaggtatcgacatcaagagctcactctccggcctgaccgtgctgaagagtaccaattcacagttttaggggtttctgagagacgaatacactacactgaaggagacttgggatagaatcctgagtaccgacgtggatgcaacctggcagtggaagaatttttccgggctgcaggaagtgcggtcccacgtgcccaagtttgatgcaacctgggcaaccgcaagggaggtgacactgaaaacctttgccgaggacaactccgctagcgtgcaggccacaatgtacaagatggccgaacagatcctggccagacagcagctgattgagactgtggagtactctctgcctaacaagcactatttcgaaatcgacctgtcctggcacaagggactgcagaatactggtaaaaacgcagaggtgttcgcccctcagagtgatcccaatggtctgatcaaatgcacagtggggagatcctctctgaagagcaagctgtaa

SEQ ID NO: 154 - DNA, 909 bp, synthetic construct
  source 1..909: mol_type = other DNA; note = Asp.Uox Variant Coding DNA (W174UAG)
atgtctgctgtgaaggccgcaagatatggcaaggataatgtgagggtgtacaaggtgcataaggacgaaaagactggcgtgcagacagtgtacgagatgaccgtgtgcgtcctgctggagggcgaaatcgagacttcttataccaaagccgacaactccgtgattgtggccacagattctatcaagaacactatctatatcaccgccaaacagaacccagtgacaccacctgaactgttcggcagcattctcggcacacactttattgagaagtacaaccacatccatgctgcacacgtgaatatcgtgtgtcatcgctggacccgcatggacatcgacggaaagccacacccccactcttttatcagagactctgaagaaaagagaaacgtgcaggtcgacgtggtggagggaaaaggtatcgacatcaagagctcactctccggcctgaccgtgctgaagagtaccaattcacagttttgggggtttctgagagacgaatacactacactgaaggagacttaggatagaatcctgagtaccgacgtggatgcaacctggcagtggaagaatttttccgggctgcaggaagtgcggtcccacgtgcccaagtttgatgcaacctgggcaaccgcaagggaggtgacactgaaaacctttgccgaggacaactccgctagcgtgcaggccacaatgtacaagatggccgaacagatcctggccagacagcagctgattgagactgtggagtactctctgcctaacaagcactatttcgaaatcgacctgtcctggcacaagggactgcagaatactggtaaaaacgcagaggtgttcgcccctcagagtgatcccaatggtctgatcaaatgcacagtggggagatcctctctgaagagcaagctgtaa

SEQ ID NO: 155 - DNA, 912 bp, synthetic construct
  source 1..912: mol_type = other DNA; note = Candida.Uox Variant Coding DNA (Y163UAG)
atgagcaccacactgagcagcagcacctatggtaaagataatgtgaaattcctgaaagtgaaaaaagatccgcagaacccgaaaaaacaagaagttatggaagcaaccgttacctgtctgctggaaggtggttttgataccagctataccgaagcagataatagcagcattgttccgaccgataccgtgaaaaataccattctggttctggcaaaaaccaccgaaatttggccgattgaacgttttgcagccaaactggcaacccattttgttgagaaatattctcatgttagcggtgtgagcgttaaaattgttcaggatcgttgggttaaatatgccgttgatggtaaaccgcatgatcacagctttattcatgaaggtggtgaaaaacgtatcaccgacctgtattacaaacgtagcggtgattataaactgtccagcgcaattaaagatctgaccgttctgaaaagcaccggcagcatgttttagggttataacaaatgcgatttcacaaccctgcagccgaccaccgatcgtattctgagcaccgatgttgatgcaacctgggtttgggataataagaaaattggtagcgtgtacgatattgccaaagcagcagataaaggcatcttcgataatgtgtataatcaggcacgtgaaattaccctgaccacctttgcactggaaaatagcccgagcgttcaggcaaccatgtttaatatggcgacccagattctggaaaaagcgtgtagcgtttatagcgttagctatgcactgccgaacaaacactattttctgattgacctgaaatggaagggccttgaaaatgataacgaactgttttatccgagtccgcatccgaatggtctgattaaatgtaccgttgtgcgtaaagagaaaaccaaactgtaa

SEQ ID NO: 156 - DNA, 912 bp, synthetic construct
  source 1..912: mol_type = other DNA; note = Candida.Uox Variant Coding DNA (Y200UAG)
atgagcaccacactgagcagcagcacctatggtaaagataatgtgaaattcctgaaagtgaaaaaagatccgcagaacccgaaaaaacaagaagttatggaagcaaccgttacctgtctgctggaaggtggttttgataccagctataccgaagcagataatagcagcattgttccgaccgataccgtgaaaaataccattctggttctggcaaaaaccaccgaaatttggccgattgaacgttttgcagccaaactggcaacccattttgttgagaaatattctcatgttagcggtgtgagcgttaaaattgttcaggatcgttgggttaaatatgccgttgatggtaaaccgcatgatcacagctttattcatgaaggtggtgaaaaacgtatcaccgacctgtattacaaacgtagcggtgattataaactgtccagcgcaattaaagatctgaccgttctgaaaagcaccggcagcatgttttatggttataacaaatgcgatttcacaaccctgcagccgaccaccgatcgtattctgagcaccgatgttgatgcaacctgggtttgggataataagaaaattggtagcgtgtaggatattgccaaagcagcagataaaggcatcttcgataatgtgtataatcaggcacgtgaaattaccctgaccacctttgcactggaaaatagcccgagcgttcaggcaaccatgtttaatatggcgacccagattctggaaaaagcgtgtagcgtttatagcgttagctatgcactgccgaacaaacactattttctgattgacctgaaatggaagggccttgaaaatgataacgaactgttttatccgagtccgcatccgaatggtctgattaaatgtaccgttgtgcgtaaagagaaaaccaaactgtaa

SEQ ID NO: 157 - DNA, 912 bp, synthetic construct
  source 1..912: mol_type = other DNA; note = Candida.Uox Variant Coding DNA (W271UAG)
atgagcaccacactgagcagcagcacctatggtaaagataatgtgaaattcctgaaagtgaaaaaagatccgcagaacccgaaaaaacaagaagttatggaagcaaccgttacctgtctgctggaaggtggttttgataccagctataccgaagcagataatagcagcattgttccgaccgataccgtgaaaaataccattctggttctggcaaaaaccaccgaaatttggccgattgaacgttttgcagccaaactggcaacccattttgttgagaaatattctcatgttagcggtgtgagcgttaaaattgttcaggatcgttgggttaaatatgccgttgatggtaaaccgcatgatcacagctttattcatgaaggtggtgaaaaacgtatcaccgacctgtattacaaacgtagcggtgattataaactgtccagcgcaattaaagatctgaccgttctgaaaagcaccggcagcatgttttatggttataacaaatgcgatttcacaaccctgcagccgaccaccgatcgtattctgagcaccgatgttgatgcaacctgggtttgggataataagaaaattggtagcgtgtacgatattgccaaagcagcagataaaggcatcttcgataatgtgtataatcaggcacgtgaaattaccctgaccacctttgcactggaaaatagcccgagcgttcaggcaaccatgtttaatatggcgacccagattctggaaaaagcgtgtagcgtttatagcgttagctatgcactgccgaacaaacactattttctgattgacctgaaatagaagggccttgaaaatgataacgaactgttttatccgagtccgcatccgaatggtctgattaaatgtaccgttgtgcgtaaagagaaaaccaaactgtaa

SEQ ID NO: 158 - DNA, 909 bp, synthetic construct
  source 1..909: mol_type = other DNA; note = Arth.Uox Variant Coding DNA (N119UAG)
atgaccgcaaccgcagaaaccagcaccggcaccaaagttgttctgggtcagaatcagtatggtaaagcagaagttcgtctggttaaagttacccgtaataccgcacgtcatgaaattcaggatctgaatgttaccagccagctgcgtggtgattttgaagcagcacataccgcaggcgataatgcacatgttgttgcaaccgatacacagaaaaacaccgtttatgcatttgcccgtgatggttttgcaaccaccgaagaatttctgctgcgtctgggtaaacatttcaccgaaggttttgattgggttaccggtggtcgttgggcagcacagcagtttttctgggatcgtatttaggatcacgatcatgcctttagccgcaataaaagcgaagtgcgtaccgcagttctggaaattagcggtagcgaacaggcaattgttgcaggtattgaaggtctgaccgttctgaaaagcaccggtagcgagtttcatggttttccgcgtgataaatacaccacactgcaagaaaccaccgatcgtattctggcaaccgatgttagcgcacgttggcgttataataccgttgaagttgattttgatgcggtttatgcaagcgttcgtggtctgctgctgaaagcatttgcagaaacccatagcctggcactgcagcagacaatgtatgaaatgggtcgtgcagttattgaaacccatccggaaattgatgagatcaaaatgagcctgccgaacaaacatcattttctggttgatctgcagccgtttggtcaggataatccgaatgaagtgttttatgcagcagatcgtccgtatggtctgattgaagcaaccattcagcgtgaaggtagccgtgcagatcatccgatttggagtaatattgcaggtttttgctaa

SEQ ID NO: 159 - DNA, 909 bp, synthetic construct
  source 1..909: mol_type = other DNA; note = Arth.Uox Variant Coding DNA (S142UAG)
atgaccgcaaccgcagaaaccagcaccggcaccaaagttgttctgggtcagaatcagtatggtaaagcagaagttcgtctggttaaagttacccgtaataccgcacgtcatgaaattcaggatctgaatgttaccagccagctgcgtggtgattttgaagcagcacataccgcaggcgataatgcacatgttgttgcaaccgatacacagaaaaacaccgtttatgcatttgcccgtgatggttttgcaaccaccgaagaatttctgctgcgtctgggtaaacatttcaccgaaggttttgattgggttaccggtggtcgttgggcagcacagcagtttttctgggatcgtattaatgatcacgatcatgcctttagccgcaataaaagcgaagtgcgtaccgcagttctggaaattagcggttaggaacaggcaattgttgcaggtattgaaggtctgaccgttctgaaaagcaccggtagcgagtttcatggttttccgcgtgataaatacaccacactgcaagaaaccaccgatcgtattctggcaaccgatgttagcgcacgttggcgttataataccgttgaagttgattttgatgcggtttatgcaagcgttcgtggtctgctgctgaaagcatttgcagaaacccatagcctggcactgcagcagacaatgtatgaaatgggtcgtgcagttattgaaacccatccggaaattgatgagatcaaaatgagcctgccgaacaaacatcattttctggttgatctgcagccgtttggtcaggataatccgaatgaagtgttttatgcagcagatcgtccgtatggtctgattgaagcaaccattcagcgtgaaggtagccgtgcagatcatccgatttggagtaatattgcaggtttttgctaa

SEQ ID NO: 160 - DNA, 909 bp, synthetic construct
  source 1..909: mol_type = other DNA; note = Arth.Uox Variant Coding DNA (E196UAG)
atgaccgcaaccgcagaaaccagcaccggcaccaaagttgttctgggtcagaatcagtatggtaaagcagaagttcgtctggttaaagttacccgtaataccgcacgtcatgaaattcaggatctgaatgttaccagccagctgcgtggtgattttgaagcagcacataccgcaggcgataatgcacatgttgttgcaaccgatacacagaaaaacaccgtttatgcatttgcccgtgatggttttgcaaccaccgaagaatttctgctgcgtctgggtaaacatttcaccgaaggttttgattgggttaccggtggtcgttgggcagcacagcagtttttctgggatcgtattaatgatcacgatcatgcctttagccgcaataaaagcgaagtgcgtaccgcagttctggaaattagcggtagcgaacaggcaattgttgcaggtattgaaggtctgaccgttctgaaaagcaccggtagcgagtttcatggttttccgcgtgataaatacaccacactgcaagaaaccaccgatcgtattctggcaaccgatgttagcgcacgttggcgttataataccgtttaggttgattttgatgcggtttatgcaagcgttcgtggtctgctgctgaaagcatttgcagaaacccatagcctggcactgcagcagacaatgtatgaaatgggtcgtgcagttattgaaacccatccggaaattgatgagatcaaaatgagcctgccgaacaaacatcattttctggttgatctgcagccgtttggtcaggataatccgaatgaagtgttttatgcagcagatcgtccgtatggtctgattgaagcaaccattcagcgtgaaggtagccgtgcagatcatccgatttggagtaatattgcaggtttttgctaa

SEQ ID NO: 161 - DNA, 25 bp, synthetic construct
  source 1..25: mol_type = other DNA; note = pBAD-AgUox_F
gccgccatggtgtctgctgtgaagg

SEQ ID NO: 162 - DNA, 25 bp, synthetic construct
  source 1..25: mol_type = other DNA; note = pBAD-AgUox_R
gccgagatctttaatggtgatggtg

SEQ ID NO: 163 - DNA, 37 bp, synthetic construct
  source 1..37: mol_type = other DNA; note = AgUox-196Amb_F
gtcgaagtccacctatacggtgttgtaacgccaacgg

SEQ ID NO: 164 - DNA, 37 bp, synthetic construct
  source 1..37: mol_type = other DNA; note = AgUox-196Amb_R
ccgttggcgttacaacaccgtataggtggacttcgac

SEQ ID NO: 165 - protein, 16 aa, synthetic construct
  source 1..16: note = AgUox-WT fregment
YNTVEVDFDAVYASVR

SEQ ID NO: 166 - protein, 16 aa, synthetic construct
  source 1..16: note = AgUox-frTet fregment
YNTVXVDFDAVYASVR

SEQ ID NO: 167 - protein, 19 aa, synthetic construct
  source 1..19: note = fregment control
AVIETHPEIDEIKMSLPNK

(19) United States
(12) Patent Application Publication
(10) Pub. No.: US 2026/0091090 A1
(43) Pub. Date: Apr. 2, 2026

(54) URATE OXIDASE-ALBUMIN CONJUGATE, PREPARATION METHOD THEREOF, AND USE THEREOF

(71) Applicant: Proabtech Inc., Gwangju (KR)

(72) Inventors: Jeong Haeng Cho, Gimpo-si Gyeonggi-do (KR); Sun Oh Shin, Gwangju (KR); Hyun Woo Kim, Seoul (KR); Hyeongseok Kim, Seoul (KR); Dong Ho Bak, Jeonju-si Jeollabuk-do (KR); Inchan Kwon, Buk-gu Gwangju (KR); Byungseop Yang, Seoul (KR)

(21) Appl. No.: 19/351,705

(22) Filed: Oct. 7, 2025

Related U.S. Application Data
(63) Continuation of application No. 17/773,516.

(30) Foreign Application Priority Data
Sep. 25, 2020 (KR) 10-2020-0125215
Jan. 29, 2021 (KR) 10-2021-0013537

(51) Int. Cl. A61K 38/44; A61K 47/54; A61K 47/60; A61K 47/64; A61P 19/06; C12N 9/06

(52) U.S. Cl. CPC A61K 38/44; A61K 47/545; A61K 47/60; A61K 47/643; A61P 19/06; C12N 9/0048; C12Y 107/03003

(57) ABSTRACT

The present specification discloses a urate oxidase-albumin conjugate, a preparation method thereof, a urate oxidase variant contained in the urate oxidase-albumin conjugate, and a preparation method thereof. The urate oxidase-albumin conjugate is characterized in that three or more albumins are conjugated to the urate oxidase variant through a linker, thereby improving half-life and reducing immunogenicity. In addition, the urate oxidase-albumin conjugate can be used to prevent or treat various diseases, disorders and/or indications caused by uric acid.

### CROSS-REFERENCE TO RELATED APPLICATIONS

This application is a continuation of U.S. patent application Ser. No. 17/773,516, filed Apr. 29, 2022, which is a 371 U.S. National Stage Application of PCT/KR2021/013037, filed Sep. 24, 2021, which claims priority to Korean Republic Application No. 10-2020-0125215, filed Sep. 25, 2020, and to Korean Republic Application No. 10-2021-0013537, filed Jan. 29, 2021. The entire contents of each of PCT Application No. PCT/KR2021/013037 and U.S. patent application Ser. No. 17/773,516 are incorporated herein in their entirety by this reference.

### SEQUENCE LISTING

The instant application contains a Sequence Listing which has been submitted electronically in XML format and is hereby incorporated by reference in its entirety. Said XML copy, created on Dec. 10, 2025, is named “P250169US_SL_fixed” and is 291,112 bytes in size.

### TECHNICAL FIELD

The present description discloses a urate oxidase variant into which a nonnatural amino acid is introduced site-specifically a preparation method thereof, a conjugate in which the urate oxidase variant and albumin are conjugated by a linker, and a preparation method thereof. In addition, the present description discloses the use of the urate oxidase-albumin conjugate.

### BACKGROUND ART

Urate oxidase (Uricase) is a type of enzyme that cannot be synthesized in primates including humans, and it functions to break down uric acid into allantoin. Urate oxidase has a direct therapeutic mechanism of decomposing uric acid, which is the main cause of gout, into an excretable form, and thus has the advantage of a strong uric acid lowering effect. However, urate oxidase has limited use for treatment of gout because 1) it can only be used as an injection due to a short half-life in the body, and 2) an immune response occurs when administered to the body because it is an externally derived protein, resulting in side effects.

On the other hand, therapeutic protein has been reported to be effective in the treatment of various diseases, and it is one of the important growth motives in the pharmaceutical industry. However, there is a problem in that therapeutic protein is continuously removed by glomerular filtration, pinocytosis, and immune response in a patient's body. Therefore, when developing a therapeutic protein, it is very important to extend the duration of the drug effect by lowering the rate at which it is removed from the patient's body due to such a phenomenon. A technique for improving the half-life to solve the problem by physically or chemically binding albumin to a therapeutic protein is called albumination.

In the present description, in order to solve the above-described problems that may occur when urate oxidase is used as a therapeutic agent, a urate oxidase-albumin conjugate produced through albumination of urate oxidase is disclosed.

### DISCLOSURE

### Technical Problem

The present description is intended to provide a urate oxidase-albumin conjugate.

The present description is intended to provide a method of preparing the urate oxidase-albumin conjugate.

The present description is intended to provide a urate oxidase variant included in the urate oxidase-albumin conjugate.

The present description is intended to provide a method of preparing the urate oxidase variant.

The present description is intended to provide a pharmaceutical composition including the urate oxidase-albumin conjugate.

The present description is intended to provide a use of the urate oxidase-albumin conjugate.

### Technical Solution

In the present description, a urate oxidase-albumin conjugate, which is represented by Formula 1: [Formula 1] Uox-[J₁-A-J₂-HSA]ₙ in which Uox is a urate oxidase variant, J₁ is a urate oxidase-linker junction, A is an anchor, J₂ is an albumin-linker junction, HSA is human serum albumin, the urate oxidase variant includes three or more nonnatural amino acids having a diene functional group, the urate oxidase-linker junction is formed by Inverse Electron Demand Diels-Alder (IEDDA) reaction of a diene functional group of the nonnatural amino acid and a dienophile functional group connected to the anchor, and n is 3 or 4.

Provided herein is a urate oxidase-albumin conjugate including: 3 or 4 albumin-subunit conjugates represented by Formula 2: [Formula 2]p′-J₁-A-J₂-HSA, where p′ is a urate oxidase variant subunit, J₁ is a urate oxidase-linker junction, A is an anchor, J₂ is an albumin-linker junction, and HSA is human serum albumin. The urate oxidase variant subunit is formed by substituting at least one amino acid in the sequence of a wild-type urate oxidase subunit with a nonnatural amino acid containing a tetrazine functional group or a triazine functional group. The urate oxidase-linker junction is formed by an inverse electron demand Diels-Alder (IEDDA) reaction between a tetrazine or triazine functional group of the nonnatural amino acid and a trans-cyclooctene functional group connected to the anchor; and Optionally, one urate oxidase variant subunit, when the urate oxidase-albumin conjugate includes three albumin-subunit complexes, and the urate oxidase-albumin conjugate includes one urate oxidase variant subunit, the urate oxidase variant subunit of each of the albumin-subunit complexes and one urate oxidase variant subunit oligomerize to form a tetramer. When the urate oxidase-albumin conjugate includes four albumin-subunit complexes, the urate oxidase-albumin conjugate includes no urate oxidase variant subunits, and the urate oxidase variant subunits of the respective albumin-subunit complexes oligomerize to form a tetramer.

The present description discloses a pharmaceutical composition for preventing or treating uric acid-related diseases, the pharmaceutical composition including: a therapeutically effective amount of the urate oxidase-albumin conjugate and a pharmaceutically acceptable carrier.

In one embodiment, the uric acid-related disease is any one of hyperuricemia, acute gouty arthritis, intermittent gout, chronic nodular gout, chronic kidney disease, and tumor lysis syndrome (TLS).

In one embodiment, the pharmaceutically acceptable carrier includes one or more selected from the followings: binders such as lactose, saccharose, sorbitol, mannitol, starch, amylopectin, cellulose or gelatin; excipients such as dicalcium phosphate and the like; disintegrants such as corn stach or sweet potato starch; lubricants such as magnesium stearate, calcium stearate, sodium stearyl fumarate or polyethylene glycol wax; sweetener; air freshener; syrup; liquid carriers such as fatty oils; sterile aqueous solution; propylene glycol; polyethylene glycol; injectable esters such as ethyl oleate; suspending agent; emulsion; freeze-dried preparations; external preparations; stabilizer; buffer; animal oil; vegetable oil; wax; paraffin; starch; tragacanth; cellulose derivatives; polyethylene glycol; silicon; bentonite; silica; talc; and zinc oxide.

The present description discloses a use of the urate oxidase-albumin conjugate as an application in preparation of a treatment agent for uric acid-related diseases.

The present description discloses a method of preparing a urate oxidase-albumin conjugate, the method including: reacting albumin and a linker, in which the linker includes a dienophile functional group, an anchor, and a thiol reactive moiety, in which the thiol reactive moiety of the linker is bound with the thiol moiety of albumin through reaction to form an albumin-linker conjugate; and reacting the albumin-linker conjugate and a urate oxidase variant, in which the urate oxidase variant is formed by substituting three or more amino acids in a sequence of a wild urate oxidase with nonnatural amino acids including a dien functional group, in which the dien functional group of the urate oxidase variant and the dienophile functional group of the albumin-linker conjugate combine through an inverse electron demand Diels-Alder (IEDDA) reaction to form a urate oxidase-albumin conjugate, in which the urate oxidase variant is characterized in that three or more albumins are bound via linkers.

The present description discloses a urate oxidase-albumin conjugate including: three or more nonnatural amino acids in a sequence thereof, in which each of the nonnatural amino acids includes a tetrazine functional group or a triazine functional group.

In one embodiment, the urate oxidase variant is a tetramer formed by oligomerization of one wild-type urate oxidase subunit and three urate oxidase variant subunits, in which each of the urate oxidase variant subunit is formed by substituting one or more amino acids in the sequence of the wild-type urate oxidase subunit with nonnatural amino acids including a tetrazine functional group or a triazine functional group.

In one embodiment, the urate oxidase variant is a tetramer formed by oligomerization of four urate oxidase variant subunits, in which each of the urate oxidase variant subunit is formed by substituting one or more amino acids in the sequence of the wild-type urate oxidase subunit with nonnatural amino acids including a tetrazine functional group or a triazine functional group.

The present description discloses a vector capable of expressing the urate oxidase variant.

The present description discloses a method of preparing a urate oxidase variant, the method including: preparing a cell line including a vector capable of expressing an orthogonal tRNA/synthetase pair and a urate oxidase variant expression vector, in which the vector capable of expressing an orthogonal tRNA/synthetase pair is a vector capable of an exogenous suppressor tRNA and an exogenous tRNA synthetase, the exogenous suppressor tRNA can recognize a specific stop codon, the exogenous tRNA synthetase can recognize a nonnatural amino acid including a tetrazine functional group and/or a triazine functional group and connect the recognized functional group to the exogenous suppressor tRNA, and the urate oxidase variant expression vector is a vector capable of expressing the urate oxidase variant, in which the location of a sequence corresponding to the nonnatural amino acid of the urate oxidase variant is a sequence part encoded by the specific stop codon; and culturing the cell line in a medium containing one or more types of nonnatural amino acid including a tetrazine functional group and/or a triazine functional group.

### Advantageous Effects

According to the technical problem and the solution thereof disclosed herein, a urate oxidase-albumin conjugate is provided. The urate oxidase-albumin conjugate has improved half-life and reduced immunogenicity compared to wild-type urate oxidase, so the urate oxidase-albumin conjugate can be used as an effective therapeutic agent for uric acid-related diseases.

### DESCRIPTION OF DRAWINGS

FIG. 1 schematically illustrates an albumin-subunit conjugate;

FIGS. 2 to 3 schematically illustrates an example of a urate oxidase-albumin conjugate; FIG. 2 is a schematic diagram illustrating a urate oxidase-albumin conjugate in which four albumin proteins are conjugated, and FIG. 3 is a schematic diagram illustrating a urate oxidase-albumin conjugate in which three albumin proteins are conjugated;

FIG. 4 shows the result of enzyme digestion electrophoresis of pTAC-Uox-W174amb;

FIG. 5 shows the result of observing the cloning sequence of pTAC-Uox-W174amb (SEQ ID NO: 154);

FIG. 6 shows the results of SDS-PAGE analysis to determine Uox-frTet expression;

FIGS. 7 and 8 show the results of primary separation purification and SDS-PAGE analysis of Uox-frTet through a DEAE column;

FIGS. 9 and 10 show the results of secondary separation purification and SDS-PAGE analysis of Uox-frTet through a phenyl fast flow column;

FIG. 11 shows the SDS-PAGE analysis result of Uox-frTet and Fasturtec;

FIG. 12 shows the results of SEC-HPLC analysis of secondary purified Uox-frTet;

FIG. 13 shows the results of determining whether frTet is introduced into Uox through a fluorescent labeling dye (TCO-Cy3);

FIG. 14 shows the results of SDS-PAGE analysis of secondary purified Uox-HAS;

FIG. 15 shows the SEC-HPLC analysis result of Uox-HSA;

FIG. 16 shows the PK profile results for each route of administration of Fasturtec and Uox-HSA, in which 1) Fasturtec_IV represents a profile for a case of intravenous administration (IV) of a wild-type urate oxidase, 2) Uox-HSA_IV represents a profile for a case of intravenous administration (IV) of a urate oxidase-albumin conjugate, 3) Uox-HSA_IP represents a profile for a case of intraperitoneal administration (IP) of the urate oxidase-albumin conjugate, and 4) Uox-HSA_IM represents a profile for a case of intramuscular administration (IM) of the urate oxidase-albumin conjugate.

FIG. 17 shows data of the PK profile results for each route of administration of Fasturtec and Uox-HSA, in which Fasturtec_IV, Uox-HSA_IV, Uox-HSA_IP, and Uox-HSA_IM are the same as described in FIG. 16, AVG represents the average value of each ICR mouse data (n=5), SD represents the standard deviation, AUC represents an area under curve of the PK profile result, T1/2 represents the half-life expressed in units of time, Tmax is the time when the blood concentration of the drug is the highest, and Cmax is the concentration of the drug at the time when the blood concentration of the drug is the highest;

FIG. 18 shows the results of a pharmacodynamic evaluation test for observation of reduction in uric acid in blood according to administration of Uox-HSA in a repeated hyperuricemia animal model, in which 1) Negative control represents a negative control, 2) Uox-HSA 1 mg/kg represents a case where a urate oxidase-albumin conjugate is administered intravenously at a dose of 1 mg/kg, 3) Uox-HSA 4 mg/kg represents a case where a urate oxidase-albumin conjugate is administered intravenously at a dose of 4 mg/kg, 4) Uox-HSA 10 mg/kg represents a case where a urate oxidase-albumin conjugate is administered intravenously at a dose of 10 mg/kg, 5) Fasturtec 1.33 mg/kg represents a case where a wild-type urate oxidase (Fasturtec) is administered intravenously at a dose of 1.33 mg/kg, and 6) Febuxostat 10 mg/kg represents a case where Febuxostat is orally administered at a dose of 1 mg/kg;

FIG. 19 shows data of the results of a pharmacodynamic evaluation test for observation of reduction in uric acid in blood according to administration of Uox-HSA in a repeated hyperuricemia animal model, in which the negative control, Uox-HSA 1 mg/kg, Uox-HSA 4 mg/kg, Uox-HSA 10 mg/kg, Fasturtec 1.33 mg/kg, and Febuxostat 10 mg/kg are the same as described in FIG. 18, and AVG is each animal model data mean value, and SD represents the standard deviation;

FIG. 20 shows a PK profile result for administration of TG Uox-HSA to a Human FcRn TG mouse, in which 1) Uox-HSA (Tetra) is a urate oxidase-albumin conjugate in which 4 albumins are conjugated per one urate oxidase, 2) Uox-HSA (tri/di) is a urate oxidase-albumin conjugate in which 2 to 3 albumins are conjugated per one urate oxidase, AUC is the Area Under Curve of the PK profile result, T1/2 is the half-life expressed in units of time, and Range is a pharmacokinetic evaluation time range;

FIG. 21 shows an immunogenicity analysis result of Uox-HSA through PBMC;

FIG. 22 shows data of the immunogenicity analysis result of Uox-HSA through PBMC, in which 1) CD4 represents data for CFSE-CD4+ T cells, and 2) CD8 represents data for CFSE-CD8+ T cells, and in which in each table, data of #1 to #3 represent values for each subject, and Mean represents the average value of all subjects;

FIG. 23 to FIG. 27 illustrate examples of nonnatural amino acids that can be introduced into urate oxidase variants;

FIG. 28 illustrates an example of a urate oxidase-linker junction structure and an example of a nonnatural amino acid related thereto, in which moiety (1) is linked to the remaining residue moiety of the nonnatural amino acid, and moiety (2) is linked to an anchor;

FIG. 29 illustrates an example of an anchor structure;

FIG. 30 illustrates an example of an albumin-linker junction structure, in which J₁ represents a urate oxidase-linker junction and J₂ represents an albumin-linker junction;

FIG. 31 illustrates an example of a linker structure;

FIG. 32 shows the SDS-PAGE results of AgUox-WT and AgUox-frTet, in which (A) represents the results of Coomassie Brilliant Blue (CBB) staining of an AgUox variant, in which Mw is a molecular weight marker, BI is the result before induction, AI is the result after induction, (B) represents a CBB-stained protein gel of a purified AgUox variant, and (C) represents a phenotypic image and CBB-stained protein gel of AgUox variants cultured in the presence or absence of TCO-Cy3;

FIG. 33 shows flight mass spectra of trypsin-digested fragments of AgUox-WT (A) and AgUox-frTet (B), the mass of YNTVEVDFDAVYASVR (SEQ ID NO: 165), which is an AgUox-WT fragment, is compared with the mass of YNTVXVDFDAVYASVR (SEQ ID NO: 166, X represents frTet), which is an AgUox-frTet fragment, and AVIETHPEIDEIKMSLPNK (SEQ ID NO: 167), which is the peak of the fragment, was used as a control;

FIG. 34 represents protein gel of a reaction mixture of AgUox-frTet and MAL-HSA (denoted by MAL) or APN-HSA (denoted by APN), in which Mw represents a molecular weight standard;

FIG. 35 represents size exclusion chromatograms and protein gels of fractions of a conjugate mixture of AgUox-frTet and MAL-HSA (A) or APN-HSA (B), in which the eluted fractions were loaded on a protein gel and stained with Coomassie Brilliant Blue; and

FIG. 36 shows the results of pharmacokinetic analysis (PK profile) of AgUox-WT and AgUox-HSA conjugates, in which serum activity of residual AgUox-WT and AgUox-HSA conjugates was measured at the early stage (0-84 h) and the late stage (84-120 h), and t^{e}1/2 and t^{l}1/2 represent early and late serum half-lives, respectively.

FIG. 37A-FIG. 37G show exemplary nonnatural amino acids and linkers.

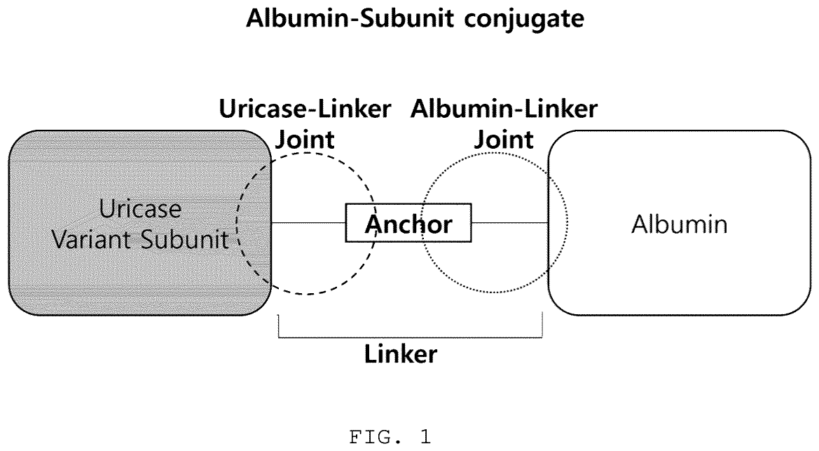

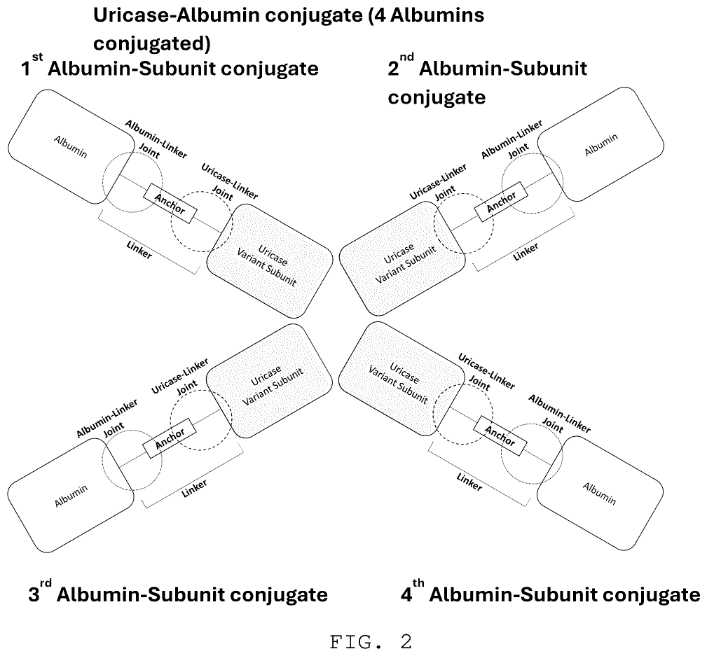

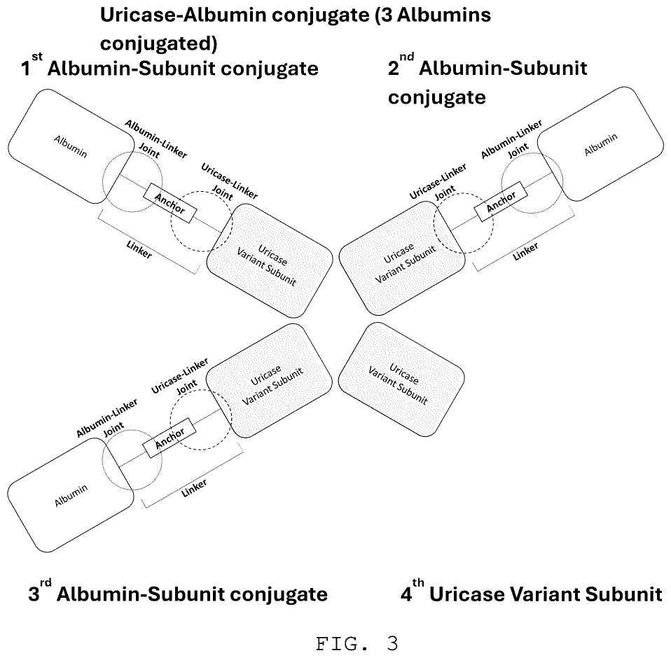

### BEST MODE

Hereinafter, with reference to the accompanying drawings, the invention will be described in more detail through specific embodiments and examples. It should be noted that the accompanying drawings include some, but not all, embodiments of the invention. The details of the invention disclosed by the present specification may be embodied in various forms and are not limited to the specific embodiments described herein. These embodiments are considered to be provided in order to satisfy the statutory requirements applicable herein. Those skilled in the art to which the invention disclosed herein pertains will come up with many modifications and other embodiments of the subject matter disclosed herein. Accordingly, it is to be understood that the subject matter disclosed herein is not limited to the specific embodiments described herein, and that modifications and other embodiments thereof also fall within the scope of the claims.

### DEFINITION OF TERMS

### About

As used herein, the term “about” refers to a degree close to a certain quantity, and it refers to an amount, level, value, number, frequency, percent, dimension, size, amount, weight, or length that varies by to the extent of 30%, 25%, 20%, 25%, 10%, 9%, 8%, 7%, 6%, 5%, 4%, 3%, 2%, or 1% with respect to a reference amount, level, value, number, frequency, percentage, dimension, size, amount, weight, or length.

### Click Chemistry

Herein, “Click Chemistry” is a term that was introduced by K. B. Sharpless in Scripps Research Institute to describe complementary chemical functional groups and chemical reactions designed such that two molecules can form a covalent bond fast and stably. The click chemistry does not mean a specific reaction but is a term for a fast and stable reaction. Click chemistry creates only byproducts that are not significant and is modular, wide in scope, high-yielding, stereospecific, biologically stable, large in thermodynamic dynamic (for example, 84 kJ/mol or more), and high in atomic economy. Example of the click chemistry include 1) Huisgen 1,3-dipolar cycloaddition (see Tomoe et al. Journal of Organic Chemistry (2002) 67: 3075-3064, etc.), 2) Diels-Alder reaction, 3) Nucleophilic addition to small strained rings such as epoxide and aziridine, 4) a nucleophilic addition reaction to an activated carbonyl group, and 5) an addition reaction to a carbon-carbon double bond or triple bond. The meaning of the click chemistry should be appropriately interpreted according to the context, and the click chemistry includes all other meanings that can be recognized by those skilled in the art.

### Bioorthogonal Reaction

Herein, the term “bioorthogonal reaction” refers to any chemical reaction in which externally introduced residues react with each other without interfering with native biochemical processes. When a certain reaction is “bioorthogonal”, the reaction has a characteristic that it is very stable in the body because in vivo intrinsic molecules are not involved in the reaction or reaction product.

### Standard Amino Acid

As used herein, the term “standard amino acid” refers to 20 amino acids synthesized through the transcription and translation processes of genes in the body of an organism. Specifically, the standard amino acid includes alanine (Ala, A), arginine (Arg, R), asparagine (Asn, N), aspartic acid (Asp, D), cysteine (Cys, C), glutamic acid (Glu, E), glutamine (Gln, Q), glycine (Gly, G), histidine (His, H), isoleucine (Ile, I), leucine (Leu, L), lysine (Lys K), methionine (Met, M), phenylalanine (Phe, F), proline (Pro, P), serine (Ser, S), threonine (Thr, T), tryptophan (Trp, W), tyrosine (Tyr, Y), and valine (Val, V). The standard amino acid has a corresponding DNA codon and can be represented by a general one-letter or three-letter notation of an amino acid. The subjects being referred to by the term standard amino acid should be appropriately interpreted according to the context, and they include all other meanings that can be recognized by those skilled in the art.

### Nonnatural Amino Acid

As used herein, the term “nonnatural amino acid” refers to an amino acid that is not synthesized in the body but synthesized artificially. The nonnatural amino acid includes, for example, 4-(1,2,3,4-tetrazin-3-yl) phenylalanine, and 4-(6-methyl-s-tetrazin-3-yl)phenylalanine. Since the nonnatural amino acid does not have a corresponding DNA codon, it cannot be represented by a general one-letter or three-letter notation of an amino acid, and it is written using other characters and explained via additional explanation. The subjects being referred to by the term nonnatural amino acids should be appropriately interpreted according to the context, and they include all other meanings that can be recognized by those skilled in the art.

### Description of Peptide Sequence

Unless otherwise stated, when describing the sequence of a peptide in the present specification, single letter notation or three letter notation of an amino acid is used, and it is written in the direction from the N-terminus to the C-terminus. For example, when expressed as RNVP, it refers to a peptide in which arginine, asparagine, valine, and proline are sequentially linked in the direction from the N-terminus to the C-terminus. For another example, when expressed as Thr-Leu-Lys, it refers to a peptide in which threonine, leucine, and lysine are sequentially linked in the direction from the N-terminus to the C-terminus. In the case of amino acids that cannot be represented by the one-letter or three-letter notation, other letters are used to describe these amino acids, and will be explained via additional explanation.

### Immunogenicity

As used herein, the term “immunogenicity” collectively refers to “the property of acting as an antigen capable of inducing an immune response” in the dictionary. There are various methods for measuring the immunogenicity of a specific antigen, and the methods may be appropriately adopted or designed according to the purpose. For example, the methods may include 1) a method for confirming whether IgG, IgA, and/or IgE type antibodies are generated in the body of a subject when the antigen is administered into the body of the subject, 2) a method for confirming the time when the IgG, IgA, and/or IgE type antibodies are generated depending on the administration cycle, 3) a method for confirming the titer of the induced antibodies to the antigen, and 4) when the mechanism of action of the induced antibodies is found, a method for measuring the effect according to the mechanism of action, but the methods are not limited thereto. The subjects being referred to by the term immunogenicity should be appropriately interpreted according to the context, and they include all other meanings that can be recognized by those skilled in the art.

### Mechanism of Treatment of Gout by Urate Oxidase

### Causes of Gout

Gout arthritis is caused by an inflammatory reaction to monosodium urate monohydrate crystals (MSU) secondary to hyperuricemia, which is a symptom in which blood uric acid concentration is higher than the normal range. Gout is caused by accumulation of uric acid in the body due to overproduction of uric acid in the liver and small intestine and/or decreased excretion of uric acid. Gout usually starts with hyperuricemia, goes through acute gouty arthritis, then goes through intermittent gout, and progresses to chronic nodular gout.

### Mechanism of Treatment of Gout by Urate Oxidase

Urate oxidase (Uricase) is a type of enzyme that cannot be synthesized in primates including humans, and it functions to break down uric acid into allantoin. The allantoin has a solubility 5 to 10 times higher than that of uric acid, so it is easy to be excreted by the kidneys. Therefore, when urate oxidase is used as a therapeutic agent, it is possible to treat gout by preventing the accumulation of uric acid, which is the main cause of gout, and by excreting uric acid from the body.

### Limitations of Urate Oxidase as a Therapeutic Agent for Gout

The urate oxidase has a direct therapeutic mechanism for decomposing uric acid, which is the main cause of gout, into an excretable form, and thus has the advantage of having a strong uric acid lowering effect. However, since the urate oxidase is a protein drug, 1) it can be used only as an injection due to its short half-life in the body. In addition, 2) since it is an externally-derived protein, an immune response occurs when administered into the body, resulting in side effects. Therefore, there are restrictions on its use as a therapeutic agent for gout

### Limitations of Conventional Art

### Limitations of Commercially Available Urate Oxidase-Based Drugs

In a urate oxidase-based drug (KRYSTEXXA; pegloticase) currently available on the market, polyethylene glycol (PEG) is randomly bound to a urate oxidase to improve the short half-life which is a restriction factor of the urate oxidase. However, it has limitations in that 1) the urate oxidase is randomly pegylated, blocking the active site of the enzyme, resulting in reduction in efficacy, and 2) the PEG has a side effect of causing an allergic reaction in the body.

### Limitations of Conventional Urate Oxidase-Albumin Conjugate Technology

The inventors have disclosed a urate oxidase-albumin conjugate in the literature “KR 1637010 B1”. The urate oxidase-albumin conjugate disclosed in the literature replaces one or more amino acids of a urate oxidase with a nonnatural amino acid and conjugates the urate oxidase with albumin using a linker having a dibenzocyclooctyne (DBCO) reactive group. However, the urate oxidase-albumin conjugate disclosed in the literature has a problem that the yield is very low due to the slow speed of the strain-promoted cycloaddition (SPAAC), which is the binding reaction of AzF and DBCO at the junction. Therefore, in the urate oxidase-albumin conjugate disclosed in the literature, despite the fact that there are at least four sites for albumin conjugation in a urate oxidase, there is a limitation in that only urate oxidase-albumin conjugates in which one or two albumins are conjugated per one urate oxidase can be obtained due to the inefficiency of the SPAAC reaction. Due to the limitations described above, the urate oxidase-albumin conjugate disclosed in the literature has problems in that 1) the effect of albumin conjugation, including an increase in the half-life or reduction in immunogenicity, is limited, 2) unexpected reactions may be occurred in the body due to exposure of the residue of AzF to which albumin is not conjugated.

### Urate Oxidase-Albumin Conjugate

### Overview of Urate Oxidase-Albumin Conjugate

Disclosed herein is a urate oxidase-albumin conjugate. The urate oxidase-albumin conjugate is a structure in which a urate oxidase variant and albumin are linked through a linker. The urate oxidase-albumin conjugate is characterized in that three or more albumins are conjugated to the urate oxidase variant through the linker. The urate oxidase variant is obtained by substituting at least one amino acid in the sequence of a wild-type urate oxidase with a nonnatural amino acid, and the linker and the albumin are bound through the residue of the nonnatural amino acid. Specifically, the urate oxidase-albumin conjugate includes: a urate oxidase variant; a urate oxidase-linker junction formed by conjugation of a urate oxidase variant and a linker; an anchor contained within the linker; an albumin-linker junction formed by conjugation of albumin and a linker; and albumin. Hereinafter, each component (urate oxidase variant, albumin, and linker) and the structure of the urate oxidase-albumin conjugate resulting from conjugation of the components will be described in more detail.

### Component 1 of Urate Oxidase-Albumin Conjugate—Urate Oxidase Variant

Urate oxidase is an enzyme that has the function of decomposing uric acid into allantoin in the body, and can be used to treat various diseases or disorders caused by the accumulation of uric acid. The urate oxidase is a tetramer formed by oligomerization of four urate oxidase subunits. The urate oxidase-albumin conjugate disclosed herein includes a urate oxidase variant, which is one or more amino acids are substituted with an nonnatural amino acid, from a wild-type urate oxidase sequence. Specifically, the urate oxidase variant is a tetrameric protein which means that three or four subunits among the four subunits are urate oxidase variant subunits. In this case, the sequence of the urate oxidase variant subunit is what one or more amino acids are substituted with nonnatural amino acids, compared to the sequence of wild-type urate oxidase subunit. The purpose of creating a urate oxidase variant by inserting a nonnatural amino acid into a wild urate oxidase is to bind a moiety of the nonnatural amino acid to a linker through a reverse electron-demand Diels-Alder reaction (IEDDA reaction).

### Component 2 of Urate Oxidase-Albumin Conjugate—Albumin

The albumin refers to human serum albumin and/or a variant of human serum albumin, and serves as a drug carrier for the urate oxidase variant. The albumin allows the urate oxidase-albumin conjugate to exhibit improved in vivo half-life and low immunogenicity compared to the case where the urate oxidase variant is present alone.

### Component 3 of Urate Oxidase-Albumin Conjugate—Linker

The linker binds the urate oxidase variant to the albumin, and includes an IEDDA reactive group capable of binding to the urate oxidase variant, a thiol reactive group capable of binding to albumin, and an anchor. In the urate oxidase-albumin conjugate, the IEDDA reactive group and the thiol reactive group are bound to the urate oxidase variant and the albumin, respectively. Therefore, the IEDDA reactive group and the thiol reactive group do not exist in original forms but exist in modified forms such as a urate oxidase-linker junction and an albumin-linker junction.

### Urate Oxidase-Albumin Conjugate 1—Subunit-Albumin Conjugate

The urate oxidase variant of the urate oxidase-albumin conjugate disclosed herein is a tetramer formed by oligomerization of four subunits, in which three or more of the subunits are conjugated to albumin through linkers. Among the subunits constituting the urate oxidase variant, the subunit conjugated to albumin through a linker is referred to as a subunit-albumin conjugate. The urate oxidase variant of the urate oxidase-albumin conjugate includes three or more urate oxidase variant subunits. In this case, some or all of the urate oxidase variant subunits are each a subunit-albumin conjugate in which the subunit is conjugated with albumin.

### Urate Oxidase-Albumin Conjugate 2—Urate Oxidase-Linker Junction

The urate oxidase-albumin conjugate disclosed herein is one in which a urate oxidase variant and albumin are conjugated through a linker. In this case, the portion where the urate oxidase variant and the linker are joined is called a urate oxidase-linker junction. The urate oxidase variant and the linker are characterized in that they are conjugated through an IEDDA reaction.

### Urate Oxidase-Albumin Conjugate 3—Albumin-Linker Junction

The urate oxidase-albumin conjugate disclosed herein is one in which a urate oxidase variant and albumin are conjugated through a linker. In this case, the portion where the albumin and the linker are joined is called an albumin-linker junction.

### Urate Oxidase-Albumin Conjugate 4—Anker

The linker includes an IEDDA reactive group capable of being conjugated to the urate oxidase variant, and a thiol reactive group capable of being conjugated to albumin, and an anchor that links the reactive groups to each other. Since the anchor is a part not involved in the reaction for linking the urate oxidase and the albumin, it is characterized in that the structure of the anchor remains unchanged in the urate oxidase-albumin conjugate.

Urate Oxidase-Albumin Conjugate Example 1—from Perspective of Urate Oxidase-Linker

In one embodiment, the urate oxidase-albumin conjugate is represented by Formula 1 below:

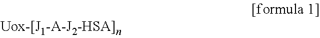

in which Uox is a urate oxidase variant,
J₁ is a urate oxidase-linker junction,
A is an anchor,
J₂ is an albumin-linker junction,
HSA is Human Serum Albumin, and
n is 3 or 4.
   Urate Oxidase-Albumin Conjugate Example 2—from Perspective of Urate Oxidase-Linker

In one embodiment, the subunit-albumin conjugate is represented by Formula 2 below:

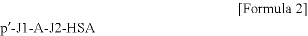

In Formula 2, p′ is a urate oxidase variant subunit, and the other parts are the same as defined above.

In one embodiment, the urate oxidase-albumin conjugate includes one wild urate oxidase subunit and three subunit-albumin conjugates. Specifically, in the urate oxidase-albumin conjugate, one wild urate oxidase subunit and three urate oxidase variant subunits included in each of the subunit-albumin conjugates oligomerize to form a tetramer.

In one embodiment, the urate oxidase-albumin conjugate includes one urate oxidase variant subunit and three subunit-albumin conjugates. Specifically, in the urate oxidase-albumin conjugate, one urate oxidase variant subunit and three urate oxidase variant subunits included in the respective subunit-albumin conjugates oligomerize to form a tetramer.

In one embodiment, the urate oxidase-albumin conjugate includes four subunit-albumin conjugates. Specifically, in the urate oxidase-albumin conjugate, the four urate oxidase urate oxidase variant subunits included in the respective subunit-albumin conjugates oligomerize to form a tetramer.

### Characteristic of Urate Oxidase-Albumin Conjugate 1—Effect of Increase in Half-Life Through Albumin Conjugation

As described above, when albumin is bound to a drug molecule, there is an effect of increasing the half-life of the drug in the body. The urate oxidase-albumin conjugate disclosed hemin is characterized in that the half-life of a urate oxidase, which is a therapeutic protein, in the body is increased by conjugating albumin to the urate oxidase. The improved half-life in the body can be confirmed through a pharmacokinetics profile experiment after the urate oxidase-albumin conjugate is administered to the body, and can be confirmed in Experimental Example 4.

### Characteristic of Urate Oxidase-Albumin Conjugate 2—Not Inhibiting Activity of Urate Oxidase

One of the limitations of the conventional art is that a drug carrier, for example, albumin, or polyethylene glycol (PEG), etc. is bound to a urate oxidase to increase efficacy, but the three-dimensional structure of the drug carrier inhibits the activity by blocking the active site of the urate oxidase, thereby reducing the drug efficacy. The urate oxidase-albumin conjugate disclosed in the present description is characterized in that albumin is site-specifically bound to a moiety that does not inhibit the activity of the urate oxidase, thereby not reducing drug efficacy.

### Characteristic of Urate Oxidase-Albumin Conjugate 3—Immunogenicity Reduction Effect

It is known that humans do not produce urate oxidase, and thus urate oxidase mainly used for treatment is an enzyme derived from microorganisms. Since such urate oxidase is a foreign protein derived from microorganisms, when administered solely in the body, the urate oxidase causes an immune response, resulting in side effects. Therefore, it is an important task to reduce the immunogenicity of the urate oxidase. The urate oxidase-albumin conjugate disclosed in the present description is characterized in that the immunogenicity of urate oxidase is reduced by conjugating the urate oxidase with albumin, which is a human plasma protein. The albumin is a protein constituting most of the plasma, is very stable in the human body, and hardly exhibits immunogenicity. Therefore, the urate oxidase-albumin conjugate exhibits significantly low immunogenicity compared to a case where urate oxidase is solely administered into the body.

### Characteristic of Urate Oxidase-Albumin Conjugate 4—Three or Four Albumins in Urate Oxidase

The conventional urate oxidase-albumin conjugate technology linked a urate oxidase variant and an albumin through a strain-promoted Alkyne-Azide Cycloaddition reaction (SPAAC reaction). The SPAAC reaction has a limitation in that it can produce a urate oxidase-albumin conjugate in which only one or two albumins are bound to a urate oxidase variant due to a relatively slow reaction rate and low yield. The urate oxidase-albumin conjugate provided herein is characterized in that three or more albumins are conjugated to one urate oxidase variant because the conjugation occurs through an IEDDA reaction which enables fast reaction between urate oxidase variants and albumins, resulting in high yield. Due to the characteristics described above, 1) the half-life improvement effect and the immunogenicity reduction effect of the urate oxidase-albumin conjugate can be maximized, and 2) the exposure of residues of nonnatural amino acids is minimized, resulting in side effects being minimized.

### Urate Oxidase Variant

### Overview of Urate Oxidase Variant

The urate oxidase variant included in the urate oxidase-albumin conjugate disclosed herein is characterized in that a portion of the sequence of amino acid of a wild urate oxidase derived from a microorganism is modified. The urate oxidase variant contains three or more nonnatural amino acids, and can be site-specifically conjugated to albumins through the moiety of each of the nonnatural amino acids. Specifically, the urate oxidase variant is a tetramer formed by oligomerization of four urate oxidase variant subunits, and each urate oxidase variant subunit is characterized in that at least one amino acid in the sequence thereof is substituted with at least one nonnatural amino acid when compared with a wild urate oxidase subunit.

Microorganisms from which Wild Urate Oxidase is Derived

The wild urate oxidase, which is the prototype of the urate oxidase variant provided herein, is derived from a microorganism. In one embodiment, the wild urate oxidase may be a urate oxidase derived from a microorganism selected from Aspergillus Flavus, Arthrobacter globiformis, and Candida utilis.

### Exemplary Sequence of Wild Urate Oxidase

The wild urate oxidase is a tetramer protein in which four wild urate oxidase subunits that are the same are oligomerized.

In one embodiment, when the wild urate oxidase is a urate oxidase derived from Aspergillus Flavus, the peptide sequence of the subunit may be

(SEQ ID NO: 1)
SAVKAARYGKDNVRVYKVHKDEKTGVQTVYEMTVCVLLEGEIETSYTKAD
NSVIVATDSIKNTIYITAKQNPVTPPELFGSILGTHFIEKYNHIHAAHVN
IVCHRWTRMDIDGKPHPHSFIRDSEEKRNVQVDVVEGKGIDIKSSLSGLT
VLKSTNSQFWGFLRDEYTTLKETWDRILSTDVDATWQWKNFSGLQEVRSH
VPKFDATWATAREVTLKTFAEDNSASVQATMYKMAEQILARQQLIETVEY
SLPNKHYFEIDLSWHKGLQNTGKNAEVFAPQSDPNGLIKCTVGRSSLKSK
L

from the N-terminus to the C-terminus.

In another embodiment, when the wild urate oxidase is a urate oxidase derived from Candida utilis, the peptide sequence of the subunit may be

(SEQ ID NO: 51)
MSTTLSSSTYGKDNVKFLKVKKDPQNPKKQEVMEATVTCLLEGGFDTSYT
EADNSSIVPTDTVKNTILVLAKTTEIWPIERFAAKLATHFVEKYSHVSGV
SVKIVQDRWVKYAVDGKPHDHSFIHEGGEKRITDLYYKRSGDYKLSSAIK
DLTVLKSTGSMFYGYNKCDFTTLQPTTDRILSTDVDATWVWDNKKIGSVY
DIAKAADKGIFDNVYNQAREITLTTFALENSPSVQATMFNMATQILEKAC
SVYSVSYALPNKHYFLIDLKWKGLENDNELFYPSPHPNGLIKCTVVRKEK
TKL

from the N-terminus to the C-terminus.

In a further embodiment, when the wild urate oxidase is a urate oxidase derived from Arthrobacter globiformis, the peptide sequence of the subunit may be

(SEQ ID NO: 118)
MTATAETSTGTKVVLGQNQYGKAEVRLVKVTRNTARHEIQDLNVTSQLRG
DFEAAHTAGDNAHVVATDTQKNTVYAFARDGFATTEEFLLRLGKHFTEGF
DWVTGGRWAAQQFFWDRINDHDHAFSRNKSEVRTAVLEISGSEQAIVAGI
EGLTVLKSTGSEFHGFPRDKYTTLQETTDRILATDVSARWRYNTVEVDFD
AVYASVRGLLLKAFAETHSLALQQTMYEMGRAVIETHPEIDEIKMSLPNK
HHFLVDLQPFGQDNPNEVFYAADRPYGLIEATIQREGSRADHPIWSNIAG
FC

from the N-terminus to the C-terminus.

### Urate Oxidase Variant Subunit

Like the wild urate oxidase, the urate oxidase variant is also a tetrameric protein including four subunits. The urate oxidase variant includes 3 or 4 urate oxidase variant subunits, and the urate oxidase variant subunit is a subunit formed by substituting one or more original amino acids with nonnatural amino acids, in a wild-type urate oxidase subunit. In one embodiment, the urate oxidase variant may include three urate oxidase variant subunits and one wild urate oxidase subunit. In another embodiment, the urate oxidase variant may include four urate oxidase variant subunits. A more specific description will be provided in the section titled “Urate Oxidase Variant Subunit”.

### Urate Oxidase Variant Preparation Method

The present description discloses a method of preparing the urate oxidase variant. The urate oxidase variant includes one or more unnatural amino acids. However, in nature, nucleic acid codons corresponding to unnatural amino acids do not exist. In order to biosynthesize a protein containing such a nonnatural amino acid in a cell, it is necessary to solve the problem that there is no nucleic acid codon corresponding to a nonnatural amino acid. Literature “Korean Patent No. 1637010 B1” discloses a method for effectively solving this problem by using the fact that three types of stop codons used in nature does not encode an amino acid. The urate oxidase variant preparation method refers to the method disclosed in the literature “KR 1637010 B1”. In the method, 1) an orthogonal tRNA/synthetase pair having a function of recognizing a stop codon and introducing a nonnatural amino acid into the sequence is used, and 2) a nucleic acid encoding a nonnatural amino acid site in the sequence of a urate oxidase variant with a stop codon is used. A more specific description will be provided in the section titled “Urate Oxidase Variant Preparation Method”.

### Vector Encoding Urate Oxidase Variant

The present description discloses a vector encoding a urate oxidase variant used in the urate oxidase variant preparation method. The vector encoding the urate oxidase variant is characterized in that in a nucleic acid sequence encoding a wild-type urate oxidase, a nucleic acid codon at a position at which the nucleic acid is to be substituted with a nonnatural amino acid is changed to a stop codon. A more specific description will be provided in the section titled “Vector Encoding Urate Oxidase Variant”.

### Urate Oxidase Variant Subunit

Urate Oxidase Variant Subunit 1—Substitution with Nonnatural Amino Acid

The urate oxidase variant subunit includes at least one nonnatural amino acid, and the nonnatural amino acid has a functional group capable of being bound to a linker through an IEDDA reaction. In one embodiment, the nonnatural amino acid may be an amino acid including a dien functional group capable of causing an IEDDA reaction. Specifically, the dien functional group may be a tetrazine functional group or a derivative thereof and/or a triazine functional group or a derivative thereof. More specifically, the nonnatural amino acid may be selected from the group consisting of 4-(1,2,3,4-tetrazin-3-yl) phenylalanine (frTet), 4-(6-methyl-s-tetrazin-3-yl)phenylalanine (Tet-v2.0), 3-(4-(1,2,4-triazin-6-yl)phenyl)-2-aminopropanoic acid, 2-amino-3-(4-(2-(6-methyl-1,2,4,5-tetrazin-3-yl)ethyl)phenyl)propanoic acid, 2-amino-3-(4-(6-phenyl-1,2,4,5-tetrazin-3-yl)phenyl)propanoic acid, 3-(4-((1,2,4,5-tetrazin-3-yl)amino)phenyl)-2-aminopropanoic acid, 3-(4-(2-(1,2,4,5-tetrazin-3-yl)ethyl)phenyl)-2-aminopropanoic acid, 3-(4-((1,2,4,5-tetrazin-3-yl)thio)phenyl)-2-aminopropanoic acid, 2-amino-3-(4-((6-methyl-1,2,4,5-tetrazin-3-yl)thio)phenyl)propanoic acid, 3-(4-((1,2,4,5-tetrazin-3-yl)oxy)phenyl)-2-aminopropanoic acid, 2-amino-3-(4-((6-methyl-1,2,4,5-tetrazin-3-yl)oxy)phenyl)propanoic acid, 3-(4′-(1,2,4,5-tetrazin-3-yl)-[1,1′-biphenyl]-4-yl)-2-aminopropanoic acid, 2-amino-3-(4′-(6-methyl-1,2,4,5-tetrazin-3-yl)-[1,1′-biphenyl]-4-yl)propanoic acid, 2-amino-3-(6-(6-(pyridin-2-yl)-1,2,4,5-tetrazin-3-yl)pyridin-3-yl)propanoic acid, 3-(4-(1,2,4,5-tetrazin-3-yl)phenyl)-2-aminopropanoic acid, and 2-amino-3-(4-(6-methyl-1,2,4,5-tetrazin-3-yl)phenyl)propanoic acid.

### Urate Oxidase Variant Subunit 2—Example of Nonnatural Amino Acid

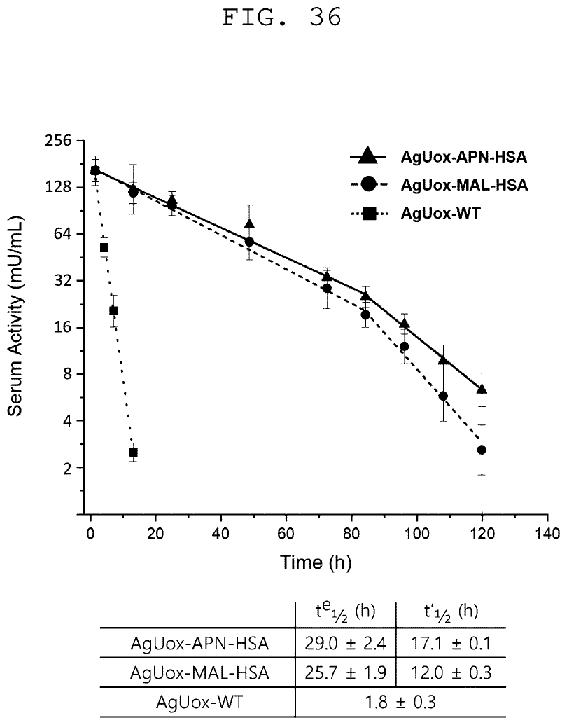

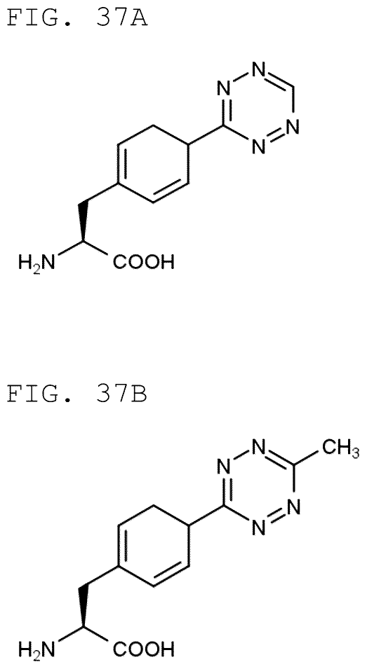

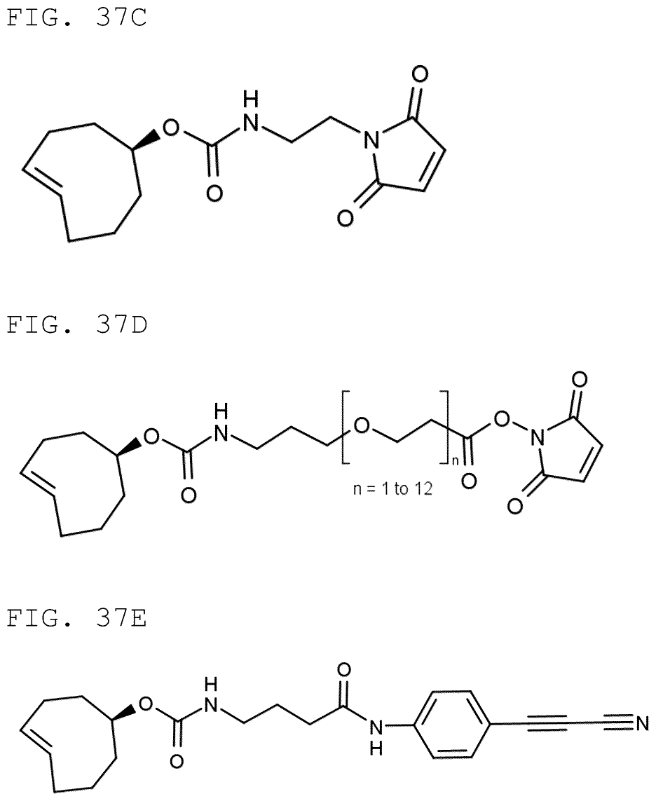

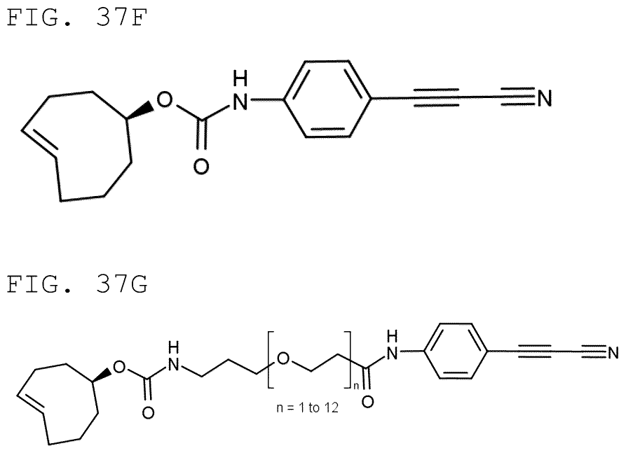

In one embodiment, the nonnatural amino acid may be selected from the following:
The nonnatural amino acid shown in FIG. 37A [UAA01],
The non natural amino acid shown in FIG. 37B [UAA02],

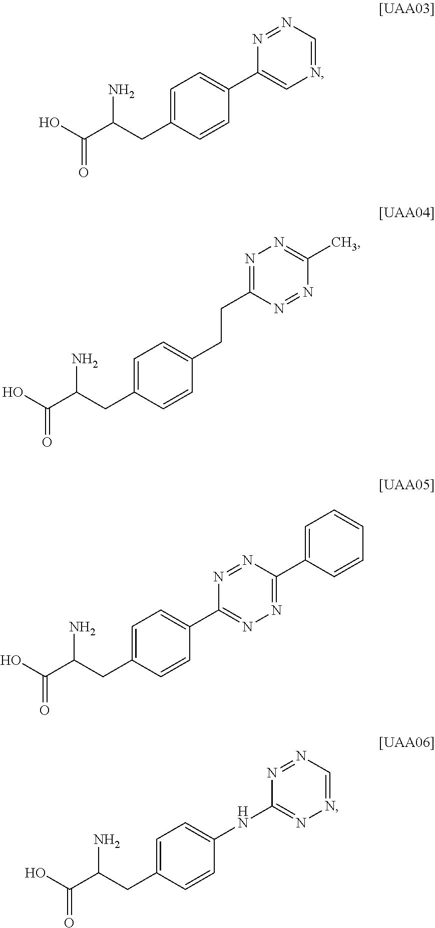 [SMILES: NC(Cc1ccc(-c2cncnn2)cc1)C(=O)O ; Cc1nnc(CCc2ccc(CC(N)C(=O)O)cc2)nn1 ; NC(Cc1ccc(-c2nnc(-c3ccccc3)nn2)cc1)C(=O)O ; NC(Cc1ccc(Nc2nncnn2)cc1)C(=O)O]

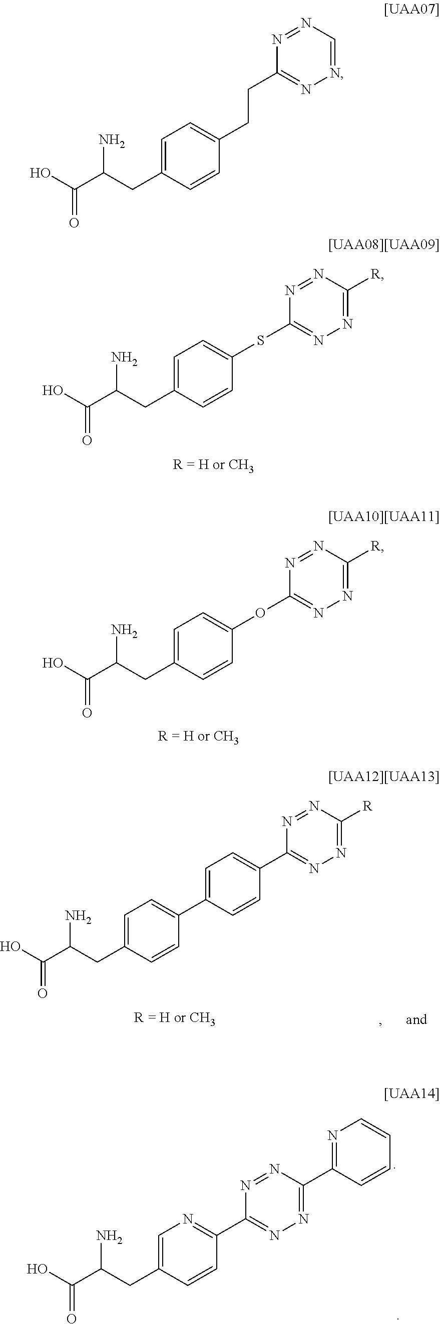

### Urate Oxidase Variant Subunit 3—Substitution Site

When a urate oxidase variant is formed by inserting a nonnatural amino acid into a wild urate oxidase, the structure and function of the original urate oxidase should not be affected as much as possible. Therefore, an amino acid that plays an important role in the activity and structure of a urate oxidase cannot be substituted with a nonnatural amino acid. In addition, since the nonnatural amino acid needs to bind to the linker during the preparation of the urate oxidase-albumin conjugate, it is advantageous to substitute the amino acid at a position with relatively high accessibility to a solvent, in the three-dimensional structure of the urate oxidase. Various methods can be used to select sites with high solvent accessibility while minimally affecting the structure and function of the wild urate oxidase. For example, molecular modeling calculations can select candidate sites that are similar in intrinsic atomic energy to the wild urate oxidase and which are high in solvent accessibility.

In one embodiment, the site for substitution with a nonnatural amino acid in the sequence of the wild urate oxidase to make a urate oxidase variant may be determined by referring to molecular modeling simulation results. Specifically, the molecular modeling simulation result may be a scoring result of the Rosetta molecular modeling package.

### Urate Oxidase Variant Subunit 4—Example of Substitution Site

In one embodiment, the urate oxidase variant subunit may be one in which one or more amino acids selected from the following are substituted with one or more nonnatural amino acids: glycine at position 137, glutamic acid at position 22, asparagine at position 92, lysine at position 23, serine at position 295, glycine at position 113, lysine at position 273, lysine at position 171, alanine at position 240, glutamic acid at position 89, lysine at position 266, threonine at position 24, lysine at position 48, serine at position 192, proline at position 202, aspartic acid at position 110, glutamine at position 243, glutamine at position 195, lysine at position 138, proline at position 115, serine at position 199, glycine at position 272, lysine 4, aspartic acid at position 112, glycine at position 267, lysine at position 114, glutamine at position 70, tryptophan at position 174, asparagine at position 223, glutamic acid at position 41, aspartic acid at position 261, glycine at position 25, serine at position 52, arginine at position 241, glutamic acid at position 213, asparagine at position 274, glutamic acid at position 221, alanine at position 206, glutamic acid at position 236, arginine at position 164, glutamine at position 269, glutamic acid at position 136, glutamic acid at position 259, glutamic acid at position 246, alanine at position 49, glycine at position 148, histidine at position 19, serine at position 296, and threonine at position 47 of the peptide sequence of SEQ ID NO: 1.

In one embodiment, the urate oxidase variant subunit may be one in which one or more amino acids selected from the following are substituted with one or more nonnatural amino acids: threonine at position 301, asparagine at position 26, leucine at position 303, lysine at position 194, serine at position 95, serine at position 140, glycine at position 116, lysine at position 302, lysine at position 167, aspartic acid at position 115, glutamic acid, proline at position 24, tryptophan at position 271, aspartic acid at position 277, aspartic acid at position 169, proline at position 118, threonine at position 177, glutamine at position 174, lysine at position 208, glutamic acid at position 275, leucine at position 266, glycine at position 273, tyrosine at 200, glutamic acid at position 92, glutamic acid at position 247, leucine at position 228, lysine at position 300, lysine at position 204, glutamic acid at position 51, aspartic acid at position 207, lysine at position 117, cysteine at position 250, proline at position 175, lysine at position 270, aspartic acid at position 268, glycine at position 44, asparagine at position 193, glycine at position 164, threonine at position 73, lysine at 29, asparagine at position 230, glutamine at position 25, asparagine at position 216, Serine at position 55, lysine at position 28, serine at position 6, proline at position 27, lysine at position 298, alanine at position 113, asparagine at position 213, glutamic acid at position 220, glycine at position 141, tyrosine at position 163, tyrosine at position 253, aspartic acid at position 178, lysine at position 93, lysine at position 103, lysine at position 144, arginine at position 139, lysine at position 138, serine 7, aspartic acid at position 151, arginine at position 297, lysine at position 272, asparagine at position 278, and phenylalanine at position 265 of the peptide sequence of SEQ ID NO: 51.

In one embodiment, the urate oxidase variant subunit may be one in which one or more amino acids selected from the following are substituted with one or more nonnatural amino acids: aspartic acid at position 80, phenylalanine at position 82, phenylalanine at position 100, aspartic acid at position 101, phenylalanine at position 114, asparagine at position 119, aspartic acid at position 120, serine at position 142, glutamic acid at position 143, glycine at position 175, valine at position 195, glutamic acid at position 196, histidine at position 218, and proline at position 238 of the peptide sequence of SEQ ID NO: 118.

### Urate Oxidase Variant Subunit 5—Exemplary Sequence

In one embodiment, when the urate oxidase variant is obtained by partially modifying the sequence of a urate oxidase derived from Aspergillus flavus, the urate oxidase variant subunit may be represented by SEQ ID NOs: 2 to 50. In this case, X in the sequence may be selected from the group consisting of 4-(1,2,4,5-tetrazin-3-yl) phenylalanine (frTet), 4-(6-methyl-s-tetrazin-3-yl)phenylalanine (Tet-v2.0), 3-(4-(1,2,4-triazin-6-yl)phenyl)-2-aminopropanoic acid, 2-amino-3-(4-(2-(6-methyl-1,2,4,5-tetrazin-3-yl)ethyl)phenyl)propanoic acid, 2-amino-3-(4-(6-phenyl-1,2,4,5-tetrazin-3-yl)phenyl)propanoic acid, 3-(4-((1,2,4,5-tetrazin-3-yl)amino)phenyl)-2-aminopropanoic acid, 3-(4-(2-(1,2,4,5-tetrazin-3-yl)ethyl)phenyl)-2-aminopropanoic acid, 3-(4-((1,2,4,5-tetrazin-3-yl)thio)phenyl)-2-aminopropanoic acid, 2-amino-3-(4-((6-methyl-1,2,4,5-tetrazin-3-yl)thio)phenyl)propanoic acid, 3-(4-((1,2,4,5-tetrazin-3-yl)oxy)phenyl)-2-aminopropanoic acid, 2-amino-3-(4-((6-methyl-1,2,4,5-tetrazin-3-yl)oxy)phenyl)propanoic acid, 3-(4′-(1,2,4,5-tetrazin-3-yl)-[1,1′-biphenyl]-4-yl)-2-aminopropanoic acid, 2-amino-3-(4′-(6-methyl-1,2,4,5-tetrazin-3-yl)-[1,1′-biphenyl]-4-yl)propanoic acid, 2-amino-3-(6-(6-(pyridin-2-yl)-1,2,4,5-tetrazin-3-yl)pyridin-3-yl)propanoic acid, 3-(4-(1,2,4,5-tetrazin-3-yl)phenyl)-2-aminopropanoic acid, 2-amino-3-(4-(6-methyl-1,2,4,5-tetrazin-3-yl)phenyl)propanoic acid.

In one embodiment, when the urate oxidase variant is a variant obtained by partially modifying the sequence of a urate oxidase derived from Candida utilis, the urate oxidase variant subunit may be represented by SEQ ID NOs: 52 to 117. In this case, X in the sequence may be selected from the group consisting of 4-(1,2,4,5-tetrazin-3-yl) phenylalanine (frTet), 4-(6-methyl-s-tetrazin-3-yl)phenylalanine (Tet-v2.0), 3-(4-(1,2,4-triazin-6-yl)phenyl)-2-aminopropanoic acid, 2-amino-3-(4-(2-(6-methyl-1,2,4,5-tetrazin-3-yl)ethyl)phenyl)propanoic acid, 2-amino-3-(4-(6-phenyl-1,2,4,5-tetrazin-3-yl)phenyl)propanoic acid, 3-(4-((1,2,4,5-tetrazin-3-yl)amino)phenyl)-2-aminopropanoic acid, 3-(4-(2-(1,2,4,5-tetrazin-3-yl)ethyl)phenyl)-2-aminopropanoic acid, 3-(4-((1,2,4,5-tetrazin-3-yl)thio)phenyl)-2-aminopropanoic acid, 2-amino-3-(4-((6-methyl-1,2,4,5-tetrazin-3-yl)thio)phenyl)propanoic acid, 3-(4-((1,2,4,5-tetrazin-3-yl)oxy)phenyl)-2-aminopropanoic acid, 2-amino-3-(4-((6-methyl-1,2,4,5-tetrazin-3-yl)oxy)phenyl)propanoic acid, 3-(4′-(1,2,4,5-tetrazin-3-yl)-[1,1′-biphenyl]-4-yl)-2-aminopropanoic acid, 2-amino-3-(4′-(6-methyl-1,2,4,5-tetrazin-3-yl)-[1,1′-biphenyl]-4-yl)propanoic acid, 2-amino-3-(6-(6-(pyridin-2-yl)-1,2,4,5-tetrazin-3-yl)pyridin-3-yl)propanoic acid, 3-(4-(1,2,4,5-tetrazin-3-yl)phenyl)-2-aminopropanoic acid, 2-amino-3-(4-(6-methyl-1,2,4,5-tetrazin-3-yl)phenyl)propanoic acid.

In one embodiment, when the urate oxidase variant is obtained by partially modifying the sequence of a urate oxidase derived from Arthrobacter globiformis, the urate oxidase variant subunit may be represented by SEQ ID NOs: 119 to 132. In this case, X in the sequence may be selected from the group consisting of 4-(1,2,4,5-tetrazin-3-yl) phenylalanine (frTet), 4-(6-methyl-s-tetrazin-3-yl)phenylalanine (Tet-v2.0), 3-(4-(1,2,4-triazin-6-yl)phenyl)-2-aminopropanoic acid, 2-amino-3-(4-(2-(6-methyl-1,2,4,5-tetrazin-3-yl)ethyl)phenyl)propanoic acid, 2-amino-3-(4-(6-phenyl-1,2,4,5-tetrazin-3-yl)phenyl)propanoic acid, 3-(4-((1,2,4,5-tetrazin-3-yl)amino)phenyl)-2-aminopropanoic acid, 3-(4-(2-(1,2,4,5-tetrazin-3-yl)ethyl)phenyl)-2-aminopropanoic acid, 3-(4-((1,2,4,5-tetrazin-3-yl)thio)phenyl)-2-aminopropanoic acid, 2-amino-3-(4-((6-methyl-1,2,4,5-tetrazin-3-yl)thio)phenyl)propanoic acid, 3-(4-((1,2,4,5-tetrazin-3-yl)oxy)phenyl)-2-aminopropanoic acid, 2-amino-3-(4-((6-methyl-1,2,4,5-tetrazin-3-yl)oxy)phenyl)propanoic acid, 3-(4′-(1,2,4,5-tetrazin-3-yl)-[1,1′-biphenyl]-4-yl)-2-aminopropanoic acid, 2-amino-3-(4′-(6-methyl-1,2,4,5-tetrazin-3-yl)-[1,1′-biphenyl]-4-yl)propanoic acid, 2-amino-3-(6-(6-(pyridin-2-yl)-1,2,4,5-tetrazin-3-yl)pyridin-3-yl)propanoic acid, 3-(4-(1,2,4,5-tetrazin-3-yl)phenyl)-2-aminopropanoic acid, and 2-amino-3-(4-(6-methyl-1,2,4,5-tetrazin-3-yl)phenyl)propanoic acid.

### Urate Oxidase Variant Subunit 6—Including a Sequence Similar to the Exemplary Sequence

In one embodiment, the urate oxidase variant subunit may have a sequence that is 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to a sequence selected from SEQ ID NOs: 1 to 132. In one embodiment, the urate oxidase variant subunit may have a sequence similar or identical to a sequence selected from SEQ ID NOs: 1 to 132 by a degree corresponding to one of the percentages described above. In one embodiment, the urate oxidase variant subunit may have a sequence similar or identical to a sequence selected from SEQ ID NOs: 1 to 132 by a degree in the range of 80% to 100%. In one embodiment, the urate oxidase variant subunit may have a sequence similar or identical to a sequence selected from SEQ ID NOs: 1 to 132 by a degree in the range of 95% or more.

### Urate Oxidase Variant Preparation Method

### Overview of Urate Oxidase Variant Preparation Method

The present description discloses a method of preparing a urate oxidase variant. The following matters are involved in the urate oxidase preparation method: a cell line to express a urate oxidase variant; an exogenous suppressor tRNA to recognize a specific stop codon; a foreign tRNA synthetase; and a vector encoding a urate oxidase variant in which a nonnatural amino acid is encoded with the stop codon. Here, the exogenous suppressor tRNA and the exogenous tRNA synthetase are not the suppressor tRNA and tRNA synthetase specific to the expression cell line but a suppressor tRNA and tRNA synthetase derived from cells different from the expression cell line. Therefore, the exogenous suppressor tRNA is characterized in that it does not react with the tRNA synthetase unique to the expression cell line. The exogenous tRNA synthetase i) reacts only with the exogenous suppressor tRNA and ii) shows activity only in the nonnatural amino acid to be included in the urate oxidase variant. As a result, when the exogenous tRNA synthetase is used, the nonnatural amino acid is specifically linked to the exogenous suppressor tRNA so that the nonnatural amino acid can be introduced into the peptide sequence.

The urate oxidase variant preparation method is a method in which 1) in the cell line, 2) the exogenous suppressor tRNA and the exogenous tRNA synthetase are involved in 4) expressing the urate oxidase variant, 3) based on a vector encoding the urate oxidase variant. In the urate oxidase variant preparation method, the order of each process is not particularly limited if the urate oxidase variant can be expressed in the cell line, and additional processes may be included if necessary.

### Cell Line Expressing Urate Oxidase Variant

The urate oxidase variant preparation method is characterized in that it is obtained by expressing a urate oxidase variant in a cell line. The urate oxidase variant expression cell line is not particularly limited if it can produce a urate oxidase variant. However, when a release factor recognizing the stop codon in the cell line normally functions, the release factor competes with the exogenous tRNA, thereby reducing the yield. Therefore, it is preferable to use a cell line in which the release factor that recognizes the stop codon is inactivated.

In one embodiment, the cell line expressing the urate oxidase variant may be selected from the following:
Escherichia genus; Erwinia genus; Serratia genus; Providencia genus; Corynebacterium genus; Pseudomonas genus; Leptospira genus; Salmonella genus; Brevibacterium genus; Hypomonas genus; Chromobacterium genus; Norcardia genus; fungi; and yeast.

In one embodiment, the cell line may be a cell line in which a release factor that recognizes a stop codon and terminates translation is inactivated. Specifically, the stop codon is any one selected from among an amber codon (5′-UAG-3′), an ocher codon (5′-UAA-3′), and an opal codon (5′-UGA-3′).

In one embodiment, the cell line expressing the urate oxidase variant may be the cell line used in the method disclosed in the literature “KR 1637010 B1”. Specifically, the cell line may be E. coli C321.ΔA.exp (Addgene, ID: 49018).

Exogenous Suppressor tRNA

The exogenous suppressor tRNA is a tRNA that recognizes a specific stop codon, and does not react with a tRNA synthetase unique to the expression cell line. The exogenous suppressor tRNA specifically reacts with the exogenous tRNA synthetase, and the exogenous tRNA synthetase functions to link a nonnatural amino acid to the exogenous suppressor tRNA. As a result, the exogenous suppressor tRNA can recognize the specific stop codon and introduce the nonnatural amino acid at the corresponding position.

Specifically, the suppressor tRNA may recognizes any one selected from among an amber codon (5′-UAG-3′), an ocher codon (5′-UAA-3′), and an opal codon (5′-UGA-3′). Preferably, the suppressor tRNA may recognize an amber codon. For example, the suppressor tRNA may be a suppressor tRNA (MjtRNA^{Tyr}_{CUA}) derived from Methanococcus jannaschii (Yang et. al, Temporal Control of Efficient In Vivo Bioconjugation Using a Genetically Encoded Tetrazine-Mediated Inverse-Electron-Demand Diels-Alder Reaction, Bioconjugate Chemistry, 2020, 2456-2464).

Exogenous tRNA Synthetase

The exogenous tRNA synthetase selectively reacts with a specific nonnatural amino acid, and functions to link the specific nonnatural amino acid to the exogenous suppressor tRNA. The exogenous tRNA synthetase does not react with the a suppressor tRNA unique to the expression cell line and specifically reacts with only the exogenous suppressor tRNA. In one embodiment, the tRNA synthetase may have a function of linking a nonnatural amino acid including a tetrazine derivative and/or a triazine derivative to the exogenous suppressor tRNA. In one embodiment, the tRNA synthetase may be a tyrosyl-tRNA synthetase (MjTyrRS) derived from Methanococcus jannaschii (Yang et. al, Temporal Control of Efficient In Vivo Bioconjugation Using a Genetically Encoded Tetrazine-Mediated Inverse-Electron-Demand Diels-Alder Reaction, Bioconjugate Chemistry, 2020, 2456-2464). Preferably, the tRNA synthetase may be a C11 variant of the MjTyrRS.

Orthogonal tRNA/Synthetase Pair

In the present description, 1) an exogenous suppressor tRNA that specifically reacts with only the exogenous tRNA synthetase, and 2) the exogenous tRNA synthetase are collectively called an orthogonal tRNA/synthetase pair. In the urate oxidase variant preparation method disclosed herein, it is important to express the orthogonal tRNA/synthetase pair in the expression cell line. The method is not particularly limited if this objective can be achieved. In one embodiment, the urate oxidase variant preparation method includes transforming the cell line with a vector capable of expressing the orthogonal tRNA/synthetase pair. Specifically, the vector capable of expressing the orthogonal tRNA/synthetase pair may be pDUle_C11 reported by Yang et. al. (Temporal Control of Efficient In Vivo Bioconjugation Using a Genetically Encoded Tetrazine-Mediated Inverse-Electron-Demand Diels-Alder Reaction, Bioconjugate Chemistry, 2020, 2456-2464).

### Vector Encoding Urate Oxidase Variant

The urate oxidase variant preparation method includes a process of introducing or transfecting a vector encoding a urate oxidase variant into an expression cell line. A more specific description will be provided in the section titled “Vector Encoding Urate Oxidase Variant”.

### Example of Urate Oxidase Variant Preparation Method

In one embodiment, a urate oxidase variant preparation method includes the following:
preparing a cell line including a vector capable of expressing an orthogonal tRNA/synthetase pair, and a vector encoding a urate oxidase variant,
in which the orthogonal tRNA/synthetase pair includes an exogenous suppressor tRNA and an exogenous tRNA synthetase,
the urate oxidase variant is a variant in which three or more amino acids in a wild-type urate oxidase sequence are substituted with nonnatural amino acids each including a tetrazine derivative or a triazine derivative, and
in the vector encoding the urate oxidase variant, the codon corresponding to the nonnatural amino acid is an amber codon (5′-UAG-3′); and
culturing the cell line in a medium which contains a nonnatural amino acid including a tetrazine functional group and/or a triazine functional group,
in which the exogenous suppressor tRNA can recognize the amber codon (5′-UAG-3′),
the exogenous tRNA synthetase may link the nonnatural amino acid to the exogenous tRNA, and
accordingly, when the cell line expresses the vector encoding the urate oxidase variant, the cell line expresses a peptide in which the nonnatural amino acid is linked to a position corresponding to the amber codon.

### Vector Encoding Urate Oxidase Variant

### Overview of Vector Encoding Urate Oxidase Variant

The present description discloses a vector encoding a urate oxidase variant. The vector encoding a urate oxidase variant is characterized in that the nonnatural amino acid in the sequence of the urate oxidase variant is encoded with a stop codon. In one embodiment, in the vector encoding a urate oxidase variant, a standard amino acid in the sequence of the urate oxidase variant is encoded with a codon corresponding to a standard amino acid found in nature, and a nonnatural amino acid may be encoded with a stop codon. For example, the stop codon is any one selected from among an amber codon (5′-UAG-3′), an ocher codon (5′-UAA-3′), and an opal codon (5′-UGA-3′). Alternatively, the stop codon may be selected from among 5′-TAG-3′, 5′-TAA-3′, and 5′-TGA-3′. In one embodiment, the vector encoding a urate oxidase variant may be codon-optimized for the expression cell line. For example, the vector encoding a urate oxidase variant may be an E. coli codon-optimized one.

### Example of Vector Sequence Encoding Urate Oxidase Variant

In one embodiment, when the urate oxidase variant is obtained by partially modifying the sequence of a urate oxidase derived from Aspergillus flavus, the vector encoding the urate oxidase variant may include a sequence selected from SEQ ID NOs: 152 to 154.

In one embodiment, when the urate oxidase variant is obtained by partially modifying the sequence of a urate oxidase derived from Candida utilis, the urate oxidase variant may include a sequence selected from SEQ ID NOs: 155 to 157.

In one embodiment, when the urate oxidase variant is obtained by partially modifying the sequence of a urate oxidase derived from Arthrobacter globiformis, the urate oxidase variant may include a sequence selected from SEQ ID NOs: 158 to 160.

### Including a Sequence Similar to an Exemplary Sequence of the Vector Encoding a Urate Oxidase Variant.

In one embodiment, the vector encoding a urate oxidase variant may include a sequence that is 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to a sequence selected from SEQ ID NOs: 145 to 160. In one embodiment, the vector encoding a urate oxidase variant may include a sequence similar or identical to a sequence selected from SEQ ID NOs: 145 to 160 by a degree corresponding to one of the percentages described above. In one embodiment, the vector encoding a urate oxidase variant subunit may include a sequence similar or identical to a sequence selected from SEQ ID NOs: 145 to 160 by a degree in the range of 80% to 100%. In one embodiment, the vector encoding a urate oxidase variant subunit may include a sequence similar or identical to a sequence selected from SEQ ID NOs: 145 to 160 by a degree in the range of 95% or more.

### Albumin

### Overview of Albumin

Albumin included in the urate oxidase-albumin conjugate disclosed herein refers to a conventional albumin protein. The albumin serves to increase the half-life of a urate oxidase by conjugating with a urate oxidase and/or to decrease immunogenicity. The albumin is not limited if it can have the above-described functions, and may be a wild-type albumin found in nature or a genetically engineered albumin (albumin variant) from a wild-type albumin.

### Example of Albumin

In one embodiment, the albumin may be mammalian albumin. Specifically, the albumin may be human serum albumin. In one embodiment, the albumin may be wild-type human serum albumin. In one embodiment, the albumin may be recombinant albumin genetically engineered from wild-type human serum albumin.

### Example of Sequence of Albumin

In one embodiment, the albumin may be represented by a sequence selected from SEQ ID NOs: 133 to 144.

### Including Sequence Similar to Exemplary Albumin Sequence

In one embodiment, the albumin may include a sequence that is 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to a sequence selected from SEQ ID NOs: 133 to 144. In one embodiment, the albumin may include a sequence similar or identical to a sequence selected from SEQ ID NOs: 133 to 144 by a degree corresponding to one of the percentages described above. For example, the albumin may include a sequence similar or identical to a sequence selected from SEQ ID NOs: 133 to 144 by a degree in the range of 80% to 100%. Alternatively, the albumin may include a sequence similar or identical to a sequence selected from SEQ ID NOs: 133 to 144 by a degree in the range of 95% or more.

### Linker

### Overview of Linker

The urate oxidase-albumin conjugate disclosed herein is one in which a urate oxidase variant and albumin are conjugated through a linker. In this case, the linker refers to a material used to link a urate oxidase variant and an albumin when preparing the urate oxidase-albumin conjugate.

Specifically, the linker includes: an IEDDA reactive group; an anchor; and a thiol reactive group capable of binding to the albumin. In the process of preparing a urate oxidase-albumin conjugate, the urate oxidase variant and the linker bind to each other via an IEDDA reactive group, and the albumin and the linker bind to each other via the thiol reactive group. Specific bonding processes can be understood by referring to the relevant paragraph. Therefore, the linker of the urate oxidase-albumin conjugate does not exist in its original form, but exists in a form of 1) a urate oxidase-linker junction, 2) an anchor, and 3) an albumin-linker junction.

### Linker Structure 1—IEDDA Reactive Group

The linker includes an IEDDA reactive group capable of causing an inverse electron-demand Diels-Alder reaction (IEDDA reaction). The IEDDA reactive group is configured to be linked to the urate oxidase variant, and reacts with residues of nonnatural amino acids of the urate oxidase variant to form a urate oxidase-linker junction. In one embodiment, the IEDDA reactive group may include a dienophile functional group. Specifically, the IEDDA reactive group may be trans-cyclooctene or a derivative thereof.

In one embodiment, the IEDDA reactive group may be selected from the following:

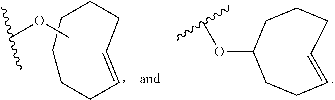

### Linker Structure 2—Thiol Reactive Group

The linker includes a thiol reactive group capable of reacting with thiol. The thiol group is configured to be linked to the albumin, and reacts with the thiol group included in the albumin to form an albumin-linker junction. In one embodiment, the thiol reactive group may be maleimide (MAL) or a derivative thereof, and/or 3-arylpropiolonitriles (APN) or a derivative thereof.

Specifically, the thiol reactive group may be selected from the following:

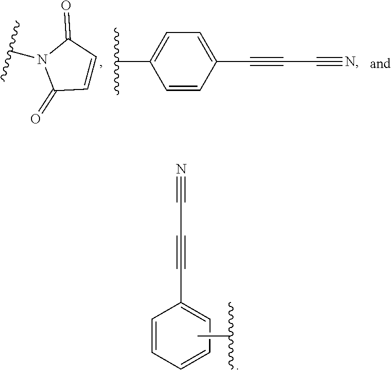

### Linker Structure 3—Anchor

The linker includes an anchor that links the IEDDA reactive group and the thiol reactive group. The anchor binds the IEDDA reactive group and the thiol reactive group into one molecule, and the structure of the anchor is not particularly limited as long as it does not affect the activity of the urate oxidase and/or albumin. In one embodiment, the anchor may have a linear structure. In another embodiment, the anchor may have a branched structure. In one embodiment, the anchor may include polyethylene glycol (PEG).

### Example of Linker

In one embodiment, the linker may be any one selected from the following:
The linker shown in FIG. 37C,
The linker shown in FIG. 37D,
The linker shown in FIG. 37E,
The linker shown in FIG. 37F, and
The linker shown in FIG. 37G.

### Urate Oxidase-Linker Junction

### Overview of Urate Oxidase-Linker Junction

The urate oxidase-albumin conjugate disclosed herein include a urate oxidase-linker junction. The urate oxidase-linker junction is generated by combining a urate oxidase variant and a linker through an IEDDA reaction. Specifically, the IEDDA reaction refers to a reaction between the residue of the nonnatural amino acid of the urate oxidase variant and the IEDDA reactive group of the linker, and, after the reaction, the structure of the urate oxidase-linker junction is determined depending on the residue of the nonnatural amino acid and the type of the IEDDA reactive group. Since the urate oxidase-albumin conjugate includes three or more albumin conjugates, the urate oxidase-albumin conjugate includes three or more urate oxidase-linker junctions. As described above, the urate oxidase-albumin conjugate includes three or more subunit-albumin conjugates. The subunit-albumin conjugate is a structure in which a urate oxidase variant subunit and an albumin are bound through a linker. Accordingly, each of the subunit-albumin conjugates includes at least one urate oxidase-linker junction.

### Position of Urate Oxidase-Linker Junction

The urate oxidase-linker junction is present at a position at which a nonnatural amino acid of a urate oxidase variant and the anchor of a linker are linked. As described above, since the urate oxidase-albumin conjugate is formed through the reaction of the residue of the nonnatural amino acid and the IEDDA reactive group, the urate oxidase-albumin conjugate is positioned to correspond to the residue of the nonnatural amino acid of the urate oxidase variant. In other words, the urate oxidase-linker junction is present at a position corresponding to the IEDDA reactive group of the linker.

### Reaction for Forming Urate Oxidase-Linker Junction

The reaction for forming the urate oxidase-linker junction is a kind of an inverse electron-demand Diels-Alder reaction (IEDDA reaction). A specific reaction mode may vary depending on the type of the functional group of the nonnatural amino acid of the urate oxidase variant and the IEDDA reactive group of the linker. In one embodiment, the urate oxidase-linker junction formation reaction may be any one of the following:

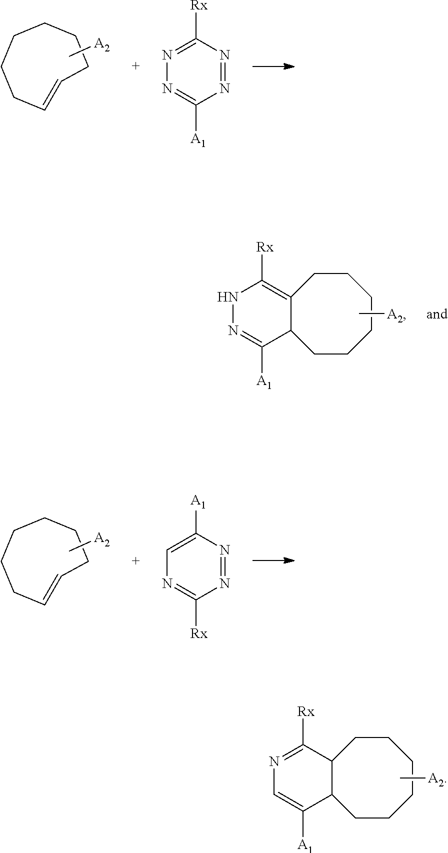

Here, A2 is a linker portion excluding the IEDDA reactive group, Rx may vary depending on the type of the nonnatural amino acid (refer to the above-described examples of nonnatural amino acids), and A1 is a urate oxidase variant portion excluding a tetrazine functional group of a nonnatural amino acid.

### Structure of Urate Oxidase-Linker Junction

In one embodiment, structure of the urate oxidase-linker junction may be anyone of the following:

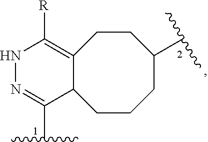

here, R is selected from H, CH₃,

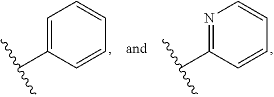 [SMILES: *c1ccccc1 ; *c1ccccn1]

the (1) part is linked to the urate oxidase variant, and the (2) part is linked to the anchor of the linker; and

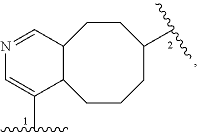 [SMILES: *C1=CN=CC2CCC(*)CCCC12]

here, the (1) part is linked to the urate oxidase variant, and the (2) part is linked to the anchor of the linker.

### Albumin-Linker Junction

### Overview of Albumin-Linker Junction

The urate oxidase-albumin conjugate disclosed herein include a urate oxidase-linker junction. The albumin-linker junction is generated by combining a thiol group included in albumin with a thiol group included in the linker. In this case, the thiol group of the albumin mediating the binding is characterized in that it is positioned to be spaced apart from the FcRn-binding domain of the albumin in order not to inhibit the half-life enhancing function of the albumin. Since the urate oxidase-albumin conjugate includes three or more albumin conjugates, the urate oxidase-albumin conjugate includes three or more albumin-linker junctions. As described above, the urate oxidase-albumin conjugate includes three or more subunit-albumin conjugates. The subunit-albumin conjugate is a structure in which a urate oxidase variant and an albumin are bound through a linker. Accordingly, each of the subunit-albumin conjugates includes at least one urate oxidase-linker junction.

### Position of Albumin-Linker Junction

The albumin-linker junction is present at a position at which the thiol moiety of the albumin and the anchor of the linker are linked. Since the urate oxidase-albumin conjugate disclosed herein has the purpose of increasing the half-life in the body by conjugating albumin to uric acid oxidase, the position where the albumin and the linker are connected must be a position spaced apart from the FcRn binding domain of albumin. As described above, since the urate oxidase-albumin conjugate is formed by reacting the thiol group of the albumin and the thiol reactive group of the linker, the albumin-linker junction is present at a position corresponding to the thiol group of the albumin. In other words, the albumin-linker junction is present at a position corresponding to the thiol reactive group of the linker. The position of the albumin-linker junction is selected from among the thiol groups included in albumin, which do not affect the structure, function, and/or activity of albumin.

### Exemplary Position of Albumin-Linker Junction

In one embodiment, the albumin-linker junction may be located in a thiol group included in the residue of the 34th cysteine of albumin represented by SEQ ID NOs: 133 to S016.

### Albumin-Linker Junction Formation Reaction

The reaction for forming the albumin-linker junction is a kind of thiol reaction. A specific reaction mode may vary depending on the type of thiol group of the linker.

In one embodiment, the urate albumin-linker junction formation reaction may be any one of the following:

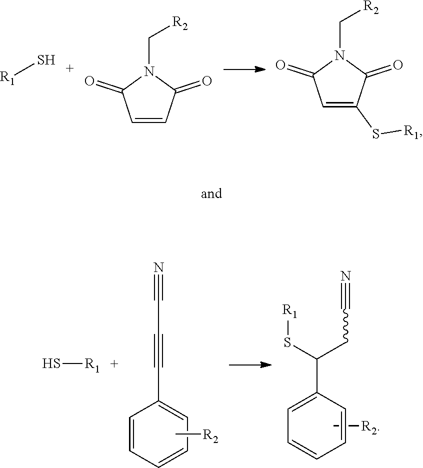

Here, R1 is an albumin moiety excluding the thiol group, and R2 is a linker moiety excluding the thiol group.

### Exemplary Structure of Albumin-Linker Junction

In one embodiment, the structure of the albumin-linker junction may be any one of the following:

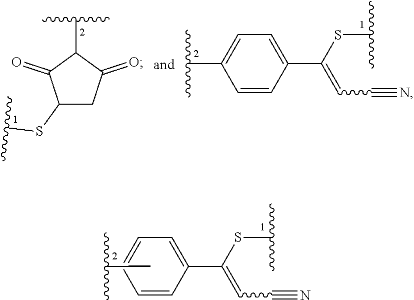

here, the (1) part is linked to albumin, and the (2) part is linked to the anchor of the linker.

### Anchor

### Overview of Anchor

The anchor disclosed herein refers to a structure connected between the urate oxidase-linker junction and the albumin-linker junction. The anchor binds the urate oxidase variant, the urate oxidase-linker junction, the albumin-linker junction, and the albumin into one structure. The anchor functions to regulate the distance between the urate oxidase variant and the albumin in the urate oxidase-albumin conjugate according to the structure thereof.

### Example of Structure of Anchor

In one embodiment, the anchor may be any one selected from the following:

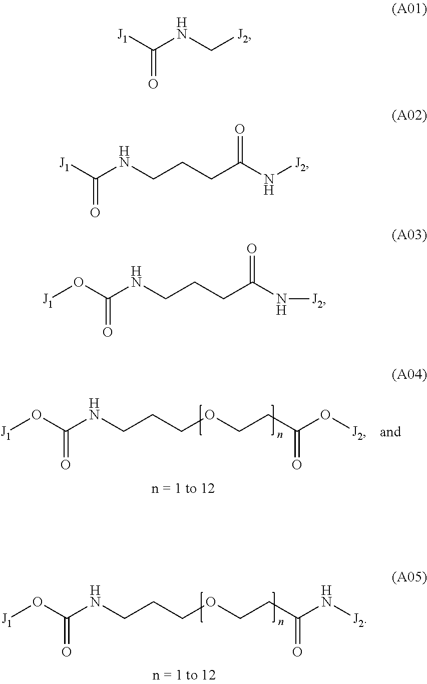

Herein J₁ is a urate oxidase-linker junction, and J₂ is an albumin-linker junction.

### Urate Oxidase-Albumin Conjugate Preparation Method

### Overview of Preparation Method for Urate Oxidase-Albumin Conjugate

The present description discloses a method of preparing a urate oxidase-albumin conjugate. The following elements are involved in preparing a urate oxidase-albumin conjugate: a urate oxidase variant; a linker; and an albumin. Herein, the details of the elements are the same as described above.

The urate oxidase-albumin conjugate preparation method is to prepare the above-described urate oxidase-albumin conjugate by appropriately reacting each of the elements. Specifically, the urate oxidase-albumin conjugate preparation method includes: reacting a nonnatural amino acid residue included in a urate oxidase variant with an IEDDA reactive group of a linker to make a urate oxidase-linker junction (urate oxidase-linker conjugation reaction); and reacting a thiol group of an albumin with a thiol reactive group of a linker to form an albumin-linker junction (albumin-linker conjugation reaction). In this case, the order in which the uric acid oxidase-linker conjugation reaction and the albumin-linker conjugation reaction occur is irrelevant, and both reactions may occur simultaneously. In addition, depending on the sequence of each reaction, intermediate products of the reaction may be produced. The urate oxidase-albumin conjugate preparation method will be described below in more detail.

### Urate Oxidase-Albumin Conjugate Preparation Method 1—Method of Binding Albumin and Linker First

In one embodiment, the urate oxidase-albumin conjugate preparation method includes the following:
reacting an albumin and a linker,
in which a thiol moiety of the albumin and a thiol reactive moiety of the linker come into contact with each other to create an albumin-linker conjugate; and
reacting the albumin-linker conjugate and the urate oxidase variant,
here, the IEDDA reactive group of the albumin-linker conjugate and the dien functional group of the nonnatural amino acid of the urate oxidase variant come into contact to produce a urate oxidase-albumin conjugate.

The urate oxidase variant, the linker, and the albumin, and elements included therein areas described above.

### Urate Oxidase-Albumin Conjugate Preparation Method 2—Method of Binding Urate Oxidase and Linker First

In one embodiment, the urate oxidase-albumin conjugate preparation method includes the following:
reacting a urate oxidase variant and a linker,
here, the IEDDA reactive group of the linker and the dien functional group of the nonnatural amino acid of the urate oxidase variant come into contact to produce a urate oxidase-linker conjugate; and
reacting the urate oxidase-linker conjugate and the albumin,
here, the thiol moiety of the albumin and the thiol moiety of the urate oxidase-linker conjugate come into contact to produce a urate oxidase-albumin conjugate.

The urate oxidase variant, the linker, and the albumin, and elements included therein areas described above.

### Urate Oxidase-Albumin Conjugate Preparation Method 3—Method of Binding Urate Oxidase, Linker, and Albumin Simultaneously

In one embodiment, the urate oxidase-albumin conjugate preparation method can be performed by adding all reactants and reacting them simultaneously.

In this case, the urate oxidase-albumin conjugate preparation method includes the following:
reacting a urate oxidase variant, a linker, and an albumin,
in which a thiol moiety of the albumin and a thiol moiety of the linker react to bind with each other,
the dien functional group contained in the nonnatural amino acid of the urate oxidase variant and the IEDDA reactive group of the linker are conjugated through a reaction, and
as a result of the reaction, the urate oxidase-albumin conjugate is produced.

The urate oxidase variant, the linker, and the albumin, and elements included therein are as described above.

### Characteristic of Urate Oxidase-Albumin Conjugate Preparation Method 1—High Stability in Body Due to Bioorthogonal Reaction

In the method for preparing the urate oxidase-albumin conjugate, the urate oxidase variant and the albumin are conjugated through an IEDDA reaction, which is a kind of bioorthogonal reaction. Since a chemical functional group involved in the conjugation reaction does not exist in a molecule in the body, the urate oxidase-albumin conjugate prepared by the preparation method has an advantage that the stability of the bond is very high even when introduced into the body.

### Characteristic of Urate Oxidase-Albumin Conjugate Preparation Method 2—High Yield Due to IEDDA Reaction

In the urate oxidase-albumin conjugate preparation method, an inverse electron demand Diels-Alder reaction (IEDDA reaction) is used for conjugation of the urate oxidase variant and the linker. Since the IEDDA reaction occurs at a very fast reaction rate and the reaction environment can be easily constructed, the yield is very high when preparing the conjugate compared to the case of using the Strain-Promoted Azide-Alkyne Cycloaddition (SPAAC) reaction.

### Characteristic of Urate Oxidase-Albumin Conjugate 3—Inclusion of Three or Four Albumins Per Conjugate

According to the urate oxidase-albumin conjugate preparation method disclosed herein, since an IEDDA reaction with a very high yield is used, a urate oxidase-albumin conjugate in which 3 or 4 albumins are conjugated per unit of urate oxidase can be obtained. This is clearly an improved characteristic compared to the limitation of the urate oxidase-albumin conjugate preparation method disclosed in the literature “KR 1637010 B1” by which only a urate oxidase-albumin conjugate in which one or two albumins are conjugated per unit of urate oxidase is obtained.

### Urate Oxidase-Linker Conjugation Method

The urate oxidase-albumin conjugate preparation method disclosed herein includes conjugating a urate oxidase variant and a linker. Specifically, the urate oxidase-linker conjugation method includes bringing the residue of the nonnatural amino acid of the urate oxidase variant into contact with the IEDDA reactive group of the linker. The urate oxidase-linker conjugation method is not affected by the site at which the albumin and the linker are conjugated or by whether the albumin and the linker are conjugated or not Therefore, the urate oxidase-linker conjugation method disclosed below is applicable to both the binding of the “linker” to the “urate oxidase variant” and the binding of the “albumin-linker conjugate” to the “urate oxidase variant”. The urate oxidase-linker conjugation method may be performed independently of the albumin-linker conjugation method.

The urate oxidase-linker conjugation method is not limited as long as it is a method capable of causing the reaction described in the section “Urate Oxidase-Linker Junction Formation Reaction”, and a person skilled in the art may use a known method capable of causing the reaction.

Here, when the IEDDA reaction between the tetrazine functional group and the trans-cyclooctene functional group is caused by the urate oxidase-linker conjugation method, the tetrazine functional group is reduced in a basic pH environment to increase the likelihood that the IEDDA reaction does not occur. Therefore, it is preferable that the IEDDA reaction proceeds in a neutral pH environment. In one embodiment, the urate oxidase-linker conjugation method may be performed in a neutral pH environment. In one embodiment, the urate oxidase-linker conjugation method may be performed in an environment of pH 8.0 or less, pH 9.0 or less, pH 10.0 or less, pH 11.0 or less, pH 11.0 or less, pH 13.0 or less, pH 14.0 or less.

### Albumin-Linker Conjugation Method

The urate oxidase-albumin conjugate preparation method disclosed herein includes conjugating an albumin with a linker. Specifically, the albumin-linker conjugation method includes bringing the thiol moiety of the albumin into contact with the thiol moiety of the liner. The albumin-linker conjugation method is not affected by the site at which the urate oxidase and the linker are conjugated or by whether the urate oxidase and the linker are conjugated or not. Therefore, the albumin-linker conjugation method disclosed below is applicable to both the binding of the “linker” to the “albumin” and the binding of the “urate oxidase-linker conjugate” to the “albumin”. The albumin-linker conjugation method may be performed independently of the urate oxidase-linker conjugation method.

### Use of Urate Oxidase-Albumin Conjugate

### Overview of Use of Urate Oxidase-Albumin Conjugate

The present description discloses a use of a urate oxidase-albumin conjugate. The urate oxidase-albumin conjugate has a long half-life in the body thereby being stable in the body, and has a low immunogenicity. Thus, the urate oxidase-albumin conjugate has an excellent uric acid lowering effect due to the above characteristics. Accordingly, the urate oxidase-albumin conjugate can be used to prevent or treat various diseases, disorders and/or indications caused by uric acid.

Preventive and/or Therapeutic Use of Urate Oxidase-Albumin Conjugate 1—Indication

In one embodiment, the uric acid oxidase-albumin conjugate may be used to prevent or treat hyperuricemia, acute gouty arthritis, intermittent gout, and chronic nodular gout, chronic kidney disease and/or tumor lysis syndrome (TLS).

Preventive and/or Therapeutic Use of Urate Oxidase-Albumin Conjugate 2—Administration Method

In one embodiment, the urate oxidase-albumin conjugate may be administered to patients through appropriate formulation to prevent or treat various diseases, disorders, and/or indications caused by uric acid. For example, the administration method may be one selected from oral administration, parenteral administration, intravenous administration, intraperitoneal administration, intramuscular administration, transdermal administration, and subcutaneous administration. Alternatively, the administration may be intravenous infusion.

Preventive and/or Therapeutic Use of Urate Oxidase 3—Albumin Conjugate—Dosage

In one embodiment, an appropriate dose of the urate oxidase-albumin conjugate may be administered to patients through appropriate formulation to prevent or treat various diseases, disorders, and/or indications caused by uric acid. For example, the dosage may be 0.01 mg/kg to 1000 mg/kg based on the urate oxidase-albumin conjugate.

Preventive and/or Therapeutic Use of Urate Oxidase-Albumin Conjugate 4—Administration Interval

In one embodiment, the urate oxidase-albumin conjugate may be administered to patients through appropriate formulation to prevent or treat various diseases, disorders, and/or indications caused by uric acid at appropriate intervals. For example, the administration interval may be once a day. That is, an interval at which the appropriate dose of the urate oxidase-albumin conjugate may be administered once a day. Alternatively, the urate oxidase-albumin conjugate may be administered two times a day. In this case, the dosage per administration is half the appropriate dose per day. Further alternatively, the administration interval may be 1 hour, 2 hours, 6 hours, 12 hours, 24 hours, 2 days, 3 days, one week, two weeks, one month, or three months for the appropriate dosage of the urate oxidase-albumin conjugate.

### Pharmaceutical Composition Including Urate Oxidase-Albumin Conjugate

### Overview of Pharmaceutical Composition Including Urate Oxidase-Albumin Conjugate

To use the urate oxidase-albumin conjugate for diseases, disorders, and/or indications caused by uric acid, the urate oxidase-albumin conjugate must undergo appropriate formulation. Disclosed herein is a pharmaceutical composition suitably formulated to use a urate oxidase-albumin conjugate as a therapeutic agent, and a pharmaceutically acceptable carrier required for formulation is disclosed. For example, the urate oxidase-albumin conjugate may be formulated for oral use, parenteral use, injection, aerosol, and/or transdermal use, and may include a pharmaceutically acceptable carrier for this purpose.

### Composition for Formulation 1—Oral Preparations

In one embodiment, the urate oxidase-albumin conjugate may be formulated as troches, lozenges, tablets, aqueous suspensions, oily suspensions, prepared powders, granules, emulsions, hard capsules, soft capsules, syrups, or elixirs.

In one embodiment, to formulating the urate oxidase-albumin conjugate as oral preparations, the following may be used: binders such as lactose, saccharose, sorbitol, mannitol, starch, amylopectin, cellulose or gelatin; excipients such as dicalcium phosphate and the like; disintegrants such as corn starch or sweet potato starch; and lubricants such as magnesium stearate, calcium stearate, sodium stearyl fumarate or polyethylene glycol wax. In addition, sweeteners, air fresheners, and syrups may be used. Furthermore, in the case of capsules, in addition to the above-mentioned substances, a liquid carrier such as fatty oil may be additionally used.

### Composition 2 for Formulation—Parenteral Preparations

In one embodiment, the urate oxidase-albumin conjugate may be formulated as an injection solution, suppository, powder for respiratory inhalation, aerosol for spray, ointment, powder for application, oil, or cream.

In one embodiment, in order to formulate the urate oxidase-albumin conjugate for parenteral administration, a sterile aqueous solution, a non-aqueous solvent, a suspension, an emulsion, a freeze-dried preparation, an external preparation, etc. may be used. As the non-aqueous solvent and the suspension, propylene glycol, polyethylene glycol, vegetable oils such as olive oil, and injectable esters such as ethyl oleate may be used.

### Composition 3 for Formulation—Injection Preparations

In one embodiment, in order to formulate the uric acid oxidase-albumin conjugate as an injection solution, the binder for the urate oxidase-albumin conjugate is mixed with a stabilizer or buffer in water to prepare a solution or suspension, and the solution or suspension may be formulated to be administered in units of an ampoule or vial.

### Composition 4 for Formulation—Aerosol Preparations

In one embodiment, the binder for the uric acid oxidase-albumin conjugate may be mixed with a propellant along with additives to prepare an aqueous dispersion concentrate or wet powder which may be subsequently formulated as aerosol preparations.

### Composition 5 for Formulation—Transdermal Preparations

In one embodiment, when the uric acid oxidase-albumin conjugate is formulated for transdermal use, animal oil, vegetable oil, wax, paraffin, starch, tragacanth, cellulose derivative, polyethylene glycol, silicone, bentonite, silica, talc, zinc oxide, etc. may be added as a carrier to the binder for the urate oxidase-albumin conjugate to prepare ointment, cream, powder for application, oil, external preparation for skin, etc.

### Composition 6 for Formulation—Adjuvants and Other Components

In one embodiment, the pharmaceutical composition including the urate oxidase-aluminum conjugate may include: water, saline, dextrose, ethanol, glycerol, sodium chloride, dextrose, mannitol, sorbitol, lactose, gelatin, albumin, aluminum hydroxide, Freund's incomplete adjuvant and complete adjuvant (Pifco Laboratories, Detroit, Mich.), Merck Adjuvant 65 (Merck and Company, Inc., Rahway, NJ.), Alhydrogel (Al(OH)₃), aluminum hydroxide gel (alum), or aluminum salt such as aluminum phosphate, AS04 series, MF, squalene, MF59, QS21, calcium, iron or zinc salt, insoluble suspension of acylated tyrosine, acylated fructose, cationically or anionically derived polysaccharides, polyphosphazenes, biodegradable microspheres, and Quil A, toll-like receptor (TLR) agonists, PHAD [Avanti polar lipid, Monophosphoryl Lipid A (synthetic)], monophosphoryl lipid A (MPL), synthetic lipid A, lipid A mimics or analogues, aluminum salts, cytokines, saponins, prolactin, growth hormone deoxycholic acid, betaglucan, polyribonucleotides, muramyl dipeptide (MDP) derivatives, CpG oligo, lipopolysaccharide (LPS) of Gram-negative bacteria, polyphosphazene, emulsion, virosome, cochleate, poly(lactide-co-glycolide)(PLG) microparticles, poloxamer particles, microparticles, liposomes, or suitable combinations thereof.

### Possible Example of Invention

### Urate Oxidase-Albumin Conjugate 1

### Example 1, Urate Oxidase-Albumin Conjugate

A urate oxidase-albumin conjugate represented by [formula 1]:

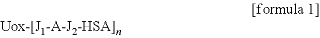

wherein Uox is a urate oxidase variant, J1 is a urate oxidase-linker junction, A is an anchor, J2 is an albumin-linker junction, and HSA is Human Serum Albumin,
the urate oxidase variant includes three or more nonnatural amino acids having a diene functional group,
the urate oxidase-linker junction is a structure in which a diene functional group of the nonnatural amino acid and a dienophile functional group connected to the anchor are bound through an IEDDA reaction, and
n is 3 or 4.

### Example 2, Limitation of Microorganism for Deriving Urate Oxidase

In Example 1, the urate oxidase variant is a substance resulting from substitution of three or more amino acids in a wild-type uric acid oxidase sequence derived from a microorganism with nonnatural amino acids, and the microorganism is selected from the following: Aspergillus Flavus, Arthrobacter globiformis, and Candida utilis.

### Example 3, Four Variant Subunits

The urate oxidase-albumin conjugate of any one of Examples 1 to 2, in which the urate oxidase variant is a tetramer formed by oligomerization of four urate oxidase variant subunits, and the urate oxidase variant subunit is a subunit obtained by substituting one or more amino acids in the sequence of a wild-type urate oxidase subunit with nonnatural amino acids.

### Example 4, Three Variant Subunits, One Wild Subunit

The urate oxidase-albumin conjugate of any one of Examples 1 to 2, in which the urate oxidase variant is a tetramer formed by oligomerization of three urate oxidase variant subunits and one wild-type urate oxidase subunit, and the urate oxidase variant subunit is a subunit obtained by substituting one or more amino acids in the sequence of a wild urate oxidase subunit with nonnatural amino acids.

### Example 5, Nonnatural Amino Acid, Limitation of Tetrazine Functional Group

The urate oxidase-albumin conjugate of any one of Examples 1 to 4, in which the dien functional group is a triazine functional group or a derivative, or a tetrazine functional group or a derivative thereof.

### Example 6, Example of Nonnatural Amino Acid (Name)

The urate oxidase-albumin of Example 5, in which the nonnatural amino acid is any one selected from the following:

4-(1,2,3,4-tetrazin-3-yl) phenylalanine (frTet), 4-(6-methyl-s-tetrazin-3-yl)phenylalanine (Tet-v2.0), 3-(4-(1,2,4-triazin-6-yl)phenyl)-2-aminopropanoic acid, 2-amino-3-(4-(2-(6-methyl-1,2,4,5-tetrazin-3-yl)ethyl)phenyl)propanoic acid, 2-amino-3-(4-(6-phenyl-1,2,4,5-tetrazin-3-yl)phenyl)propanoic acid, 3-(4-((1,2,4,5-tetrazin-3-yl)amino)phenyl)-2-aminopropanoic acid, 3-(4-(2-(1,2,4,5-tetrazin-3-yl)ethyl)phenyl)-2-aminopropanoic acid, 3-(4-((1,2,4,5-tetrazin-3-yl)thio)phenyl)-2-aminopropanoic acid, 2-amino-3-(4-((6-methyl-1,2,4,5-tetrazin-3-yl)thio)phenyl)propanoic acid, 3-(4-((1,2,4,5-tetrazin-3-yl)oxy)phenyl)-2-aminopropanoic acid, 2-amino-3-(4-((6-methyl-1,2,4,5-tetrazin-3-yl)oxy)phenyl)propanoic acid, 3-(4′-(1,2,4,5-tetrazin-3-yl)-[1,1′-biphenyl]-4-yl)-2-aminopropanoic acid, 2-amino-3-(4′-(6-methyl-1,2,4,5-tetrazin-3-yl)-[1,1′-biphenyl]-4-yl)propanoic acid, 2-amino-3-(6-(6-(pyridin-2-yl)-1,2,4,5-tetrazin-3-yl)pyridin-3-yl)propanoic acid, 3-(4-(1,2,4,5-tetrazin-3-yl)phenyl)-2-aminopropanoic acid, and 2-amino-3-(4-(6-methyl-1,2,4,5-tetrazin-3-yl)phenyl)propanoic acid.

### Example 7, Example of Nonnatural Amino Acid (Chemical Formula)

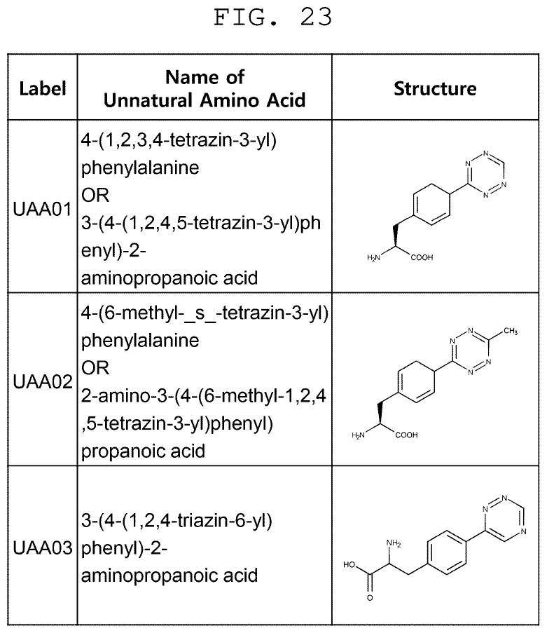

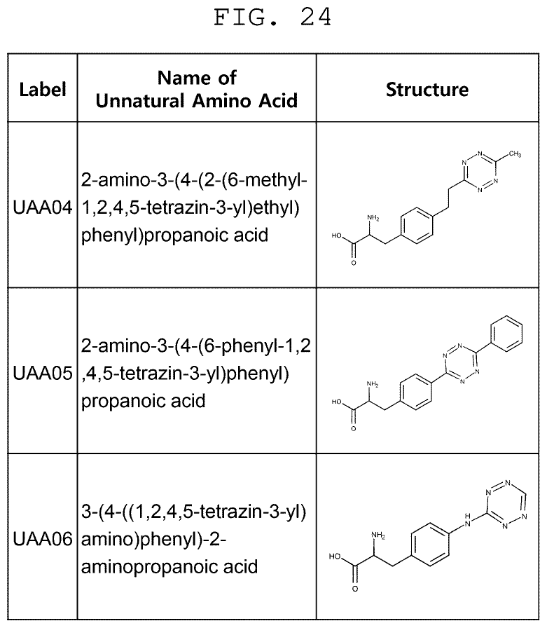

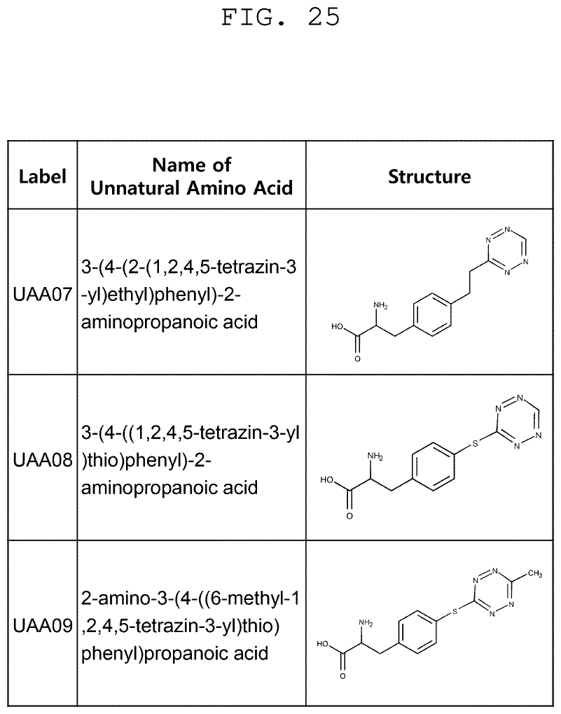

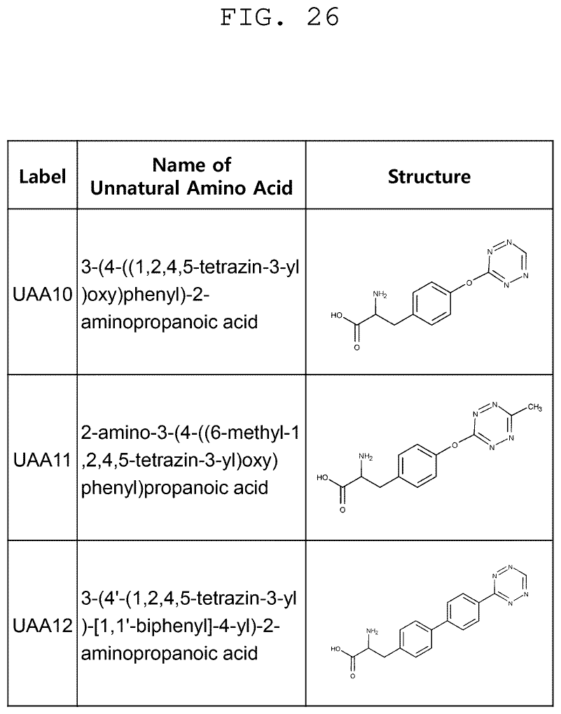

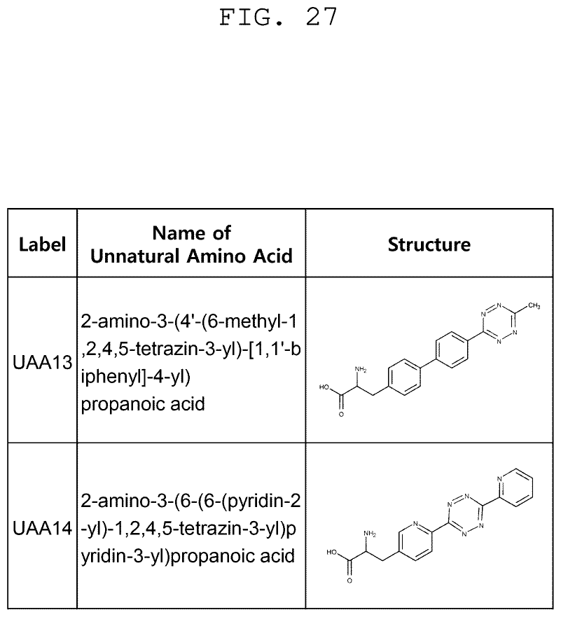

The urate oxidase-albumin conjugate of Example 5, in which the nonnatural amino acids are each independently selected from the tables of FIGS. 23 to 27.

### Example 8, Subunit Sequence Example 1, Asp.Uox

The urate oxidase-albumin conjugate of any one of Examples 2 to 7, in which the urate oxidase variant subunit is represented by a sequence selected from the following:

(SEQ ID NO: 2)
SAVKAARYGKDNVRVYKVHKDEKTGVQTVYEMTVCVLLEGEIETSYTKADNSVIVA
TDSIKNTIYITAKQNPVTPPELFGSILGTHFIEKYNHIHAAHVNIVCHRWTRMDIDGKPHPHSFIR
DSEEKRNVQVDVVEXKGIDIKSSLSGLTVLKSTNSQFWGFLRDEYTTLKETWDRILSTDVDAT
WQWKNFSGLQEVRSHVPKFDATWATAREVTLKTFAEDNSASVQATMYKMAEQILARQQLIE
TVEYSLPNKHYFEIDLSWHKGLQNTGKNAEVFAPQSDPNGLIKCTVGRSSLKSKL;
(SEQ ID NO: 3)
SAVKAARYGKDNVRVYKVHKDXKTGVQTVYEMTVCVLLEGEIETSYTKADNSVIVA
TDSIKNTIYITAKQNPVTPPELFGSILGTHFIEKYNHIHAAHVNIVCHRWTRMDIDGKPHPHSFIR
DSEEKRNVQVDVVEGKGIDIKSSLSGLTVLKSTNSQFWGFLRDEYTTLKETWDRILSTDVDAT
WQWKNFSGLQEVRSHVPKFDATWATAREVTLKTFAEDNSASVQATMYKMAEQILARQQLIE
TVEYSLPNKHYFEIDLSWHKGLQNTGKNAEVFAPQSDPNGLIKCTVGRSSLKSKL;
(SEQ ID NO: 4)
SAVKAARYGKDNVRVYKVHKDEKTGVQTVYEMTVCVLLEGEIETSYTKADNSVIVA
TDSIKNTIYITAKQNPVTPPELFGSILGTHFIEKYXHIHAAHVNIVCHRWTRMDIDGKPHPHSFIR
DSEEKRNVQVDVVEGKGIDIKSSLSGLTVLKSTNSQFWGFLRDEYTTLKETWDRILSTDVDAT
WQWKNFSGLQEVRSHVPKFDATWATAREVTLKTFAEDNSASVQATMYKMAEQILARQQLIE
TVEYSLPNKHYFEIDLSWHKGLQNTGKNAEVFAPQSDPNGLIKCTVGRSSLKSKL;
(SEQ ID NO: 5)
SAVKAARYGKDNVRVYKVHKDEXTGVQTVYEMTVCVLLEGEIETSYTKADNSVIVA
TDSIKNTIYITAKQNPVTPPELFGSILGTHFIEKYNHIHAAHVNIVCHRWTRMDIDGKPHPHSFIR
DSEEKRNVQVDVVEGKGIDIKSSLSGLTVLKSTNSQFWGFLRDEYTTLKETWDRILSTDVDAT
WQWKNFSGLQEVRSHVPKFDATWATAREVTLKTFAEDNSASVQATMYKMAEQILARQQLIE
TVEYSLPNKHYFEIDLSWHKGLQNTGKNAEVFAPQSDPNGLIKCTVGRSSLKSKL;
(SEQ ID NO: 6)
SAVKAARYGKDNVRVYKVHKDEKTGVQTVYEMTVCVLLEGEIETSYTKADNSVIVA
TDSIKNTIYITAKQNPVTPPELFGSILGTHFIEKYNHIHAAHVNIVCHRWTRMDIDGKPHPHSFIR
DSEEKRNVQVDVVEGKGIDIKSSLSGLTVLKSTNSQFWGFLRDEYTTLKETWDRILSTDVDAT
WQWKNFSGLQEVRSHVPKFDATWATAREVTLKTFAEDNSASVQATMYKMAEQILARQQLIE
TVEYSLPNKHYFEIDLSWHKGLQNTGKNAEVFAPQSDPNGLIKCTVGRXSLKSKL;
(SEQ ID NO: 7)
SAVKAARYGKDNVRVYKVHKDEKTGVQTVYEMTVCVLLEGEIETSYTKADNSVIVA
TDSIKNTIYITAKQNPVTPPELFGSILGTHFIEKYNHIHAAHVNIVCHRWTRMDIDXKPHPHSFIR
DSEEKRNVQVDVVEGKGIDIKSSLSGLTVLKSTNSQFWGFLRDEYTTLKETWDRILSTDVDAT
WQWKNFSGLQEVRSHVPKFDATWATAREVTLKTFAEDNSASVQATMYKMAEQILARQQLIE
TVEYSLPNKHYFEIDLSWHKGLQNTGKNAEVFAPQSDPNGLIKCTVGRSSLKSKL;
(SEQ ID NO: 8)
SAVKAARYGKDNVRVYKVHKDEKTGVQTVYEMTVCVLLEGEIETSYTKADNSVIVA
TDSIKNTIYITAKQNPVTPPELFGSILGTHFIEKYNHIHAAHVNIVCHRWTRMDIDGKPHPHSFIR
DSEEKRNVQVDVVEGKGIDIKSSLSGLTVLKSTNSQFWGFLRDEYTTLKETWDRILSTDVDAT
WQWKNFSGLQEVRSHVPKFDATWATAREVTLKTFAEDNSASVQATMYKMAEQILARQQLIE
TVEYSLPNKHYFEIDLSWHKGLQNTGXNAEVFAPQSDPNGLIKCTVGRSSLKSKL;
(SEQ ID NO: 9)
SAVKAARYGKDNVRVYKVHKDEKTGVQTVYEMTVCVLLEGEIETSYTKADNSVIVA
TDSIKNTIYITAKQNPVTPPELFGSILGTHFIEKYNHIHAAHVNIVCHRWTRMDIDGKPHPHSFIR
DSEEKRNVQVDVVEGKGIDIKSSLSGLTVLKSTNSQFWGFLRDEYTTLXETWDRILSTDVDAT
WQWKNFSGLQEVRSHVPKFDATWATAREVTLKTFAEDNSASVQATMYKMAEQILARQQLIE
TVEYSLPNKHYFEIDLSWHKGLQNTGKNAEVFAPQSDPNGLIKCTVGRSSLKSKL;
(SEQ ID NO: 10)
SAVKAARYGKDNVRVYKVHKDEKTGVQTVYEMTVCVLLEGEIETSYTKADNSVIVA
TDSIKNTIYITAKQNPVTPPELFGSILGTHFIEKYNHIHAAHVNIVCHRWTRMDIDGKPHPHSFIR
DSEEKRNVQVDVVEGKGIDIKSSLSGLTVLKSTNSQFWGFLRDEYTTLKETWDRILSTDVDAT
WQWKNFSGLQEVRSHVPKFDATWATAREVTLKTFAEDNSASVQATMYKMAEQILXRQQLIE
TVEYSLPNKHYFEIDLSWHKGLQNTGKNAEVFAPQSDPNGLIKCTVGRSSLKSKL;
(SEQ ID NO: 11)
SAVKAARYGKDNVRVYKVHKDEKTGVQTVYEMTVCVLLEGEIETSYTKADNSVIVA
TDSIKNTIYITAKQNPVTPPELFGSILGTHFIXKYNHIHAAHVNIVCHRWTRMDIDGKPHPHSFIR
DSEEKRNVQVDVVEGKGIDIKSSLSGLTVLKSTNSQFWGFLRDEYTTLKETWDRILSTDVDAT
WQWKNFSGLQEVRSHVPKFDATWATAREVTLKTFAEDNSASVQATMYKMAEQILARQQLIE
TVEYSLPNKHYFEIDLSWHKGLQNTGKNAEVFAPQSDPNGLIKCTVGRSSLKSKL;
(SEQ ID NO: 12)
SAVKAARYGKDNVRVYKVHKDEKTGVQTVYEMTVCVLLEGEIETSYTKADNSVIVA
TDSIKNTIYITAKQNPVTPPELFGSILGTHFIEKYNHIHAAHVNIVCHRWTRMDIDGKPHPHSFIR
DSEEKRNVQVDVVEGKGIDIKSSLSGLTVLKSTNSQFWGFLRDEYTTLKETWDRILSTDVDAT
WQWKNFSGLQEVRSHVPKFDATWATAREVTLKTFAEDNSASVQATMYKMAEQILARQQLIE
TVEYSLPNKHYFEIDLSWHXGLQNTGKNAEVFAPQSDPNGLIKCTVGRSSLKSKL;
(SEQ ID NO: 13)
SAVKAARYGKDNVRVYKVHKDEKXGVQTVYEMTVCVLLEGEIETSYTKADNSVIVA
TDSIKNTIYITAKQNPVTPPELFGSILGTHFIEKYNHIHAAHVNIVCHRWTRMDIDGKPHPHSFIR
DSEEKRNVQVDVVEGKGIDIKSSLSGLTVLKSTNSQFWGFLRDEYTTLKETWDRILSTDVDAT
WQWKNFSGLQEVRSHVPKFDATWATAREVTLKTFAEDNSASVQATMYKMAEQILARQQLIE
TVEYSLPNKHYFEIDLSWHKGLQNTGKNAEVFAPQSDPNGLIKCTVGRSSLKSKL;
(SEQ ID NO: 14)
SAVKAARYGKDNVRVYKVHKDEKTGVQTVYEMTVCVLLEGEIETSYTXADNSVIVA
TDSIKNTIYITAKQNPVTPPELFGSILGTHFIEKYNHIHAAHVNIVCHRWTRMDIDGKPHPHSFIR
DSEEKRNVQVDVVEGKGIDIKSSLSGLTVLKSTNSQFWGFLRDEYTTLKETWDRILSTDVDAT
WQWKNFSGLQEVRSHVPKFDATWATAREVTLKTFAEDNSASVQATMYKMAEQILARQQLIE
TVEYSLPNKHYFEIDLSWHKGLQNTGKNAEVFAPQSDPNGLIKCTVGRSSLKSKL;
(SEQ ID NO: 15)
SAVKAARYGKDNVRVYKVHKDEKTGVQTVYEMTVCVLLEGEIETSYTKADNSVIVA
TDSIKNTIYITAKQNPVTPPELFGSILGTHFIEKYNHIHAAHVNIVCHRWTRMDIDGKPHPHSFIR
DSEEKRNVQVDVVEGKGIDIKSSLSGLTVLKSTNSQFWGFLRDEYTTLKETWDRILSTDVDAT
WQWKNFXGLQEVRSHVPKFDATWATAREVTLKTFAEDNSASVQATMYKMAEQILARQQLIE
TVEYSLPNKHYFEIDLSWHKGLQNTGKNAEVFAPQSDPNGLIKCTVGRSSLKSKL;
(SEQ ID NO: 16)
SAVKAARYGKDNVRVYKVHKDEKTGVQTVYEMTVCVLLEGEIETSYTKADNSVIVA
TDSIKNTIYITAKQNPVTPPELFGSILGTHFIEKYNHIHAAHVNIVCHRWTRMDIDGKPHPHSFIR
DSEEKRNVQVDVVEGKGIDIKSSLSGLTVLKSTNSQFWGFLRDEYTTLKETWDRILSTDVDAT
WQWKNFSGLQEVRSHVXKFDATWATAREVTLKTFAEDNSASVQATMYKMAEQILARQQLIE
TVEYSLPNKHYFEIDLSWHKGLQNTGKNAEVFAPQSDPNGLIKCTVGRSSLKSKL;
(SEQ ID NO: 17)
SAVKAARYGKDNVRVYKVHKDEKTGVQTVYEMTVCVLLEGEIETSYTKADNSVIVA
TDSIKNTIYITAKQNPVTPPELFGSILGTHFIEKYNHIHAAHVNIVCHRWTRMXIDGKPHPHSFIR
DSEEKRNVQVDVVEGKGIDIKSSLSGLTVLKSTNSQFWGFLRDEYTTLKETWDRILSTDVDAT
WQWKNFSGLQEVRSHVPKFDATWATAREVTLKTFAEDNSASVQATMYKMAEQILARQQLIE
TVEYSLPNKHYFEIDLSWHKGLQNTGKNAEVFAPQSDPNGLIKCTVGRSSLKSKL;
(SEQ ID NO: 18)
SAVKAARYGKDNVRVYKVHKDEKTGVQTVYEMTVCVLLEGEIETSYTKADNSVIVA
TDSIKNTIYITAKQNPVTPPELFGSILGTHFIEKYNHIHAAHVNIVCHRWTRMDIDGKPHPHSFIR
DSEEKRNVQVDVVEGKGIDIKSSLSGLTVLKSTNSQFWGFLRDEYTTLKETWDRILSTDVDAT
WQWKNFSGLQEVRSHVPKFDATWATAREVTLKTFAEDNSASVQATMYKMAEQILARQXLIE
TVEYSLPNKHYFEIDLSWHKGLQNTGKNAEVFAPQSDPNGLIKCTVGRSSLKSKL;
(SEQ ID NO: 19)
SAVKAARYGKDNVRVYKVHKDEKTGVQTVYEMTVCVLLEGEIETSYTKADNSVIVA
TDSIKNTIYITAKQNPVTPPELFGSILGTHFIEKYNHIHAAHVNIVCHRWTRMDIDGKPHPHSFIR
DSEEKRNVQVDVVEGKGIDIKSSLSGLTVLKSTNSQFWGFLRDEYTTLKETWDRILSTDVDAT
WQWKNFSGLXEVRSHVPKFDATWATAREVTLKTFAEDNSASVQATMYKMAEQILARQQLIE
TVEYSLPNKHYFEIDLSWHKGLQNTGKNAEVFAPQSDPNGLIKCTVGRSSLKSKL;
(SEQ ID NO: 20)
SAVKAARYGKDNVRVYKVHKDEKTGVQTVYEMTVCVLLEGEIETSYTKADNSVIVA
TDSIKNTIYITAKQNPVTPPELFGSILGTHFIEKYNHIHAAHVNIVCHRWTRMDIDGKPHPHSFIR
DSEEKRNVQVDVVEGXGIDIKSSLSGLTVLKSTNSQFWGFLRDEYTTLKETWDRILSTDVDAT
WQWKNFSGLQEVRSHVPKFDATWATAREVTLKTFAEDNSASVQATMYKMAEQILARQQLIE
TVEYSLPNKHYFEIDLSWHKGLQNTGKNAEVFAPQSDPNGLIKCTVGRSSLKSKL;
(SEQ ID NO: 21)
SAVKAARYGKDNVRVYKVHKDEKTGVQTVYEMTVCVLLEGEIETSYTKADNSVIVA
TDSIKNTIYITAKQNPVTPPELFGSILGTHFIEKYNHIHAAHVNIVCHRWTRMDIDGKXHPHSFIR
DSEEKRNVQVDVVEGKGIDIKSSLSGLTVLKSTNSQFWGFLRDEYTTLKETWDRILSTDVDAT
WQWKNFSGLQEVRSHVPKFDATWATAREVTLKTFAEDNSASVQATMYKMAEQILARQQLIE
TVEYSLPNKHYFEIDLSWHKGLQNTGKNAEVFAPQSDPNGLIKCTVGRSSLKSKL;
(SEQ ID NO: 22)
SAVKAARYGKDNVRVYKVHKDEKTGVQTVYEMTVCVLLEGEIETSYTKADNSVIVA
TDSIKNTIYITAKQNPVTPPELFGSILGTHFIEKYNHIHAAHVNIVCHRWTRMDIDGKPHPHSFIR
DSEEKRNVQVDVVEGKGIDIKSSLSGLTVLKSTNSQFWGFLRDEYTTLKETWDRILSTDVDAT
WQWKNFSGLQEVRXHVPKFDATWATAREVTLKTFAEDNSASVQATMYKMAEQILARQQLIE
TVEYSLPNKHYFEIDLSWHKGLQNTGKNAEVFAPQSDPNGLIKCTVGRSSLKSKL;
(SEQ ID NO: 23)
SAVKAARYGKDNVRVYKVHKDEKTGVQTVYEMTVCVLLEGEIETSYTKADNSVIVA
TDSIKNTIYITAKQNPVTPPELFGSILGTHFIEKYNHIHAAHVNIVCHRWTRMDIDGKPHPHSFIR
DSEEKRNVQVDVVEGKGIDIKSSLSGLTVLKSTNSQFWGFLRDEYTTLKETWDRILSTDVDAT
WQWKNFSGLQEVRSHVPKFDATWATAREVTLKTFAEDNSASVQATMYKMAEQILARQQLIE
TVEYSLPNKHYFEIDLSWHKGLQNTXKNAEVFAPQSDPNGLIKCTVGRSSLKSKL;
(SEQ ID NO: 24)
SAVXAARYGKDNVRVYKVHKDEKTGVQTVYEMTVCVLLEGEIETSYTKADNSVIVA
TDSIKNTIYITAKQNPVTPPELFGSILGTHFIEKYNHIHAAHVNIVCHRWTRMDIDGKPHPHSFIR
DSEEKRNVQVDVVEGKGIDIKSSLSGLTVLKSTNSQFWGFLRDEYTTLKETWDRILSTDVDAT
WQWKNFSGLQEVRSHVPKFDATWATAREVTLKTFAEDNSASVQATMYKMAEQILARQQLIE
TVEYSLPNKHYFEIDLSWHKGLQNTGKNAEVFAPQSDPNGLIKCTVGRSSLKSKL;
(SEQ ID NO: 25)
SAVKAARYGKDNVRVYKVHKDEKTGVQTVYEMTVCVLLEGEIETSYTKADNSVIVA
TDSIKNTIYITAKQNPVTPPELFGSILGTHFIEKYNHIHAAHVNIVCHRWTRMDIXGKPHPHSFIR
DSEEKRNVQVDVVEGKGIDIKSSLSGLTVLKSTNSQFWGFLRDEYTTLKETWDRILSTDVDAT
WQWKNFSGLQEVRSHVPKFDATWATAREVTLKTFAEDNSASVQATMYKMAEQILARQQLIE
TVEYSLPNKHYFEIDLSWHKGLQNTGKNAEVFAPQSDPNGLIKCTVGRSSLKSKL;
(SEQ ID NO: 26)
SAVKAARYGKDNVRVYKVHKDEKTGVQTVYEMTVCVLLEGEIETSYTKADNSVIVA
TDSIKNTIYITAKQNPVTPPELFGSILGTHFIEKYNHIHAAHVNIVCHRWTRMDIDGKPHPHSFIR
DSEEKRNVQVDVVEGKGIDIKSSLSGLTVLKSTNSQFWGFLRDEYTTLKETWDRILSTDVDAT
WQWKNFSGLQEVRSHVPKFDATWATAREVTLKTFAEDNSASVQATMYKMAEQILARQQLIE
TVEYSLPNKHYFEIDLSWHKXLQNTGKNAEVFAPQSDPNGLIKCTVGRSSLKSKL;
(SEQ ID NO: 27)
SAVKAARYGKDNVRVYKVHKDEKTGVQTVYEMTVCVLLEGEIETSYTKADNSVIVA
TDSIKNTIYITAKQNPVTPPELFGSILGTHFIEKYNHIHAAHVNIVCHRWTRMDIDGXPHPHSFIR
DSEEKRNVQVDVVEGKGIDIKSSLSGLTVLKSTNSQFWGFLRDEYTTLKETWDRILSTDVDAT
WQWKNFSGLQEVRSHVPKFDATWATAREVTLKTFAEDNSASVQATMYKMAEQILARQQLIE
TVEYSLPNKHYFEIDLSWHKGLQNTGKNAEVFAPQSDPNGLIKCTVGRSSLKSKL;
(SEQ ID NO: 28)
SAVKAARYGKDNVRVYKVHKDEKTGVQTVYEMTVCVLLEGEIETSYTKADNSVIVA
TDSIKNTIYITAKXNPVTPPELFGSILGTHFIEKYNHIHAAHVNIVCHRWTRMDIDGKPHPHSFIR
DSEEKRNVQVDVVEGKGIDIKSSLSGLTVLKSTNSQFWGFLRDEYTTLKETWDRILSTDVDAT
WQWKNFSGLQEVRSHVPKFDATWATAREVTLKTFAEDNSASVQATMYKMAEQILARQQLIE
TVEYSLPNKHYFEIDLSWHKGLQNTGKNAEVFAPQSDPNGLIKCTVGRSSLKSKL;
(SEQ ID NO: 29)
SAVKAARYGKDNVRVYKVHKDEKTGVQTVYEMTVCVLLEGEIETSYTKADNSVIVA
TDSIKNTIYITAKQNPVTPPELFGSILGTHFIEKYNHIHAAHVNIVCHRWTRMDIDGKPHPHSFIR
DSEEKRNVQVDVVEGKGIDIKSSLSGLTVLKSTNSQFWGFLRDEYTTLKETXDRILSTDVDAT
WQWKNFSGLQEVRSHVPKFDATWATAREVTLKTFAEDNSASVQATMYKMAEQILARQQLIE
TVEYSLPNKHYFEIDLSWHKGLQNTGKNAEVFAPQSDPNGLIKCTVGRSSLKSKL;
(SEQ ID NO: 30)
SAVKAARYGKDNVRVYKVHKDEKTGVQTVYEMTVCVLLEGEIETSYTKADNSVIVA
TDSIKNTIYITAKQNPVTPPELFGSILGTHFIEKYNHIHAAHVNIVCHRWTRMDIDGKPHPHSFIR
DSEEKRNVQVDVVEGKGIDIKSSLSGLTVLKSTNSQFWGFLRDEYTTLKETWDRILSTDVDAT
WQWKNFSGLQEVRSHVPKFDATWATAREVTLKTFAEDXSASVQATMYKMAEQILARQQLIE
TVEYSLPNKHYFEIDLSWHKGLQNTGKNAEVFAPQSDPNGLIKCTVGRSSLKSKL;
(SEQ ID NO: 31)
SAVKAARYGKDNVRVYKVHKDEKTGVQTVYEMTVCVLLEGXIETSYTKADNSVIVA
TDSIKNTIYITAKQNPVTPPELFGSILGTHFIEKYNHIHAAHVNIVCHRWTRMDIDGKPHPHSFIR
DSEEKRNVQVDVVEGKGIDIKSSLSGLTVLKSTNSQFWGFLRDEYTTLKETWDRILSTDVDAT
WQWKNFSGLQEVRSHVPKFDATWATAREVTLKTFAEDNSASVQATMYKMAEQILARQQLIE
TVEYSLPNKHYFEIDLSWHKGLQNTGKNAEVFAPQSDPNGLIKCTVGRSSLKSKL;
(SEQ ID NO: 32)
SAVKAARYGKDNVRVYKVHKDEKTGVQTVYEMTVCVLLEGEIETSYTKADNSVIVA
TDSIKNTIYITAKQNPVTPPELFGSILGTHFIEKYNHIHAAHVNIVCHRWTRMDIDGKPHPHSFIR
DSEEKRNVQVDVVEGKGIDIKSSLSGLTVLKSTNSQFWGFLRDEYTTLKETWDRILSTDVDAT
WQWKNFSGLQEVRSHVPKFDATWATAREVTLKTFAEDNSASVQATMYKMAEQILARQQLIE
TVEYSLPNKHYFEIXLSWHKGLQNTGKNAEVFAPQSDPNGLIKCTVGRSSLKSKL;
(SEQ ID NO: 33)
SAVKAARYGKDNVRVYKVHKDEKTXVQTVYEMTVCVLLEGEIETSYTKADNSVIVA
TDSIKNTIYITAKQNPVTPPELFGSILGTHFIEKYNHIHAAHVNIVCHRWTRMDIDGKPHPHSFIR
DSEEKRNVQVDVVEGKGIDIKSSLSGLTVLKSTNSQFWGFLRDEYTTLKETWDRILSTDVDAT
WQWKNFSGLQEVRSHVPKFDATWATAREVTLKTFAEDNSASVQATMYKMAEQILARQQLIE
TVEYSLPNKHYFEIDLSWHKGLQNTGKNAEVFAPQSDPNGLIKCTVGRSSLKSKL;
(SEQ ID NO: 34)
SAVKAARYGKDNVRVYKVHKDEKTGVQTVYEMTVCVLLEGEIETSYTKADNXVIVA
TDSIKNTIYITAKQNPVTPPELFGSILGTHFIEKYNHIHAAHVNIVCHRWTRMDIDGKPHPHSFIR
DSEEKRNVQVDVVEGKGIDIKSSLSGLTVLKSTNSQFWGFLRDEYTTLKETWDRILSTDVDAT
WQWKNFSGLQEVRSHVPKFDATWATAREVTLKTFAEDNSASVQATMYKMAEQILARQQLIE
TVEYSLPNKHYFEIDLSWHKGLQNTGKNAEVFAPQSDPNGLIKCTVGRSSLKSKL;
(SEQ ID NO: 35)
SAVKAARYGKDNVRVYKVHKDEKTGVQTVYEMTVCVLLEGEIETSYTKADNSVIVA
TDSIKNTIYITAKQNPVTPPELFGSILGTHFIEKYNHIHAAHVNIVCHRWTRMDIDGKPHPHSFIR
DSEEKRNVQVDVVEGKGIDIKSSLSGLTVLKSTNSQFWGFLRDEYTTLKETWDRILSTDVDAT
WQWKNFSGLQEVRSHVPKFDATWATAREVTLKTFAEDNSASVQATMYKMAEQILAXQQLIE
TVEYSLPNKHYFEIDLSWHKGLQNTGKNAEVFAPQSDPNGLIKCTVGRSSLKSKL;
(SEQ ID NO: 36)
SAVKAARYGKDNVRVYKVHKDEKTGVQTVYEMTVCVLLEGEIETSYTKADNSVIVA
TDSIKNTIYITAKQNPVTPPELFGSILGTHFIEKYNHIHAAHVNIVCHRWTRMDIDGKPHPHSFIR
DSEEKRNVQVDVVEGKGIDIKSSLSGLTVLKSTNSQFWGFLRDEYTTLKETWDRILSTDVDAT
WQWKNFSGLQEVRSHVPKFDATWATARXVTLKTFAEDNSASVQATMYKMAEQILARQQLIE
TVEYSLPNKHYFEIDLSWHKGLQNTGKNAEVFAPQSDPNGLIKCTVGRSSLKSKL;
(SEQ ID NO: 37)
SAVKAARYGKDNVRVYKVHKDEKTGVQTVYEMTVCVLLEGEIETSYTKADNSVIVA
TDSIKNTIYITAKQNPVTPPELFGSILGTHFIEKYNHIHAAHVNIVCHRWTRMDIDGKPHPHSFIR
DSEEKRNVQVDVVEGKGIDIKSSLSGLTVLKSTNSQFWGFLRDEYTTLKETWDRILSTDVDAT
WQWKNFSGLQEVRSHVPKFDATWATAREVTLKTFAEDNSASVQATMYKMAEQILARQQLIE
TVEYSLPNKHYFEIDLSWHKGLQNTGKXAEVFAPQSDPNGLIKCTVGRSSLKSKL;
(SEQ ID NO: 38)
SAVKAARYGKDNVRVYKVHKDEKTGVQTVYEMTVCVLLEGEIETSYTKADNSVIVA
TDSIKNTIYITAKQNPVTPPELFGSILGTHFIEKYNHIHAAHVNIVCHRWTRMDIDGKPHPHSFIR
DSEEKRNVQVDVVEGKGIDIKSSLSGLTVLKSTNSQFWGFLRDEYTTLKETWDRILSTDVDAT
WQWKNFSGLQEVRSHVPKFDATWATAREVTLKTFAXDNSASVQATMYKMAEQILARQQLIE
TVEYSLPNKHYFEIDLSWHKGLQNTGKNAEVFAPQSDPNGLIKCTVGRSSLKSKL;
(SEQ ID NO: 39)
SAVKAARYGKDNVRVYKVHKDEKTGVQTVYEMTVCVLLEGEIETSYTKADNSVIVA
TDSIKNTIYITAKQNPVTPPELFGSILGTHFIEKYNHIHAAHVNIVCHRWTRMDIDGKPHPHSFIR
DSEEKRNVQVDVVEGKGIDIKSSLSGLTVLKSTNSQFWGFLRDEYTTLKETWDRILSTDVDAT
WQWKNFSGLQEVRSHVPKFDXTWATAREVTLKTFAEDNSASVQATMYKMAEQILARQQLIE
TVEYSLPNKHYFEIDLSWHKGLQNTGKNAEVFAPQSDPNGLIKCTVGRSSLKSKL;
(SEQ ID NO: 40)
SAVKAARYGKDNVRVYKVHKDEKTGVQTVYEMTVCVLLEGEIETSYTKADNSVIVA
TDSIKNTIYITAKQNPVTPPELFGSILGTHFIEKYNHIHAAHVNIVCHRWTRMDIDGKPHPHSFIR
DSEEKRNVQVDVVEGKGIDIKSSLSGLTVLKSTNSQFWGFLRDEYTTLKETWDRILSTDVDAT
WQWKNFSGLQEVRSHVPKFDATWATAREVTLKTFAEDNSASVQATMYKMAXQILARQQLIE
TVEYSLPNKHYFEIDLSWHKGLQNTGKNAEVFAPQSDPNGLIKCTVGRSSLKSKL;
(SEQ ID NO: 41)
SAVKAARYGKDNVRVYKVHKDEKTGVQTVYEMTVCVLLEGEIETSYTKADNSVIVA
TDSIKNTIYITAKQNPVTPPELFGSILGTHFIEKYNHIHAAHVNIVCHRWTRMDIDGKPHPHSFIR
DSEEKRNVQVDVVEGKGIDIKSSLSGLTVLKSTNSQFWGFLXDEYTTLKETWDRILSTDVDAT
WQWKNFSGLQEVRSHVPKFDATWATAREVTLKTFAEDNSASVQATMYKMAEQILARQQLIE
TVEYSLPNKHYFEIDLSWHKGLQNTGKNAEVFAPQSDPNGLIKCTVGRSSLKSKL;
(SEQ ID NO: 42)
SAVKAARYGKDNVRVYKVHKDEKTGVQTVYEMTVCVLLEGEIETSYTKADNSVIVA
TDSIKNTIYITAKQNPVTPPELFGSILGTHFIEKYNHIHAAHVNIVCHRWTRMDIDGKPHPHSFIR
DSEEKRNVQVDVVEGKGIDIKSSLSGLTVLKSTNSQFWGFLRDEYTTLKETWDRILSTDVDAT
WQWKNFSGLQEVRSHVPKFDATWATAREVTLKTFAEDNSASVQATMYKMAEQILARQQLIE
TVEYSLPNKHYFEIDLSWHKGLXNTGKNAEVFAPQSDPNGLIKCTVGRSSLKSKL;
(SEQ ID NO: 43)
SAVKAARYGKDNVRVYKVHKDEKTGVQTVYEMTVCVLLEGEIETSYTKADNSVIVA
TDSIKNTIYITAKQNPVTPPELFGSILGTHFIEKYNHIHAAHVNIVCHRWTRMDIDGKPHPHSFIR
DSEEKRNVQVDVVXGKGIDIKSSLSGLTVLKSTNSQFWGFLRDEYTTLKETWDRILSTDVDAT
WQWKNFSGLQEVRSHVPKFDATWATAREVTLKTFAEDNSASVQATMYKMAEQILARQQLIE
TVEYSLPNKHYFEIDLSWHKGLQNTGKNAEVFAPQSDPNGLIKCTVGRSSLKSKL;
(SEQ ID NO: 44)
SAVKAARYGKDNVRVYKVHKDEKTGVQTVYEMTVCVLLEGEIETSYTKADNSVIVA
TDSIKNTIYITAKQNPVTPPELFGSILGTHFIEKYNHIHAAHVNIVCHRWTRMDIDGKPHPHSFIR
DSEEKRNVQVDVVEGKGIDIKSSLSGLTVLKSTNSQFWGFLRDEYTTLKETWDRILSTDVDAT
WQWKNFSGLQEVRSHVPKFDATWATAREVTLKTFAEDNSASVQATMYKMAEQILARQQLIE
TVEYSLPNKHYFXIDLSWHKGLQNTGKNAEVFAPQSDPNGLIKCTVGRSSLKSKL;
(SEQ ID NO: 45)
SAVKAARYGKDNVRVYKVHKDEKTGVQTVYEMTVCVLLEGEIETSYTKADNSVIVA
TDSIKNTIYITAKQNPVTPPELFGSILGTHFIEKYNHIHAAHVNIVCHRWTRMDIDGKPHPHSFIR
DSEEKRNVQVDVVEGKGIDIKSSLSGLTVLKSTNSQFWGFLRDEYTTLKETWDRILSTDVDAT
WQWKNFSGLQEVRSHVPKFDATWATAREVTLKTFAEDNSASVQATMYKMAEQILARQQLIX
TVEYSLPNKHYFEIDLSWHKGLQNTGKNAEVFAPQSDPNGLIKCTVGRSSLKSKL;
(SEQ ID NO: 46)
SAVKAARYGKDNVRVYKVHKDEKTGVQTVYEMTVCVLLEGEIETSYTKXDNSVIVA
TDSIKNTIYITAKQNPVTPPELFGSILGTHFIEKYNHIHAAHVNIVCHRWTRMDIDGKPHPHSFIR
DSEEKRNVQVDVVEGKGIDIKSSLSGLTVLKSTNSQFWGFLRDEYTTLKETWDRILSTDVDAT
WQWKNFSGLQEVRSHVPKFDATWATAREVTLKTFAEDNSASVQATMYKMAEQILARQQLIE
TVEYSLPNKHYFEIDLSWHKGLQNTGKNAEVFAPQSDPNGLIKCTVGRSSLKSKL;
(SEQ ID NO: 47)
SAVKAARYGKDNVRVYKVHKDEKTGVQTVYEMTVCVLLEGEIETSYTKADNSVIVA
TDSIKNTIYITAKQNPVTPPELFGSILGTHFIEKYNHIHAAHVNIVCHRWTRMDIDGKPHPHSFIR
DSEEKRNVQVDVVEGKGIDIKSSLSXLTVLKSTNSQFWGFLRDEYTTLKETWDRILSTDVDAT
WQWKNFSGLQEVRSHVPKFDATWATAREVTLKTFAEDNSASVQATMYKMAEQILARQQLIE
TVEYSLPNKHYFEIDLSWHKGLQNTGKNAEVFAPQSDPNGLIKCTVGRSSLKSKL;
(SEQ ID NO: 48)
SAVKAARYGKDNVRVYKVXKDEKTGVQTVYEMTVCVLLEGEIETSYTKADNSVIVA
TDSIKNTIYITAKQNPVTPPELFGSILGTHFIEKYNHIHAAHVNIVCHRWTRMDIDGKPHPHSFIR
DSEEKRNVQVDVVEGKGIDIKSSLSGLTVLKSTNSQFWGFLRDEYTTLKETWDRILSTDVDAT
WQWKNFSGLQEVRSHVPKFDATWATAREVTLKTFAEDNSASVQATMYKMAEQILARQQLIE
TVEYSLPNKHYFEIDLSWHKGLQNTGKNAEVFAPQSDPNGLIKCTVGRSSLKSKL;
(SEQ ID NO: 49)
SAVKAARYGKDNVRVYKVHKDEKTGVQTVYEMTVCVLLEGEIETSYTKADNSVIVA
TDSIKNTIYITAKQNPVTPPELFGSILGTHFIEKYNHIHAAHVNIVCHRWTRMDIDGKPHPHSFIR
DSEEKRNVQVDVVEGKGIDIKSSLSGLTVLKSTNSQFWGFLRDEYTTLKETWDRILSTDVDAT
WQWKNFSGLQEVRSHVPKFDATWATAREVTLKTFAEDNSASVQATMYKMAEQILARQQLIE
TVEYSLPNKHYFEIDLSWHKGLQNTGKNAEVFAPQSDPNGLIKCTVGRSXLKSKL;
and
(SEQ ID NO: 50)
SAVKAARYGKDNVRVYKVHKDEKTGVQTVYEMTVCVLLEGEIETSYXKADNSVIVA
TDSIKNTIYITAKQNPVTPPELFGSILGTHFIEKYNHIHAAHVNIVCHRWTRMDIDGKPHPHSFIR
DSEEKRNVQVDVVEGKGIDIKSSLSGLTVLKSTNSQFWGFLRDEYTTLKETWDRILSTDVDAT
WQWKNFSGLQEVRSHVPKFDATWATAREVTLKTFAEDNSASVQATMYKMAEQILARQQLIE
TVEYSLPNKHYFEIDLSWHKGLQNTGKNAEVFAPQSDPNGLIKCTVGRSSLKSKL,

where X is selected from the nonnatural amino acids disclosed in the tables of FIGS. 23 to 27.

### Example 9, Subunit Sequence Example 2, Candida.Uox

The urate oxidase-albumin conjugate of any one of Examples 2 to 7, in which the urate oxidase variant subunit is represented by a sequence selected from the following:

(SEQ ID NO: 52)
MSTTLSSSTYGKDNVKFLKVKKDPQNPKKQEVMEATVTCLLEGGFDTSYTEADNSSIV
PTDTVKNTILVLAKTTEIWPIERFAAKLATHFVEKYSHVSGVSVKIVQDRWVKYAVDGKPHDH
SFIHEGGEKRITDLYYKRSGDYKLSSAIKDLTVLKSTGSMFYGYNKCDFTTLQPTTDRILSTDV
DATWVWDNKKIGSVYDIAKAADKGIFDNVYNQAREITLTTFALENSPSVQATMFNMATQILE
KACSVYSVSYALPNKHYFLIDLKWKGLENDNELFYPSPHPNGLIKCTVVRKEKXKL;
(SEQ ID NO: 53)
MSTTLSSSTYGKDNVKFLKVKKDPQXPKKQEVMEATVTCLLEGGFDTSYTEADNSSIV
PTDTVKNTILVLAKTTEIWPIERFAAKLATHFVEKYSHVSGVSVKIVQDRWVKYAVDGKPHDH
SFIHEGGEKRITDLYYKRSGDYKLSSAIKDLTVLKSTGSMFYGYNKCDFTTLQPTTDRILSTDV
DATWVWDNKKIGSVYDIAKAADKGIFDNVYNQAREITLTTFALENSPSVQATMFNMATQILE
KACSVYSVSYALPNKHYFLIDLKWKGLENDNELFYPSPHPNGLIKCTVVRKEKTKL;
(SEQ ID NO: 54)
MSTTLSSSTYGKDNVKFLKVKKDPQNPKKQEVMEATVTCLLEGGFDTSYTEADNSSIV
PTDTVKNTILVLAKTTEIWPIERFAAKLATHFVEKYSHVSGVSVKIVQDRWVKYAVDGKPHDH
SFIHEGGEKRITDLYYKRSGDYKLSSAIKDLTVLKSTGSMFYGYNKCDFTTLQPTTDRILSTDV
DATWVWDNKKIGSVYDIAKAADKGIFDNVYNQAREITLTTFALENSPSVQATMFNMATQILE
KACSVYSVSYALPNKHYFLIDLKWKGLENDNELFYPSPHPNGLIKCTVVRKEKTKX;
(SEQ ID NO: 55)
MSTTLSSSTYGKDNVKFLKVKKDPQNPKKQEVMEATVTCLLEGGFDTSYTEADNSSIV
PTDTVKNTILVLAKTTEIWPIERFAAKLATHFVEKYSHVSGVSVKIVQDRWVKYAVDGKPHDH
SFIHEGGEKRITDLYYKRSGDYKLSSAIKDLTVLKSTGSMFYGYNKCDFTTLQPTTDRILSTDV
DATWVWDNXKIGSVYDIAKAADKGIFDNVYNQAREITLTTFALENSPSVQATMFNMATQILE
KACSVYSVSYALPNKHYFLIDLKWKGLENDNELFYPSPHPNGLIKCTVVRKEKTKL;
(SEQ ID NO: 56)
MSTTLSSSTYGKDNVKFLKVKKDPQNPKKQEVMEATVTCLLEGGFDTSYTEADNSSIV
PTDTVKNTILVLAKTTEIWPIERFAAKLATHFVEKYXHVSGVSVKIVQDRWVKYAVDGKPHD
HSFIHEGGEKRITDLYYKRSGDYKLSSAIKDLTVLKSTGSMFYGYNKCDFTTLQPTTDRILSTD
VDATWVWDNKKIGSVYDIAKAADKGIFDNVYNQAREITLTTFALENSPSVQATMFNMATQIL
EKACSVYSVSYALPNKHYFLIDLKWKGLENDNELFYPSPHPNGLIKCTVVRKEKTKL;
(SEQ ID NO: 57)
MSTTLSSSTYGKDNVKFLKVKKDPQNPKKQEVMEATVTCLLEGGFDTSYTEADNSSIV
PTDTVKNTILVLAKTTEIWPIERFAAKLATHFVEKYSHVSGVSVKIVQDRWVKYAVDGKPHDH
SFIHEGGEKRITDLYYKRXGDYKLSSAIKDLTVLKSTGSMFYGYNKCDFTTLQPTTDRILSTDV
DATWVWDNKKIGSVYDIAKAADKGIFDNVYNQAREITLTTFALENSPSVQATMFNMATQILE
KACSVYSVSYALPNKHYFLIDLKWKGLENDNELFYPSPHPNGLIKCTVVRKEKTKL;
(SEQ ID NO: 58)
MSTTLSSSTYGKDNVKFLKVKKDPQNPKKQEVMEATVTCLLEGGFDTSYTEADNSSIV
PTDTVKNTILVLAKTTEIWPIERFAAKLATHFVEKYSHVSGVSVKIVQDRWVKYAVDXKPHDH
SFIHEGGEKRITDLYYKRSGDYKLSSAIKDLTVLKSTGSMFYGYNKCDFTTLQPTTDRILSTDV
DATWVWDNKKIGSVYDIAKAADKGIFDNVYNQAREITLTTFALENSPSVQATMFNMATQILE
KACSVYSVSYALPNKHYFLIDLKWKGLENDNELFYPSPHPNGLIKCTVVRKEKTKL;
(SEQ ID NO: 59)
MSTTLSSSTYGKDNVKFLKVKKDPQNPKKQEVMEATVTCLLEGGFDTSYTEADNSSIV
PTDTVKNTILVLAKTTEIWPIERFAAKLATHFVEKYSHVSGVSVKIVQDRWVKYAVDGKPHDH
SFIHEGGEKRITDLYYKRSGDYKLSSAIKDLTVLKSTGSMFYGYNKCDFTTLQPTTDRILSTDV
DATWVWDNKKIGSVYDIAKAADKGIFDNVYNQAREITLTTFALENSPSVQATMFNMATQILE
KACSVYSVSYALPNKHYFLIDLKWKGLENDNELFYPSPHPNGLIKCTVVRKEKTXL;
(SEQ ID NO: 60)
MSTTLSSSTYGKDNVKFLKVKKDPQNPKKQEVMEATVTCLLEGGFDTSYTEADNSSIV
PTDTVKNTILVLAKTTEIWPIERFAAKLATHFVEKYSHVSGVSVKIVQDRWVKYAVDGKPHDH
SFIHEGGEKRITDLYYKRSGDYKLSSAIKDLTVLKSTGSMFYGYNXCDFTTLQPTTDRILSTDV
DATWVWDNKKIGSVYDIAKAADKGIFDNVYNQAREITLTTFALENSPSVQATMFNMATQILE
KACSVYSVSYALPNKHYFLIDLKWKGLENDNELFYPSPHPNGLIKCTVVRKEKTKL;
(SEQ ID NO: 61)
MSTTLSSSTYGKDNVKFLKVKKDPQNPKKQEVMEATVTCLLEGGFDTSYTEADNSSIV
PTDTVKNTILVLAKTTEIWPIERFAAKLATHFVEKYSHVSGVSVKIVQDRWVKYAVXGKPHDH
SFIHEGGEKRITDLYYKRSGDYKLSSAIKDLTVLKSTGSMFYGYNKCDFTTLQPTTDRILSTDV
DATWVWDNKKIGSVYDIAKAADKGIFDNVYNQAREITLTTFALENSPSVQATMFNMATQILE
KACSVYSVSYALPNKHYFLIDLKWKGLENDNELFYPSPHPNGLIKCTVVRKEKTKL;
(SEQ ID NO: 62)
MSTTLSSSTYGKDNVKFLKVKKDPQNPKKQEVMEATVTCLLEGGFDTSYTEADNSSIV
PTDTVKNTILVLAKTTEIWPIERFAAKLATHFVEKYSHVSGVSVKIVQDRWVKYAVDGKPHDH
SFIHEGGEKRITDLYYKRSGDYKLSSAIKDLTVLKSTGSMFYGYNKCDFTTLQPTTDRILSTDV
DATWVWDNKKIGSVYDIAKAADKGIFDNVYNQAREITLTTFALENSPSVQATMFNMATQILE
KACSVYSVSYALPNKHYFLIDLKWKGLENDNELFYPSPHPNGLIKCTVVRKXKTKL;
(SEQ ID NO: 63)
MSTTLSSSTYGKDNVKFLKVKKDXQNPKKQEVMEATVTCLLEGGFDTSYTEADNSSI
VPTDTVKNTILVLAKTTEIWPIERFAAKLATHFVEKYSHVSGVSVKIVQDRWVKYAVDGKPHD
HSFIHEGGEKRITDLYYKRSGDYKLSSAIKDLTVLKSTGSMFYGYNKCDFTTLQPTTDRILSTD
VDATWVWDNKKIGSVYDIAKAADKGIFDNVYNQAREITLTTFALENSPSVQATMFNMATQIL
EKACSVYSVSYALPNKHYFLIDLKWKGLENDNELFYPSPHPNGLIKCTVVRKEKTKL;
(SEQ ID NO: 64)
MSTTLSSSTYGKDNVKFLKVKKDPQNPKKQEVMEATVTCLLEGGFDTSYTEADNSSIV
PTDTVKNTILVLAKTTEIWPIERFAAKLATHFVEKYSHVSGVSVKIVQDRWVKYAVDGKPHDH
SFIHEGGEKRITDLYYKRSGDYKLSSAIKDLTVLKSTGSMFYGYNKCDFTTLQPTTDRILSTDV
DATWVWDNKKIGSVYDIAKAADKGIFDNVYNQAREITLTTFALENSPSVQATMFNMATQILE
KACSVYSVSYALPNKHYFLIDLKXKGLENDNELFYPSPHPNGLIKCTVVRKEKTKL;
(SEQ ID NO: 65)
MSTTLSSSTYGKDNVKFLKVKKDPQNPKKQEVMEATVTCLLEGGFDTSYTEADNSSIV
PTDTVKNTILVLAKTTEIWPIERFAAKLATHFVEKYSHVSGVSVKIVQDRWVKYAVDGKPHDH
SFIHEGGEKRITDLYYKRSGDYKLSSAIKDLTVLKSTGSMFYGYNKCDFTTLQPTTDRILSTDV
DATWVWDNKKIGSVYDIAKAADKGIFDNVYNQAREITLTTFALENSPSVQATMFNMATQILE
KACSVYSVSYALPNKHYFLIDLKWKGLENXNELFYPSPHPNGLIKCTVVRKEKTKL;
(SEQ ID NO: 66)
MSTTLSSSTYGKDNVKFLKVKKDPQNPKKQEVMEATVTCLLEGGFDTSYTEADNSSIV
PTDTVKNTILVLAKTTEIWPIERFAAKLATHFVEKYSHVSGVSVKIVQDRWVKYAVDGKPHDH
SFIHEGGEKRITDLYYKRSGDYKLSSAIKDLTVLKSTGSMFYGYNKCXFTTLQPTTDRILSTDV
DATWVWDNKKIGSVYDIAKAADKGIFDNVYNQAREITLTTFALENSPSVQATMFNMATQILE
KACSVYSVSYALPNKHYFLIDLKWKGLENDNELFYPSPHPNGLIKCTVVRKEKTKL;
(SEQ ID NO: 67)
MSTTLSSSTYGKDNVKFLKVKKDPQNPKKQEVMEATVTCLLEGGFDTSYTEADNSSIV
PTDTVKNTILVLAKTTEIWPIERFAAKLATHFVEKYSHVSGVSVKIVQDRWVKYAVDGKXHD
HSFIHEGGEKRITDLYYKRSGDYKLSSAIKDLTVLKSTGSMFYGYNKCDFTTLQPTTDRILSTD
VDATWVWDNKKIGSVYDIAKAADKGIFDNVYNQAREITLTTFALENSPSVQATMFNMATQIL
EKACSVYSVSYALPNKHYFLIDLKWKGLENDNELFYPSPHPNGLIKCTVVRKEKTKL;
(SEQ ID NO: 68)
MSTTLSSSTYGKDNVKFLKVKKDPQNPKKQEVMEATVTCLLEGGFDTSYTEADNSSIV
PTDTVKNTILVLAKTTEIWPIERFAAKLATHFVEKYSHVSGVSVKIVQDRWVKYAVDGKPHDH
SFIHEGGEKRITDLYYKRSGDYKLSSAIKDLTVLKSTGSMFYGYNKCDFTTLQPTXDRILSTDV
DATWVWDNKKIGSVYDIAKAADKGIFDNVYNQAREITLTTFALENSPSVQATMFNMATQILE
KACSVYSVSYALPNKHYFLIDLKWKGLENDNELFYPSPHPNGLIKCTVVRKEKTKL;
(SEQ ID NO: 69)
MSTTLSSSTYGKDNVKFLKVKKDPQNPKKQEVMEATVTCLLEGGFDTSYTEADNSSIV
PTDTVKNTILVLAKTTEIWPIERFAAKLATHFVEKYSHVSGVSVKIVQDRWVKYAVDGKPHDH
SFIHEGGEKRITDLYYKRSGDYKLSSAIKDLTVLKSTGSMFYGYNKCDFTTLXPTTDRILSTDV
DATWVWDNKKIGSVYDIAKAADKGIFDNVYNQAREITLTTFALENSPSVQATMFNMATQILE
KACSVYSVSYALPNKHYFLIDLKWKGLENDNELFYPSPHPNGLIKCTVVRKEKTKL;
(SEQ ID NO: 70)
MSTTLSSSTYGKDNVKFLKVKKDPQNPKKQEVMEATVTCLLEGGFDTSYTEADNSSIV
PTDTVKNTILVLAKTTEIWPIERFAAKLATHFVEKYSHVSGVSVKIVQDRWVKYAVDGKPHDH
SFIHEGGEKRITDLYYKRSGDYKLSSAIKDLTVLKSTGSMFYGYNKCDFTTLQPTTDRILSTDV
DATWVWDNKKIGSVYDIAKAADXGIFDNVYNQAREITLTTFALENSPSVQATMFNMATQILE
KACSVYSVSYALPNKHYFLIDLKWKGLENDNELFYPSPHPNGLIKCTVVRKEKTKL;
(SEQ ID NO: 71)
MSTTLSSSTYGKDNVKFLKVKKDPQNPKKQEVMEATVTCLLEGGFDTSYTEADNSSIV
PTDTVKNTILVLAKTTEIWPIERFAAKLATHFVEKYSHVSGVSVKIVQDRWVKYAVDGKPHDH
SFIHEGGEKRITDLYYKRSGDYKLSSAIKDLTVLKSTGSMFYGYNKCDFTTLQPTTDRILSTDV
DATWVWDNKKIGSVYDIAKAADKGIFDNVYNQAREITLTTFALENSPSVQATMFNMATQILE
KACSVYSVSYALPNKHYFLIDLKWKGLXNDNELFYPSPHPNGLIKCTVVRKEKTKL;
(SEQ ID NO: 72)
MSTTLSSSTYGKDNVKFLKVKKDPQNPKKQEVMEATVTCLLEGGFDTSYTEADNSSIV
PTDTVKNTILVLAKTTEIWPIERFAAKLATHFVEKYSHVSGVSVKIVQDRWVKYAVDGKPHDH
SFIHEGGEKRITDLYYKRSGDYKLSSAIKDLTVLKSTGSMFYGYNKCDFTTLQPTTDRILSTDV
DATWVWDNKKIGSVYDIAKAADKGIFDNVYNQAREITLTTFALENSPSVQATMFNMATQILE
KACSVYSVSYALPNKHYFXIDLKWKGLENDNELFYPSPHPNGLIKCTVVRKEKTKL;
(SEQ ID NO: 73)
MSTTLSSSTYGKDNVKFLKVKKDPQNPKKQEVMEATVTCLLEGGFDTSYTEADNSSIV
PTDTVKNTILVLAKTTEIWPIERFAAKLATHFVEKYSHVSGVSVKIVQDRWVKYAVDGKPHDH
SFIHEGGEKRITDLYYKRSGDYKLSSAIKDLTVLKSTGSMFYGYNKCDFTTLQPTTDRILSTDV
DATWVWDNKKIGSVYDIAKAADKGIFDNVYNQAREITLTTFALENSPSVQATMFNMATQILE
KACSVYSVSYALPNKHYFLIDLKWKXLENDNELFYPSPHPNGLIKCTVVRKEKTKL;
(SEQ ID NO: 74)
MSTTLSSSTYGKDNVKFLKVKKDPQNPKKQEVMEATVTCLLEGGFDTSYTEADNSSIV
PTDTVKNTILVLAKTTEIWPIERFAAKLATHFVEKYSHVSGVSVKIVQDRWVKYAVDGKPHDH
SFIHEGGEKRITDLYYKRSGDYKLSSAIKDLTVLKSTGSMFYGYNKCDFTTLQPTTDRILSTDV
DATWVWDNKKIGSVXDIAKAADKGIFDNVYNQAREITLTTFALENSPSVQATMFNMATQILE
KACSVYSVSYALPNKHYFLIDLKWKGLENDNELFYPSPHPNGLIKCTVVRKEKTKL;
(SEQ ID NO: 75)
MSTTLSSSTYGKDNVKFLKVKKDPQNPKKQEVMEATVTCLLEGGFDTSYTEADNSSIV
PTDTVKNTILVLAKTTEIWPIERFAAKLATHFVXKYSHVSGVSVKIVQDRWVKYAVDGKPHD
HSFIHEGGEKRITDLYYKRSGDYKLSSAIKDLTVLKSTGSMFYGYNKCDFTTLQPTTDRILSTD
VDATWVWDNKKIGSVYDIAKAADKGIFDNVYNQAREITLTTFALENSPSVQATMFNMATQIL
EKACSVYSVSYALPNKHYFLIDLKWKGLENDNELFYPSPHPNGLIKCTVVRKEKTKL;
(SEQ ID NO: 76)
MSTTLSSSTYGKDNVKFLKVKKDPQNPKKQEVMEATVTCLLEGGFDTSYTEADNSSIV
PTDTVKNTILVLAKTTEIWPIERFAAKLATHFVEKYSHVSGVSVKIVQDRWVKYAVDGKPHDH
SFIHEGGEKRITDLYYKRSGDYKLSSAIKDLTVLKSTGSMFYGYNKCDFTTLQPTTDRILSTDV
DATWVWDNKKIGSVYDIAKAADKGIFDNVYNQAREITLTTFALENSPSVQATMFNMATQILX
KACSVYSVSYALPNKHYFLIDLKWKGLENDNELFYPSPHPNGLIKCTVVRKEKTKL;
(SEQ ID NO: 77)
MSTTLSSSTYGKDNVKFLKVKKDPQNPKKQEVMEATVTCLLEGGFDTSYTEADNSSIV
PTDTVKNTILVLAKTTEIWPIERFAAKLATHFVEKYSHVSGVSVKIVQDRWVKYAVDGKPHDH
SFIHEGGEKRITDLYYKRSGDYKLSSAIKDLTVLKSTGSMFYGYNKCDFTTLQPTTDRILSTDV
DATWVWDNKKIGSVYDIAKAADKGIFDNVYNQAREITLTTFAXENSPSVQATMFNMATQILE
KACSVYSVSYALPNKHYFLIDLKWKGLENDNELFYPSPHPNGLIKCTVVRKEKTKL;
(SEQ ID NO: 78)
MSTTLSSSTYGKDNVKFLKVKKDPQNPKKQEVMEATVTCLLEGGFDTSYTEADNSSIV
PTDTVKNTILVLAKTTEIWPIERFAAKLATHFVEKYSHVSGVSVKIVQDRWVKYAVDGKPHDH
SFIHEGGEKRITDLYYKRSGDYKLSSAIKDLTVLKSTGSMFYGYNKCDFTTLQPTTDRILSTDV
DATWVWDNKKIGSVYDIAKAADKGIFDNVYNQAREITLTTFALENSPSVQATMFNMATQILE
KACSVYSVSYALPNKHYFLIDLKWKGLENDNELFYPSPHPNGLIKCTVVRKEXTKL;
(SEQ ID NO: 79)
MSTTLSSSTYGKDNVKFLKVKKDPQNPKKQEVMEATVTCLLEGGFDTSYTEADNSSIV
PTDTVKNTILVLAKTTEIWPIERFAAKLATHFVEKYSHVSGVSVKIVQDRWVKYAVDGKPHDH
SFIHEGGEKRITDLYYKRSGDYKLSSAIKDLTVLKSTGSMFYGYNKCDFTTLQPTTDRILSTDV
DATWVWDNKKIGSVYDIAXAADKGIFDNVYNQAREITLTTFALENSPSVQATMFNMATQILE
KACSVYSVSYALPNKHYFLIDLKWKGLENDNELFYPSPHPNGLIKCTVVRKEKTKL;
(SEQ ID NO: 80)
MSTTLSSSTYGKDNVKFLKVKKDPQNPKKQEVMEATVTCLLEGGFDTSYTXADNSSIV
PTDTVKNTILVLAKTTEIWPIERFAAKLATHFVEKYSHVSGVSVKIVQDRWVKYAVDGKPHDH
SFIHEGGEKRITDLYYKRSGDYKLSSAIKDLTVLKSTGSMFYGYNKCDFTTLQPTTDRILSTDV
DATWVWDNKKIGSVYDIAKAADKGIFDNVYNQAREITLTTFALENSPSVQATMFNMATQILE
KACSVYSVSYALPNKHYFLIDLKWKGLENDNELFYPSPHPNGLIKCTVVRKEKTKL;
(SEQ ID NO: 81)
MSTTLSSSTYGKDNVKFLKVKKDPQNPKKQEVMEATVTCLLEGGFDTSYTEADNSSIV
PTDTVKNTILVLAKTTEIWPIERFAAKLATHFVEKYSHVSGVSVKIVQDRWVKYAVDGKPHDH
SFIHEGGEKRITDLYYKRSGDYKLSSAIKDLTVLKSTGSMFYGYNKCDFTTLQPTTDRILSTDV
DATWVWDNKKIGSVYDIAKAAXKGIFDNVYNQAREITLTTFALENSPSVQATMFNMATQILE
KACSVYSVSYALPNKHYFLIDLKWKGLENDNELFYPSPHPNGLIKCTVVRKEKTKL;
(SEQ ID NO: 82)
MSTTLSSSTYGKDNVKFLKVKKDPQNPKKQEVMEATVTCLLEGGFDTSYTEADNSSIV
PTDTVKNTILVLAKTTEIWPIERFAAKLATHFVEKYSHVSGVSVKIVQDRWVKYAVDGXPHDH
SFIHEGGEKRITDLYYKRSGDYKLSSAIKDLTVLKSTGSMFYGYNKCDFTTLQPTTDRILSTDV
DATWVWDNKKIGSVYDIAKAADKGIFDNVYNQAREITLTTFALENSPSVQATMFNMATQILE
KACSVYSVSYALPNKHYFLIDLKWKGLENDNELFYPSPHPNGLIKCTVVRKEKTKL;
(SEQ ID NO: 83)
MSTTLSSSTYGKDNVKFLKVKKDPQNPKKQEVMEATVTCLLEGGFDTSYTEADNSSIV
PTDTVKNTILVLAKTTEIWPIERFAAKLATHFVEKYSHVSGVSVKIVQDRWVKYAVDGKPHDH
SFIHEGGEKRITDLYYKRSGDYKLSSAIKDLTVLKSTGSMFYGYNKCDFTTLQPTTDRILSTDV
DATWVWDNKKIGSVYDIAKAADKGIFDNVYNQAREITLTTFALENSPSVQATMFNMATQILE
KAXSVYSVSYALPNKHYFLIDLKWKGLENDNELFYPSPHPNGLIKCTVVRKEKTKL;
(SEQ ID NO: 84)
MSTTLSSSTYGKDNVKFLKVKKDPQNPKKQEVMEATVTCLLEGGFDTSYTEADNSSIV
PTDTVKNTILVLAKTTEIWPIERFAAKLATHFVEKYSHVSGVSVKIVQDRWVKYAVDGKPHDH
SFIHEGGEKRITDLYYKRSGDYKLSSAIKDLTVLKSTGSMFYGYNKCDFTTLQXTTDRILSTDV
DATWVWDNKKIGSVYDIAKAADKGIFDNVYNQAREITLTTFALENSPSVQATMFNMATQILE
KACSVYSVSYALPNKHYFLIDLKWKGLENDNELFYPSPHPNGLIKCTVVRKEKTKL;
(SEQ ID NO: 85)
MSTTLSSSTYGKDNVKFLKVKKDPQNPKKQEVMEATVTCLLEGGFDTSYTEADNSSIV
PTDTVKNTILVLAKTTEIWPIERFAAKLATHFVEKYSHVSGVSVKIVQDRWVKYAVDGKPHDH
SFIHEGGEKRITDLYYKRSGDYKLSSAIKDLTVLKSTGSMFYGYNKCDFTTLQPTTDRILSTDV
DATWVWDNKKIGSVYDIAKAADKGIFDNVYNQAREITLTTFALENSPSVQATMFNMATQILE
KACSVYSVSYALPNKHYFLIDLXWKGLENDNELFYPSPHPNGLIKCTVVRKEKTKL;
(SEQ ID NO: 86)
MSTTLSSSTYGKDNVKFLKVKKDPQNPKKQEVMEATVTCLLEGGFDTSYTEADNSSIV
PTDTVKNTILVLAKTTEIWPIERFAAKLATHFVEKYSHVSGVSVKIVQDRWVKYAVDGKPHDH
SFIHEGGEKRITDLYYKRSGDYKLSSAIKDLTVLKSTGSMFYGYNKCDFTTLQPTTDRILSTDV
DATWVWDNKKIGSVYDIAKAADKGIFDNVYNQAREITLTTFALENSPSVQATMFNMATQILE
KACSVYSVSYALPNKHYFLIXLKWKGLENDNELFYPSPHPNGLIKCTVVRKEKTKL;
(SEQ ID NO: 87)
MSTTLSSSTYGKDNVKFLKVKKDPQNPKKQEVMEATVTCLLEGXFDTSYTEADNSSIV
PTDTVKNTILVLAKTTEIWPIERFAAKLATHFVEKYSHVSGVSVKIVQDRWVKYAVDGKPHDH
SFIHEGGEKRITDLYYKRSGDYKLSSAIKDLTVLKSTGSMFYGYNKCDFTTLQPTTDRILSTDV
DATWVWDNKKIGSVYDIAKAADKGIFDNVYNQAREITLTTFALENSPSVQATMFNMATQILE
KACSVYSVSYALPNKHYFLIDLKWKGLENDNELFYPSPHPNGLIKCTVVRKEKTKL;
(SEQ ID NO: 88)
MSTTLSSSTYGKDNVKFLKVKKDPQNPKKQEVMEATVTCLLEGGFDTSYTEADNSSIV
PTDTVKNTILVLAKTTEIWPIERFAAKLATHFVEKYSHVSGVSVKIVQDRWVKYAVDGKPHDH
SFIHEGGEKRITDLYYKRSGDYKLSSAIKDLTVLKSTGSMFYGYNKCDFTTLQPTTDRILSTDV
DATWVWDXKKIGSVYDIAKAADKGIFDNVYNQAREITLTTFALENSPSVQATMFNMATQILE
KACSVYSVSYALPNKHYFLIDLKWKGLENDNELFYPSPHPNGLIKCTVVRKEKTKL;
(SEQ ID NO: 89)
MSTTLSSSTYGKDNVKFLKVKKDPQNPKKQEVMEATVTCLLEGGFDTSYTEADNSSIV
PTDTVKNTILVLAKTTEIWPIERFAAKLATHFVEKYSHVSGVSVKIVQDRWVKYAVDGKPHDH
SFIHEGGEKRITDLYYKRSGDYKLSSAIKDLTVLKSTGSMFYXYNKCDFTTLQPTTDRILSTDV
DATWVWDNKKIGSVYDIAKAADKGIFDNVYNQAREITLTTFALENSPSVQATMFNMATQILE
KACSVYSVSYALPNKHYFLIDLKWKGLENDNELFYPSPHPNGLIKCTVVRKEKTKL;
(SEQ ID NO: 90)
MSTTLSSSTYGKDNVKFLKVKKDPQNPKKQEVMEATVTCLLEGGFDTSYTEADNSSIV
PTDTVKNTILVLAKXTEIWPIERFAAKLATHFVEKYSHVSGVSVKIVQDRWVKYAVDGKPHD
HSFIHEGGEKRITDLYYKRSGDYKLSSAIKDLTVLKSTGSMFYGYNKCDFTTLQPTTDRILSTD
VDATWVWDNKKIGSVYDIAKAADKGIFDNVYNQAREITLTTFALENSPSVQATMFNMATQIL
EKACSVYSVSYALPNKHYFLIDLKWKGLENDNELFYPSPHPNGLIKCTVVRKEKTKL;
(SEQ ID NO: 91)
MSTTLSSSTYGKDNVKFLKVKKDPQNPKXQEVMEATVTCLLEGGFDTSYTEADNSSIV
PTDTVKNTILVLAKTTEIWPIERFAAKLATHFVEKYSHVSGVSVKIVQDRWVKYAVDGKPHDH
SFIHEGGEKRITDLYYKRSGDYKLSSAIKDLTVLKSTGSMFYGYNKCDFTTLQPTTDRILSTDV
DATWVWDNKKIGSVYDIAKAADKGIFDNVYNQAREITLTTFALENSPSVQATMFNMATQILE
KACSVYSVSYALPNKHYFLIDLKWKGLENDNELFYPSPHPNGLIKCTVVRKEKTKL;
(SEQ ID NO: 92)
MSTTLSSSTYGKDNVKFLKVKKDPQNPKKQEVMEATVTCLLEGGFDTSYTEADNSSIV
PTDTVKNTILVLAKTTEIWPIERFAAKLATHFVEKYSHVSGVSVKIVQDRWVKYAVDGKPHDH
SFIHEGGEKRITDLYYKRSGDYKLSSAIKDLTVLKSTGSMFYGYNKCDFTTLQPTTDRILSTDV
DATWVWDNKKIGSVYDIAKAADKGIFDNVYNQAREITLTTFALEXSPSVQATMFNMATQILE
KACSVYSVSYALPNKHYFLIDLKWKGLENDNELFYPSPHPNGLIKCTVVRKEKTKL;
(SEQ ID NO: 93)
MSTTLSSSTYGKDNVKFLKVKKDPXNPKKQEVMEATVTCLLEGGFDTSYTEADNSSIV
PTDTVKNTILVLAKTTEIWPIERFAAKLATHFVEKYSHVSGVSVKIVQDRWVKYAVDGKPHDH
SFIHEGGEKRITDLYYKRSGDYKLSSAIKDLTVLKSTGSMFYGYNKCDFTTLQPTTDRILSTDV
DATWVWDNKKIGSVYDIAKAADKGIFDNVYNQAREITLTTFALENSPSVQATMFNMATQILE
KACSVYSVSYALPNKHYFLIDLKWKGLENDNELFYPSPHPNGLIKCTVVRKEKTKL;
(SEQ ID NO: 94)
MSTTLSSSTYGKDNVKFLKVKKDPQNPKKQEVMEATVTCLLEGGFDTSYTEADNSSIV
PTDTVKNTILVLAKTTEIWPIERFAAKLATHFVEKYSHVSGVSVKIVQDRWVKYAVDGKPHDH
SFIHEGGEKRITDLYYKRSGDYKLSSAIKDLTVLKSTGSMFYGYNKCDFTTLQPTTDRILSTDV
DATWVWDNKKIGSVYDIAKAADKGIFDNVYXQAREITLTTFALENSPSVQATMFNMATQILE
KACSVYSVSYALPNKHYFLIDLKWKGLENDNELFYPSPHPNGLIKCTVVRKEKTKL;
(SEQ ID NO: 95)
MSTTLSSSTYGKDNVKFLKVKKDPQNPKKQEVMEATVTCLLEGGFDTSYTEADNXSI
VPTDTVKNTILVLAKTTEIWPIERFAAKLATHFVEKYSHVSGVSVKIVQDRWVKYAVDGKPHD
HSFIHEGGEKRITDLYYKRSGDYKLSSAIKDLTVLKSTGSMFYGYNKCDFTTLQPTTDRILSTD
VDATWVWDNKKIGSVYDIAKAADKGIFDNVYNQAREITLTTFALENSPSVQATMFNMATQIL
EKACSVYSVSYALPNKHYFLIDLKWKGLENDNELFYPSPHPNGLIKCTVVRKEKTKL;
(SEQ ID NO: 96)
MSTTLSSSTYGKDNVKFLKVKKDPQNPXKQEVMEATVTCLLEGGFDTSYTEADNSSIV
PTDTVKNTILVLAKTTEIWPIERFAAKLATHFVEKYSHVSGVSVKIVQDRWVKYAVDGKPHDH
SFIHEGGEKRITDLYYKRSGDYKLSSAIKDLTVLKSTGSMFYGYNKCDFTTLQPTTDRILSTDV
DATWVWDNKKIGSVYDIAKAADKGIFDNVYNQAREITLTTFALENSPSVQATMFNMATQILE
KACSVYSVSYALPNKHYFLIDLKWKGLENDNELFYPSPHPNGLIKCTVVRKEKTKL;
(SEQ ID NO: 97)
MSTTLXSSTYGKDNVKFLKVKKDPQNPKKQEVMEATVTCLLEGGFDTSYTEADNSSI
VPTDTVKNTILVLAKTTEIWPIERFAAKLATHFVEKYSHVSGVSVKIVQDRWVKYAVDGKPHD
HSFIHEGGEKRITDLYYKRSGDYKLSSAIKDLTVLKSTGSMFYGYNKCDFTTLQPTTDRILSTD
VDATWVWDNKKIGSVYDIAKAADKGIFDNVYNQAREITLTTFALENSPSVQATMFNMATQIL
EKACSVYSVSYALPNKHYFLIDLKWKGLENDNELFYPSPHPNGLIKCTVVRKEKTKL;
(SEQ ID NO: 98)
MSTTLSSSTYGKDNVKFLKVKKDPQNXKKQEVMEATVTCLLEGGFDTSYTEADNSSI
VPTDTVKNTILVLAKTTEIWPIERFAAKLATHFVEKYSHVSGVSVKIVQDRWVKYAVDGKPHD
HSFIHEGGEKRITDLYYKRSGDYKLSSAIKDLTVLKSTGSMFYGYNKCDFTTLQPTTDRILSTD
VDATWVWDNKKIGSVYDIAKAADKGIFDNVYNQAREITLTTFALENSPSVQATMFNMATQIL
EKACSVYSVSYALPNKHYFLIDLKWKGLENDNELFYPSPHPNGLIKCTVVRKEKTKL;
(SEQ ID NO: 99)
MSTTLSSSTYGKDNVKFLKVKKDPQNPKKQEVMEATVTCLLEGGFDTSYTEADNSSIV
PTDTVKNTILVLAKTTEIWPIERFAAKLATHFVEKYSHVSGVSVKIVQDRWVKYAVDGKPHDH
SFIHEGGEKRITDLYYKRSGDYKLSSAIKDLTVLKSTGSMFYGYNKCDFTTLQPTTDRILSTDV
DATWVWDNKKIGSVYDIAKAADKGIFDNVYNQAREITLTTFALENSPSVQATMFNMATQILE
KACSVYSVSYALPNKHYFLIDLKWKGLENDNELFYPSPHPNGLIKCTVVRXEKTKL;
(SEQ ID NO: 100)
MSTTLSSSTYGKDNVKFLKVKKDPQNPKKQEVMEATVTCLLEGGFDTSYTEADNSSIV
PTDTVKNTILVLAKTTEIWPIERFAAKLATHFVEKYSHVSGVSVKIVQDRWVKYXVDGKPHDH
SFIHEGGEKRITDLYYKRSGDYKLSSAIKDLTVLKSTGSMFYGYNKCDFTTLQPTTDRILSTDV
DATWVWDNKKIGSVYDIAKAADKGIFDNVYNQAREITLTTFALENSPSVQATMFNMATQILE
KACSVYSVSYALPNKHYFLIDLKWKGLENDNELFYPSPHPNGLIKCTVVRKEKTKL;
(SEQ ID NO: 101)
MSTTLSSSTYGKDNVKFLKVKKDPQNPKKQEVMEATVTCLLEGGFDTSYTEADNSSIV
PTDTVKNTILVLAKTTEIWPIERFAAKLATHFVEKYSHVSGVSVKIVQDRWVKYAVDGKPHDH
SFIHEGGEKRITDLYYKRSGDYKLSSAIKDLTVLKSTGSMFYGYNKCDFTTLQPTTDRILSTDV
DATWVWDNKKIGSVYDIAKAADKGIFDXVYNQAREITLTTFALENSPSVQATMFNMATQILE
KACSVYSVSYALPNKHYFLIDLKWKGLENDNELFYPSPHPNGLIKCTVVRKEKTKL;
(SEQ ID NO: 102)
MSTTLSSSTYGKDNVKFLKVKKDPQNPKKQEVMEATVTCLLEGGFDTSYTEADNSSIV
PTDTVKNTILVLAKTTEIWPIERFAAKLATHFVEKYSHVSGVSVKIVQDRWVKYAVDGKPHDH
SFIHEGGEKRITDLYYKRSGDYKLSSAIKDLTVLKSTGSMFYGYNKCDFTTLQPTTDRILSTDV
DATWVWDNKKIGSVYDIAKAADKGIFDNVYNQARXITLTTFALENSPSVQATMFNMATQILE
KACSVYSVSYALPNKHYFLIDLKWKGLENDNELFYPSPHPNGLIKCTVVRKEKTKL;
(SEQ ID NO: 103)
MSTTLSSSTYGKDNVKFLKVKKDPQNPKKQEVMEATVTCLLEGGFDTSYTEADNSSIV
PTDTVKNTILVLAKTTEIWPIERFAAKLATHFVEKYSHVSGVSVKIVQDRWVKYAVDGKPHDH
SFIHEGGEKRITDLYYKRSXDYKLSSAIKDLTVLKSTGSMFYGYNKCDFTTLQPTTDRILSTDV
DATWVWDNKKIGSVYDIAKAADKGIFDNVYNQAREITLTTFALENSPSVQATMFNMATQILE
KACSVYSVSYALPNKHYFLIDLKWKGLENDNELFYPSPHPNGLIKCTVVRKEKTKL;
(SEQ ID NO: 104)
MSTTLSSSTYGKDNVKFLKVKKDPQNPKKQEVMEATVTCLLEGGFDTSYTEADNSSIV
PTDTVKNTILVLAKTTEIWPIERFAAKLATHFVEKYSHVSGVSVKIVQDRWVKYAVDGKPHDH
SFIHEGGEKRITDLYYKRSGDYKLSSAIKDLTVLKSTGSMFXGYNKCDFTTLQPTTDRILSTDV
DATWVWDNKKIGSVYDIAKAADKGIFDNVYNQAREITLTTFALENSPSVQATMFNMATQILE
KACSVYSVSYALPNKHYFLIDLKWKGLENDNELFYPSPHPNGLIKCTVVRKEKTKL;
(SEQ ID NO: 105)
MSTTLSSSTYGKDNVKFLKVKKDPQNPKKQEVMEATVTCLLEGGFDTSYTEADNSSIV
PTDTVKNTILVLAKTTEIWPIERFAAKLATHFVEKYSHVSGVSVKIVQDRWVKYAVDGKPHDH
SFIHEGGEKRITDLYYKRSGDYKLSSAIKDLTVLKSTGSMFYGYNKCDFTTLQPTTDRILSTDV
DATWVWDNKKIGSVYDIAKAADKGIFDNVYNQAREITLTTFALENSPSVQATMFNMATQILE
KACSVXSVSYALPNKHYFLIDLKWKGLENDNELFYPSPHPNGLIKCTVVRKEKTKL;
(SEQ ID NO: 106)
MSTTLSSSTYGKDNVKFLKVKKDPQNPKKQEVMEATVTCLLEGGFDTSYTEADNSSIV
PTDTVKNTILVLAKTTEIWPIERFAAKLATHFVEKYSHVSGVSVKIVQDRWVKYAVDGKPHDH
SFIHEGGEKRITDLYYKRSGDYKLSSAIKDLTVLKSTGSMFYGYNKCDFTTLQPTTXRILSTDV
DATWVWDNKKIGSVYDIAKAADKGIFDNVYNQAREITLTTFALENSPSVQATMFNMATQILE
KACSVYSVSYALPNKHYFLIDLKWKGLENDNELFYPSPHPNGLIKCTVVRKEKTKL;
(SEQ ID NO: 107)
MSTTLSSSTYGKDNVKFLKVKKDPQNPKKQEVMEATVTCLLEGGFDTSYTEADNSSIV
PTDTVKNTILVLAKTTEIWPIERFAAKLATHFVEXYSHVSGVSVKIVQDRWVKYAVDGKPHDH
SFIHEGGEKRITDLYYKRSGDYKLSSAIKDLTVLKSTGSMFYGYNKCDFTTLQPTTDRILSTDV
DATWVWDNKKIGSVYDIAKAADKGIFDNVYNQAREITLTTFALENSPSVQATMFNMATQILE
KACSVYSVSYALPNKHYFLIDLKWKGLENDNELFYPSPHPNGLIKCTVVRKEKTKL;
(SEQ ID NO: 108)
MSTTLSSSTYGKDNVKFLKVKKDPQNPKKQEVMEATVTCLLEGGFDTSYTEADNSSIV
PTDTVKNTILVLAKTTEIWPIERFAAKLATHFVEKYSHVSGVSVXIVQDRWVKYAVDGKPHDH
SFIHEGGEKRITDLYYKRSGDYKLSSAIKDLTVLKSTGSMFYGYNKCDFTTLQPTTDRILSTDV
DATWVWDNKKIGSVYDIAKAADKGIFDNVYNQAREITLTTFALENSPSVQATMFNMATQILE
KACSVYSVSYALPNKHYFLIDLKWKGLENDNELFYPSPHPNGLIKCTVVRKEKTKL;
(SEQ ID NO: 109);
MSTTLSSSTYGKDNVKFLKVKKDPQNPKKQEVMEATVTCLLEGGFDTSYTEADNSSIV
PTDTVKNTILVLAKTTEIWPIERFAAKLATHFVEKYSHVSGVSVKIVQDRWVKYAVDGKPHDH
SFIHEGGEKRITDLYYKRSGDYXLSSAIKDLTVLKSTGSMFYGYNKCDFTTLQPTTDRILSTDV
DATWVWDNKKIGSVYDIAKAADKGIFDNVYNQAREITLTTFALENSPSVQATMFNMATQILE
KACSVYSVSYALPNKHYFLIDLKWKGLENDNELFYPSPHPNGLIKCTVVRKEKTKL
(SEQ ID NO: 110)
MSTTLSSSTYGKDNVKFLKVKKDPQNPKKQEVMEATVTCLLEGGFDTSYTEADNSSIV
PTDTVKNTILVLAKTTEIWPIERFAAKLATHFVEKYSHVSGVSVKIVQDRWVKYAVDGKPHDH
SFIHEGGEKRITDLYYKXSGDYKLSSAIKDLTVLKSTGSMFYGYNKCDFTTLQPTTDRILSTDV
DATWVWDNKKIGSVYDIAKAADKGIFDNVYNQAREITLTTFALENSPSVQATMFNMATQILE
KACSVYSVSYALPNKHYFLIDLKWKGLENDNELFYPSPHPNGLIKCTVVRKEKTKL;
(SEQ ID NO: 111)
MSTTLSSSTYGKDNVKFLKVKKDPQNPKKQEVMEATVTCLLEGGFDTSYTEADNSSIV
PTDTVKNTILVLAKTTEIWPIERFAAKLATHFVEKYSHVSGVSVKIVQDRWVKYAVDGKPHDH
SFIHEGGEKRITDLYYXRSGDYKLSSAIKDLTVLKSTGSMFYGYNKCDFTTLQPTTDRILSTDV
DATWVWDNKKIGSVYDIAKAADKGIFDNVYNQAREITLTTFALENSPSVQATMFNMATQILE
KACSVYSVSYALPNKHYFLIDLKWKGLENDNELFYPSPHPNGLIKCTVVRKEKTKL;
(SEQ ID NO: 112)
MSTTLSXSTYGKDNVKFLKVKKDPQNPKKQEVMEATVTCLLEGGFDTSYTEADNSSI
VPTDTVKNTILVLAKTTEIWPIERFAAKLATHFVEKYSHVSGVSVKIVQDRWVKYAVDGKPHD
HSFIHEGGEKRITDLYYKRSGDYKLSSAIKDLTVLKSTGSMFYGYNKCDFTTLQPTTDRILSTD
VDATWVWDNKKIGSVYDIAKAADKGIFDNVYNQAREITLTTFALENSPSVQATMFNMATQIL
EKACSVYSVSYALPNKHYFLIDLKWKGLENDNELFYPSPHPNGLIKCTVVRKEKTKL;
(SEQ ID NO: 113)
MSTTLSSSTYGKDNVKFLKVKKDPQNPKKQEVMEATVTCLLEGGFDTSYTEADNSSIV
PTDTVKNTILVLAKTTEIWPIERFAAKLATHFVEKYSHVSGVSVKIVQDRWVKYAVDGKPHDH
SFIHEGGEKRITDLYYKRSGDYKLSSAIKXLTVLKSTGSMFYGYNKCDFTTLQPTTDRILSTDV
DATWVWDNKKIGSVYDIAKAADKGIFDNVYNQAREITLTTFALENSPSVQATMFNMATQILE
KACSVYSVSYALPNKHYFLIDLKWKGLENDNELFYPSPHPNGLIKCTVVRKEKTKL;
(SEQ ID NO: 114)
MSTTLSSSTYGKDNVKFLKVKKDPQNPKKQEVMEATVTCLLEGGFDTSYTEADNSSIV
PTDTVKNTILVLAKTTEIWPIERFAAKLATHFVEKYSHVSGVSVKIVQDRWVKYAVDGKPHDH
SFIHEGGEKRITDLYYKRSGDYKLSSAIKDLTVLKSTGSMFYGYNKCDFTTLQPTTDRILSTDV
DATWVWDNKKIGSVYDIAKAADKGIFDNVYNQAREITLTTFALENSPSVQATMFNMATQILE
KACSVYSVSYALPNKHYFLIDLKWKGLENDNELFYPSPHPNGLIKCTVVXKEKTKL;
(SEQ ID NO: 115)
MSTTLSSSTYGKDNVKFLKVKKDPQNPKKQEVMEATVTCLLEGGFDTSYTEADNSSIV
PTDTVKNTILVLAKTTEIWPIERFAAKLATHFVEKYSHVSGVSVKIVQDRWVKYAVDGKPHDH
SFIHEGGEKRITDLYYKRSGDYKLSSAIKDLTVLKSTGSMFYGYNKCDFTTLQPTTDRILSTDV
DATWVWDNKKIGSVYDIAKAADKGIFDNVYNQAREITLTTFALENSPSVQATMFNMATQILE
KACSVYSVSYALPNKHYFLIDLKWXGLENDNELFYPSPHPNGLIKCTVVRKEKTKL;
(SEQ ID NO: 116)
MSTTLSSSTYGKDNVKFLKVKKDPQNPKKQEVMEATVTCLLEGGFDTSYTEADNSSIV
PTDTVKNTILVLAKTTEIWPIERFAAKLATHFVEKYSHVSGVSVKIVQDRWVKYAVDGKPHDH
SFIHEGGEKRITDLYYKRSGDYKLSSAIKDLTVLKSTGSMFYGYNKCDFTTLQPTTDRILSTDV
DATWVWDNKKIGSVYDIAKAADKGIFDNVYNQAREITLTTFALENSPSVQATMFNMATQILE
KACSVYSVSYALPNKHYFLIDLKWKGLENDXELFYPSPHPNGLIKCTVVRKEKTKL;
and
(SEQ ID NO: 117)
MSTTLSSSTYGKDNVKFLKVKKDPQNPKKQEVMEATVTCLLEGGFDTSYTEADNSSIV
PTDTVKNTILVLAKTTEIWPIERFAAKLATHFVEKYSHVSGVSVKIVQDRWVKYAVDGKPHDH
SFIHEGGEKRITDLYYKRSGDYKLSSAIKDLTVLKSTGSMFYGYNKCDFTTLQPTTDRILSTDV
DATWVWDNKKIGSVYDIAKAADKGIFDNVYNQAREITLTTFALENSPSVQATMFNMATQILE
KACSVYSVSYALPNKHYXLIDLKWKGLENDNELFYPSPHPNGLIKCTVVRKEKTKL,

where X is selected from the nonnatural amino acids disclosed in the tables of FIGS. 23 to 27.

### Example 10, Subunit Sequence Example 3, Arth.Uox

The urate oxidase-albumin conjugate of anyone of Examples 2 to 7, wherein the urate oxidase variant subunit is represented by a sequence selected from the following:

(SEQ ID NO: 119)
MTATAETSTGTKVVLGQNQYGKAEVRLVKVTRNTARHEIQDLNVTSQLRGDFEAAHT
AGDNAHVVATDTQKNTVYAFARXGFATTEEFLLRLGKHFTEGFDWVTGGRWAAQQFFWDRI
NDHDHAFSRNKSEVRTAVLEISGSEQAIVAGIEGLTVLKSTGSEFHGFPRDKYTTLQETTDRILA
TDVSARWRYNTVEVDFDAVYASVRGLLLKAFAETHSLALQQTMYEMGRAVIETHPEIDEIKM
SLPNKHHFLVDLQPFGQDNPNEVFYAADRPYGLIEATIQREGSRADHPIWSNIAGFC;
(SEQ ID NO: 120)
MTATAETSTGTKVVLGQNQYGKAEVRLVKVTRNTARHEIQDLNVTSQLRGDFEAAHT
AGDNAHVVATDTQKNTVYAFARDGXATTEEFLLRLGKHFTEGFDWVTGGRWAAQQFFWDR
INDHDHAFSRNKSEVRTAVLEISGSEQAIVAGIEGLTVLKSTGSEFHGFPRDKYTTLQETTDRIL
ATDVSARWRYNTVEVDFDAVYASVRGLLLKAFAETHSLALQQTMYEMGRAVIETHPEIDEIK
MSLPNKHHFLVDLQPFGQDNPNEVFYAADRPYGLIEATIQREGSRADHPIWSNIAGFC;
(SEQ ID NO: 121)
MTATAETSTGTKVVLGQNQYGKAEVRLVKVTRNTARHEIQDLNVTSQLRGDFEAAHT
AGDNAHVVATDTQKNTVYAFARDGFATTEEFLLRLGKHFTEGXDWVTGGRWAAQQFFWDR
INDHDHAFSRNKSEVRTAVLEISGSEQAIVAGIEGLTVLKSTGSEFHGFPRDKYTTLQETTDRIL
ATDVSARWRYNTVEVDFDAVYASVRGLLLKAFAETHSLALQQTMYEMGRAVIETHPEIDEIK
MSLPNKHHFLVDLQPFGQDNPNEVFYAADRPYGLIEATIQREGSRADHPIWSNIAGFC;
(SEQ ID NO: 122)
MTATAETSTGTKVVLGQNQYGKAEVRLVKVTRNTARHEIQDLNVTSQLRGDFEAAHT
AGDNAHVVATDTQKNTVYAFARDGFATTEEFLLRLGKHFTEGFXWVTGGRWAAQQFFWDRI
NDHDHAFSRNKSEVRTAVLEISGSEQAIVAGIEGLTVLKSTGSEFHGFPRDKYTTLQETTDRILA
TDVSARWRYNTVEVDFDAVYASVRGLLLKAFAETHSLALQQTMYEMGRAVIETHPEIDEIKM
SLPNKHHFLVDLQPFGQDNPNEVFYAADRPYGLIEATIQREGSRADHPIWSNIAGFC;
(SEQ ID NO: 123)
MTATAETSTGTKVVLGQNQYGKAEVRLVKVTRNTARHEIQDLNVTSQLRGDFEAAHT
AGDNAHVVATDTQKNTVYAFARDGFATTEEFLLRLGKHFTEGFDWVTGGRWAAQQFXWDR
INDHDHAFSRNKSEVRTAVLEISGSEQAIVAGIEGLTVLKSTGSEFHGFPRDKYTTLQETTDRIL
ATDVSARWRYNTVEVDFDAVYASVRGLLLKAFAETHSLALQQTMYEMGRAVIETHPEIDEIK
MSLPNKHHFLVDLQPFGQDNPNEVFYAADRPYGLIEATIQREGSRADHPIWSNIAGFC;
(SEQ ID NO: 124)
MTATAETSTGTKVVLGQNQYGKAEVRLVKVTRNTARHEIQDLNVTSQLRGDFEAAHT
AGDNAHVVATDTQKNTVYAFARDGFATTEEFLLRLGKHFTEGFDWVTGGRWAAQQFFWDRI
XDHDHAFSRNKSEVRTAVLEISGSEQAIVAGIEGLTVLKSTGSEFHGFPRDKYTTLQETTDRILA
TDVSARWRYNTVEVDFDAVYASVRGLLLKAFAETHSLALQQTMYEMGRAVIETHPEIDEIKM
SLPNKHHFLVDLQPFGQDNPNEVFYAADRPYGLIEATIQREGSRADHPIWSNIAGFC;
(SEQ ID NO: 125)
MTATAETSTGTKVVLGQNQYGKAEVRLVKVTRNTARHEIQDLNVTSQLRGDFEAAHT
AGDNAHVVATDTQKNTVYAFARDGFATTEEFLLRLGKHFTEGFDWVTGGRWAAQQFFWDRI
NXHDHAFSRNKSEVRTAVLEISGSEQAIVAGIEGLTVLKSTGSEFHGFPRDKYTTLQETTDRILA
TDVSARWRYNTVEVDFDAVYASVRGLLLKAFAETHSLALQQTMYEMGRAVIETHPEIDEIKM
SLPNKHHFLVDLQPFGQDNPNEVFYAADRPYGLIEATIQREGSRADHPIWSNIAGFC;
(SEQ ID NO: 126)
MTATAETSTGTKVVLGQNQYGKAEVRLVKVTRNTARHEIQDLNVTSQLRGDFEAAHT
AGDNAHVVATDTQKNTVYAFARDGFATTEEFLLRLGKHFTEGFDWVTGGRWAAQQFFWDRI
NDHDHAFSRNKSEVRTAVLEISGXEQAIVAGIEGLTVLKSTGSEFHGFPRDKYTTLQETTDRILA
TDVSARWRYNTVEVDFDAVYASVRGLLLKAFAETHSLALQQTMYEMGRAVIETHPEIDEIKM
SLPNKHHFLVDLQPFGQDNPNEVFYAADRPYGLIEATIQREGSRADHPIWSNIAGFC;
(SEQ ID NO: 127)
MTATAETSTGTKVVLGQNQYGKAEVRLVKVTRNTARHEIQDLNVTSQLRGDFEAAHT
AGDNAHVVATDTQKNTVYAFARDGFATTEEFLLRLGKHFTEGFDWVTGGRWAAQQFFWDRI
NDHDHAFSRNKSEVRTAVLEISGSXQAIVAGIEGLTVLKSTGSEFHGFPRDKYTTLQETTDRILA
TDVSARWRYNTVEVDFDAVYASVRGLLLKAFAETHSLALQQTMYEMGRAVIETHPEIDEIKM
SLPNKHHFLVDLQPFGQDNPNEVFYAADRPYGLIEATIQREGSRADHPIWSNIAGFC;
(SEQ ID NO: 128)
MTATAETSTGTKVVLGQNQYGKAEVRLVKVTRNTARHEIQDLNVTSQLRGDFEAAHT
AGDNAHVVATDTQKNTVYAFARDGFATTEEFLLRLGKHFTEGFDWVTGGRWAAQQFFWDRI
NDHDHAFSRNKSEVRTAVLEISGSEQAIVAGIEGLTVLKSTGSEFHGFPRDKYTTLXETTDRILA
TDVSARWRYNTVEVDFDAVYASVRGLLLKAFAETHSLALQQTMYEMGRAVIETHPEIDEIKM
SLPNKHHFLVDLQPFGQDNPNEVFYAADRPYGLIEATIQREGSRADHPIWSNIAGFC;
(SEQ ID NO: 129)
MTATAETSTGTKVVLGQNQYGKAEVRLVKVTRNTARHEIQDLNVTSQLRGDFEAAHT
AGDNAHVVATDTQKNTVYAFARDGFATTEEFLLRLGKHFTEGFDWVTGGRWAAQQFFWDRI
NDHDHAFSRNKSEVRTAVLEISGSEQAIVAGIEGLTVLKSTGSEFHGFPRDKYTTLQETTDRILA
TDVSARWRYNTXEVDFDAVYASVRGLLLKAFAETHSLALQQTMYEMGRAVIETHPEIDEIKM
SLPNKHHFLVDLQPFGQDNPNEVFYAADRPYGLIEATIQREGSRADHPIWSNIAGFC;
(SEQ ID NO: 130)
MTATAETSTGTKVVLGQNQYGKAEVRLVKVTRNTARHEIQDLNVTSQLRGDFEAAHT
AGDNAHVVATDTQKNTVYAFARDGFATTEEFLLRLGKHFTEGFDWVTGGRWAAQQFFWDRI
NDHDHAFSRNKSEVRTAVLEISGSEQAIVAGIEGLTVLKSTGSEFHGFPRDKYTTLQETTDRILA
TDVSARWRYNTVXVDFDAVYASVRGLLLKAFAETHSLALQQTMYEMGRAVIETHPEIDEIKM
SLPNKHHFLVDLQPFGQDNPNEVFYAADRPYGLIEATIQREGSRADHPIWSNIAGFC;
(SEQ ID NO: 131)
MTATAETSTGTKVVLGQNQYGKAEVRLVKVTRNTARHEIQDLNVTSQLRGDFEAAHT
AGDNAHVVATDTQKNTVYAFARDGFATTEEFLLRLGKHFTEGFDWVTGGRWAAQQFFWDRI
NDHDHAFSRNKSEVRTAVLEISGSEQAIVAGIEGLTVLKSTGSEFHGFPRDKYTTLQETTDRILA
TDVSARWRYNTVEVDFDAVYASVRGLLLKAFAETXSLALQQTMYEMGRAVIETHPEIDEIKM
SLPNKHHFLVDLQPFGQDNPNEVFYAADRPYGLIEATIQREGSRADHPIWSNIAGFC;
and
(SEQ ID NO: 132)
MTATAETSTGTKVVLGQNQYGKAEVRLVKVTRNTARHEIQDLNVTSQLRGDFEAAHT
AGDNAHVVATDTQKNTVYAFARDGFATTEEFLLRLGKHFTEGFDWVTGGRWAAQQFFWDRI
NDHDHAFSRNKSEVRTAVLEISGSEQAIVAGIEGLTVLKSTGSEFHGFPRDKYTTLQETTDRILA
TDVSARWRYNTVEVDFDAVYASVRGLLLKAFAETHSLALQQTMYEMGRAVIETHXEIDEIKM
SLPNKHHFLVDLQPFGQDNPNEVFYAADRPYGLIEATIQREGSRADHPIWSNIAGFC,

where X is selected from the nonnatural amino acids disclosed in the tables of FIGS. 23 to 27.

### Example 11, Example of Urate Oxidase-Linker Junction

The urate oxidase-albumin conjugate of any one of Examples 1 to 10, wherein the urate oxidase-linker junction is any one selected from the following:

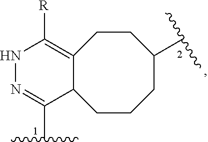

here, R is any one selected from H, CH₃,

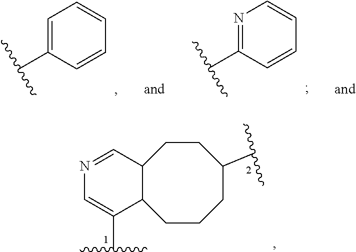 [SMILES: *c1ccccc1 ; *c1ccccn1 ; *C1=CN=CC2CCC(*)CCCC12]

here, the (1) part is linked to the urate oxidase variant, and the (2) part is linked to the anchor of the linker.

### Example 12, Limitation of Albumin Sequence

The urate oxidase-albumin conjugate of any one of Examples 1 to 11, wherein the albumin is represented by a sequence selected from the following:

(SEQ ID NO: 133)
DAHKSEVAHRFKDLGEENFKALVLIAFAQYLQQCPFEDHVKLVNEVTEFAKTCVADE
SAENCDKSLHTLFGDKLCTVATLRETYGEMADCCAKQEPERNECFLQHKDDNPNLPRLVRPE
VDVMCTAFHDNEETFLKKYLYEIARRHPYFYAPELLFFAKRYKAAFTECCQAADKAACLLPK
LDELRDEGKASSAKQRLKCASLQKFGERAFKAWAVARLSQRFPKAEFAEVSKLVTDLTKVHT
ECCHGDLLECADDRADLAKYICENQDSISSKLKECCEKPLLEKSHCIAEVENDEMPADLPSLAA
DFVESKDVCKNYAEAKDVFLGMFLYEYARRHPDYSVVLLLRLAKTYETTLEKCCAAADPHE
CYAKVFDEFKPLVEEPQNLIKQNCELFEQLGEYKFQNALLVRYTKKVPQVSTPTLVEVSRNLG
KVGSKCCKHPEAKRMPCAEDYLSVVLNQLCVLHEKTPVSDRVTKCCTESLVNRRPCFSALEV
DETYVPKEFNAETFTFHADICTLSEKERQIKKQTALVELVKHKPKATKEQLKAVMDDFAAFVE
KCCKADDKETCFAEEGKKLVAASQAALGL;
(SEQ ID NO: 134)
DAHKSEVAHRFKDLGEENFKALVLIAFAQYLQQCPFEDHVKLVNEVTEFAKTCVADE
SAENCDKSLHTLFGDKLCTVATLRETYGEMADCCAKQEPERNECFLQHKDDNPNLPRLVRPE
VDVMCTAFHDNEETFLKKYLYEIARRHPYFYAPELLFFAKRYKAAFTECCQAADKAACLLPK
LDELRDEGKASSAKQRLKCASLQKFGERAFKAWAVARLSQRFPKAEFAEVSKLVTDLTKVHT
ECCHGDLLECADDRADLAKYICENQDSISSKLKECCEKPLLEKSHCIAEVENDEMPADLPSLAA
DFVESKDVCKNYAEAKDVFLGMFLYEYARRHPDYSVVLLLRLAKTYETTLEKCCAAADPHE
CYAKVFDEFKPLVEEPQNLIKQNCELFEQLGEYKFQNALLVRYTKKVPQMSTPTLVEVSRNLG
KVGSKCCKHPEAKRMPCAEDYLSVVLNQLCVLHEKTPVSDRVTKCCTESLVNRRPCFSALEV
DETYVPKEFNAETFTFHADICTLSEKERQIKKQTALVELVKHKPKATKEQLKAVMDDFAAFVE
KCCKADDKETCFAEEGKKLVAASQAALGL;
(SEQ ID NO: 135)
DAHKSEVAHRFKDLGEENFKALVLIAFAQYLQQCPFEDHVKLVNEVTEFAKTCVADE
SAENCDKSLHTLFGDKLCTVATLRETYGEMADCCAKQEPERNECFLQHKDDNPNLPRLVRPE
VDVMCTAFHDNEETFLKKYLYEIARRHPYFYAPELLFFAKRYKAAFTECCQAADKAACLLPK
LDELRDEGKASSAKQRLKCASLQKFGERAFKAWAVARLSQRFPKAEFAEVSKLVTDLTKVHT
ECCHGDLLECADDRADLAKYICENQDSISSKLKECCEKPLLEKSHCIAEVENDEMPADLPSLAA
DFVESKDVCKNYAEAKDVFLGMFLYEYARRHPDYSVVLLLRLAKTYETTLEKCCAAADPHE
CYAKVFDEFKPLVEEPQNLIKQNCELFEQLGEYKFQNALLVRYTKKVPQVSAPTLVEVSRNLG
KVGSKCCKHPEAKRMPCAEDYLSVVLNQLCVLHEKTPVSDRVTKCCTESLVNRRPCFSALEV
DETYVPKEFNAETFTFHADICTLSEKERQIKKQTALVELVKHKPKATKEQLKAVMDDFAAFVE
KCCKADDKETCFAEEGKKLVAASQAALGL;
(SEQ ID NO: 136)
DAHKSEVAHRFKDLGEENFKALVLIAFAQYLQQCPFEDHVKLVNEVTEFAKTCVADE
SAENCDKSLHTLFGDKLCTVATLRETYGEMADCCAKQEPERNECFLQHKDDNPNLPRLVRPE
VDVMCTAFHDNEETFLKKYLYEIARRHPYFYAPELLFFAKRYKAAFTECCQAADKAACLLPK
LDELRDEGKASSAKQRLKCASLQKFGERAFKAWAVARLSQRFPKAEFAEVSKLVTDLTKVHT
ECCHGDLLECADDRADLAKYICENQDSISSKLKECCEKPLLEKSHCIAEVENDEMPADLPSLAA
DFVESKDVCKNYAEAKDVFLGMFLYEYARRHPDYSVVLLLRLAKTYETTLEKCCAAADPHE
CYAKVFDEFKPLVEEPQNLIKQNCELFEQLGEYKFQNALLVRYTKKVPQVSTPTLVEVSRNLG
KVGSKCCKHPEAKRMPCAEDYLSVVLNQLCVLHEKTPVSDRVTKCCTESLVNRRPCFSALEV
DETYVPKEFNARTFTFHADICTLSEKERQIKKQTALVELVKHKPKATKEQLKAVMDDFAAFVE
KCCKADDKETCFAEEGKKLVAASQAALGL;
(SEQ ID NO: 137)
DAHKSEVAHRFKDLGEENFKALVLIAFAQYLQQCPFEDHVKLVNEVTEFAKTCVADE
SAENCDKSLHTLFGDKLCTVATLRETYGEMADCCAKQEPERNECFLQHKDDNPNLPRLVRPE
VDVMCTAFHDNEETFLKKYLYEIARRHPYFYAPELLFFAKRYKAAFTECCQAADKAACLLPK
LDELRDEGKASSAKQRLKCASLQKFGERAFKAWAVARLSQRFPKAEFAEVSKLVTDLTKVHT
ECCHGDLLECADDRADLAKYICENQDSISSKLKECCEKPLLEKSHCIAEVENDEMPADLPSLAA
DFVESKDVCKNYAEAKDVFLGMFLYEYARRHPDYSVVLLLRLAKTYETTLEKCCAAADPHE
CYAKVFDEFKPLVEEPQNLIKQNCELFEQLGEYKFQNALLVRYTKKVPQVSTPTLVEVSRNLG
KVGSKCCKHPEAKRMPCAEDYLSVVLNQLCVLHEKTPVSDRVTKCCTESLVNRRPCFSALEV
DETYVPKEFNAGTFTFHADICTLSEKERQIKKQTALVELVKHKPKATKEQLKAVMDDFAAFVE
KCCKADDKETCFAEEGKKLVAASQAALGL;
(SEQ ID NO: 138)
DAHKSEVAHRFKDLGEENFKALVLIAFAQYLQQCPFEDHVKLVNEVTEFAKTCVADE
SAENCDKSLHTLFGDKLCTVATLRETYGEMADCCAKQEPERNECFLQHKDDNPNLPRLVRPE
VDVMCTAFHDNEETFLKKYLYEIARRHPYFYAPELLFFAKRYKAAFTECCQAADKAACLLPK
LDELRDEGKASSAKQRLKCASLQKFGERAFKAWAVARLSQRFPKAEFAEVSKLVTDLTKVHT
ECCHGDLLECADDRADLAKYICENQDSISSKLKECCEKPLLEKSHCIAEVENDEMPADLPSLAA
DFVESKDVCKNYAEAKDVFLGMFLYEYARRHPDYSVVLLLRLAKTYETTLEKCCAAADPHE
CYAKVFDEFKPLVEEPQNLIKQNCELFEQLGEYKFQNALLVRYTKKVPQVSTPTLVEVSRNLG
KVGSKCCKHPEAKRMPCAEDYLSVVLNQLCVLHEKTPVSDRVTKCCTESLVNRRPCFSALEV
DETYVPKEFNAETFTFHADICTLSEKERQIKKQTALVELVKHKPKATKEQLKAAMDDFAAFVE
KCCKADDKETCFAEEGKKLVAASQAALGL;
(SEQ ID NO: 139)
DAHKSEVAHRFKDLGEENFKALVLIAFAQYLQQCPFEDHVKLVNEVTEFAKTCVADE
SAENCDKSLHTLFGDKLCTVATLRETYGEMADCCAKQEPERNECFLQHKDDNPNLPRLVRPE
VDVMCTAFHDNEETFLKKYLYEIARRHPYFYAPELLFFAKRYKAAFTECCQAADKAACLLPK
LDELRDEGKASSAKQRLKCASLQKFGERAFKAWAVARLSQRFPKAEFAEVSKLVTDLTKVHT
ECCHGDLLECADDRADLAKYICENQDSISSKLKECCEKPLLEKSHCIAEVENDEMPADLPSLAA
DFVESKDVCKNYAEAKDVFLGMFLYEYARRHPDYSVVLLLRLAKTYETTLEKCCAAADPHE
CYAKVFDEFKPLVEEPQNLIKQNCELFEQLGEYKFQNALLVRYTKKVPQVSTPTLVEVSRNLG
KVGSKCCKHPEAKRMPCAEDYLSVVLNQLCVLHEKTPVSDRVTKCCTESLVNRRPCFSALEV
DETYVPKEFNAETFTFHADICTLSEKERQIKKQTALVELVKHKPKATKEQLKAVMDDFAAFVE
KCCKADDKETCFAEEGYKLVAASQAALGL;
(SEQ ID NO: 140)
DAHKSEVAHRFKDLGEENFKALVLIAFAQYLQQCPFEDHVKLVNEVTEFAKTCVADE
SAENCDKSLHTLFGDKLCTVATLRETYGEMADCCAKQEPERNECFLQHKDDNPNLPRLVRPE
VDVMCTAFHDNEETFLKKYLYEIARRHPYFYAPELLFFAKRYKAAFTECCQAADKAACLLPK
LDELRDEGKASSAKQRLKCASLQKFGERAFKAWAVARLSQRFPKAEFAEVSKLVTDLTKVHT
ECCHGDLLECADDRADLAKYICENQDSISSKLKECCEKPLLEKSHCIAEVENDEMPADLPSLAA
DFVESKDVCKNYAEAKDVFLGMFLYEYARRHPDYSVVLLLRLAKTYETTLEKCCAAADPHE
CYAKVFDEFKPLVEEPQNLIKQNCELFEQLGEYKFQNALLVRYTKKVPQVSTPTLIEVSRNLGK
VGSKCCKHPEAKRMPCAEDYLSVVLNQLCVLHEKTPVSDRVTKCCTESLVNRRPCFSALEVD
ETYVPKEFNAETFTFHADICTLSEKERQIKKQTALVELVKHKPKATKEQLKAVMDDFAAFVEK
CCKADDKETCFAEEGKKLVAASQAALGL;
(SEQ ID NO: 141)
DAHKSEVAHRFKDLGEENFKALVLIAFAQYLQQCPFEDHVKLVNEVTEFAKTCVADE
SAENCDKSLHTLFGDKLCTVATLRETYGEMADCCAKQEPERNECFLQHKDDNPNLPRLVRPE
VDVMCTAFHDNEETFLKKYLYEIARRHPYFYAPELLFFAKRYKAAFTECCQAADKAACLLPK
LDELRDEGKASSAKQRLKCASLQKFGERAFKAWAVARLSQRFPKAEFAEVSKLVTDLTKVHT
ECCHGDLLECADDRADLAKYICENQDSISSKLKECCEKPLLEKSHCIAEVENDEMPADLPSLAA
DFVESKDVCKNYAEAKDVFLGMFLYEYARRHPDYSVVLLLRLAKTYETTLEKCCAAADPHE
CYAKVFDEFKPLVEEPQNLIKQNCELFEQLGEYKFQNALLVRYTKKVPQVSTPTLVEVSRDLG
KVGSKCCKHPEAKRMPCAEDYLSVVLNQLCVLHEKTPVSDRVTKCCTESLVNRRPCFSALEV
DETYVPKEFNAETFTFHADICTLSEKERQIKKQTALVELVKHKPKATKEQLKAVMDDFAAFVE
KCCKADDKETCFAEEGKKLVAASQAALGL;
(SEQ ID NO: 142)
DAHKSEVAHRFKDLGEENFKALVLIAFAQYLQQCPFEDHVKLVNEVTEFAKTCVADE
SAENCDKSLHTLFGDKLCTVATLRETYGEMADCCAKQEPERNECFLQHKDDNPNLPRLVRPE
VDVMCTAFHDNEETFLKKYLYEIARRHPYFYAPELLFFAKRYKAAFTECCQAADKAACLLPK
LDELRDEGKASSAKQRLKCASLQKFGERAFKAWAVARLSQRFPKAEFAEVSKLVTDLTKVHT
ECCHGDLLECADDRADLAKYICENQDSISSKLKECCEKPLLEKSHCIAEVENDEMPADLPSLAA
DFVESKDVCKNYAEAKDVFLGMFLYEYARRHPDYSVVLLLRLAKTYETTLEKCCAAADPHE
CYAKVFDEFKPLVEEPQNLIKQNCELFEQLGEYKFQNALLVRYTKKVPQVSTPTLVEVSRNLG
KVGSKCCKHPEAKRMPCVEDYLSVVLNQLCVLHEKTPVSDRVTKCCTESLVNRRPCFSALEV
DETYVPKEFNAETFTFHADICTLSEKERQIKKQTALVELVKHKPKATKEQLKAVMDDFAAFVE
KCCKADDKETCFAEEGKKLVAASQAALGL;
(SEQ ID NO: 143)
DAHKSEVAHRFKDLGEENFKALVLIAFAQYLQQCPFEDHVKLVNEVTEFAKTCVADE
SAENCDKSLHTLFGDKLCTVATLRETYGEMADCCAKQEPERNECFLQHKDDNPNLPRLVRPE
VDVMCTAFHDNEETFLKKYLYEIARRHPYFYAPELLFFAKRYKAAFTECCQAADKAACLLPK
LDELRDEGKASSAKQRLKCASLQKFGERAFKAWAVARLSQRFPKAEFAEVSKLVTDLTKVHT
ECCHGDLLECADDRADLAKYICENQDSISSKLKECCEKPLLEKSHCIAEVENDEMPADLPSLAA
DFVESKDVCKNYAEAKDVFLGMFLYEYARRHPDYSVVLLLRLAKTYETTLEKCCAAADPHE
CYAKVFDEFKPLVEEPQNLIKQNCELFEQLGEYKFQNALLVRYTKKVPQVSTPTLVEVSRNLG
KVGSKCCKHPEAKRMPCAEDYLSVVLNQLCVLHEKMPVSDRVTKCCTESLVNRRPCFSALEV
DETYVPKEFNAETFTFHADICTLSEKERQIKKQTALVELVKHKPKATKEQLKAVMDDFAAFVE
KCCKADDKETCFAEEGKKLVAASQAALGL;
and
(SEQ ID NO: 144)
DAHKSEVAHRFKDLGEENFKALVLIAFAQYLQQCPFEDHVKLVNEVTEFAKTCVADE
SAENCDKSLHTLFGDKLCTVATLRETYGEMADCCAKQEPERNECFLQHKDDNPNLPRLVRPE
VDVMCTAFHDNEETFLKKYLYEIARRHPYFYAPELLFFAKRYKAAFTECCQAADKAACLLPK
LDELRDEGKASSAKQRLKCASLQKFGERAFKAWAVARLSQRFPKAEFAEVSKLVTDLTKVHT
ECCHGDLLECADDRADLAKYICENQDSISSKLKECCEKPLLEKSHCIAEVENDEMPADLPSLAA
DFVESKDVCKNYAEAKDVFLGMFLYEYARRHPDYSVVLLLRLAKTYETTLEKCCAAADPHE
CYAKVFDEFKPLVEEPQNLIKQNCELFEQLGEYKFQNALLVRYTKKVPQVSTPTLVEVSRNLG
KVGSKCCKHPEAKRMPCAEDYLSVVLNQLCVLHEKTPVSDRVTKCCTESLVNRRPCFSALEV
DETYVPKEFNAETFTFHADICTLSEKERQIKKQTALVELVKHKPKATKEQLKAVMDDFTAFVE
KCCKADDKETCFAEEGKKLVAASQAALGL.

### Example 13, Limitation of Albumin-Linker Junction

The urate oxidase-albumin conjugate of any one of Examples 1 to 12, wherein the albumin-linker junction formed by reaction of the thiol moiety of the albumin and the thiol moiety connected to the anchor.

### Example 14, Limitation of Position of Albumin Bonding

The urate oxidase-albumin conjugate of Example 13, wherein the albumin sequence is selected from SEQ ID NO: 133 to 144, and the albumin-linker junction is formed by reaction the thiol moiety of the residue of cysteine at position 34 of the albumin sequence and the thiol reactive group connected to the anchor.

### Example 15, Limitation of Albumin-Linker Junction

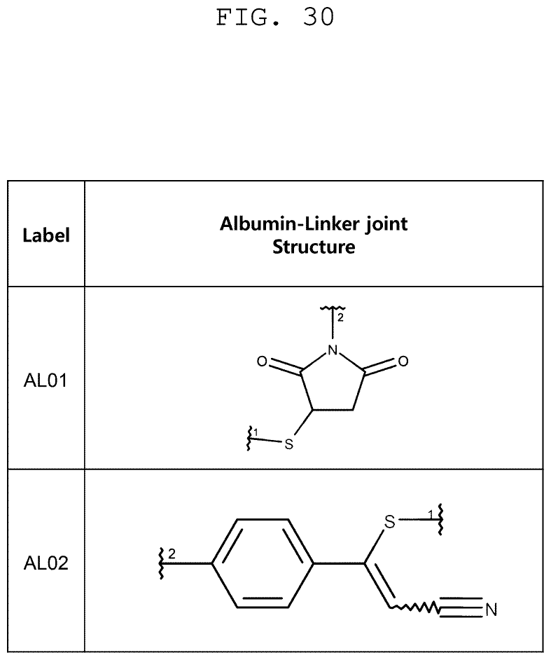

The urate oxidase-albumin conjugate of any one of Examples 13 to 14, wherein the urate oxidase-linker junction is any one selected from the examples in a table of FIG. 30.

### Example 16, Limitation of Anchor

The urate oxidase-albumin conjugate of any one of Examples 1 to 15, wherein the anchor is selected from the examples in a table of FIG. 40.

### Urate Oxidase-Albumin Conjugate 2

### Example 17, Urate Oxidase-Albumin Conjugate, Perspective from Subunit

A urate oxidase-albumin conjugate including the following:
3 or 4 albumin-subunit conjugates, in which each of the albumin-subunit conjugates is represented by Formula 2 below:

p′-J₁-A-J₂-HSA, [Formula 2]
in which p′ is a urate oxidase variant subunit, J₁ is a urate oxidase-linker junction, A is an anchor, J₂ is an albumin-linker junction, and HSA is human serum albumin, and
in which in the urate oxidase variant subunit, at least one amino acid in the sequence of a wild-type urate oxidase subunit is substituted with a nonnatural amino acid including a tetrazin functional group or a triazine functional group,
in which in the urate oxidase-linker junction, the junction is formed by undergoing an inverse electron demand Diels-Alder (IEDDA) reaction between the tetrazine or triazine functional group of the nonnatural amino acid and the trans-cyclooctene functional group connected to the anchor; and
optionally one urate oxidase variant subunit,
in which, when the urate oxidase-albumin conjugate includes three albumin-subunit complexes, the urate oxidase-albumin conjugate includes one urate oxidase variant subunit, and the urate oxidase variant subunits included in the respective albumin-subunit complexes and one urate oxidase variant subunit oligomerize to form a tetramer,
in which, when the urate oxidase-albumin conjugate includes four albumin-subunit conjugate the urate oxidase-albumin conjugate includes no urate oxidase variant subunits, and the urate oxidase variant subunits included in the respective albumin-subunit complexes oligomerize to form a tetramer.

### Example 18, Conjugation of Four Albumins

The urate oxidase-albumin conjugate of Example 17, including the following:
a first albumin-subunit conjugate,
in which the first albumin-subunit conjugate includes a first urate oxidase variant subunit, a first urate oxidase-linker junction, a first anchor, a first albumin-linker junction, and a first albumin;
a second albumin-subunit conjugate,
in which the second albumin-subunit conjugate includes a second urate oxidase variant subunit, a second urate oxidase-linker junction, a second anchor, a second albumin-linker junction, and a second albumin;
a third albumin-subunit conjugate,
in which the third albumin-subunit conjugate includes a third urate oxidase variant subunit, a third urate oxidase-linker junction, a third anchor, a third albumin-linker junction, and a third albumin;
a fourth albumin-subunit conjugate,
in which the fourth albumin-subunit conjugate includes a fourth urate oxidase variant subunit, a fourth urate oxidase-linker junction, a fourth anchor, a fourth albumin-linker junction, and a fourth albumin; and
in which the first urate oxidase variant subunit, the second urate oxidase variant subunit, the third urate oxidase variant subunit, and the fourth urate oxidase variant subunit oligomerize to form a tetramer.

### Example 19, Conjugation of Four Albumins, Constitutional Elements, Markush Claim

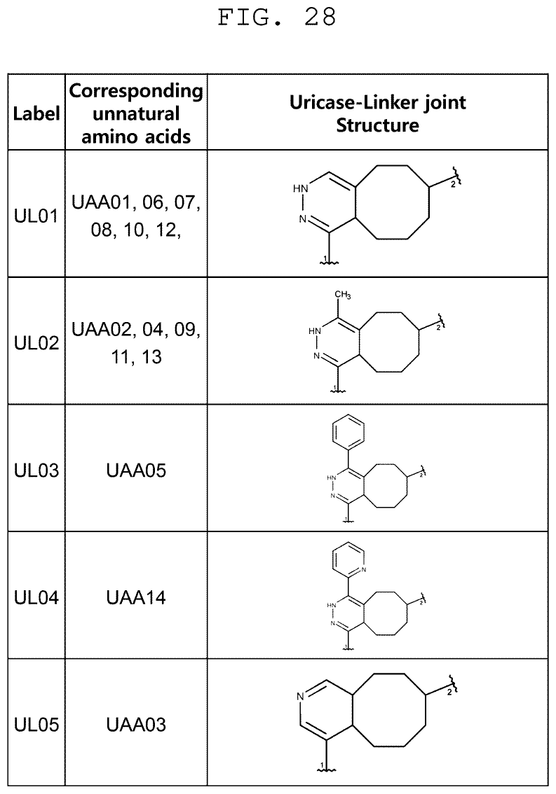

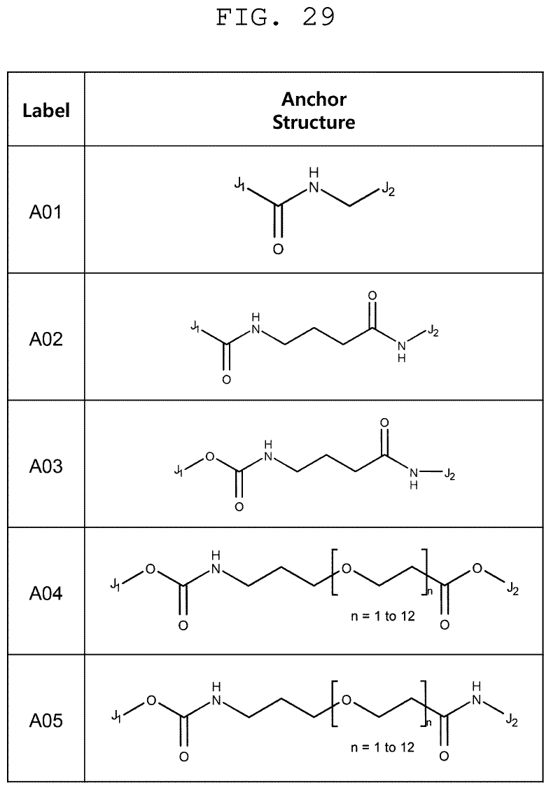

The urate oxidase-albumin conjugate of Example 18, having the following characteristics:
in which the first urate oxidase variant subunit is represented by a sequence selected from SEQ ID NOs:1 to 132 or a sequence that is at least 80% identical to the selected sequence, in which X included in the selected sequence is a nonnatural amino acid selected from tables of FIGS. 23 to 27,
the first urate oxidase-linker junction is one disclosed in the table of FIG. 28 according to a nonnatural amino acid selected from the tables of FIGS. 23 to 27,
in which the second urate oxidase variant subunit is represented by a sequence selected from SEQ ID NOs:1 to 132 or a sequence that is at least 80% identical to the selected sequence, in which X included in the selected sequence is a nonnatural amino acid selected from the tables of FIGS. 23 to 27,
the second urate oxidase-linker junction is one disclosed in the table of FIG. 28 according to a nonnatural amino acid selected from the tables of FIGS. 23 to 27,
in which the third urate oxidase variant subunit is represented by a sequence selected from SEQ ID NOs:1 to 132 or a sequence that is at least 80% identical to the selected sequence, in which X included in the selected sequence is a nonnatural amino acid selected from tables of FIGS. 23 to 27,
the third urate oxidase-linker junction is one disclosed in the table of FIG. 28 according to a nonnatural amino acid selected from the tables of FIGS. 23 to 27,
in which the fourth urate oxidase variant subunit is represented by a sequence selected from SEQ ID NOs:1 to 132 or a sequence that is at least 80% identical to the selected sequence, in which X included in the selected sequence is a nonnatural amino acid selected from the tables of FIGS. 23 to 27,
the fourth urate oxidase-linker junction is one disclosed in the table of FIG. 28 according to a nonnatural amino acid selected from the tables of FIGS. 23 to 27,
the first anchor, the second anchor, the third anchor, and the fourth anchor are each independently selected from the table of FIG. 29,
the first albumin-linker junction, the second albumin-linker junction, the third albumin-linker junction, and the fourth albumin linker junction are each independently selected from the table of FIG. 30,
the first albumin, the second albumin, the third albumin, and the fourth albumin are each independently represented by a sequence selected from SEQ ID NOs: 133 to SEQ ID NO: 145, or a sequence that is 80% or more identical to the selected sequence,
the first albumin-linker junction is formed by reaction of a thiol reactive moiety connected to the first anchor and a thiol moiety of cysteine at position 34 in the first albumin sequence,
the second albumin-linker junction is formed by reaction of a thiol reactive moiety connected to the second anchor and a thiol moiety of cysteine at position 34 in the second albumin sequence,
the third albumin-linker junction is formed by reaction of a thiol reactive moiety connected to the third anchor and a thiol moiety of cysteine at position 34 in the third albumin sequence,
the fourth albumin-linker junction is formed by reaction of a thiol reactive moiety connected to the fourth anchor and a thiol moiety of cysteine at position 34 in the fourth albumin.

### Example 20, Conjugation of Four Albumins, Limitation of Derivation of Urate Oxidase

The urate oxidase-albumin conjugate of Example 19, in which the first urate oxidase variant subunit, the second urate oxidase variant subunit, the third urate oxidase variant subunit, and the fourth urate oxidase variant subunit are all derived from any one microorganism selected from Aspergillus Flavus, Candida utilis, and Arthrobacter globiformis.

### Example 21, Conjugation of Three Albumins

The urate oxidase-albumin conjugate of Example 17, including the following:
a first albumin-subunit conjugate,
in which the first albumin-subunit conjugate includes a first urate oxidase variant subunit, a first urate oxidase-linker junction, a first anchor, a first albumin-linker junction, and a first albumin;
a second albumin-subunit conjugate,
in which the second albumin-subunit conjugate includes a second urate oxidase variant subunit, a second urate oxidase-linker junction, a second anchor, a second albumin-linker junction, and a second albumin;
a third albumin-subunit conjugate,
in which the third albumin-subunit conjugate includes a third urate oxidase variant subunit, a third urate oxidase-linker junction, a third anchor, a third albumin-linker junction, and a third albumin;
a fourth urate oxidase variant subunit,
in which the first urate oxidase mutant subunit, the second urate oxidase mutant subunit, the third urate oxidase mutant subunit, and the fourth urate oxidase mutant subunit oligomerize to form a tetramer.

### Example 22, Conjugation of Three Albumins, Constitutional Elements, Markush Claim

The urate oxidase-albumin conjugate of Example 21, having the following characteristics:
in which the first urate oxidase variant subunit is represented by a sequence selected from SEQ ID NOs:1 to 132 or a sequence that is at least 80% identical to the selected sequence, in which X included in the selected sequence is a nonnatural amino acid selected from tables of FIGS. 23 to 27,
the first urate oxidase-linker junction is one disclosed in the table of FIG. 28 according to a nonnatural amino acid selected from the tables of FIGS. 23 to 27,
in which the second urate oxidase variant subunit is represented by a sequence selected from SEQ ID NOs:1 to 132 or a sequence that is at least 80% identical to the selected sequence, in which X included in the selected sequence is a nonnatural amino acid selected from the tables of FIGS. 23 to 27,
the second urate oxidase-linker junction is one disclosed in the table of FIG. 28 according to a nonnatural amino acid selected from the tables of FIGS. 23 to 27,
in which the first urate oxidase variant subunit is represented by a sequence selected from SEQ ID NOs: 3 to 132 or a sequence that is at least 80% identical to the selected sequence, in which X included in the selected sequence is a nonnatural amino acid selected from tables of FIGS. 23 to 27,
the third urate oxidase-linker junction is one disclosed in the table of FIG. 28 according to a nonnatural amino acid selected from the tables of FIGS. 23 to 27,
in which the first urate oxidase variant subunit is represented by a sequence selected from SEQ ID NOs:1 to 132 or a sequence that is at least 80% identical to the selected sequence, in which X included in the selected sequence is a nonnatural amino acid selected from tables of FIGS. 23 to 27,
the first anchor, the second anchor, and the third anchor are each independently selected from the table of FIG. 29,
the first albumin-linker junction, the second albumin-linker junction, and the third albumin-linker junction are each independently selected from the table of FIG. 30,
the first albumin, the second albumin, the third albumin, and the fourth albumin are each independently represented by a sequence selected from SEQ ID NOs: 133 to SEQ ID NO: 145, or a sequence that is 80% or more identical to the selected sequence,
the first albumin-linker junction is formed by reaction of a thiol reactive moiety connected to the first anchor and a thiol moiety of cysteine at position 34 in the first albumin sequence,
the second albumin-linker junction is formed by reaction of a thiol reactive moiety connected to the second anchor and a thiol moiety of cysteine at position 34 in the second albumin sequence,
the third albumin-linker junction is formed by reaction of a thiol reactive moiety connected to the third anchor and a thiol moiety of cysteine at position 34 in the third albumin.

### Example 23, Conjugation of Four Albumins, Limitation of Derivation of Urate Oxidase

The urate oxidase-albumin conjugate of Example 22, in which the first urate oxidase variant subunit, the second urate oxidase variant subunit, the third urate oxidase variant subunit, and the fourth urate oxidase variant subunit are all derived from any one microorganism selected from Aspergillus Flavus, Candida utilis, and Arthrobacter Globiformis.

### Pharmaceutical Composition Including Urate Oxidase-Albumin Conjugate

### Example 24. Claim of Pharmaceutical Composition

A pharmaceutical composition for preventing or treating uric acid-related diseases, the pharmaceutical composition including:
a therapeutically effective amount of the urate oxidase-albumin conjugate of any one of Examples 1 to 23; and
a pharmaceutically acceptable carrier.

### Example 25, Limitation of Indications

The pharmaceutical composition of Example 24, in which the uric acid-related disease is any one of hyperuricemia, acute gouty arthritis, intermittent gout, chronic nodular gout, chronic kidney disease, and Tumor Lysis Syndrome (TLS).

### Example 26, Example of Carrier

The pharmaceutical composition according to any one of Examples 24 to 25, in which the pharmaceutically acceptable carrier includes one or more of the following:

binders such as lactose, saccharose, sorbitol, mannitol, starch, amylopectin, cellulose or gelatin; excipients such as dicalcium phosphate and the like; disintegrants such as corn starch or sweet potato starch; lubricants such as magnesium stearate, calcium stearate, sodium stearyl fumarate or polyethylene glycol wax; sweetener; air freshener; syrup; liquid carriers such as fatty oils; sterile aqueous solution; propylene glycol; polyethylene glycol; injectable esters such as ethyl oleate; suspending agent; emulsion; freeze-dried preparations; external preparations; stabilizer; buffer; animal oil; vegetable oil; wax; paraffin; starch; tragacanth; cellulose derivatives; polyethylene glycol; silicon; bentonite; silica; talc; and zinc oxide.

### A Treatment Method Using Urate Oxidase-Albumin Conjugate

### Example 27, Treatment Method Using Pharmaceutical Composition

A method for preventing or treating uric acid-related disease, the method comprising:
Administering the pharmaceutical composition of any one of Examples 24-26 into the body of a patient

### Example 28, Limitation of Indications

The pharmaceutical composition of claim 27, in which the uric acid-related disease is any one of hyperuricemia, acute gouty arthritis, intermittent gout, chronic nodular gout, chronic kidney disease, and Tumor Lysis Syndrome (TLS).

### Example 29, Limitation of Administration Method

The method of any one of Examples 27 to 28, in which the administration method is selected from oral administration, parenteral administration, intravenous administration, intravenous infusion, intraperitoneal administration, intramuscular administration, transdermal administration, and subcutaneous administration.

### Example 30, Limitation of Dosage

The method according to any one of Examples 27 to 29, in which the pharmaceutical composition is administered into the body of the patient at a dose of 0.01 mg/kg to 1000 mg/kg.

### Example 31, Limitation of Administration Interval 1

The method of any one of Examples 27 to 30, in which the pharmaceutical composition is administered once a day.

### Example 32, Limitation of Administration Interval 2

The method according to any one of Examples 27 to 30, in which the pharmaceutical composition is administered twice a day.

### Use of Urate Oxidase-Albumin Conjugate

### Example 33, Use as Preparation of Therapeutic Agent

A use of the urate oxidase-albumin conjugate of any one of Examples 1 to 23 for preparation of a therapeutic agent for Uric Acid-related Diseases

### Example 34, Limitation of Indications

The use of Example 33, in which the uric acid-related disease is any one of hyperuricemia, acute gouty arthritis, intermittent gout, chronic nodular gout, chronic kidney disease, and Tumor Lysis Syndrome (TLS).

### Urate Oxidase-Albumin Conjugate Preparation Method

### Example 35, Albumin-Linker Conjugation First

A method for preparing a urate oxidase-albumin conjugate, the method comprising:
reacting an albumin and a linker,
wherein the linker comprises a dienophile functional group, an anchor, and a thiol reactive moiety,
wherein the thiol reactive moiety of the linker is bound with thiol moiety of albumin through reaction to form an albumin-linker conjugate; and
reacting the albumin-linker conjugate and the urate oxidase variant,
in which, the urate oxidase variant is one in which three or more amino acids in the sequence of a wild-type urate oxidase are substituted with non-natural amino acids containing a diene functional group,
a diene functional group of the urate oxidase variant and a dienophile functional group of the linker of the albumin-linker conjugate bind to each other through an Inverse Electron Demand Diels-Alder (IEDDA) reaction to form a urate oxidase-albumin conjugate, and
the urate oxidase-albumin conjugate is characterized in that three or more albumins are conjugated to the urate oxidase variant through the linkers.

### Example 36, Urate Oxidase-Albumin Conjugation First

A method for preparing a urate oxidase-albumin conjugate, the method comprising:
reacting a urate oxidase variant and a linker,
in which, the urate oxidase variant is one in which three or more amino acids in the sequence of a wild-type urate oxidase are substituted with non-natural amino acids containing a diene functional group,
the linker includes a dienophile functional group, an anchor, and a thiol reactive moiety,
the diene functional group of the urate oxidase variant and the dienophile functional group of the linker bind to each other through an Inverse Electron Demand Diels-Alder (IEDDA) reaction to produce a urate oxidase-linker conjugated, and
the urate oxidase-linker conjugate is characterized in that three or more linkers are conjugated to the urate oxidase variants; and
reacting the urate oxidase-linker conjugate with albumin,
in which the thiol-reactive group of the linker of the urate oxidase-linker conjugate and the thiol group of the albumin are combined through a reaction to generate a urate oxidase-albumin conjugate, and
the urate oxidase-albumin conjugate is characterized in that three or more albumins are conjugated to the urate oxidase-linker conjugates.

### Example 37, Urate Oxidase, Linker, and Albumin Simultaneously

A method for preparing a urate oxidase-albumin conjugate, the method comprising:
reacting a urate oxidase variant, a linker, and an albumin,
in which, the urate oxidase variant is one in which three or more amino acids in the sequence of a wild-type urate oxidase are substituted with non-natural amino acids containing a diene functional group,
the linker includes a dienophile functional group, an anchor, and a thiol reactive moiety,
in which a thiol moiety of the albumin and a thiol moiety of the linker react to bind to each other,
the dien functional group contained in the nonnatural amino acid of the urate oxidase variant and the IEDDA reactive group of the linker are conjugated through a reaction, and
as a result of the reaction, the urate oxidase-albumin conjugate is produced.

### Example 38, Limitation of Urate Oxidase Variant

The urate oxidase-albumin conjugate preparation method of any one of Examples 36 to 37, in which the urate oxidase variant is a tetramer obtained by oligomerization of four urate oxidase variant subunits, and the urate oxidase variant subunit is a subunit obtained by substituting one or more amino acids in the sequence of a wild urate oxidase subunit with one or more nonnatural amino acids.

### Example 39, Limitation of Dien Functional Group of Nonnatural Amino Acid

The urate oxidase-albumin conjugate preparation method of anyone of Examples 35 to 37, in which the dien function group of the nonnatural amino acid is a tetrazine function group or a triazine functional group.

### Example 40, Limitation of Dienophile Functional Group

The urate oxidase-albumin conjugate preparation method of any one of Examples 35 to 37, in which the dienophile function group of the linker is selected from trans-cyclooctene and derivatives thereof.

### Example 41, Limitation of Thiol Reactive Group

The urate oxidase-albumin conjugate preparation method of any one of Examples 35 to 37, in which the thiol reactive group is selected from maleimide or derivatives thereof; and 3-arylpropiolonitriles or derivatives thereof.

### Example 42, Limitation of Reaction Condition of Urate Oxidase-Linker

The urate oxidase-albumin conjugate preparation method of any one of Examples 35 to 37, in which the reacting of the urate oxidase variant and the liner is performed in a neutral pH environment.

### Example 43: Markush Claim of Pharmaceutical Composition

The method of any one of Examples 35 to 37,
in which the urate oxidase variant is a tetramer formed by oligomerization of four urate oxidase variant subunits represented by a sequence, wherein the sequence of each subunit is independently selected from SEQ ID NOs: 1 to 132 or a sequence 80% or more identical to the selected sequence, in which X in the selected sequence is a nonnatural amino acid selected from the tables of FIGS. 23 to 27,
the linker is selected from the table of FIG. 31,
the albumin is represented by a sequence selected from SEQ ID NOs: 133 to 144 or a sequence 80% or more identical to the selected sequence, and
the thiol moiety of the albumin is a thiol moiety of cysteine at position 34 in a sequence selected from SEQ ID NOs:133 to 144 or in a sequence that is 80% or more identical to the selected sequence.

### Urate Oxidase Variant

### Example 44, Urate Oxidase Variant

A urate oxidase variant including three or more unnatural amino acids in the sequence thereof, in which each of the nonnatural amino acids includes a tetrazine functional group or a triazine functional group.

### Example 45, Inclusion of Three Urate Oxidase Variant Subunit

The urate oxidase variant of Example 44, in which the urate oxidase variant is a tetramer formed by oligomerization of one wild-type urate oxidase subunit and three urate oxidase variant subunits,
in which the urate oxidase variant subunit is a subunit obtained by substituting at least one amino acid in the sequence of a wild-type urate oxidase subunit with a nonnatural amino acid including a tetrazin functional group or a triazine functional group.

### Example 46, Inclusion of Four Urate Oxidase Variant Subunits

The urate oxidase variant of Example 44, in which the urate oxidase variant is a tetramer formed by oligomerization of four urate oxidase variant subunits, and
in which the urate oxidase variant subunit is a subunit obtained by substituting at least one amino acid in the sequence of a wild-type urate oxidase subunit with a nonnatural amino acid including a tetrazin functional group or a triazine functional group.

### Example 47, Exemplifying Origins of Urate Oxidase

The urate oxidase variant of Example 46, in which each of the urate oxidase variant subunits is obtained by modifying the sequence of a wild-type urate oxidase subunit derived from a microorganism selected from Aspergillus Flavus, Candida utilis, and Arthrobacter globiformis.

### Example 48, Limitation of Position of Substitution of Urate Oxidase Variant

The urate oxidase variant of Example 46, in which each of the urate oxidase variant subunits is obtained by substituting one or more amino acids at appropriate positions for substitution in the sequence of a wild-type urate oxidase subunit with the nonnatural amino acid, and
the appropriate positions for substitution are determined as positions that do not affect the function and structure of urate oxidase subunits and at which accessibility to a solvent is high.

### Example 49, Limitation of Substitution Position Determination Method for Urate Oxidase Variant

The urate oxidase variant of Example 48, in which the appropriate position for substitution is determined preferably by referring to a scoring result of a Rosetta molecule modeling package as a result of a molecule modeling simulation result.

### Example 50, Aspergillus Flavus Uox, Sequence Selection

The urate oxidase variant of Example 47, in which the urate oxidase variant includes a first urate oxidase variant subunit, a second urate oxidase variant subunit, a third urate oxidase variant subunit, and a fourth urate oxidase variant subunit,
the first urate oxidase variant subunit, the second urate oxidase variant subunit, the third urate oxidase variant subunit, and the fourth urate oxidase variant subunit are each independently represented by a sequence selected from SEQ ID NOs: 2 to SEQ ID NO: 50, or a sequence that is 80% or more identical to the selected sequence, and
X in each sequence is a nonnatural amino acid including a tetrazine functional group or a triazine functional group.

### Example 51, Aspergillus Flavus Uox, Selection of Nonnatural Amino Acid

The urate oxidase variant of Example 50, in which in each of the urate oxidase variant subunits, X is a nonnatural amino acid selected independently from the tables of FIGS. 23 to 27.

### Example 52, Aspergillus Flavus Uox, Sequence Unification

The urate oxidase variant of Example 51, in which the first urate oxidase variant, the second urate oxidase variant, the third urate oxidase variant, and the fourth urate oxidase variant have the identical sequence.

### Example 53, Candida utilis Uox, Sequence Selection

The urate oxidase variant of Example 47, in which the urate oxidase variant includes a first urate oxidase variant subunit, a second urate oxidase variant subunit, a third urate oxidase variant subunit, and a fourth urate oxidase variant subunit,
the first urate oxidase variant subunit, the second urate oxidase variant subunit, the third urate oxidase variant subunit, and the fourth urate oxidase variant subunit are each independently represented by a sequence selected from SEQ ID NOs: 52 to 117, or a sequence that is 80% or more identical to the selected sequence, and
X in each sequence is a nonnatural amino acid including a tetrazine functional group or a triazine functional group.

### Example 54, Candida utilis Uox, Selection of Nonnatural Amino Acid

The urate oxidase variant of Example 53, in which in each of the urate oxidase variant subunits, X is a nonnatural amino acid selected independently from the tables of FIGS. 23 to 27.

### Example 55, Candida utilis Uox, Sequence Unification

The urate oxidase variant of Example 54, in which the first urate oxidase variant, the second urate oxidase variant, the third urate oxidase variant, and the fourth urate oxidase variant have the identical sequence.

### Example 56, Arthrobacter globiformis Uox, Sequence Selection

The urate oxidase variant of Example 47, in which the urate oxidase variant includes a first urate oxidase variant subunit, a second urate oxidase variant subunit, a third urate oxidase variant subunit, and a fourth urate oxidase variant subunit,
the first urate oxidase variant subunit, the second urate oxidase variant subunit, the third urate oxidase variant subunit, and the fourth urate oxidase variant subunit are each independently represented by a sequence selected from SEQ ID NOs: 119 to 132, or a sequence that is 80% or more identical to the selected sequence, and
X in each sequence is a nonnatural amino acid including a tetrazine functional group or a triazine functional group.

### Example 57, Arthrobacter Globiformis Uox, Selection of Nonnatural Amino Acid

The urate oxidase variant of Example 56, in which in each of the urate oxidase variant subunits, X is a nonnatural amino acid selected independently from the tables of FIGS. 23 to 27.

### Example 58, Arthrobacter Globiformis Uox, Sequence Unification

The urate oxidase variant of Example 57, in which the first urate oxidase variant, the second urate oxidase variant, the third urate oxidase variant, and the fourth urate oxidase variant have the identical sequence.

### Vector Expressing Urate Oxidase Variant

### Example 59, Vector Expressing Urate Oxidase Variant

A vector capable of expressing the urate oxidase variant of anyone of Examples 44 to 58, in which a part corresponding to a nonnatural amino acid in the urate oxidase variant is encoded with any one selected from an ambber codon (5′-UAG-3′), an ocher codon (5′-UAA-3′), and an opal codon (5′-UGA-3′).

### Example 60, Limitation of Vector Expressing Urate Oxidase Variant

The vector of Example 59, in which the vector includes one or more sequences selected from the following or includes sequences that are 80% or more identical to the selected sequences:

(SEQ ID NO: 152)
5’-ATGTCTGCTGTGAAGGCCGCAAGATATGGCAAGGATAATGTGAGGGTGTACAAGGTGCAT
AAGGACGAAAAGACTGGCGTGCAGACAGTGTACGAGATGACCGTGTGCGTCCTGCTGGA
GGGCGAAATCGAGACTTCTTATACCAAAGCCGACAACTCCGTGATTGTGGCCACAGATTC
TATCAAGAACACTATCTATATCACCGCCAAACAGAACCCAGTGACACCACCTGAACTGTT
CGGCAGCATTCTCGGCACACACTTTATTGAGAAGTACAACCACATCCATGCTGCACACGT
GAATATCGTGTGTCATCGCTGGACCCGCATGGACATCTAGGGAAAGCCACACCCCCACTC
TTTTATCAGAGACTCTGAAGAAAAGAGAAACGTGCAGGTCGACGTGGTGGAGGGAAAAG
GTATCGACATCAAGAGCTCACTCTCCGGCCTGACCGTGCTGAAGAGTACCAATTCACAGTT
TTGGGGGTTTCTGAGAGACGAATACACTACACTGAAGGAGACTTGGGATAGAATCCTGAG
TACCGACGTGGATGCAACCTGGCAGTGGAAGAATTTTTCCGGGCTGCAGGAAGTGCGGTC
CCACGTGCCCAAGTTTGATGCAACCTGGGCAACCGCAAGGGAGGTGACACTGAAAACCTT
TGCCGAGGACAACTCCGCTAGCGTGCAGGCCACAATGTACAAGATGGCCGAACAGATCCT
GGCCAGACAGCAGCTGATTGAGACTGTGGAGTACTCTCTGCCTAACAAGCACTATTTCGA
AATCGACCTGTCCTGGCACAAGGGACTGCAGAATACTGGTAAAAACGCAGAGGTGTTCGC
CCCTCAGAGTGATCCCAATGGTCTGATCAAATGCACAGTGGGGAGATCCTCTCTGAAGAG
CAAGCTGTAA-3’;
(SEQ ID NO: 153)
5’-ATGTCTGCTGTGAAGGCCGCAAGATATGGCAAGGATAATGTGAGGGTGTACAAGGTGCAT
AAGGACGAAAAGACTGGCGTGCAGACAGTGTACGAGATGACCGTGTGCGTCCTGCTGGA
GGGCGAAATCGAGACTTCTTATACCAAAGCCGACAACTCCGTGATTGTGGCCACAGATTC
TATCAAGAACACTATCTATATCACCGCCAAACAGAACCCAGTGACACCACCTGAACTGTT
CGGCAGCATTCTCGGCACACACTTTATTGAGAAGTACAACCACATCCATGCTGCACACGT
GAATATCGTGTGTCATCGCTGGACCCGCATGGACATCGACGGAAAGCCACACCCCCACTC
TTTTATCAGAGACTCTGAAGAAAAGAGAAACGTGCAGGTCGACGTGGTGGAGGGAAAAG
GTATCGACATCAAGAGCTCACTCTCCGGCCTGACCGTGCTGAAGAGTACCAATTCACAGTT
TTAGGGGTTTCTGAGAGACGAATACACTACACTGAAGGAGACTTGGGATAGAATCCTGAG
TACCGACGTGGATGCAACCTGGCAGTGGAAGAATTTTTCCGGGCTGCAGGAAGTGCGGTC
CCACGTGCCCAAGTTTGATGCAACCTGGGCAACCGCAAGGGAGGTGACACTGAAAACCTT
TGCCGAGGACAACTCCGCTAGCGTGCAGGCCACAATGTACAAGATGGCCGAACAGATCCT
GGCCAGACAGCAGCTGATTGAGACTGTGGAGTACTCTCTGCCTAACAAGCACTATTTCGA
AATCGACCTGTCCTGGCACAAGGGACTGCAGAATACTGGTAAAAACGCAGAGGTGTTCGC
CCCTCAGAGTGATCCCAATGGTCTGATCAAATGCACAGTGGGGAGATCCTCTCTGAAGAG
CAAGCTGTAA-3’;
(SEQ ID NO: 154)
5’-ATGTCTGCTGTGAAGGCCGCAAGATATGGCAAGGATAATGTGAGGGTGTACAAGGTGCAT
AAGGACGAAAAGACTGGCGTGCAGACAGTGTACGAGATGACCGTGTGCGTCCTGCTGGA
GGGCGAAATCGAGACTTCTTATACCAAAGCCGACAACTCCGTGATTGTGGCCACAGATTC
TATCAAGAACACTATCTATATCACCGCCAAACAGAACCCAGTGACACCACCTGAACTGTT
CGGCAGCATTCTCGGCACACACTTTATTGAGAAGTACAACCACATCCATGCTGCACACGT
GAATATCGTGTGTCATCGCTGGACCCGCATGGACATCGACGGAAAGCCACACCCCCACTC
TTTTATCAGAGACTCTGAAGAAAAGAGAAACGTGCAGGTCGACGTGGTGGAGGGAAAAG
GTATCGACATCAAGAGCTCACTCTCCGGCCTGACCGTGCTGAAGAGTACCAATTCACAGTT
TTGGGGGTTTCTGAGAGACGAATACACTACACTGAAGGAGACTTAGGATAGAATCCTGAG
TACCGACGTGGATGCAACCTGGCAGTGGAAGAATTTTTCCGGGCTGCAGGAAGTGCGGTC
CCACGTGCCCAAGTTTGATGCAACCTGGGCAACCGCAAGGGAGGTGACACTGAAAACCTT
TGCCGAGGACAACTCCGCTAGCGTGCAGGCCACAATGTACAAGATGGCCGAACAGATCCT
GGCCAGACAGCAGCTGATTGAGACTGTGGAGTACTCTCTGCCTAACAAGCACTATTTCGA
AATCGACCTGTCCTGGCACAAGGGACTGCAGAATACTGGTAAAAACGCAGAGGTGTTCGC
CCCTCAGAGTGATCCCAATGGTCTGATCAAATGCACAGTGGGGAGATCCTCTCTGAAGAG
CAAGCTGTAA-3’;
(SEQ ID NO: 155)
5’-ATGAGCACCACACTGAGCAGCAGCACCTATGGTAAAGATAATGTGAAATTCCTGAAAGTG
AAAAAAGATCCGCAGAACCCGAAAAAACAAGAAGTTATGGAAGCAACCGTTACCTGTCT
GCTGGAAGGTGGTTTTGATACCAGCTATACCGAAGCAGATAATAGCAGCATTGTTCCGAC
CGATACCGTGAAAAATACCATTCTGGTTCTGGCAAAAACCACCGAAATTTGGCCGATTGA
ACGTTTTGCAGCCAAACTGGCAACCCATTTTGTTGAGAAATATTCTCATGTTAGCGGTGTG
AGCGTTAAAATTGTTCAGGATCGTTGGGTTAAATATGCCGTTGATGGTAAACCGCATGATC
ACAGCTTTATTCATGAAGGTGGTGAAAAACGTATCACCGACCTGTATTACAAACGTAGCG
GTGATTATAAACTGTCCAGCGCAATTAAAGATCTGACCGTTCTGAAAAGCACCGGCAGCA
TGTTTTAGGGTTATAACAAATGCGATTTCACAACCCTGCAGCCGACCACCGATCGTATTCT
GAGCACCGATGTTGATGCAACCTGGGTTTGGGATAATAAGAAAATTGGTAGCGTGTACGA
TATTGCCAAAGCAGCAGATAAAGGCATCTTCGATAATGTGTATAATCAGGCACGTGAAAT
TACCCTGACCACCTTTGCACTGGAAAATAGCCCGAGCGTTCAGGCAACCATGTTTAATATG
GCGACCCAGATTCTGGAAAAAGCGTGTAGCGTTTATAGCGTTAGCTATGCACTGCCGAAC
AAACACTATTTTCTGATTGACCTGAAATGGAAGGGCCTTGAAAATGATAACGAACTGTTTT
ATCCGAGTCCGCATCCGAATGGTCTGATTAAATGTACCGTTGTGCGTAAAGAGAAAACCA
AACTGTAA-3';
(SEQ ID NO: 156)
5’-ATGAGCACCACACTGAGCAGCAGCACCTATGGTAAAGATAATGTGAAATTCCTGAAAGTG
AAAAAAGATCCGCAGAACCCGAAAAAACAAGAAGTTATGGAAGCAACCGTTACCTGTCT
GCTGGAAGGTGGTTTTGATACCAGCTATACCGAAGCAGATAATAGCAGCATTGTTCCGAC
CGATACCGTGAAAAATACCATTCTGGTTCTGGCAAAAACCACCGAAATTTGGCCGATTGA
ACGTTTTGCAGCCAAACTGGCAACCCATTTTGTTGAGAAATATTCTCATGTTAGCGGTGTG
AGCGTTAAAATTGTTCAGGATCGTTGGGTTAAATATGCCGTTGATGGTAAACCGCATGATC
ACAGCTTTATTCATGAAGGTGGTGAAAAACGTATCACCGACCTGTATTACAAACGTAGCG
GTGATTATAAACTGTCCAGCGCAATTAAAGATCTGACCGTTCTGAAAAGCACCGGCAGCA
TGTTTTATGGTTATAACAAATGCGATTTCACAACCCTGCAGCCGACCACCGATCGTATTCT
GAGCACCGATGTTGATGCAACCTGGGTTTGGGATAATAAGAAAATTGGTAGCGTGTAGGA
TATTGCCAAAGCAGCAGATAAAGGCATCTTCGATAATGTGTATAATCAGGCACGTGAAAT
TACCCTGACCACCTTTGCACTGGAAAATAGCCCGAGCGTTCAGGCAACCATGTTTAATATG
GCGACCCAGATTCTGGAAAAAGCGTGTAGCGTTTATAGCGTTAGCTATGCACTGCCGAAC
AAACACTATTTTCTGATTGACCTGAAATGGAAGGGCCTTGAAAATGATAACGAACTGTTTT
ATCCGAGTCCGCATCCGAATGGTCTGATTAAATGTACCGTTGTGCGTAAAGAGAAAACCA
AACTGTAA-3’;
(SEQ ID NO: 157)
5’-ATGAGCACCACACTGAGCAGCAGCACCTATGGTAAAGATAATGTGAAATTCCTGAAAGTG
AAAAAAGATCCGCAGAACCCGAAAAAACAAGAAGTTATGGAAGCAACCGTTACCTGTCT
GCTGGAAGGTGGTTTTGATACCAGCTATACCGAAGCAGATAATAGCAGCATTGTTCCGAC
CGATACCGTGAAAAATACCATTCTGGTTCTGGCAAAAACCACCGAAATTTGGCCGATTGA
ACGTTTTGCAGCCAAACTGGCAACCCATTTTGTTGAGAAATATTCTCATGTTAGCGGTGTG
AGCGTTAAAATTGTTCAGGATCGTTGGGTTAAATATGCCGTTGATGGTAAACCGCATGATC
ACAGCTTTATTCATGAAGGTGGTGAAAAACGTATCACCGACCTGTATTACAAACGTAGCG
GTGATTATAAACTGTCCAGCGCAATTAAAGATCTGACCGTTCTGAAAAGCACCGGCAGCA
TGTTTTATGGTTATAACAAATGCGATTTCACAACCCTGCAGCCGACCACCGATCGTATTCT
GAGCACCGATGTTGATGCAACCTGGGTTTGGGATAATAAGAAAATTGGTAGCGTGTACGA
TATTGCCAAAGCAGCAGATAAAGGCATCTTCGATAATGTGTATAATCAGGCACGTGAAAT
TACCCTGACCACCTTTGCACTGGAAAATAGCCCGAGCGTTCAGGCAACCATGTTTAATATG
GCGACCCAGATTCTGGAAAAAGCGTGTAGCGTTTATAGCGTTAGCTATGCACTGCCGAAC
AAACACTATTTTCTGATTGACCTGAAATAGAAGGGCCTTGAAAATGATAACGAACTGTTTT
ATCCGAGTCCGCATCCGAATGGTCTGATTAAATGTACCGTTGTGCGTAAAGAGAAAACCA
AACTGTAA-3’;
(SEQ ID NO: 158)
5’-ATGACCGCAACCGCAGAAACCAGCACCGGCACCAAAGTTGTTCTGGGTCAGAATCAGTAT
GGTAAAGCAGAAGTTCGTCTGGTTAAAGTTACCCGTAATACCGCACGTCATGAAATTCAG
GATCTGAATGTTACCAGCCAGCTGCGTGGTGATTTTGAAGCAGCACATACCGCAGGCGAT
AATGCACATGTTGTTGCAACCGATACACAGAAAAACACCGTTTATGCATTTGCCCGTGATG
GTTTTGCAACCACCGAAGAATTTCTGCTGCGTCTGGGTAAACATTTCACCGAAGGTTTTGA
TTGGGTTACCGGTGGTCGTTGGGCAGCACAGCAGTTTTTCTGGGATCGTATTTAGGATCAC
GATCATGCCTTTAGCCGCAATAAAAGCGAAGTGCGTACCGCAGTTCTGGAAATTAGCGGT
AGCGAACAGGCAATTGTTGCAGGTATTGAAGGTCTGACCGTTCTGAAAAGCACCGGTAGC
GAGTTTCATGGTTTTCCGCGTGATAAATACACCACACTGCAAGAAACCACCGATCGTATTC
TGGCAACCGATGTTAGCGCACGTTGGCGTTATAATACCGTTGAAGTTGATTTTGATGCGGT
TTATGCAAGCGTTCGTGGTCTGCTGCTGAAAGCATTTGCAGAAACCCATAGCCTGGCACTG
CAGCAGACAATGTATGAAATGGGTCGTGCAGTTATTGAAACCCATCCGGAAATTGATGAG
ATCAAAATGAGCCTGCCGAACAAACATCATTTTCTGGTTGATCTGCAGCCGTTTGGTCAGG
ATAATCCGAATGAAGTGTTTTATGCAGCAGATCGTCCGTATGGTCTGATTGAAGCAACCAT
TCAGCGTGAAGGTAGCCGTGCAGATCATCCGATTTGGAGTAATATTGCAGGTTTTTGCTAA-3’;
(SEQ ID NO: 159)
5’-ATGACCGCAACCGCAGAAACCAGCACCGGCACCAAAGTTGTTCTGGGTCAGAATCAGTAT
GGTAAAGCAGAAGTTCGTCTGGTTAAAGTTACCCGTAATACCGCACGTCATGAAATTCAG
GATCTGAATGTTACCAGCCAGCTGCGTGGTGATTTTGAAGCAGCACATACCGCAGGCGAT
AATGCACATGTTGTTGCAACCGATACACAGAAAAACACCGTTTATGCATTTGCCCGTGATG
GTTTTGCAACCACCGAAGAATTTCTGCTGCGTCTGGGTAAACATTTCACCGAAGGTTTTGA
TTGGGTTACCGGTGGTCGTTGGGCAGCACAGCAGTTTTTCTGGGATCGTATTAATGATCAC
GATCATGCCTTTAGCCGCAATAAAAGCGAAGTGCGTACCGCAGTTCTGGAAATTAGCGGT
TAGGAACAGGCAATTGTTGCAGGTATTGAAGGTCTGACCGTTCTGAAAAGCACCGGTAGC
GAGTTTCATGGTTTTCCGCGTGATAAATACACCACACTGCAAGAAACCACCGATCGTATTC
TGGCAACCGATGTTAGCGCACGTTGGCGTTATAATACCGTTGAAGTTGATTTTGATGCGGT
TTATGCAAGCGTTCGTGGTCTGCTGCTGAAAGCATTTGCAGAAACCCATAGCCTGGCACTG
CAGCAGACAATGTATGAAATGGGTCGTGCAGTTATTGAAACCCATCCGGAAATTGATGAG
ATCAAAATGAGCCTGCCGAACAAACATCATTTTCTGGTTGATCTGCAGCCGTTTGGTCAGG
ATAATCCGAATGAAGTGTTTTATGCAGCAGATCGTCCGTATGGTCTGATTGAAGCAACCAT
TCAGCGTGAAGGTAGCCGTGCAGATCATCCGATTTGGAGTAATATTGCAGGTTTTTGCTAA-3’;
and
(SEQ ID NO: 160)
5’-ATGACCGCAACCGCAGAAACCAGCACCGGCACCAAAGTTGTTCTGGGTCAGAATCAGTAT
GGTAAAGCAGAAGTTCGTCTGGTTAAAGTTACCCGTAATACCGCACGTCATGAAATTCAG
GATCTGAATGTTACCAGCCAGCTGCGTGGTGATTTTGAAGCAGCACATACCGCAGGCGAT
AATGCACATGTTGTTGCAACCGATACACAGAAAAACACCGTTTATGCATTTGCCCGTGATG
GTTTTGCAACCACCGAAGAATTTCTGCTGCGTCTGGGTAAACATTTCACCGAAGGTTTTGA
TTGGGTTACCGGTGGTCGTTGGGCAGCACAGCAGTTTTTCTGGGATCGTATTAATGATCAC
GATCATGCCTTTAGCCGCAATAAAAGCGAAGTGCGTACCGCAGTTCTGGAAATTAGCGGT
AGCGAACAGGCAATTGTTGCAGGTATTGAAGGTCTGACCGTTCTGAAAAGCACCGGTAGC
GAGTTTCATGGTTTTCCGCGTGATAAATACACCACACTGCAAGAAACCACCGATCGTATTC
TGGCAACCGATGTTAGCGCACGTTGGCGTTATAATACCGTTTAGGTTGATTTTGATGCGGT
TTATGCAAGCGTTCGTGGTCTGCTGCTGAAAGCATTTGCAGAAACCCATAGCCTGGCACTG
CAGCAGACAATGTATGAAATGGGTCGTGCAGTTATTGAAACCCATCCGGAAATTGATGAG
ATCAAAATGAGCCTGCCGAACAAACATCATTTTCTGGTTGATCTGCAGCCGTTTGGTCAGG
ATAATCCGAATGAAGTGTTTTATGCAGCAGATCGTCCGTATGGTCTGATTGAAGCAACCAT
TCAGCGTGAAGGTAGCCGTGCAGATCATCCGATTTGGAGTAATATTGCAGGTTTTTGCTAA-3’.

### Urate Oxidase Variant Preparation Method

### Example 61, Urate Oxidase Variant Preparation Method

A method for preparing a urate oxidase variant, the method comprising:
preparing a cell line comprising a vector capable of expressing an orthogonal tRNA/synthetase pair, and a urate oxidase variant expression vector,
wherein the vector capable of expressing orthogonal tRNA/synthetase is capable of expressing an exogenous suppressor tRNA and an exogenous tRNA synthetase,
wherein the exogenous suppressor tRNA is capable of recognizing a specific stop codon,
the exogenous tRNA synthetase is capable of recognizing a nonnatural amino acid containing a tetrazine functional group and/or a triazine functional group and of linking the recognized amino acid to the exogenous suppressor tRNA,
wherein the urate oxidase variant expression vector is capable of expressing the urate oxidase variant of one of Examples 44 to 58 and is formed such that a position corresponding to the nonnatural amino acid in the urate oxidase variant is encoded with the specific stop codon; and
culturing the cell line in a medium containing at least one kind of a nonnatural amino acid comprising a tetrazine functional group and/or a triazine functional group.

### Example 62, Limitation of Stop Codon

The method of Example 61, in which the specific stop codon is any one selected from among an amber codon (5′-UAG-3′), an ocher codon (5′-UAA-3′), and an opal codon (5′-UGA-3′).

### Example 63, Cell Line Mutation

The method of Example 62, in which the cell line is a cell line in which a release factor recognizing the specific stop codon is inactivated.

### Example 64, Limitation of Cell Line

The method of Example 63, in which the cell line is E. coli C321.ΔA.exp (Addgene, ID:49018).

### Example 65, Limitation of Orthogonal tRNA/synthetase Pair

The method of Example 61, in which the orthogonal tRNA/synthetase pair is Methanococcus jannaschii-derived suppressor tRNA (MjtRNA^{Tyr}_{CUA}) and Methanococcus jannaschii-derived tyrosyl-tRNA synthetase (MjTyrRS).

### Example 66, Limitation of Vector Expressing tRNA/Synthetase Pair

The method of Example 65, in which the vector capable of expressing the orthogonal tRNA/synthetase pair may be pDule_C11 reported by Yang et. al. (Temporal Control of Efficient In Vivo Bioconjugation Using a Genetically Encoded Tetrazine-Mediated Inverse-Electron-Demand Diels-Alder Reaction, Bioconjugate Chemistry, 2020, 2456-2464).

### Example 67, Limitation of Urate Oxidase Variant

The method of Example 61, in which the vector expressing the urate oxidase variant is capable of expressing the urate oxidase variant of one of Example 51, Example 54, and Example 57, the nonnatural amino acid in the sequence of the urate oxidase variant is encoded with the specific stop codon.

### Mode for Invention

Hereinafter, the invention provided by the present description will be described in more detail through experimental examples and examples. These examples are only for illustrative purposes, and it will be apparent to those skilled in the art that the scope of the disclosure of the present description is not limited by these examples.

### Experimental Example 1: Obtainment of Urate Oxidase Variant

### Experimental Example 1.1: Preparation of Vector for Expression of Urate Oxidase Variant

A pTAC_Uox plasmid was constructed using the gene encoding Uox derived from Aspergillus flavus as a template. In order to apply a TAC promoter, the sequence information of the pTAC-MAT-TAG-1 expression vector (Sigma, E5530) was referred to, and the 5′-TTTGTTAACTTAAGAAGGAGA-3′ (Sequence ID NO: 151), which is a Ribosome binding site (RBS) sequence extended compared to the existing pQE80 vector, was applied. For recombinant protein expression of a pTAC vector, transcription control, rmBt1 terminator sequence, and rmBt2 terminator sequence were applied.

The DNA synthesis of the sequence of the rrnbT1-rrnbT2 terminator from the TAC promoter was performed by Macrogen at the request of the inventors, and cloning was performed on the pQE80L vector to prepare a pTAC-empty vector. Cloning of pTAC-empty was carried out through infusion cloning, and cloning was completed using an Infusion® HD cloning kit (Takara Korea Biomedical). The prepared pTAC-empty vector underwent sequencing analysis for verification.

Each sequence used for vector construction is shown in Table 1 below.

TABLE 1

| | | SEQ | | | |
|---|---|---|---|---|---|
| | Sequence | ID | | GC | TM |
| Label | (5′ to 3′) | NO | bp | (%) | (° C.) |
| pTAC | CAA GCT TGG | 145 | 20 | 55 | 64 |
| linearize- | CTG TTT TGG | | | | |
| F | CG | | | | |
| pTAC | CTA TAT CTC | 146 | 25 | 28 | 53 |
| linearize- | CTT CTT AAA | | | | |
| R | GTT AAA C | | | | |
| tacP-RBS- | AAG AAG GAG | 147 | 34 | 47 | 62 |
| MCS- | ATA TAG ATG | | | | |
| rrnBt1t2- | TCT GCT GTG | | | | |
| F | AAG GCC G | | | | |
| tacP-RBS- | AAA CAG CCA | 148 | 36 | 44 | 59 |
| MCS- | AGC TTG TTA | | | | |
| rrnBt1t2- | CAG CTT GCT | | | | |
| R | CTT CAG AGA | | | | |
| pTAC- | GCC TAG AGC | 149 | 20 | 55 | 57 |
| sequencing- | AAG ACG TTT | | | | |
| F | CC | | | | |
| pTAC- | TTA ATG CAG | 150 | 19 | 53 | 58 |
| sequencing- | CTG GCA CGA | | | | |
| R | C | | | | |

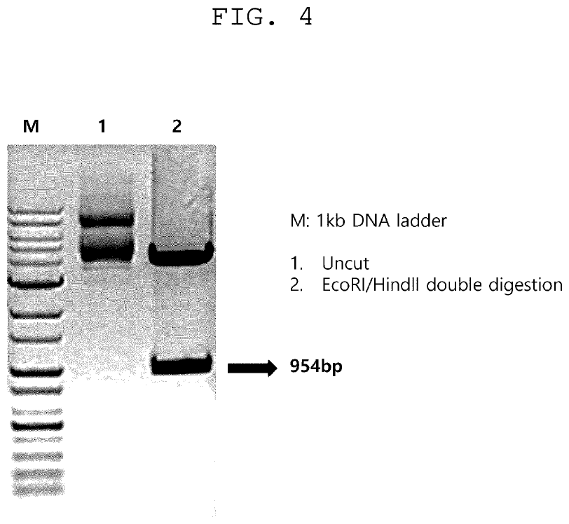

In order to amplify the gene encoding Uox derived from Aspergillus flavus as a template, infusion cloning was performed on the pTAC-empty vector prepared by performing PCR-amplification on the previously cloned pQE80-Uox-W174amb vector. The infusion reaction was reflected by insert (Uox_W174amb) of 22 ng into 50 ng of vector, and was transformed into E. coli DH5a after the reaction at 50° C. for 15 minutes. Subsequently, a single colony was picked up and inoculated in a 4-mL LB broth medium to perform mini-prep. EcoRI/HindIII restriction enzyme digestion was performed to investigate the band (953 bp) of Uox-W174amb, which is a cloning insert gene sequence (FIG. 4). The cloned pTAC-Uox-W174amb was requested for sequencing, and the cloning result was investigated using the NCBI's BLAST program (FIG. 5) (SEQ ID NO: 154).

### Experimental Example 1.2: Expression and Purification of Urate Oxidase Variant

Since position 174 in Uox was previously reported to have no structural/functional role (Lim, 2015), little or no perturbation to maintain enzymatic activity was expected even upon HSA conjugation. Therefore, for the conjugation of HSA and Uox, one reactive site was selected per monomer in Uox composed of a tetramer, so that a total of 4 reactive sites were secured. Specifically, Uox is a tetrameric protein in which four monomers are oligomerized. When one site is inserted per monomer, the oligomerized tetramer has four reactive sites.

Specifically, in order to express Uox-frTet, C321delAexp Escherichia coli host cells were co-transformed using the pDule_C11 and pTAC_Uox-174Amb plasmids prepared in Experimental Example 1.1 (C321delA.exp [pDule_C11] [pTAC_Uox-174Amb]), and the cells were cultured in a 2×YT medium. Expression of Uox-frTet was performed using a protocol in which 1-3 mM frTet, tetracycline (10 μg/mL), and kanamycin (35 μg/mL) were added. Next, IPTG was added to promote the expression of Uox-frTet, and the presence or absence of Uox-frTet expression was checked for each expression induction time.

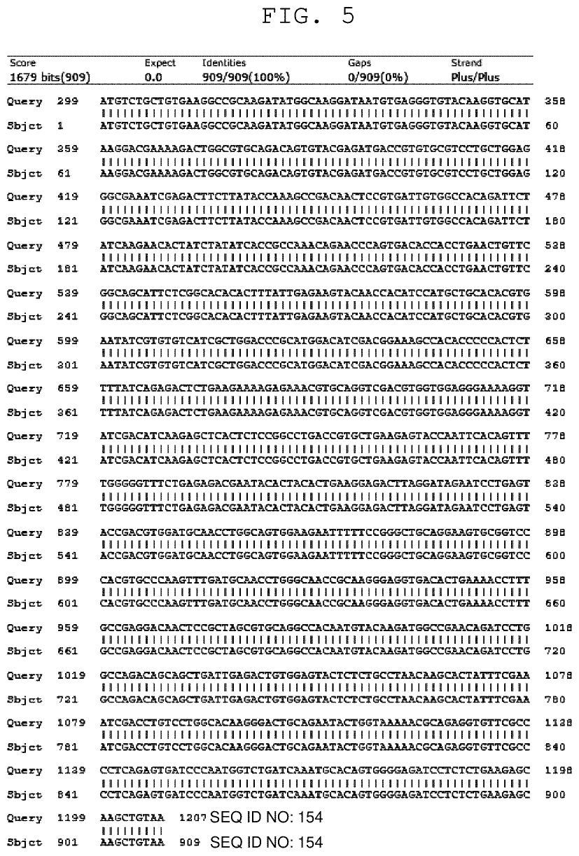

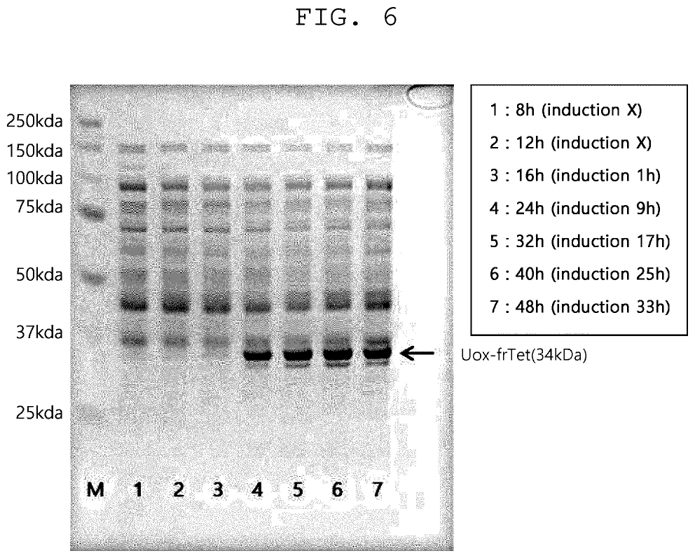

As a result of SDS-PAGE analysis, it was found that a protein band with a molecular weight of about 34 kDa exists, it was confirmed that the tendency of the protein band to become stronger with each induction time. The findings are consistent with the expected molecular weight (34 kDa) of the monomer Uox. Expression of Uox-frTet was confirmed through the above results (See FIG. 6).

For separation and purification of Uox-frTet, the obtained cells were mixed with a buffer (20 mM Tris-HCl pH 9.0) at a ratio of 1:5 (w/w %), and cell disruption was performed using a sonicator. After cell disruption, centrifugation was performed at 9,500 rpm for 40 minutes to remove microbial debris, and the supernatant was collected.

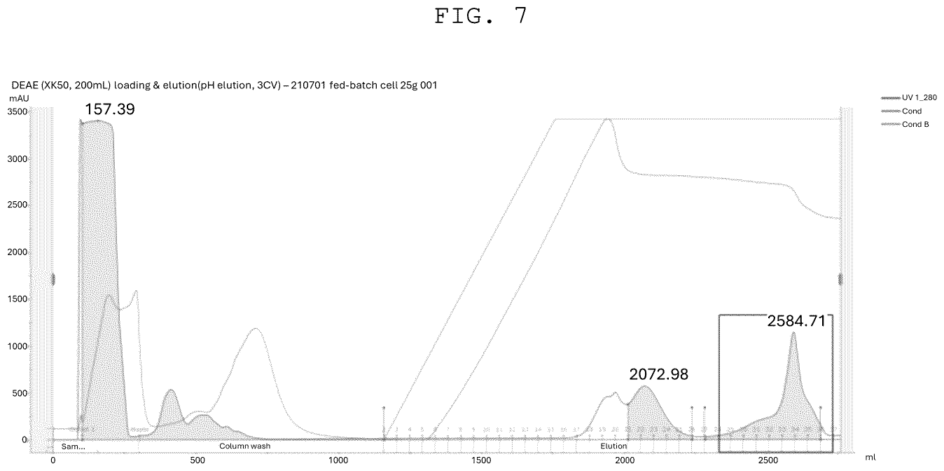

After filtering the collected supernatant through a 0.45-μm filter, primary separation and purification was performed with a DEAE Sepharose Fast Flow column (Cytiva, MA, USA) (refer to FIGS. 7 and 8), using an equilibration buffer (20 mM Tris-HCl pH 9.0) and an elution buffer (20 mM sodium phosphate pH 6.0).

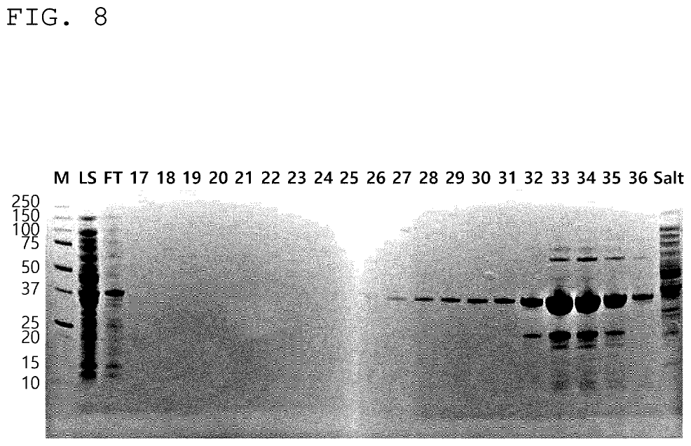

Fractions resulting from the primary purification were collected and secondary separation and purification was performed with a phenyl Fast Flow column (Cytiva, MA, USA), using an equilibration buffer (20 mM Tris-HCl pH 9.0+1M Ammonium sulfate) and an elution buffer (20 mM Tris-HCl pH 9.0) (See FIG. 9 and FIG. 10).

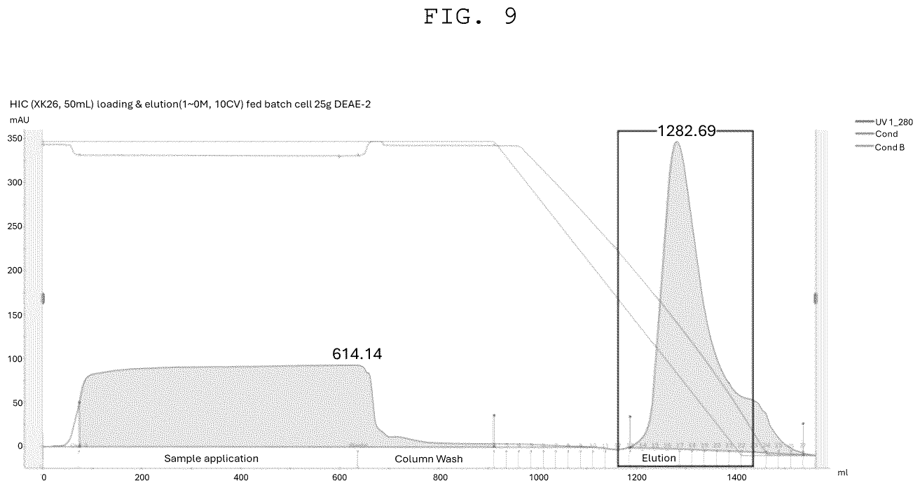

The fractions resulting from the secondary purification were collected, and it was confirmed that highly pure Uox-frTest was obtained through analysis. After purification on Coomassie blue-stained protein gel, a single band with a molecular weight of about 34 kDa was present in the elution lane after the purification. In addition, the SDS-PAGE analysis result (FIG. 11) revealed that there was a molecular weight band matching with “FASTURTEC (Rasburicase, sanofi-aventis)”, which is a commercially available urate oxidase.

### Experimental Example 1.3: Verification of Purity of Urate Oxidase Variant

To verify the purity of Uox-frTet, the Uox-frTet was analyzed using a high performance liquid chromatography (HPLC). The analysis column was TSKgel G3000SWXL (TOSOH). The analysis was performed with a mobile phase of 20 mM sodium phosphate pH 7.0+0.3M NaCl at a rate of 0.6 mL/min at UV 220 nm.

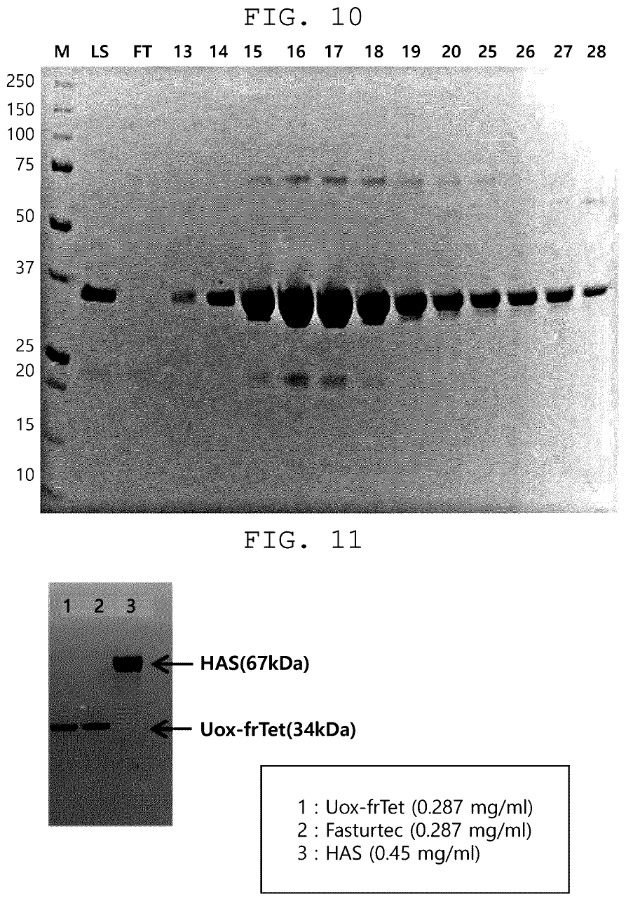

As a result, the secondary purified Uox-frTet was detected as a main peak at 14.7 minutes, and the purity was observed to be greater than 99%. It was confirmed that Uox-frTet was purified to a high purity (FIG. 12).

### Experimental Example 1.4 Verification of Introduction of frTet into Urate Oxidase Variant

Fluorescently labeled dye conjugation was used to investigate whether the genetically encoded frTet exhibits IEDDA reactivity. Specifically, for fluorescence labeling analysis, purified Uox-WT and Uox-frTet were mixed and reacted with Trans-Cyclooct-2-ene (TCO)-Cy3 dye in a molar ratio of 1:2 in PBS (pH 7.4) for 2 hours at room temperature. The reaction mixture was analyzed by SDS-PAGE. The fluorescence intensity of the gel was detected using a ChemiDoc XRS+ system (302 nm, filter 510/610 nm illumination; Bio-Rad Laboratories, Hercules, CA, USA) and then analyzed using Image Lab software (Bio-Rad Laboratories). Uox-WT samples with or without TCO-Cy3 were used as controls to verify IEDDA reactivity.

After fluorescence labeling analysis, Coomassie blue staining was performed for protein visualization. Protein gels were stained using Coomassie Brilliant Blue R-250 dye, and bands were detected using ChemiDoc XRS+ system (white illumination).

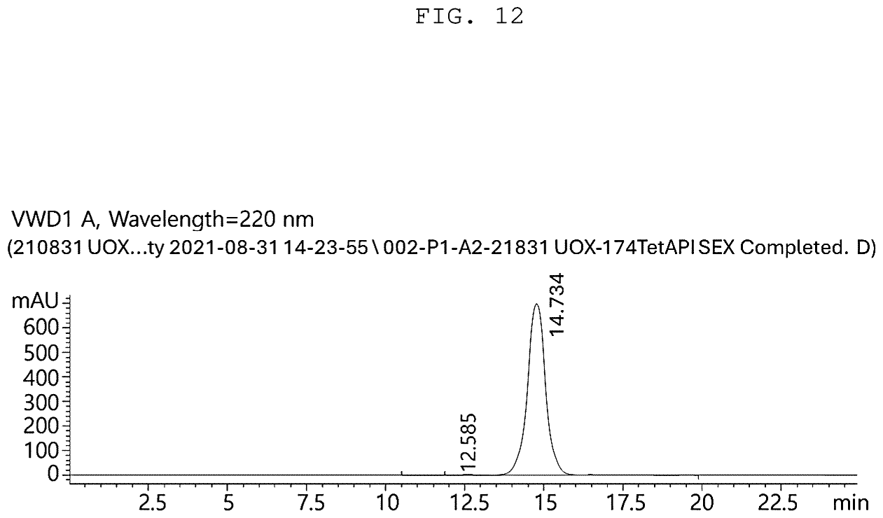

As a result, bands were identified in Uox-frTet incubated along with TCO-Cy3. Single protein bands were identified in all Uox variants after Coomassie blue staining regardless of incubation with TCO-Cy3. These results show that frTet introduced into Uox exhibits IEDDA reactivity (FIG. 13).

### Experimental Example 2: Obtainment of Urate Oxidase-Albumin Conjugate

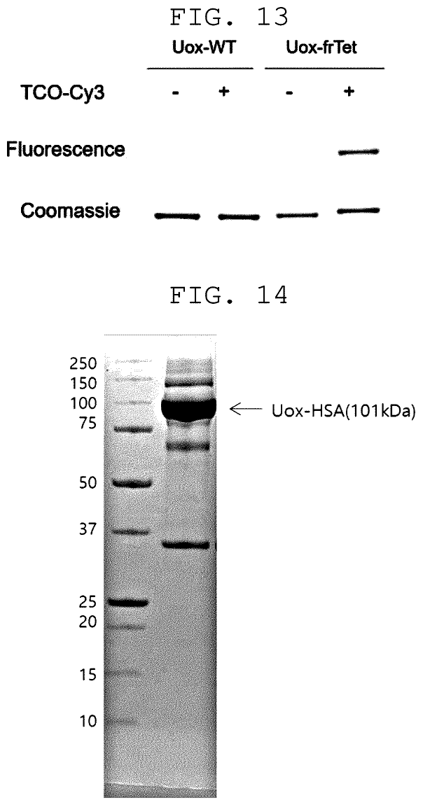

For the preparation of Uox-HSA, HSA and a TCO-Maleimide linker were combined at a ratio of 1:4 (molar ratio) at room temperature for 4 hours. To remove the unreacted remaining TCO-MAL linker, the reaction mixture was removed and desalted with a PBS buffer (pH 7.4) using a HiPrep 26/10 Desalting column. Thereafter, HSA-TCO and Uox-frTet were reacted at room temperature for 15 hours at a ratio of 5:1 (molar ratio). After filtering the sample through a 0.45-um filter, buffer exchange was performed with 20 mM sodium phosphate (pH 6.0), the sample was injected into an SP Sepharose column (Cytiva, MA. USA), and primary separation and purification was performed with an elution buffer (20 mM sodium phosphate, pH 6.0+1 M NaCl). The fractions resulting from the primary purification were collected, buffer exchange was performed with 20 mM Bis-Tris (pH 6.5), the collected sample was injected into a Q sepharose column, and secondary separation and purification was performed using an elution buffer (20 mM Bis-Tris pH 6.5+1 M NaCl). As a result of analysis of the secondary purified sample on Coomassie blue stained protein gel, a main band with a molecular weight of about 101 kDa was found to exist. This was consistent with the expected molecular weight (101 kDa) of the monomer Uox-HSA (FIG. 14). The secondary purified fractions were collected, and tertiary separation and purification was performed using a Superdex 200 Increase 10/300 GL column (Cytiva, MA, USA). As a result of analysis of the collected tertiary purified fractions, it was confirmed that high purity Uox-HSA was obtained. In addition, when analyzed with SEC-HPLC, a single peak showing a purity of 100% was determined (FIG. 15). Through this, a high-purity uric acid oxidase-albumin conjugate (Uox-HSA) was obtained.

### Experimental Example 3: In Vitro Enzymatic Activity Analysis of Urate Oxidase-Albumin Conjugate

To evaluate the in vitro enzymatic activity of Uox-frTet and Uox-HSA, the sample was diluted to a concentration of 10 nM, mixed with 111.1 uM of uric acid in a ratio of 1:9 (v/v %), and placed in a microplate. Then, absorbance 293 nm was measured at 15 second intervals for 10 minutes. As a result of measuring the in vitro enzymatic activity of Uox-frTet and Uox-HSA, uric acid showed a tendency to decrease, the slope was measured, and the activity was evaluated using the formula below.

$$\begin{matrix}U / mL = \frac{( initial rate \times tota1 vo1ume ( mL ) )}{( uric acid \times path length ( cm ) \times volume ( m L ) )} \\ U / mg = \frac{1 ml ( U / mL )}{final enzyme concentration}\end{matrix}$$

As a result, the activity per unit dose of Uox-frTet was 0.072 U/mL, and the activity per unit dose of Uox-HSA was 0.067 U/mL. It was confirmed that even though 4 albumins were bound to Uox-frTet, it did not significantly affect the decrease in enzymatic activity. In addition, the Uox-frTet exhibited a specific activity of 53 U/mg, and the Uox-HAS exhibited a specific activity of 16.6 U/mg.

### Experimental Example 4: Pharmacokinetics (PK) Evaluation Of Urate Oxidase-Albumin Conjugates

To evaluate the half-life of Uox-HSA, PK analysis was performed using ICR mice (n=5). The half-life of Uox-HSA was observed according to administration methods including intravenous (IV) administration, intraperitoneal (IP) administration, and intramuscular (IM) administration. As a control group, Fasturtec, which is a wild-type urate oxidase, was used for comparison. The dosage of Uox-HSA was 6.0 mg/kg (14.6 nmol/kg) (when administered by IV, IP, and IM), and the dosage of Fasturtec was 2.0 mg/kg (14.6 nmol/kg)(when administered by IV).

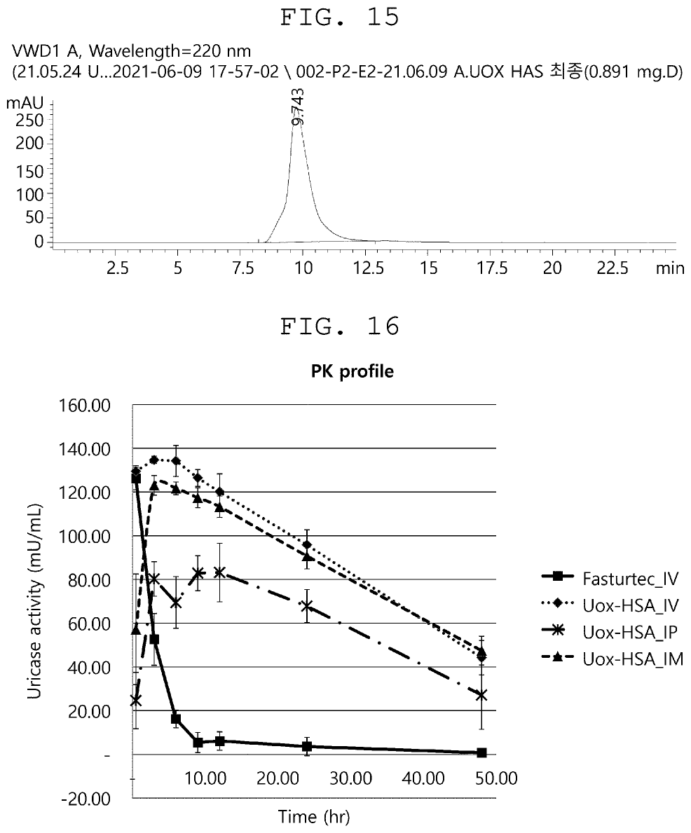

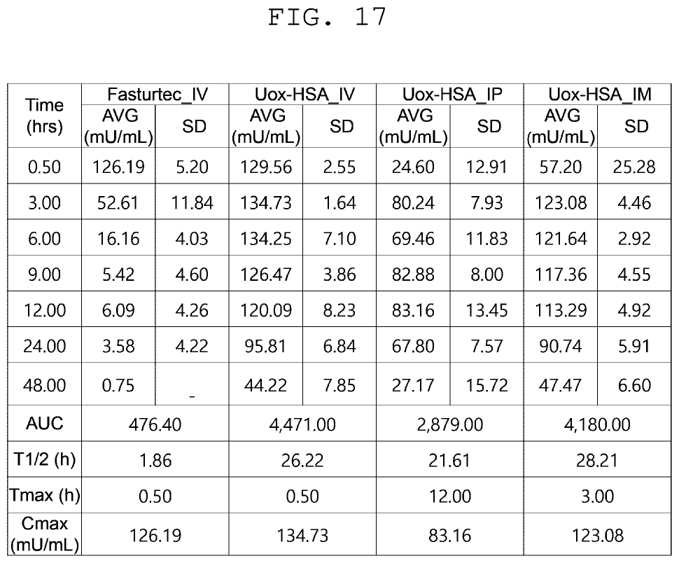

As a result, the area under curves (AUC) for the respective administration routes were higher in order of IV (4,471 mU/mL×h), IP (4,180 mU/mL×h), and IM (2,879 mU/mL×h). That is, the AUC was highest when administered by IV. The AUC of Uox-WT was significantly lower than 476 mU/mL×h. In addition, the half-life of Uox-HSA was found to be 26.22 hours in the case of IV administration, 28.2 hours in the case of IP administration, and 21.61 hours in the case of IM administration, and the half-life of Fasturtec was 1.86 hours in the case of IV administration. That is, in the case of IV administration, it was confirmed that the half-life of Uox-HSA was improved by about 14 times compared to that of Fasturtec (FIGS. 16 to 17).

### Experimental Example 5. Blood Uric Acid Reduction Effect of Urate Oxidase-Albumin Conjugate in Animal Model of Hyperuricemia

A hyperuricemia animal model (Winster-SD rat) was prepared using hypoxanthine, a precursor of uric acid (Hypoxanthine, 500 mg/kg), which is a uric acid precursor and potassium oxonate (250 mg/kg), which is a urate oxidase inhibitor. Then, a pharmacodynamic evaluation test was performed to check the blood uric acid level by treating the animal model with the prepared Uox-HSA drug.

Hyperuricemia was induced twice before administration of the test drug and re-induced twice in 24 hours and 48 hours, respectively, after the administration of the test drug. At each observation point, the blood uric acid reduction effect and persistence were checked. Each of the dosage was Uox-HSA 1.0 mg/kg (2.4 nmol/kg), Uox-HSA 4.0 mg/kg (9.8 nmol/kg), Uox-HSA 10 mg/kg (24.6 nmol/kg), and Fasturtec (Rasburicase) 1.33 mg/kg (9.8 nmol/kg). These were administered intravenously. Febuxostat was administered orally at a dose of 10 mg/kg (positive control). As a result, the initial blood uric acid level after induction of hyperuricemia was 12 mg/dL in the negative control group (Hyperuricemia, negative control). That is, it was confirmed that hyperuricemia was induced in rats. In thirty minutes after drug administration, it was confirmed that the uric acid level was lowered to be below the normal level (6 mg/dL) in all drug administration groups, and the level continued for up to 12 hours.

Secondary induction (in 24 hours after drug administration) was performed to check the drug's persistence. As a result, the blood uric acid level was maintained low in the Uox-HSA group, whereas the uric acid level increased in the group administered with Uox-WT and Febuxostat as a positive control.

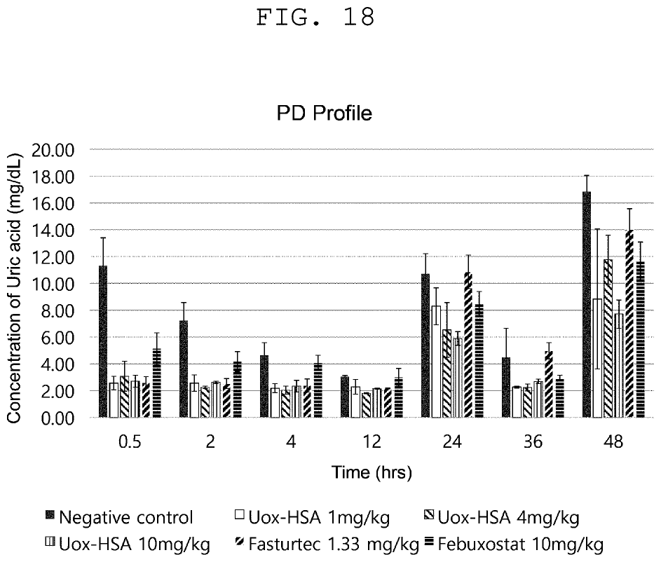

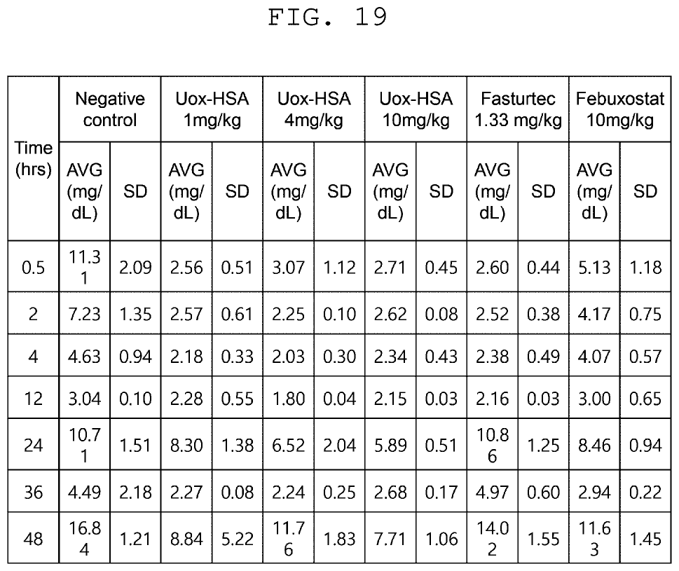

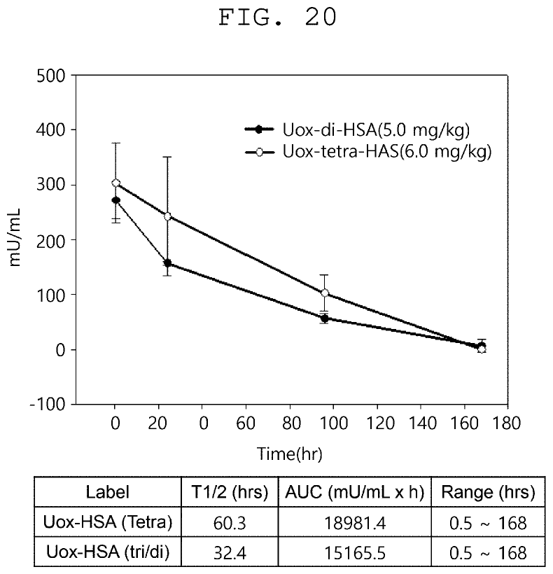

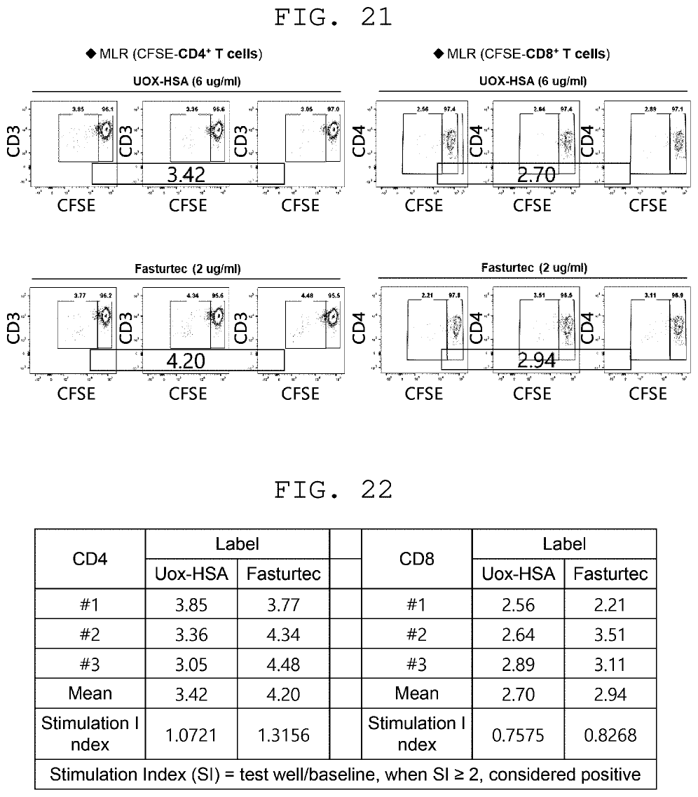

As a result of the third induction in 48 hours after drug administration, the Uox-HSA administration group showed an effect of reducing blood uric acid. It was confirmed that Uox-HSA continuously reduced blood uric acid in an animal model of hyperuricemia (FIGS. 18 to 19).

### Experimental Example 6. Pharmacokinetic Evaluation (PK Profile) Using Human FcRn (+/+) TG Mice

Uox-HSA is a drug to which human-derived albumin is bound, and its half-life improvement is not significant due to the poor binding ability to a mouse FcRn. Therefore, when using a mouse in which human FcRn is transgenic, the FcRn recycling effect can be expected due to the human-derived albumin. To evaluate this, the half-life of the prepared Uox-HSA was evaluated using Tg32 Alb−/− (human FcRn+/+) mice (n=4) (JAX #025201, The Jackson Laboratory). Uox-HSA (tetra-HSA) containing 4 albumins was administered by a single IV at a dose of 6.0 mg/kg, and Uox-HSA (tri/di-HSA) containing 2 to 3 albumins was administered by a single IV at a dose of 5.0 mg/kg. The activity of Uricase in the blood was checked by collecting blood in 0.5 hour, 1 day, 4 days, and 7 days after the single IV administration. As a result, in the case of Uox-HSA (tetra-HSA) in which 4 albumins were bound, the half-life was 60.3 hours. That is, the half-life increased by 2.3 times when confirmed in ICR mice, and the AUC increased by 4.2 times. In addition, in the case of Uox-HSA (tri/di-HSA) in which 2 to 3 albumins were bound, the half-life was 32.4 hours. It was confirmed that there was a large difference in the half-life according to the number of albumins. Specifically, when looking at the results of human FcRn TG mice, the half-life is expected to increase further when Uox-HSA (tetra-HSA) is administered to the human body.

### Experimental Example 7. Human-Derived PBMC-Based In Vitro Immunogenicity Assay

Although early prediction of the immunogenicity of biological products is an essential factor in determining the success or failure of the development of biological products, there is no laboratory animal model from which the immunogenicity of biological products can be reliably predicted before clinical trials due to differences in the immune systems between humans and laboratory animals. Therefore, immunogenicity analysis was performed using the immunogenicity analysis technique using human PBMC. Through this experiment, it is possible to obtain a reliable evaluation result for the immunogenicity of Uox-HSA before clinical trials.

After inducting differentiation from human mononuclear cells into dendritic cells which are known to be the most reliable so far, the reactivity (CD4+, CD8+ T cell activation) of immune cells induced by Uox-HSA was measured. As a result of CD4+ T cell activity analysis, it was obtained that Uox-HSA (6 ug/ml) and Fasturtec (2 ug/ml) both had SI values equal to or smaller than (≤2), indicating that they would not show immunogenicity in the HLA types below. In a relative comparison, Fasturtec (2 ug/ml) showed slightly higher CD4+ T cell activity than Uox-HSA (6 ug/ml), but the result was not statistically significant. As a result of CD8+ T cell activity analysis, it was obtained that Uox-HSA (6 ug/ml) and Fasturtec (2 ug/ml) both had SI values equal to or smaller than (≤2), indicating that they would not show immunogenicity in the HLA types below. As a result, it was analyzed that the immunogenicity would be lower than that of the original drug due to the binding of human albumins.

### Experimental Example 8: Preparation of Urate Oxidase-Albumin Conjugate

### Experimental Example 8.1: Preparation of Vector for Expression of Urate Oxidase Variant

A vector for expression of a urate oxidase variant is prepared by the method disclosed in Experimental Example 1.1. Here, the sequence of the vector of the urate oxidase variant to be expressed includes one or more sequences selected from SEQ ID NOs: 152 to 160.

### Experimental Example 8.2: Expression and Purification of Urate Oxidase Variant

The urate oxidase variant is expressed and purified by the method disclosed in Experimental Example 1.2, using the vector for expression of the urate oxidase variant of Experimental Example 6.1.

### Experimental Example 8.3: Obtainment and Verification of Urate Oxidase Variant

By the method disclosed in Experimental Examples 1.3 to 1.4, the purity of the urate oxidase variant obtained in Experimental Example 8.2 is evaluated and whether the nonnatural amino acid is well introduced into the urate oxidase variant was determined.

### Experimental Example 8.4: Preparation of Linker

The linker for conjugating the urate oxidase variant and the albumin is not limited if it has the structure disclosed above, and a person skilled in the art can use commercial linkers purchased or appropriately prepare linkers using a known method.

When using a linker containing tranc-cyclooctene (TCO) as an IEDDA reactive group that reacts with the urate oxidase variant and using 3-arylpropiolonitriles (APN) as a thiol reactive group that reacts with albumin, it is prepared according to the following method and is then used:

1) TCO-NHS ester (for example, purchased from CONJU-PROBE) and APN-amine (for example, purchased from CONJU-PROBE) are reacted in a 1:1 molar ratio in a dimethyl sulfoxide (DMSO) solvent at room temperature.

2) The reaction product is separated and purified by column chromatography using silica gel. In this case, the separation and purification degree is set to 95% or more.

### Experimental Example 8.5: Obtainment of Urate Oxidase-Albumin Conjugate

Using the urate oxidase variant obtained in Experimental Example 8.2 and the linker obtained in Experimental Example 8.4, a urate oxidase-albumin conjugate is obtained through the method disclosed in Experimental Example 2.

### Experimental Example 8.6: In Vitro Enzymatic Activity Analysis of Urate Oxidase-Albumin Conjugate

Using the urate oxidase-albumin conjugate obtained in Experimental Example 8.5, the in vitro enzymatic activity of the urate oxidase-albumin conjugate is analyzed by the method disclosed in Experimental Example 3.

### Experimental Example 8.7: Pharmacokinetic Evaluation of Urate Oxidase-Albumin Conjugate

Using the urate oxidase-albumin conjugate obtained in Experimental Example 8.5, a pharmacokinetic evaluation experiment for the urate oxidase-albumin conjugate is performed through the method disclosed in Experimental Example 4.

### Experimental Example 8.8: Blood Uric Acid Reduction Effect of Urate Oxidase-Albumin Conjugate in Animal Model of Hyperuricemia

Using the urate oxidase-albumin conjugate obtained in Experimental Example 8.5, the blood uric acid reduction effect of the urate oxidase-albumin conjugate is analyzed by the method disclosed in Experimental Example 5.

### Experimental Example 8.9: Pharmacokinetic Evaluation (PK Profile) Using Human FcRn (+/+) TG Mice

Using the urate oxidase-albumin conjugate obtained in Experimental Example 8.5, the pharmacokinetic evaluation (PK profile) of the urate oxidase-albumin conjugate using Human FcRn (+/+) TG mice is performed by the method disclosed in Experimental Example 6.

### Experimental Example 8.10: Human-Derived PBMC-Based In Vitro Immunogenicity Assay

Using the urate oxidase-albumin conjugate obtained in Experimental Example 8.5, the pharmacokinetic evaluation (PK profile) of the urate oxidase-albumin conjugate using Human FcRn (+/+) TG mice is performed by the method disclosed in Experimental Example 6.

### Experimental Example 9: Obtainment of Arthrobacter Globiformis-Derived Urate Oxidase-Albumin Conjugate and Verification of Effect Thereof

### Experimental Example 9.1: Test Material

4-(1,2,3,4-tetrazin-3-yl)phenylalanine (frTet) was purchased from Aldlab Chemicals (Woburn, MA, USA). Trans-cylooctene (TCO)-Cy3 was purchased from AAT Bioquest (Sunnyvale, CA, USA). TCO-PEG4-maleimide (TCO-PEG4-MAL) and amine-axially substituted TCO (TCO-amine) were purchased from FutureChem (Seoul, Korea). Pentafluor-ophenyl ester (PFP)-PEG4-APN was purchased from CONJU-PROBE (San Diego, CA, USA). Disposable PD-10 desalting columns and Superdex 200 10/300 GL increase columns were purchased from Cytiva (Uppsala, Sweden). Vivaspin 6 centrifugal concentrators with molecular weight cut-off (MWCO) of 10 and 100 kDA were purchased from Sartorius (Göt-tingen, Germany). Human serum albumin (HSA) and all other chemical reagents were purchased from Sigma-Aldrich, unless otherwise noted herein.

### Experimental Example 9.2: Vector Preparation for Obtaining Urate Oxidase Variant (AgUox-frTet) Derived from Arthrobacter Globiformis

The gene encoding Arthrobacter globiformis-derived urate oxidase (AgUox) and its variants was synthesized by Macrogen (Seoul, South Korea) at the request of the inventors. In order to express wild-type AgUox (AgUox-WT), or an AgUox variant (AgUox-frTet) having a sequence into which the nonnatural amino acid “frTet” is introduced, the synthesized gene was used as a template, and amplified through a polymerase chain reaction (PCR) using the primers “pBAD-AgUox_F (5′-GCCGCCATGGTGTCTGCTGTGAAGG-3′, SEQ ID NO: 161)” and “pBAD-AgUox_R (5′-GCCGAGATCTITAATGGTGATGGTG-3′, SEQ ID NO: 162)”. The amplified gene was digested with two restriction enzymes (NcoI and BglIII), and the gene was inserted into the NcoI and BgIII sites of the pBAD vector to synthesize pBAD_AgUox. To replace the glutamic acid codon at position 196 in the AgUox-WT sequence with an amber codon (UAG), the pBAD-AgUox was used as a template, and the primers “AgUox-196Amb_F (5′-GTCGAAGTCCACCTATACGGTGTTGTAACGCCAACGG-3′, SEQ ID NO: 163)” and “AgUox-196Amb_R (5′-CCGTTGGCGTTACAACACCGTATAGGTGGACTTCGAC-3′, SEQ ID NO: 164)” was used to prepare pBAD-AgUox_196amb.

### Experimental Example 9.3: Obtainment of AgUox-frTet

To express AgUox-frTet, the method disclosed in Bioconjugate Chemistry, 2020, 2456-2464 (by Yang et. al, titled “Temporal Control of Efficient In Vivo Bioconjugation Using a Genetically Encoded Tetrazine-Mediated Inverse-Electron-Demand Diels-Alder Reaction”) was referred. Thus, AgUox-frTet was expressed in a manner described below, using C321ΔA.exp, pDule_C11, and pBAD_AgUox-196amb.

E. coli cells containing MjtRNATyr/MjTyrRS optimized for frTet were prepared. E. coli cells cultured in a Luria Broth (LB) medium containing ampicillin (100 g/mL) and tetracycline (10 μg/mL) were inoculated into a 2×YT medium under the same conditions under shaking overnight at 37° C. After 2.5 hours of shaking culture, when the medium containing the cells reached an optical density of 0.5 at 600 nm, frTet and L-(+)-arabinose were added to the medium such that the final concentrations thereof became 1 mM and 0.4% (w/v), respectively. After incubation for 5 hours, the cells were centrifuged at 5000 rpm at 4° C. for 10 minutes to obtain AgUox-frTet. The AgUox-frTet was purified through immobilized metal affinity chromatography at 4° C. according to the manufacturer's protocol (Qiagen). The purified AgUox-frTet was desalted with PBS (pH 7.4) using a PD-10 column. The expression and purification of AgUox-WT were performed in a similar manner to the expression and purification of the AgUox-frTet, except that tetracycline and frTet were not added to the culture medium during the expression step.

### Experimental Example 9.3: Analysis and Verification of Prepared AgUox-frTet

To identify the prepared AgUox-frTet and AgUox-WT, AgUox-frTet and AgUox-WT were digested with trypsin according to the manufacturer's protocol. A total of 0.4 mg/mL of Uox variants (AgUox-WT and AgUox-frTet) were digested at 37° C. overnight and then desalted using ZipTip C18. The trypsinized mixture was mixed with a 2,5-dihydroxybenzoic acid (DHB) matrix solution (30:70 (v/v) acetonitrile:DHB 20 mg/mL in 0.1% trifluoroacetic acid), followed by analysis using Microflex MALDI-TOF/MS instrument (Bruker Corporation, Billerica, MA, USA).

### Experimental Example 9.4: Site-Specific Fluorescent Dye Labeling of AgUox-WT and AgUox-frTet

Purified AgUox-WT and AgUox-frTet were reacted with TCO-Cy3 fluorescent dye in a molar ratio of 1:2 in PBS (pH 7.4) at room temperature. After 2 hours, the reaction mixture was subjected to sodium dodecyl sulfate polyacrylamide gel electrophoresis (SDS-PAGE). Fluorescence images of protein gels were obtained using a ChemiDoc XRS+ system (illumination at 302 nm, 510-610 nm filters, Bio-Rad Laboratories, Hercules, CA, USA). After fluorescence analysis, the protein gels were stained with Coomassie Brilliant Blue R-250 dye. Protein gel images were obtained using a ChemiDoc XRS+ system with white light illumination.

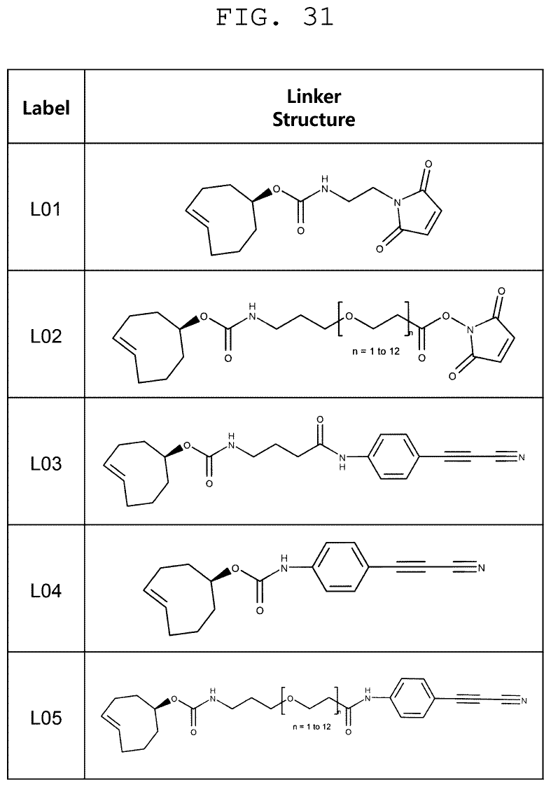

The results of analysis of AgUox-WT and AgUox-frTet prepared in Experimental Examples 9.3 to 9.4 are shown in FIGS. 32 and 33.

### Experimental Example 9.5: AgUox-HSA Conjugate Preparation (AgUox-MAL-HSA and AgUox-APN-HSA)

To perform site-specific albumination on AgUox, HSA was purified via anion exchange chromatography using a HiTrap Q HP anion exchange column. After desalting the purified HSA with PBS (pH 7.0), the HSA was reacted with TCO-MAL in a molar ratio of 1:4 in PBS (pH 7.0) at room temperature. After 2 hours, the reaction mixture was desalted with PBS (pH 7.4) using a PD-10 column, and unreacted TCO-PEG4-MAL linkers were removed to obtain a MAL-HSA conjugate. Purified Uox-frTet was reacted with MAL-HSA in a molar ratio of 1:4 in PBS (pH 7.4) at room temperature for 5 hours. After conjugation, the reaction mixture was applied to a size exclusion chromatography (SEC) using an NGC Quest 10 Plus chromatography system (Bio-Rad Laboratories Inc., Berkeley, CA, USA). The molecular weight and purity of the eluted fractions were analyzed using SDS-PAGE, and fractions corresponding to AgUox-frTet conjugated to four MAL-HSA molecules (AgUox-MAL-HSA) were selected and concentrated for further analysis.

To generate AgUox-HSA conjugates via a hetero-bifunctional cross-linker containing TCO and APN, TCO-amine was reacted with PFP-PEG4-APN. The reaction was performed in DMSO in a molar ratio of 1:1 at room temperature for 30 minutes. The purified HSA was buffer-exchanged with a 50 mM sodium borate buffer (pH 9.0). In a 50 mM sodium borate buffer (pH 9.0), purified HSA was reacted with TCO-PEG4-APN in a molar ratio of 1:4 at room temperature for 2 hours. To remove unreacted TCO-APN linker, the reaction mixture was desalted with PBS (pH 7.4) using a PD-10 column. Conjugation (AgUox-APN-HSA) and purification of AgUox-frTet conjugated to four HSA molecules via linkers containing APN were performed in a similar manner to AgUox-MAL-HSA.

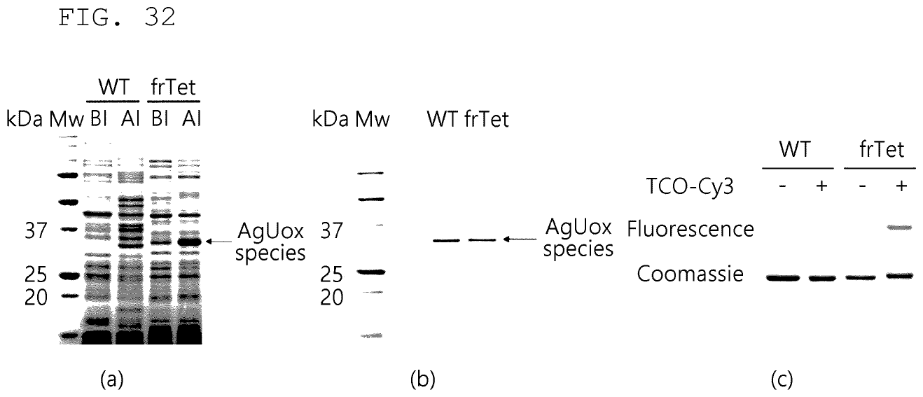

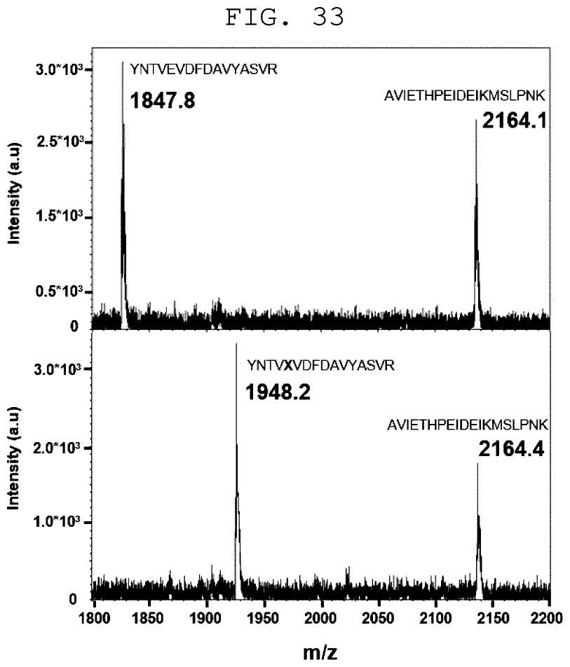

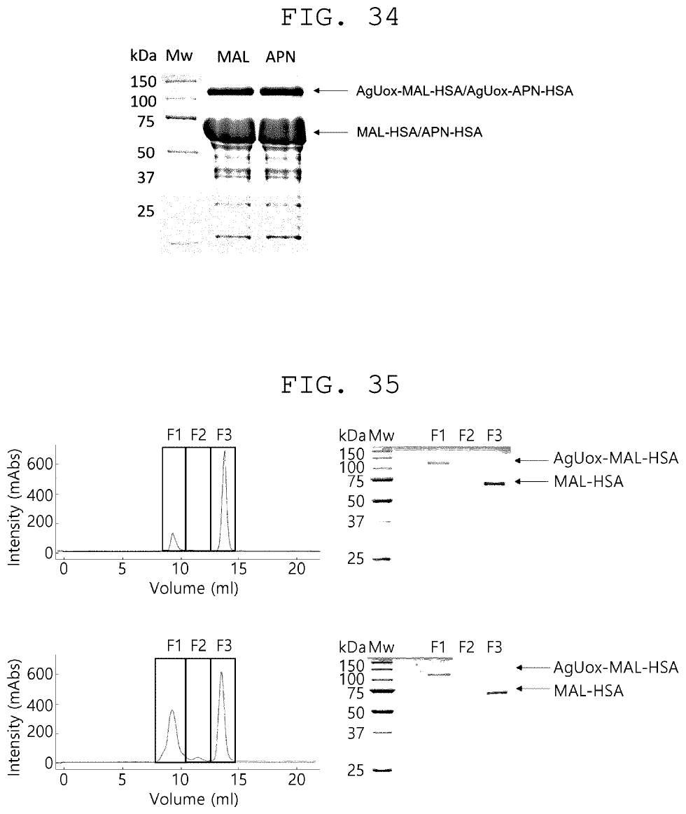

The results of preparation of the AgUox-HSA conjugate according to Experimental Example 9.5 are shown in FIGS. 34 and 35.

### Experimental Example 9.6: In Vivo Half-Life Evaluation (PK Profile) of AgUox-HSA Conjugate

Stability analysis of AgUox-HSA conjugates in mice was performed according to the guidelines of the Animal Care and Use Committee of Gwangju Institute of Science and Technology (GIST-2020-037). Each of AgUox-WT, AgUox-MAL-HSA, and AgUox-APN-HSA was injected into the tail vein of young female BALB/c mice (n=4) in an amount corresponding to 5.0 nmol of AgUox in 200 μL PBS at pH 7.4. In the case of AgUox-WT, blood samples were taken through retro-orbital bleeding after 15 minutes, 3 hours, 6 hours, and 12 hours, and in the case of AgUox-HSA conjugates, blood samples were collected in the same manner at 15 minutes, 12 hours, 24 hours, 48 hours, 72 hours, 84 hours, 96 hours, 108 hours, and 120 hours. After separating the serum from the collected blood, the serum activity of each of AgUox-WT, AgUox-MAL-HSA, and AgUox-APN-HSA was measured. The serum activity was measured by adding 100 μL of enzyme activity assay buffer containing 100 μM uric acid to 100 μL enzyme activity buffer containing 5 μL of serum, and measuring the change in absorbance at 293 nm.

The in vivo half-life evaluation results are shown in FIG. 36.

As a result of the experiment, it was confirmed that AgUox-APN-HSA and AgUox-MAL-HSA exhibited a significant increase in half-life compared to AgUox-WT which is not conjugated with albumin.

### INDUSTRIAL APPLICABILITY

The present description discloses a urate oxidase-albumin conjugate, a method of preparing the same, a urate oxidase variant included in the urate oxidase-albumin conjugate, and a method of preparing the same. The urate oxidase-albumin conjugate can be used to prevent or treat various diseases, disorders, or indications caused by uric acid.

1-24. (canceled)

25. A urate oxidase variant which is a tetramer of four urate oxidase variant subunits,
wherein each of the urate oxidase variant subunits is a peptide independently selected from SEQ ID NOs: 2 to 50, or a peptide which is 90% or more identical to the peptide selected from SEQ ID NOs:2 to 50,
wherein each of the sequences of SEQ ID NOs: 2 to 50 comprises a nonnatural amino acid X which is independently selected from the group consisting of:
4-(1,2,3,4-tetrazin-3-yl) phenylalanine (frTet); 4-(6-methyl-s-tetrazin-3-yl)phenylalanine (Tet-v2.0); 3-(4-(1,2,4-triazin-6-yl)phenyl)-2-aminopropanoic acid; 2-amino-3-(4-(2-(6-methyl-1,2,4,5-tetrazin-3-yl)ethyl)phenyl)propanoic acid; 2-amino-3-(4-(6-phenyl-1,2,4,5-tetrazin-3-yl)phenyl)propanoic acid; 3-(4-((1,2,4,5-tetrazin-3-yl)amino)phenyl)-2-aminopropanoic acid; 3-(4-(2-(1,2,4,5-tetrazin-3-yl)ethyl)phenyl)-2-aminopropanoic acid; 3-(4-((1,2,4,5-tetrazin-3-yl)thio)phenyl)-2-aminopropanoic acid; 2-amino-3-(4-((6-methyl-1,2,4,5-tetrazin-3-yl)thio)phenyl)propanoic acid; 3-(4-((1,2,4,5-tetrazin-3-yl)oxy)phenyl)-2-aminopropanoic acid; 2-amino-3-(4-((6-methyl-1,2,4,5-tetrazin-3-yl)oxy)phenyl)propanoic acid; 3-(4′-(1,2,4,5-tetrazin-3-yl)-[1,1′-biphenyl]-4-yl)-2-aminopropanoic acid; 2-amino-3-(4′-(6-methyl-1,2,4,5-tetrazin-3-yl)-[1,1′-biphenyl]-4-yl)propanoic acid; 2-amino-3-(6-(6-(pyridin-2-yl)-1,2,4,5-tetrazin-3-yl)pyridin-3-yl)propanoic acid; 3-(4-(1,2,4,5-tetrazin-3-yl)phenyl)-2-aminopropanoic acid; and 2-amino-3-(4-(6-methyl-1,2,4,5-tetrazin-3-yl)phenyl)propanoic acid.

26. The urate oxidase variant of claim 25, wherein all four urate oxidase variant subunits are identical.

27. The urate oxidase variant of claim 25, wherein each of the four urate oxidase variant subunits comprises 4-(1,2,3,4-tetrazin-3-yl) phenylalanine (frTet) as the nonnatural amino acid X.

28. The urate oxidase variant of claim 25, wherein each of the four urate oxidase variant subunits is a peptide of SEQ ID NO: 29, wherein each of the four urate oxidase variant subunits comprises 4-(1,2,3,4-tetrazin-3-yl) phenylalanine (frTet) as the nonnatural amino acid X.

29. A urate oxidase variant which is a tetramer of one urate oxidase subunit and three urate oxidase variant subunits, wherein the urate oxidase subunit is a peptide of SEQ ID NO: 1, or a peptide which is 90% or more identical to the peptide of SEQ ID NO: 1,
wherein each of the urate oxidase variant subunits is a peptide independently selected from SEQ ID NOs: 2 to 50, or a peptide which is 90% or more identical to the peptide selected from SEQ ID NOs: 2 to 50,
wherein each of the sequences of SEQ ID NOs: 2 to 50 comprises a nonnatural amino acid X which is independently selected from the group consisting of:
4-(1,2,3,4-tetrazin-3-yl) phenylalanine (frTet); 4-(6-methyl-s-tetrazin-3-yl)phenylalanine (Tet-v2.0); 3-(4-(1,2,4-triazin-6-yl)phenyl)-2-aminopropanoic acid; 2-amino-3-(4-(2-(6-methyl-1,2,4,5-tetrazin-3-yl)ethyl)phenyl)propanoic acid; 2-amino-3-(4-(6-phenyl-1,2,4,5-tetrazin-3-yl)phenyl)propanoic acid; 3-(4-((1,2,4,5-tetrazin-3-yl)amino)phenyl)-2-aminopropanoic acid; 3-(4-(2-(1,2,4,5-tetrazin-3-yl)ethyl)phenyl)-2-aminopropanoic acid; 3-(4-((1,2,4,5-tetrazin-3-yl)thio)phenyl)-2-aminopropanoic acid; 2-amino-3-(4-((6-methyl-1,2,4,5-tetrazin-3-yl)thio)phenyl)propanoic acid; 3-(4-((1,2,4,5-tetrazin-3-yl)oxy)phenyl)-2-aminopropanoic acid; 2-amino-3-(4-((6-methyl-1,2,4,5-tetrazin-3-yl)oxy)phenyl)propanoic acid; 3-(4′-(1,2,4,5-tetrazin-3-yl)-[1,1′-biphenyl]-4-yl)-2-aminopropanoic acid; 2-amino-3-(4′-(6-methyl-1,2,4,5-tetrazin-3-yl)-[1,1′-biphenyl]-4-yl)propanoic acid; 2-amino-3-(6-(6-(pyridin-2-yl)-1,2,4,5-tetrazin-3-yl)pyridin-3-yl)propanoic acid; 3-(4-(1,2,4,5-tetrazin-3-yl)phenyl)-2-aminopropanoic acid; and 2-amino-3-(4-(6-methyl-1,2,4,5-tetrazin-3-yl)phenyl)propanoic acid.

30. The urate oxidase variant of claim 29, wherein all three urate oxidase variant subunits are identical.

31. The urate oxidase variant of claim 29, wherein each of the four urate oxidase variant subunits comprises 4-(1,2,3,4-tetrazin-3-yl) phenylalanine (frTet) as the nonnatural amino acid X.

32. The urate oxidase variant of claim 29, wherein each of the three urate oxidase variant subunits is a peptide of SEQ ID NO: 29, wherein each of the three urate oxidase variant subunits comprises 4-(1,2,3,4-tetrazin-3-yl) phenylalanine (frTet) as the nonnatural amino acid X.

33. A vector encoding a peptide of SEQ ID NOs: 2 to 50, wherein an X of SEQ ID NOs: 2 to 50 is nonnatural amino acid, and the sequence corresponding to the X is encoded by a stop codon in the vector.

34. The vector of claim 33, wherein the stop codon is 5′-TAG-3′.

35. A method for manufacturing a urate oxidase variant, the method comprising:
(a) preparing a cell line comprising a vector capable of expressing an orthogonal tRNA/synthetase pair, and a urate oxidase variant expression vector;
wherein the vector capable of expressing orthogonal tRNA/synthetase is capable of expressing an exogenous suppressor tRNA and an exogenous tRNA synthetase,
wherein the exogenous suppressor tRNA is capable of recognizing a specific stop codon,
the exogenous tRNA synthetase is capable of recognizing a nonnatural amino acid containing a tetrazine functional group and/or a triazine functional group and of linking the recognized amino acid to the exogenous suppressor tRNA,
wherein the urate oxidase variant expression vector is capable of expressing a urate oxidase variant subunit represented by a sequence selected from SEQ ID NO: 2 to 50 or a sequence 90% or more identical with the sequence selected from SEQ ID NO: 2 to 50,
wherein the sequence corresponding to the nonnatural amino acid of urate oxidase variant is encoded by the specific stop codon; and
(b) culturing the cell line in a medium containing at least one kind of a nonnatural amino acid comprising a tetrazine functional group and/or a triazine functional group.

36. The method of claim 35, wherein the cell line is cultured in a medium containing a nonnatural amino acid selected from the following:
4-(1,2,3,4-tetrazin-3-yl) phenylalanine (frTet); 4-(6-methyl-s-tetrazin-3-yl)phenylalanine (Tet-v2.0); 3-(4-(1,2,4-triazin-6-yl)phenyl)-2-aminopropanoic acid; 2-amino-3-(4-(2-(6-methyl-1,2,4,5-tetrazin-3-yl)ethyl)phenyl)propanoic acid; 2-amino-3-(4-(6-phenyl-1,2,4,5-tetrazin-3-yl)phenyl)propanoic acid; 3-(4-((1,2,4,5-tetrazin-3-yl)amino)phenyl)-2-aminopropanoic acid; 3-(4-(2-(1,2,4,5-tetrazin-3-yl)ethyl)phenyl)-2-aminopropanoic acid; 3-(4-((1,2,4,5-tetrazin-3-yl)thio)phenyl)-2-aminopropanoic acid; 2-amino-3-(4-((6-methyl-1,2,4,5-tetrazin-3-yl)thio)phenyl)propanoic acid; 3-(4-((1,2,4,5-tetrazin-3-yl)oxy)phenyl)-2-aminopropanoic acid; 2-amino-3-(4-((6-methyl-1,2,4,5-tetrazin-3-yl)oxy)phenyl)propanoic acid; 3-(4′-(1,2,4,5-tetrazin-3-yl)-[1,1′-biphenyl]-4-yl)-2-aminopropanoic acid; 2-amino-3-(4′-(6-methyl-1,2,4,5-tetrazin-3-yl)-[1,1′-biphenyl]-4-yl)propanoic acid; 2-amino-3-(6-(6-(pyridin-2-yl)-1,2,4,5-tetrazin-3-yl)pyridin-3-yl)propanoic acid; 3-(4-(1,2,4,5-tetrazin-3-yl)phenyl)-2-aminopropanoic acid; and 2-amino-3-(4-(6-methyl-1,2,4,5-tetrazin-3-yl)phenyl)propanoic acid.

37. The method of claim 35, wherein the specific stop codon is 5′-TAG-3′.
